(19) United States
(12) Patent Application Publication
(10) Pub. No.: US 2024/0133884 A1
(43) Pub. Date: Apr. 25, 2024

(54) COMPOSITIONS AND METHODS FOR DETECTING TRYPANOSOMA CRUZI INFECTION

(71) Applicant: University of Georgia Research Foundation, Inc., Athens, GA (US)

(72) Inventors: Rick Tarleton, Bogart, GA (US); HuiFeng Shen, Athens, GA (US)

(21) Appl. No.: 18/357,065

(22) Filed: Jul. 21, 2023

Related U.S. Application Data
(60) Provisional application No. 63/391,316, filed on Jul. 21, 2022.

(51) Int. Cl. G01N 33/569; A61K 31/4168; A61K 31/554; A61K 45/06; A61P 33/02; C07K 14/44; C07K 17/00

(52) U.S. Cl. CPC G01N 33/56905; A61K 31/4168; A61K 31/554; A61K 45/06; A61P 33/02; C07K 14/44; C07K 17/00; G01N 2333/44

(57) ABSTRACT

Combinations of Trypanosoma cruzi polypeptides, fusion protein formed therefrom, and compositions and methods of use thereof for improved detection of antibodies against T. cruzi are disclosed. Preferred polypeptide combinations include two or more polypeptides selected from Table 1, or a variant or fragment thereof. In particularly preferred embodiments, the polypeptide combinations include the two polypeptide as paired in Table 2 or Table 3, or variants or fragments thereof. Preferably, the one, or more preferably both, of the polypeptides are antigenic to T. cruzi antibodies. The polypeptide combination can unfused or fused for form fusion proteins. Methods of using the disclosed compositions, including methods of detecting anti-T. cruzi antibodies, diagnosing T. cruzi infections, and monitoring disease status and treatment efficacy are also provided.

### CROSS-REFERENCE TO RELATED APPLICATIONS

This application claims the benefit of and priority to U.S. Provisional Application No. 63/391,316, filed Jul. 21, 2022, which is hereby incorporated herein by reference in its entirety.

### STATEMENT REGARDING FEDERALLY SPONSORED RESEARCH

This invention was made with government support under grant number R01 AI125738 awarded by the National Institutes of Health. The government has certain rights in the invention. (37 CFR 401.14 f (4)).

### REFERENCE TO THE SEQUENCE LISTING

The Sequence Listing submitted as a text file named “UGA_2021-115-02_US_ST26.xml” created on Jul. 21, 2023, and having a size of 182,485 bytes is hereby incorporated by reference pursuant to 37 C.F.R. § 1.834(c)(1).

### FIELD OF THE INVENTION

The field of the invention generally relates to compositions and methods for detecting Trypanosoma cruzi infection, and uses related thereto.

### BACKGROUND OF THE INVENTION

Chagas disease (American trypanosomiasis), a consequence of infection by the protozoan parasite Trypanosoma cruzi, is the highest impact infectious disease in Latin America and a growing threat in the United States. T. cruzi infection has its greatest human impact in areas of Latin America where housing conditions bring people, infected animals, and vector insects into close proximity (Cohen, et al. Science, 293: 694-698 (2001)). However, increasing travel and immigration has brought T. cruzi infection into the spotlight globally, even in areas where transmission has previously been absent or very low. In these latter situations, congenital and transfusion/transplantation-related transmissions are becoming recognized as significant threats (Young, et al., Transfusion 47: 540-544 (2007), Chagas disease after organ transplantation—Los Angeles, California, 2006. MMWR Morb Mortal Wkly Rep 55: 798-800 (2006), Munoz, et al., Trans R Soc Trop Med Hyg 101: 1161-1162 (2007)).

Diagnosis of T. cruzi infection is challenging for a number of reasons. See, e.g., Cooley, et al., “High Throughput Selection of Effective Serodiagnostics for Trypanosoma cruzi infection,” PLOS (2008), doi.org/10.1371/journal.pntd.0000316, which is specifically incorporated by reference herein in its entirety. The initial infection is seldom detected except in cases where infective doses are high and acute symptoms very severe, as in localized outbreaks resulting from oral transmissions (Aguilar, et al., Mem Inst Oswaldo Cruz 102: Suppl 147-56 (2007), Shikanai-Yasuda, et al., Rev Inst Med Trop Sao Paulo 33: 351-357 (1991), Benchimol, et al., Int J Cardiol 112: 132-133 (2006)). Classical signs of inflammation at proposed sites of parasite entry (e.g. “Romaña's sign”) or clinical symptoms other than fever, are infrequently reported (Nicholls, et al., Biomedica 27: Suppl 18-17 (2007)). As a result, diagnosis is very rarely sought early in the infection, when direct detection of parasites may be possible. In the vast majority of human cases, T. cruzi infection evolves undiagnosed into a well-controlled chronic infection wherein circulating parasites or their products are difficult to detect.

Unfortunately, multiple studies from geographically distinct areas and utilizing a wide range of tests and test formats have shown diagnostics to be far from dependable (Pirard, et al., Transfusion 45: 554-561 (2005), Salomone, et al., Emerg Infect Dis 9: 1558-1562 (2003), Avila, et al., J Clin Microbiol 31: 2421-2426 (1993), Castro, et al., Parasitol Res 88: 894-900 (2002), Caballero, et al., Clin Vaccine Immunol. Vol. 14, No. 8, Pages 1045-1049 (2008), Silveira-Lacerda, et al., Vox Sang 87: 204-207 (2004), Wincker, et al., Am J Trop Med Hyg 51: 771-777 (1994), Gutierrez, et al., Parasitology 129: 439-444 (2004), Marcon, et al., Diagn Microbiol Infect Dis 43: 39-43 (2002), Picka, et al., Braz J Infect Dis 11: 226-233 (2007), Zarate-Blades, et al., Diagn Microbiol Infect Dis 57: 229-232 (2007)).

For example, diagnosis can include polymerase chain reaction detection of parasite DNA. However, the extremely low parasite level in the blood of infected hosts make this approach insensitive and undependable. The primary alternative diagnostic approach for this and many infectious diseases is to measure anti-pathogen antibodies in the blood (serology). Serology is convenient because blood is generally easily accessible. Serological assays also have potential to track the efficacy of cure, as anti-T. cruzi antibody titers decline over time after curative treatment. Many of the most widely employed serological tests, including one approved by the United States Food and Drug Administration for use as a blood screening test in the U.S. (Tobler, et al., Transfusion 47: 90-96 (2007)), use crude or semi-purified parasite preparations, often derived from parasite stages present in insects but not in infected humans. Other tests have incorporated more defined parasite components, including multiple fusion proteins containing epitopes from various parasite proteins, which, individually have shown some promise as diagnostics (Silveira-Lacerda, et al., Vox Sang 87: 204-207 (2004), da Silveira, et al., Trends Parasitol 17: 286-291 (2001), Chang, et al., Transfusion 46: 1737-1744 (2006)). Unfortunately, when using conventional serologic tests composed of bulk parasites antigen (whole parasites or lysates) or single antigens (usually in an ELISA format), infection can be missed and changes in titers can take years to greater than a decade to be dependably measured.

Additionally, in the absence of a true gold standard, the sensitivity of new tests is generally determined using sera that have been shown to be unequivocally positive on multiple other serologic tests, but rarely with sera that are borderline or equivocal on one or more tests. This approach assures only that the test being evaluated is no worse, but not necessarily any more sensitive, than the existing tests. A “conclusive” diagnosis of T. cruzi infection is often reached only after multiple serological tests and in combination with epidemiological data and (occasionally) clinical symptoms. See also, U.S. Published Application Nos. 2015/0352160, 2012/0316209, 2010/0323909, 2010/0297173, 2008/0019995, 2007/0178100, and 2005/0158347, and U.S. Pat. Nos. 9,907,822, 8,329,411, 7,892,555, 7,888,135, 7,780,969, 7,309,784, 6,875,584, and 6,368,827.

Thus, a key factor preventing the broader use of current drugs and the development of safer and more effective new drugs is the absence of a means to discriminate active infection from a resolved (cured) infection.

Therefore, it is an object of the invention to provide improved materials and methods for the detection of T. cruzi infections.

It is a further object of the invention to utilize the improved materials and methods for the detection of T. cruzi infections in diagnosis of subjects, the testing of potential treatments, and other diagnostic, therapeutic, and research-based application.

### SUMMARY OF THE INVENTION

Combinations of Trypanosoma cruzi polypeptides, fusion protein formed therefrom, and compositions and methods of use thereof for improved detection of antibodies against T. cruzi are disclosed. The disclosed compositions, devices, and methods typically utilize a combination of two or more antigenic T. cruzi polypeptides. Preferred polypeptide combinations include two or more polypeptides selected from Table 1, or a variant or fragment thereof. In particularly preferred embodiments, the polypeptide combinations include the two or three polypeptides as paired in Table 2; TcBrA4_0101970, TcYC6_0077100, and TcYC6_0078140 or fragment(s) thereof e.g., a fusion of aa 150-260 for TcBrA4_0101970, TcYC6_0077100, and TcYC6_0078140 with linkers in between); or variants or fragments thereof. In some embodiments, the fusion protein is a fusion of Table 3, or variant or fragment thereof. Preferably, the one, or more preferably both or more, of the polypeptides are antigenic to T. cruzi antibodies.

Fusion proteins including a combination of two or more polypeptides are also provided. The fusion proteins are typically antigenic to one or more anti-T. cruzi antibodies, optionally, but preferably wherein the antibodies are collected from a subject. The subject can be any subject that can raise antibodies against T. cruzi, either through natural exposure to T. cruzi, immunization thereto, or a combination thereof. Preferably, the anti-T. cruzi antibody or antibodies are from a subject or subjects infected with T. cruzi. Exemplary subjects include humans, non-human primates, and dogs. In some embodiments, the antibodies are from a pool of subjects, e.g., two or more humans, non-human primates, dogs, or a combination thereof. Preferably, the fusion proteins have a broad and strong pattern of recognition across a large set of sera (i.e., specific for antibodies found in multiple individual serum samples). In some embodiments, the pool of subjects is between 2 and 100,000, or any integer range or specific integer there between. In some embodiments, the number of subjects forming the pool is at least 1,000. In some embodiments, the antibodies are from the subject's or subjects' blood, or another body fluid in which the antibodies reside.

Typically, the antibody or antibodies specifically bind to the fusion protein. In preferred embodiments, the number of antibodies, the binding affinity of the antibodies, the specificity of the antibodies, or a combination thereof for the fusion protein is higher than for: one of the polypeptides of the fusion protein as a single (i.e., individual) polypeptide in the absence of being linked to the other polypeptide; both of the polypeptides as single (i.e., individual) polypeptides in the absence of being linked to each other; and/or the additive result of both of the polypeptides as single (i.e., individual) polypeptides in the absence of being linked to each other.

In some embodiments, the fusion proteins include linkage of any two T. cruzi polypeptides selected from SEQ ID NOS:1-22 and 53-59, variants thereof with at least 70% sequence identity thereto, and fragments of the foregoing including at least 15 amino acids. In some embodiments, the fusion protein has the formula N—R₁-R₂-R₃-C, wherein “N” indicates the N-terminal end and “C” indicates the C-terminal end of the fusion protein; R₁ is a first polypeptide selected from SEQ ID NOS:1-22 or 53-59 or Table 1, variants thereof with at least 70% sequence identity thereto, and fragments of the foregoing having at least 15 amino acids; R₂ is an optional linker; and R₃ is a second polypeptide selected from SEQ ID NOS:1-22 or 53-59 or Table 1, variants thereof with at least 70% sequence identity thereto, and fragments of the foregoing having at least 15 amino acids. Typically, the two or more polypeptides of the fusion protein are different polypeptides. For example, typically the two or more polypeptides are derived from, or are, different polypeptides of SEQ ID NOS:1-22 and 53-59. The two or more polypeptides of the fusion are typically linked by a linker such as a chemical linker or a polypeptide linker. In some embodiments, the two or more polypeptides are chemically conjugated. In some embodiments, the linker is a polypeptide linker having two or more amino acids.

In preferred embodiments, the fusion proteins are composed of a combination of two or more polypeptides derived from, including, or consisting of the paired polypeptides outlined in Table 2. For example, in some embodiments, the two polypeptides are:
SEQ ID NOS:1 and 12, variants thereof with at least 70% sequence identity thereto, or fragments of the foregoing comprising at least 15 amino acids;
SEQ ID NOS:2 and 13, variants thereof with at least 70% sequence identity thereto, or fragments of the foregoing comprising at least 15 amino acids;
SEQ ID NOS:3 and 14, variants thereof with at least 70% sequence identity thereto, or fragments of the foregoing comprising at least 15 amino acids;
SEQ ID NOS:4 and 15, variants thereof with at least 70% sequence identity thereto, or fragments of the foregoing comprising at least 15 amino acids;
SEQ ID NOS:5 and 16, variants thereof with at least 70% sequence identity thereto, or fragments of the foregoing comprising at least 15 amino acids;
SEQ ID NOS:6 and 17, variants thereof with at least 70% sequence identity thereto, or fragments of the foregoing comprising at least 15 amino acids;
SEQ ID NOS:7 and 18, variants thereof with at least 70% sequence identity thereto, or fragments of the foregoing comprising at least 15 amino acids;
SEQ ID NOS:8 and 19, variants thereof with at least 70% sequence identity thereto, or fragments of the foregoing comprising at least 15 amino acids;
SEQ ID NOS:9 and 20, variants thereof with at least 70% sequence identity thereto, or fragments of the foregoing comprising at least 15 amino acids;
SEQ ID NOS:10 and 21, variants thereof with at least 70% sequence identity thereto, or fragments of the foregoing comprising at least 15 amino acids; or
SEQ ID NOS:11 and 22, variants thereof with at least 70% sequence identity thereto, or fragments of the foregoing comprising at least 15 amino acids.
SEQ ID NOS:53 and 54, variants thereof with at least 70% sequence identity thereto, or fragments of the foregoing comprising at least 15 amino acids.
SEQ ID NOS:55 and 56, variants thereof with at least 70% sequence identity thereto, or fragments of the foregoing comprising at least 15 amino acids.
SEQ ID NOS:57, 58 and 59, variants thereof with at least 70% sequence identity thereto, or fragments of the foregoing comprising at least 15 amino acids.

In particular embodiments, the fusion proteins are composed of a combination of two polypeptides including or consisting of the paired polypeptides optionally linked by a linker:
SEQ ID NOS:1 and 12;
SEQ ID NOS:2 and 13;
SEQ ID NOS:3 and 14;
SEQ ID NOS:4 and 15;
SEQ ID NOS:5 and 16;
SEQ ID NOS:6 and 17;
SEQ ID NOS:7 and 18;
SEQ ID NOS:8 and 19;
SEQ ID NOS:9 and 20;
SEQ ID NOS:10 and 21;
SEQ ID NOS:11 and 22;
SEQ ID NOS:53 and 54;
SEQ ID NOS:55 and 56; or
SEQ ID NOS:57, 58 and 59.

In some embodiments, the polypeptide derived from, or including, or consisting of the first SEQ ID NO in the foregoing combinations is N-terminal to the polypeptide derived from, or including, or consisting of the second SEQ ID NO in the foregoing combinations. In other embodiments, the polypeptide derived from, or including, or consisting of the second SEQ ID NO in the foregoing lists is N-terminal to the polypeptide derived from, or including, or consisting of the first SEQ ID NO in the foregoing combinations.

Exemplary fusion proteins include those of Table 3 and SEQ ID NOS:67-80, variants thereof with at least 70% sequence identity thereto, and fragments of the foregoing having at least 15 amino acids.

Also provided are substrates with combinations of polypeptides and/or fusion proteins immobilized thereon. Thus, in some embodiments, the substrates include a combination(s) of unfused T. cruzi polypeptides, one or more T. cruzi fusion proteins including to or more fused T. cruzi polypeptides, or a combination thereof. Substrates can be composed of any suitable material or materials including, but not limited to, glass, metal, and plastic. In some embodiments, the substrate is a slide, plate, paper, or bead. Additionally or alternatively the combinations of polypeptides and/or fusion proteins can be immobilized on a support. Supports include, for example, a carboxymethyl-cellulose, starch, collagen, modified sepharose, ion exchange resins, active charcoal, silica, clay, aluminum oxide, titanium, diatomaceous earth, hydroxyapatite, ceramic, celite, agarose, treated porous glass, or a polymer. Substrates and/or supports can include two or more different polypeptides and/or fusion proteins.

In particular embodiments, the combination of polypeptides and/or fusion protein or proteins is/are bound to a bead, or to a series or array of beads. In some embodiments, the beads are individually addressed for each different fusion protein or combination of peptides, optionally wherein the different individually addressed beads are different wells of a microtiter plate or arrayed on a microchip.

Nucleic acids including a nucleic acid sequence encoding the fusion proteins are also provided. In some embodiments, the nucleic acids include an expression control sequence operably linked to the nucleic acid sequence encoding the fusion protein. The nucleic acid can be, e.g., DNA or RNA. The nucleic acid can be single stranded or double strand, linear or circular. For example, in some embodiments, the nucleic acid is an mRNA or a DNA vector. Exemplary nucleic acids encoding SEQ ID NOS:1-22 and 53-59 are provided in Table 1 as SEQ ID NOS:23-44 and 60-66. Exemplary nucleic acids encoding SEQ ID NOS:67-80 are provided in Table 3 as SEQ ID NOS:81-108. Cells including the nucleic acids are also provided and can be, for example, cells for expression of the fusion proteins.

Methods of detection and diagnosis are also provided. For example, methods of detecting one or more anti-Trypanosoma cruzi antibodies in a sample can include contacting the sample with two or more polypeptides, e.g., selected from Table 1, or a variant or fragment thereof under conditions suitable for antibodies specific for the polypeptides to bind thereto, and detecting the bound antibodies. In some embodiments, the polypeptide combinations include the two or three polypeptides as paired in Table 2 or Table 3, or variants or fragments thereof. In some embodiments, the two, three, or more of the polypeptides are fused and thus presented in the form of a fusion protein. In some embodiments, the polypeptides are presented unfused combinations. In some embodiments, the polypeptides and/or fusion proteins are immobilized on a substrates. In some embodiments, the methods include removing unbound antibodies prior to detection. Such a removal step may be, for example, a wash step. Typically, the sample is a biological sample, preferably a biological fluid sample, such as whole blood, plasma, serum, urine, saliva, tears, or lymphatic fluid. In some embodiments, the biological sample is from a subject selected from humans, non-human primates, dogs, and combinations thereof.

In some embodiments, the method of detection is an immunoassay, such as indirect immunofluorescence assay (IFA), enzyme-linked immunosorbent assay (ELISA), radioimmunoassay (RIA), fluorescent bead technology, Western blot, etc. For example, detecting the bound antibodies can include contacting the bound antibodies with a secondary antibody that binds specifically to the anti-Trypanosoma cruzi antibodies of the sample. IN some embodiments, the secondary antibody binds to the Fc region of the anti-Trypanosoma cruzi antibodies. The secondary antibody can be conjugated to an enzyme (e.g., horseradish peroxidase (HRP) or alkaline phosphatase (AP)) or a fluorescent dye (e.g., fluorescein isothiocyanate (FITC), rhodamine derivatives, Alexa Fluor dyes, etc.).

Methods of diagnosing a subject with a Trypanosoma cruzi infection are also provided and can include, detecting anti-Trypanosoma cruzi antibodies according to the provided methods of detection, followed by diagnosing the subject as positive for a Trypanosoma cruzi infection if anti-Trypanosoma cruzi antibodies are detected. In additional or alternative methods, the subject can be diagnosed as negative for a Trypanosoma cruzi infection if anti-Trypanosoma cruzi antibodies are not detected.

Any of the methods, most particularly the methods of diagnosis, can include treating subjects positive for a Trypanosoma cruzi infection. The treatment can include administering the subject an effective amount of an antiparasitic drug. Preferred antiparasitic drugs include benznidazole and nifurtimox. In some subjects, the antiparasitic drug may cause side effects, and methods may thus further include administering the subject an effective amount of an antihistamine or corticosteroid to reduce the side effects. Some embodiments include treating the subject for one or more symptoms of Chagas disease. Exemplary symptoms include cardiac damage, gastrointestinal damage, or a combination thereof.

Methods of monitoring disease progression and/or therapeutic efficacy are also provided. For example, the therapeutic efficacy of a treatment for a T. cruzi infection and/or Chagas disease can be assessed by quantifying the level of T. cruzi antibodies in an individual's biological sample over the course of treatment. Levels of T. cruzi antibodies present in a biological sample from the individual can be determined prior to treatment and subsequently at various time intervals during treatment. Disease progression can be monitored in a similar fashion, without intervening treatment.

### BRIEF DESCRIPTION OF THE DRAWINGS

FIGS. 1A-1E show selected benzoxaboroles are active against T. cruzi in vitro and in vivo. (FIG. 1A) Structure of key compounds evaluated, including the ultimate clinical candidate, AN15368. (FIG. 1B) C57BL/6 mice received a single oral dose of AN14353 at the indicated concentration at 2 dpi with 2.5×10⁵ tdTomato-expressing T. cruzi infection in the footpads. The relative increase in the fluorescent intensity of the footpads between 2 and 4 dpi was assessed by in vivo imaging (untreated: n=8; AN15368: n=10 individual feet). Asterisks represent the statistical significance of the difference between each treated group and the untreated control. (FIG. 1C) C57BL/6 mice infected in the footpads with 2.5×10⁵ Luciferase-expressing parasites received a single oral dose of AN14353 (25 mg/kg) or no treatment at 3 dpi and the bioluminescence signal of the feet was measured over 72 hrs (untreated: n=2; AN15368: n=6 individual feet). (FIG. 1D) Daily oral treatment with AN14353 at 10 or 25 mg/kg was administered for 40 consecutive days to C57BL/6 mice starting at 15 days post-intraperitoneal infection with 10⁴ T. cruzi trypomastigotes. Parasite load in skeletal muscle was determined by qPCR following cyclophosphamide-induced immunosuppression (untreated and treated groups: n=4 mice; naïve n=1). (FIG. 1E) IFN-gamma deficient mice intraperitoneally infected with T. cruzi received a daily oral treatment with AN14353 (25 mg/kg) for 40 days (AN15368: n=4; untreated: n=9 mice; naïve n=3). Parasite load in skeletal muscle after immunosuppression with cyclophosphamide was assessed by qPCR. Untreated animals in (FIG. 1E) were terminated and tissue samples collected before the end of the experiment due to the high susceptibility to T. cruzi infection and the lethal pathology developed by IFN-gamma deficient animals. Red filled symbols denote animals in which parasites were detected by fresh blood smears or hemoculture. Data are presented as mean values+/−SEM. Dotted lines represent the cutoff in the qPCR assays. For FIGS. 1B, 1D and 1E: Mann Whitney test, two tailed; for c: Unpaired t test, 95% confidence interval. *=p<0.05; **=p<0.01; ***=p<0.001.

FIGS. 2A-2D show T. cruzi serine carboxypeptidases activate lead benzoxaborole compounds. (FIG. 2A) IC50 of parent compound AN14353 and predicted peptidase cleavage product AN14667 on intracellular and extracellular amastigotes. Three replicates were performed at each concentration: assays were repeated >3 times. (FIG. 2B) Generation of serine carboxypeptidase (CBP) KO lines in epimastigotes. WT and KO alleles was amplified using gene-specific primers respectively (left panel; see Methods). Clones with only KO but not WT allele are considered to be KO lines. Western blot validated the absence of TcCBP protein in the KO line using a TcCBP-specific antibody and tubulin detection as a control (right panel; TcCBP protein and tubulin were detected in different gels). (FIG. 2C) Impact of CBP disruption on activity of AN14353 on extracellular amastigotes (FIG. 2D) Uptake and conversion of AN15368 to CBP cleaved product AN14667 in WT and CBP-disrupted epimastigotes (top; treated with 10 uM AN15368) and amastigotes (bottom; treated with 50 nM AN15368) of T. cruzi. Data are presented as mean values+/−SEM; FIG. 2B and FIG. 2C: n=3 biological replicates; FIG. 2D: data are presented as individual values of 3 technical replicates.

FIGS. 3A-3E show AN15368 cures T. cruzi infection in mice. (FIG. 3A) Fluorescence intensity of C57BL/6 mice infected in the footpads with 2.5×10⁵ tdTomato-expressing T. cruzi was measured by in vivo imaging directly before (2 dpi) and 2 days after (4 dpi) a single oral dose of the compounds (50 mg/kg). The parasite proliferation index after treatment was calculated as described in the Materials & Methods section. (n=10 individual feet). (FIG. 3B) A daily oral treatment of the indicated compounds (10 mg/kg) was administered for 40 days to C57BL/6 mice acutely infected with T. cruzi beginning at 17 dpi. Mice were immunosuppressed at the end of the treatment and samples were collected for parasitemia, hemoculture and qPCR of skeletal muscle. Dotted line represents the background level from naïve mice (untreated, AN14817 and AN15129: n=8; AN15368 and AN14353: n=7; naïve: n=3 mice). (FIG. 3C) Acutely infected mice (20 dpi) were treated orally with 10 mg/kg of compounds for 20 days. Parasite load in skeletal muscle was quantified by qPCR following immunosuppression at the end of the treatment. Dotted line represents the cutoff for the qPCR assay (untreated, AN16109, AN15368 and AN14353: n=6; AN14817: n=5; naive: n=3 mice). (FIG. 3D) On days 13-33 post infection, oral doses of AN15368 were administered at the indicated concentrations to hairless mice infected i.p. with 5×10⁴ Luciferase-expressing T. cruzi. Bioluminiscence signals from whole mouse ventral images were acquired and quantified throughout the treatment by in vivo imaging (untreated=7 mice; treated=5 mice). (FIG. 3E) C57BL/6 mice infected with 10⁴ T. cruzi i.p. were daily treated for 40 days, starting at 15 dpi with AN16109 or AN15368 (both at 2.5 mg/kg). Parasite load was determined by qPCR of skeletal muscle samples after immunosuppression and blood samples were analyzed by light microscopy and hemoculture (AN16109: n=8; AN15368: n=7; naive: n=3; untreated: n=2 mice). Red symbols denote animals in which parasites were detected by microscopy and/or hemoculture. Data are presented as mean values+/−SEM. Asterisks represent the statistical significance of the difference between each treated group and the untreated control. For FIGS. 3A, 3B and 3C: Mann Whitney test, two tailed; for e: Unpaired t test, 95% confidence interval. *=p<0.05; **=p<0.01; ***=p<0.001.

FIGS. 4A-4K show AN15368 uniformly cures chronically infected rhesus macaques. (FIG. 4A) Structure of drug trial with indicated treatment period and sampling dates. (FIG. 4A) Summary of post-treatment detection of infection using PCR of parasite DNA in blood or tissue (either from individual tissue samples or pools of 5 individual samples/determination—see M&M for details) and hemoculture in drug-treated (T1-T19) and untreated controls (C1-3) macaques. Samples were taken at the 7 time points indicated in (FIG. 4A) and yearly thereafter for animals T1-T19, that were not euthanized and thus not tissue sampled for PCR. Fractions in table (e.g. “0/7”) indicate number of positive results per total number of samples analyzed. (FIG. 4C) Representative pre- and -post treatment IgG responses to recombinant T. cruzi proteins in control animal C3 and treated animal T19 and (FIGS. 4D-4K) for remaining animals (see Methods for identification). ND=not done.

FIG. 5A-5F shows AN15368 is activated by a T. cruzi serine carboxypeptidase and targets CPSF3. (FIG. 5A) Overexpression of CPSF3 increases resistance to AN15368(CPSF3-OE: n=3 biological replicates; WT: n=2 biological replicates), (FIG. 5B) as well as to related benzoxaboroles. All assays were carried out in intracellular amastigotes. (FIG. 5C) Suppression of parasite mRNA relative to host cell (African Green Monkey (Vero)) mRNA by exposure to AN14353 as indicated by the decline in the fraction of sequence reads mappable to parasite genome relative to host cell genome. (FIG. 5D) Engineered Asn to His mutation at amino acid position 232 of CPSF3 conveys resistance to AN15368 by intracellular amastigotes (n=3 biological replicates) (FIG. 5E) Disruption of CBPs leads to a 1000× increased resistance to AN15368 in intracellular amastigotes(TcCBP KO: n=3 biological replicates; WT: n=2 biological replicates). (FIG. 5F) Some AN15368 analogues lacking activity to intracellular T. cruzi (intra-) are effective killers of the T. congolense (extracellular) and of T. cruzi extracellular amastigotes (extra-). Data are presented as mean values+/−SEM.

FIG. 6A is a bar graph showing fold change of CPSF3-1 and CPSF3-2 when CPSF3 is overexpressed. FIGS. 6B-6D are curves showing impact of CPSF3 and TcCBP on benzoxaborole analogues.

FIG. 7 is a bar graph showing the detection of antibody levels via Luminex assay to previously utilized recombinant T. cruzi proteins (left set), positive and negative control proteins (a T. cruzi lysate and green fluorescent protein (GFP), respectively) and the fusion proteins shown in Table 2 in 17 seropositive subjects. The mean of all 17 and the maximum response among those is shown.

FIG. 8A is a schematic representation of prophylactic treatment, infection, and monitoring of mice. WT and IFNγ deficient mice were treated biweekly with 100 mg/kg of BNZ over 24 weeks. After the second week of treatment, mice were infected (5-6 animals/group) intraperitoneally with 10³ trypomastigotes of the Luciferase-expressing Colombiana strain of T. cruzi. Control groups of mice were infected and left untreated. FIG. 8B is a series of representative images showing bioluminescent signal in mice (ventral view) at 14 days post-infection exposure. FIG. 8C is a blot showing parasite bioluminescence quantification following D-luciferin injection was measured at the indicated times in the study period. Each data point represents the mean of bioluminescence from 3-6 mice expressed on a logarithmic scale as mean photons/second ±/− standard error of the mean. Grayed box marks the prophylaxis period. * p<0.03 relative to both WT groups; ** p<0.008 relative to both BNZ-treated groups; *** p<0.005 relative to BNZ-treated IFNγ KO group; n.s. bracketed data points are not statistically different. FIG. 8D is a series of plots showing the percentage of CD8+ blood lymphocytes specific for the T. cruzi TSKb20 epitope (top) and expressing the T cell central memory marker CD127 (bottom) during the study course FIG. 8E is a series of plots showing T. cruzi DNA determined by quantitative real time polymerase chain reaction in the skeletal muscle, heart, and intestine of untreated and BNZ-treated mice at week 33 of the study (˜30 weeks post-infection).

FIGS. 9A-9C show Initial screening results with multiplex serology and PCR to identify T. cruzi-negative dogs to enroll in the prophylaxis study. Heatmaps of antibody levels (mean fluorescence intensity; MFI) and qPCR measurement of T. cruzi DNA in blood were used to classify dogs as uninfected (FIG. 9A), T. cruzi-infected (FIG. 9B), or seronegative but with a blood PCR result that did not reach the cut-off for being considered positive (FIG. 9C). Recombinant T. cruzi proteins are defined in the Materials and Methods; lysate=a sonicate of T. cruzi trypomastigotes and amastigotes; GFP=recombinant green fluorescent protein (negative antigen control); Parvo=Parvovirus vaccine antigen (positive serological control).

FIG. 10 shows multiplex serological and blood PCR changes over the transmission season in dogs receiving or not BNZ prophylaxis. Serological and PCR negative dogs (see FIGS. 9A-9C) were randomly assigned to the untreated or BNZ treatment groups and then rescreened at ˜12 and −24 weeks after the beginning of prophylaxis. One dog in each group (gray fill) was moved to BNZ treatment protocol after the 12 week sampling point due to high serum cardiac troponin I (cTnI) levels. One dog in each group was lost to follow-up before the 24 week sampling time due to death or sold by owner. Antigens used in the multiplex assay are as described in FIGS. 9A-9C and the Materials and Methods. Blood PCR was considered positive if below the cut-off Ct value of 35.

FIG. 11 shows multiplex serological and blood PCR changes over the transmission season in dogs that were seronegative but PCR positive in the pre-transmission season screening (see FIGS. 9A-9C). Antigens used in the multiplex assay are as described in FIGS. 9A-9C and the Materials and Methods. * moved to a high dose BNZ treatment protocol after 12 week sample point.

### DETAILED DESCRIPTION OF THE INVENTION

### I. Definitions

As used herein, the term “polypeptides” includes proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “fusion protein” refers to a polypeptide formed by the joining of two or more polypeptides through a peptide bond formed between the amino terminus of one polypeptide and the carboxyl terminus of another polypeptide or through linking of one polypeptide to another through reactions between amino acid side chains (for example disulfide bonds between cysteine residues on each polypeptide). The fusion protein can be formed by the chemical coupling of the constituent polypeptides or it can be expressed as a single polypeptide from a nucleic acid sequence encoding the single contiguous fusion protein. Fusion proteins can be prepared using conventional techniques in molecular biology to join the two genes in frame into a single nucleic acid sequence, and then expressing the nucleic acid in an appropriate host cell under conditions in which the fusion protein is produced.

As used herein, the term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides disclosed and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamnine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

As used herein, “identity,” as known in the art, is a relationship between two or more polypeptide or polynucleotide sequences, as determined by comparing the sequences. In the art, “identity” also means the degree of sequence relatedness between polypeptide or polynucleotide as determined by the match between strings of such sequences. “Identity” can also mean the degree of sequence relatedness of a polypeptide polynucleotide compared to the full-length of a reference polypeptide. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide or polynucleotide sequence may be identical to the reference sequence, that is be 100% identical, or it may include up to a certain integer number of amino acid alterations or nucleotides as compared to the reference sequence such that the % identity is less than 100%. Such alterations are selected from: at least one amino acid or nucleic acid deletion, substitution, or addition or insertion wherein said alterations may occur at the amino- or carboxy-terminal (or 5′ or 3′) positions of the reference polypeptide (or polynucleotide) sequence or anywhere between those terminal positions, interspersed either individually among the amino acids (or nucleotides) in the reference sequence or in one or more contiguous groups within the reference sequence. Substitutions include conservative and non-conservative substitutions. The number of amino acid or nucleotide alterations for a given % identity is determined by multiplying the total number of amino acids or nucleotides in the reference polypeptide by the numerical percent of the respective percent identity (divided by 100) and then subtracting that product from said total number of amino acids or nucleotides in the reference polypeptide.

As used herein, an “amino acid sequence alteration” can be, for example, a substitution, a deletion, or an insertion of one or more amino acids.

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences.

As used herein, an “expression control sequence” is a DNA sequence that controls and regulates the transcription and/or translation of another DNA sequence.

As used herein, “operably linked” means incorporated into a genetic construct so that expression control sequences effectively control expression of a coding sequence of interest.

As used herein, a “fragment” of a polypeptide refers to any subset of the polypeptide that is a shorter polypeptide of the full-length protein. Generally, fragments will be five or more amino acids in length.

As used herein, “valency” refers to the number of binding sites available per molecule.

As used herein, “conservative” amino acid substitutions are substitutions wherein the substituted amino acid has similar structural or chemical properties.

As used herein, “non-conservative” amino acid substitutions are those in which the charge, hydrophobicity, or bulk of the substituted amino acid is significantly altered.

As used herein, the term “host cell” refers to prokaryotic and eukaryotic cells into which a recombinant expression vector can be introduced.

As used herein, “transformed” and “transfected” encompass the introduction of a nucleic acid (e.g., a vector) into a cell by a number of techniques known in the art.

As used herein, the term “antibody” refers to natural or synthetic antibodies that bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that bind the target antigen. These include Fab and F(ab′)₂ fragments which lack the Fc fragment of an intact antibody.

The terms “antigen” and “antigenic” as used herein is defined as a molecule capable of being recognized or bound by an antibody, B-cell receptor or T-cell receptor. An “immunogen” and “immunogenic” is an antigen that is additionally capable of provoking an immune response against itself (e.g., upon administration to a mammal, optionally in conjunction with an adjuvant). This immune response can involve either antibody production, or the activation of specific immunologically-competent cells, or both. Any macromolecule, including virtually all proteins or peptides as well as lipids and oligo- and polysaccharides, can serve as an antigen or immunogen. Furthermore, antigens/immunogens can be derived from recombinant or genomic DNA. Any DNA that includes a nucleotide sequences or a partial nucleotide sequence encoding a protein or peptide that elicits an immune response therefore encodes an “immunogen” as that term is used herein. An antigen/immunogen need not be encoded solely by a full-length nucleotide sequence of a gene. An antigen/immunogen need not be encoded by a “gene” at all. An antigen/immunogen can be generated, synthesized, or can be derived from a biological sample. Such a biological sample can include, but is not limited to a tissue sample, a tumor sample, a cell or a biological fluid.

As used herein, the terms “individual”, “host”, “subject”, and “patient” are used interchangeably herein, and refer to a mammal, including, but not limited to, humans, rodents, such as mice and rats, and other laboratory animals.

As used herein the term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered. As used herein the term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered.

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “reduce”, “inhibit”, “alleviate” or “decrease” are used relative to a control. One of skill in the art would readily identify the appropriate control to use for each experiment. For example, a decreased response in a subject or cell treated with a compound can be compared to a response in subject or cell that is not treated with the compound.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

### II. Compositions

A. Fusion Proteins

Fusion proteins including two or more T. cruzi polypeptides, or variants or fragments thereof are provided. Typically, the fusion protein includes a first T. cruzi polypeptide, or variant or fragment thereof linked to a second T. cruzi polypeptide, or variant or fragment thereof. Additional T. cruzi polypeptides, or variants or fragments thereof can also be included. This the fusion proteins can include not only two, but also three, four, five, or more T. cruzi polypeptides, or variants or fragments thereof. The fusion proteins also optionally contain a peptide or polypeptide linker domain that separates the first polypeptide domain from the antigen-binding domain.

Preferably, the fusion proteins can be bound by one or more anti-T. cruzi antibodies, and are thus antigenic to anti-T. cruzi antibodies. As used herein, “anti-T. cruzi antibodies” refers to antibodies that recognize and preferably specifically bind to, a molecule made by T. cruzi. For example, in preferred embodiments, anti-T. cruzi antibodies bind specifically to a protein encoded by the T. cruzi genome.

The disclosed fusions proteins are thus referred to herein as antigenic fusion proteins or fusion protein antigens with respect to anti-T. cruzi antibodies. The fusion proteins are typically composed of two or more T. cruzi polypeptides (or variants or fragments thereof), and thus can be multivalent constructs that include epitopes formed from two or more different antigenic polypeptides of T. cruzi.

A preferred antigenic fusion protein is one that detectably binds antibodies in a bodily fluid of a subject who is known to be infected or to have been infected by T. cruzi. In some embodiments, the subject is one whose bodily fluid is seronegative when assayed by conventional means. A bodily fluid that is seronegative when assayed by conventional means is one that, for example, does not show a positive reaction (antibody binding) when exposed to antigens from either whole or semi-purified parasite lysates, for example those from epimastigotes, in conventional diagnostic tests. A subject who shows evidence of T. cruzi infection using, for example, a T cell assay, polymerase chain reaction (PCR), hemoculture, or xenodiagonstic techniques, is considered to be known to be infected or to have been infected by T. cruzi, even if the subject shows a negative response to a conventional serodiagnostic test.

Another preferred antigenic fusion protein is one that detectably binds antibodies in a bodily fluid of a subject who is seropositive when assayed by conventional means, regardless of whether the polypeptide also exhibits detectable binding to antibodies in a bodily fluid of a subject who is known to be infected or to have been infected by T. cruzi, but whose bodily fluid is seronegative when assayed by conventional means.

The antigenic fusion proteins typically bind antibodies in a bodily fluid of a subject, such as blood, plasma or sera, thereby providing evidence of exposure to T. cruzi.

In some embodiments, one or more of the fusion proteins form part or all of a panel that reacts to antibodies in the sera of individuals infected with T. cruzi.

For example, in some embodiments, fusion proteins are of formula I:

N—R₁-R₂-R₃-C

wherein “N” represents the N-terminus of the fusion protein, “C” represents the C-terminus of the fusion protein, “R₁” is a first T. cruzi protein, or variant or fragment thereof, “R₂” is an optional linker domain, for example a peptide/polypeptide linker domain, and “R₃” is a second T. cruzi protein, or variant or fragment thereof. In other embodiments, R₁” is the second T. cruzi protein, or variant or fragment thereof, “R₂” is an optional linker domain, for example a peptide/polypeptide linker domain, and “R₃” is the first T. cruzi protein, or variant or fragment thereof.

Optionally, the fusion proteins additionally contain a domain that functions to dimerize or multimerize two or more fusion proteins. The domain that functions to dimerize or multimerize the fusion proteins can either be a separate domain, or alternatively can be contained within one of one of the other domains of the fusion protein. Thus, in some embodiments, the fusion proteins can be dimerized or multimerized. Dimerization or multimerization can occur between or among two or more fusion proteins through dimerization or multimerization domains. Alternatively, dimerization or multimerization of fusion proteins can occur by chemical crosslinking. The dimers or multimers that are formed can be homodimeric/homomultimeric or heterodimeric/heteromultimeric.

The modular nature of the fusion proteins and their ability to dimerize or multimerize in different combinations provides a wealth of options for presenting binding molecules to anti-T. cruzi antibodies.

1. Preferred T. cruzi Polypeptides

Examples of preferred T. cruzi polypeptides suitable for inclusion in the disclosed fusion proteins or as unfused combinations in the substrates, supports, and methods provided herein are listed in Tables 1 and 2, in the Examples, below. The “Gene ID Numbers” represent gene numbers assigned by annotators of the T. cruzi genome and can accessed via the TriTrypDB database on the worldwide web at “TriTrypDB.org”. Each of the Gene ID Numbers and the sequences provided therein are specifically incorporated by reference herein in their entireties.

In some embodiment, the antigenic fusion protein or combination includes at least two, and optionally three, four, five, or more polypeptides of Table 1 (e.g., any two or more of SEQ ID NOS:1-22 and 53-59), or fragments or variants thereof.

For example, variants may include one or more substitution(s), addition(s), or deletion(s) relative to its naturally occurring T. cruzi polypeptide counterpart. In some embodiments, the variant comprises at least 50%, 60%, 70%, 75%, 80%, 85%, 90%, 95%, 96%, 97%, 98%, or 99% sequence identity to any one of SEQ ID NOS:1-22 and 53-59.

Fragments of naturally occurring T. cruzi polypeptides or variants thereof can be truncated at either or both of the N-terminus or C-terminus. Fragments of an antigenic T. cruzi protein contain at least about eight amino acids, preferably at least about 12 amino acids, more preferably at least about 20 amino acids. In some embodiments, the fragment includes between 8 and 200 amino acids, or any integer subrange, or specific integer number therebetween (inclusive). Preferably, the variants and fragments have the ability to detect serum antibodies against T. cruzi. Preferably, the fusion protein contains an epitope recognized by a host B cell or T cell.

TABLE 1
Sequences

| | | Amino Acid (SEQ ID NOS: 1-22, 53-59) and |
|---|---|---|
| Gene ID | Gene name | Predicted DNA Sequence (SEQ ID NOS: 22-44, 60-66) |
| TcBrA4_0116860 | 60S acidic ribosomal | MSSKQQLACTYAALILADSGKTDMDSLLKVTKAAGVDVSKGMASAFASILKNVDINDVLSKVSFGGVAPAAGGATAAPAA |
| | protein putative | AAAAAAPAAAAAKKEEEEEDDDMGFGLFD (SEQ ID NO: 1) |
| | | ATGTCCTCCAAACAGCAGCTTGCCTGCACCTACGCCGCCCTGATTCTTGCCGATAGCGGCAAGACGGATATGGACAGCCT |
| | | GTTGAAAGTGACAAAGGCCGCCGGTGTTGACGTCAGCAAAGGGATGGCCTCGGCGTTTGCCAGCATCCTCAAGAACGTTG |
| | | ACATCAACGACGTGCTCTCCAAAGTGAGCTTTGGTGGTGTTGCTCCTGCTGCCGGTGGTGCCACCGCTGCTCCTGCTGCT |
| | | GCTGCTGCTGCCGCCGCCCCTGCCGCCGCCGCCGCAAAGAAGGAAGAGGAAGAGGAAGACGACGATATGGGCTTTGGTCT |
| | | GTTTGACTAG (SEQ ID NO: 23) |
| TcBrA4_0088420 | 60S ribosomal | MVSLKLQARLAADILRCGRHRVWLDPNEASEISNANSRKSVRKLIKDGLIIRKPVKVHSRSRWRHMKEAKSMGRHEGAGR |
| | protein L19 putative | REGTREARMPSKELWMRRLRILRRLLRKYREEKKIDRHIYRELYVKAKGNVFRNKRNLMEHIHKVKNEKKKERQLAEQLA |
| | | AKRLKDEQHRHKARKQELRKREKDRERARREDAAAAAAAKQKAAAKKAAAPSGKKSAKASAPAKAATAPAKATAAPAKAA |
| | | AAPAKATAAPAKATAAPAKAAAAPAKATAAPAKAAAAPAKAAAAPAKAAAAPAKAAAAPAKATAAPAKAAAAPAKVAAAP |
| | | AKAAAAPVGKKAGGKK (SEQ ID NO: 2) |
| | | ATGGTGTCGCTGAAGCTGCAGGCTCGTTTGGCGGCGGACATTCTCCGCTGCGGTCGCCACCGTGTGTGGCTGGACCCTAA |
| | | TGAGGCCTCTGAGATTTCCAATGCAAACTCGCGCAAGAGCGTGCGCAAGTTGATCAAGGATGGTCTGATTATTCGCAAGC |
| | | CTGTCAAGGTGCACTCGCGCTCCCGCTGGCGCCACATGAAGGAGGCGAAGAGCATGGGCCGCCACGAGGGCGCTGGGCGC |
| | | CGCGAGGGTACCCGCGAAGCCCGCATGCCGAGCAAGGAGCTGTGGATGCGCCGTCTGCGCATTCTCCGCCGCCTGCTGCG |
| | | CAAGTACCGCGAGGAGAAGAAGATTGACCGCCACATTTACCGCGAGCTGTACGTGAAGGCGAAGGGGAACGTGTTTCGCA |
| | | ACAAGCGTAACCTCATGGAGCACATCCACAAGGTGAAGAACGAGAAGAAGAAGGAAAGGCAGCTGGCTGAGCAGCTCGCG |
| | | GCGAAGCGCCTGAAGGATGAGCAGCACCGTCACAAGGCCCGCAAGCAGGAGCTGCGTAAGCGCGAGAAGGACCGCGAGCG |
| | | TGCGCGTCGCGAAGATGCTGCTGCTGCCGCCGCCGCGAAGCAGAAGGCAGCTGCGAAGAAGGCCGCTGCTCCCTCTGGCA |
| | | AGAAGTCCGCGAAGGCTTCTGCACCTGCCAAGGCCGCTACTGCACCCGCGAAGGCCACTGCCGCACCCGCGAAGGCTGCT |
| | | GCTGCACCCGCGAAGGCCACTGCTGCACCCGCGAAGGCCACTGCTGCACCCGCGAAGGCTGCTGCTGCACCCGCGAAGGC |
| | | CACTGCTGCACCCGCGAAGGCCGCTGCTGCACCCGCGAAGGCTGCTGCTGCACCCGCGAAGGCCGCTGCTGCACCCGCGA |
| | | AGGCTGCTGCTGCACCCGCGAAGGCCACTGCTGCACCCGCGAAGGCCGCTGCTGCACCTGCGAAGGTCGCTGCTGCACCC |
| | | GCGAAGGCTGCTGCCGCTCCCGTTGGAAAGAAGGCTGGTGGCAAGAAGTGA (SEQ ID NO: 24) |
| TcBrA4_0104680 | RNA-binding | MPAKSANKPASKPAAKPAAKPAAKAPAPKAAAPAPKAAAAAPKPAVRDAKQRSDAANHNGLYVKNWGQGSVDDARALFGT |
| | protein putative | AGKVVGVRVRRRRYAIIFFENAAAVKKAIDLFNGKEFMGNVLSVVPAKTTPKPDPHANSSVVFVSPIFRASTTKKQILEL |
| | | FSGMKVLRLRTYRNNYAYVYLDTPAAAQRAVKEKNGAEFRGKQLRVALSTRSLAKDRARAERARLLIAAQKFNKRKNHTK |
| | | (SEQ ID NO: 3) |
| | | ATGCCCGCCAAGTCTGCCAACAAGCCTGCATCCAAGCCTGCCGCCAAGCCCGCTGCGAAGCCTGCCGCCAAGGCTCCCGC |
| | | ACCCAAAGCTGCTGCCCCTGCTCCCAAGGCTGCTGCGGCTGCGCCCAAGCCAGCTGTGAGGGACGCAAAGCAGCGCTCTG |
| | | ATGCCGCCAATCACAACGGCTTGTACGTGAAGAACTGGGGCCAGGGTTCTGTGGACGACGCCAGGGCGCTTTTTGGCACT |
| | | GCTGGGAAGGTTGTGGGTGTGAGAGTGCGTCGTCGCCGTTACGCCATTATCTTCTTTGAGAACGCAGCGGCTGTGAAGAA |
| | | GGCCATTGATCTTTTCAACGGGAAAGAATTTATGGGCAATGTTTTGTCCGTTGTTCCCGCCAAGACGACTCCGAAGCCGG |
| | | ATCCGCATGCGAACTCCTCTGTTGTGTTTGTTTCCCCGATATTCCGCGCGTCGACTACAAAGAAGCAGATTCTTGAGCTT |
| | | TTTTCAGGCATGAAGGTACTGCGCCTGCGCACGTACCGCAACAACTACGCATACGTCTATCTGGACACCCCAGCTGCCGC |
| | | GCAAAGGGCTGTGAAGGAGAAGAACGGTGCAGAGTTCCGTGGCAAGCAACTCAGAGTTGCCCTCTCGACTCGTTCTCTTG |
| | | CGAAGGACAGGGCTCGTGCGGAGCGTGCAAGACTTCTTATAGCCGCCCAAAAGTTCAACAAGAGAAAGAACCACACGAAG |
| | | TAA (SEQ ID NO: 25) |
| TcBrA4_0028480 | reticulon domain | MAFCIISESRGMSLWDMLAWHRPKVTGVLLGTVLSVLTFFCLMKYTMVTFLCRILQLVLLAGVLLGFTNRWHLTSDDIHE |
| | protein putative | AVNRLVDCATPRLVTALESMHQLVTWRDYRRSGLVTLVSFVVALLGNLVSDAAFLTFFLLLAFTVPAVYEKKKDLIDKWI |
| | | SAATAQVEKYMGKIKTKVEEATKKKE (SEQ ID NO: 4) |
| | | ATGGCGTTTTGTATCATTTCTGAGAGCAGGGGCATGTCTCTGTGGGATATGCTAGCGTGGCACCGCCCAAAAGTTACGGG |
| | | TGTACTTCTTGGAACCGTACTTTCCGTCCTGACGTTTTTTTGCCTTATGAAATACACAATGGTGACGTTCCTCTGCCGCA |
| | | TCCTGCAGTTGGTCCTATTGGCCGGCGTTCTGTTGGGCTTCACGAATCGATGGCACCTCACCTCCGACGACATCCACGAG |
| | | GCCGTCAACCGCCTTGTGGACTGCGCCACGCCCCGGCTGGTGACGGCCCTTGAGTCCATGCACCAACTCGTGACGTGGCG |
| | | TGACTACCGCCGCTCCGGGCTCGTCACGCTGGTGAGCTTCGTGGTTGCTCTTCTCGGCAACCTCGTCTCCGACGCCGCCT |
| | | TTCTCACGTTTTTTCTTTTGTTGGCCTTCACCGTTCCTGCGGTGTACGAGAAGAAGAAGGATTTGATCGACAAGTGGATC |
| | | AGCGCTGCCACGGCTCAGGTGGAGAAGTACATGGGGAAGATCAAAACAAAGGTGGAAGAGGCGACCAAGAAGAAAGAGTA |
| | | A (SEQ ID NO: 26) |
| TcYC6_0100010 | 60S ribosomal | MPGKEVKKAAKPAAKTAAKPAAKSAAKPAAKPAAKPAAKTAAKPAAKTAAKPAKKPAVKPTVKPAAKAAAPYKKPAAISP |
| | protein L7a putative | FVARPKNFGIGHDVPYARDLSRFMRWPTFVTMQRKKRVLQRRLKVPPALHQFTKVLDRSSRNELLKLVKKYPSETRRARR |
| | | QRLFDVATEKKKNPEAASKKAPLSVVTGLQEVTRTIEKKTARLVMIANNVDPIELVLWMPTLCRANKVPYAIVKDKARLG |
| | | DAVGRKTATCVAITDVNAEDEAALKNLIRSVNARFLARSDVIRRQWGGLQLSLRSRAELRKKRARNAGKDAAAVM (SEQ |
| | | ID NO: 5) |
| | | ATGCCCGGCAAGGAAGTGAAAAAGGCCGCCAAGCCCGCTGCCAAGACTGCTGCAAAGCCTGCTGCCAAGTCTGCTGCCAA |
| | | GCCAGCTGCCAAGCCAGCTGCCAAGCCAGCCGCGAAGACCGCTGCGAAGCCGGCCGCGAAGACTGCTGCCAAGCCCGCTA |
| | | AGAAGCCCGCTGTGAAGCCCACTGTCAAGCCTGCTGCCAAGGCAGCCGCGCCCTACAAGAAGCCTGCGGCCATCTCACCT |
| | | TTTGTGGCGCGGCCGAAAAACTTTGGTATTGGCCACGATGTTCCGTACGCCCGTGATCTTTCTCGCTTTATGCGGTGGCC |
| | | CACGTTTGTGACGATGCAGCGGAAGAAGCGTGTACTGCAGCGCCGTCTGAAGGTGCCGCCCGCGCTCCACCAATTTACGA |
| | | AGGTGCTTGACCGCTCCAGTCGCAACGAGCTGCTGAAGCTGGTGAAGAAGTATCCTTCCGAGACGCGCAGGGCCCGCAGG |
| | | CAGCGCCTGTTTGACGTGGCGACTGAGAAAAAGAAGAATCCAGAGGCGGCGTCCAAGAAGGCCCCGCTCAGCGTCGTTAC |
| | | CGGTCTGCAGGAGGTAACCCGCACCATTGAGAAGAAGACCGCACGCCTTGTGATGATCGCGAACAATGTGGACCCCATTG |
| | | AGCTGGTGCTGTGGATGCCGACTTTGTGCCGTGCCAACAAAGTCCCATACGCGATTGTGAAGGACAAGGCACGTCTCGGC |
| | | GACGCGGTGGGCCGGAAGACCGCCACGTGCGTTGCAATCACCGATGTGAATGCCGAGGACGAGGCCGCTTTGAAGAATCT |
| | | CATCCGCTCTGTGAATGCACGCTTCCTGGCCCGTAGCGATGTTATCCGTCGCCAATGGGGAGGCCTGCAGCTCTCACTGC |
| | | GTTCTCGAGCCGAGCTGCGCAAGAAGCGTGCCCGCAACGCCGGCAAGGATGCTGCCGCCGTAATGTGA (SEQ ID |
| | | NO: 27) |
| TcYC6_0043560 | 40S ribosomal | MTTIGTYNEEGVNVDLYIPRKCHATNNLITSYDHSAVQIAIANVDANGVINGTTTTFCIAGYLRRQAESDHAINHLAISK |
| | protein S21 putative | GIIRIKTGKKPRAKKLKNVKGLGVRGLPRGALQQRGARVLPTQRGVAQRGGAQKGNVRKLQPQPQKQRSQLNQRSQQQHG |
| | | ARPTRKEEGGRTQRGGRDAPQARKQQGRNEPQARRQQGRNEPQARRQQGRNEPQARKQQGRDAPQARKQQGRNAPRSQKA |
| | | (SEQ ID NO: 6) |
| | | ATGACGACAATCGGTACGTACAACGAGGAGGGTGTTAACGTGGACCTGTACATCCCACGCAAGTGCCACGCGACAAACAA |
| | | CCTTATCACGTCATACGACCACTCCGCCGTGCAGATTGCCATTGCGAATGTGGACGCCAACGGTGTGCTAAACGGCACGA |
| | | CGACAACCTTCTGCATTGCTGGCTATCTTCGTCGCCAGGCTGAGTCTGACCACGCAATCAACCACCTGGCGATTTCGAAG |
| | | GGCATTATCCGCATCAAGACCGGCAAGAAGCCTCGCGCGAAGAAGCTTAAGAATGTGAAGGGCCTTGGCGTACGCGGCTT |
| | | ACCAAGGGGTGCTCTGCAACAGAGGGGAGCTCGTGTCCTCCCAACCCAGAGGGGTGTCGCGCAGCGTGGCGGCGCTCAGA |
| | | AGGGCAACGTCCGCAAGCTGCAGCCACAGCCGCAGAAGCAAAGGTCACAGCTGAATCAAAGGTCACAGCAGCAGCACGGC |
| | | GCCCGGCCGACCCGGAAGGAAGAGGGCGGTCGCACGCAGCGTGGTGGCAGGGATGCGCCTCAAGCTCGCAAGCAGCAAGG |
| | | CAGGAACGAGCCTCAAGCTCGCAGGCAGCAAGGCAGGAACGAGCCTCAAGCTCGCAGGCAGCAAGGCAGGAACGAGCCTC |
| | | AAGCTCGCAAGCAGCAAGGCAGGGATGCGCCTCAAGCTCGTAAGCAGCAAGGCAGGAATGCACCTCGTTCCCAGAAGGCA |
| | | TAG (SEQ ID NO: 28) |
| TcYC6_0083710 | 40S ribosomal | MGIVRSRLHKRKITGGKTKIHRKRMKAELGRLPAHTKLGARRVSPVRARGGNFKLRGLRLDTGNFAWGTEAIAQRARILD |
| | protein S8 putative | VVYNATSNELVRTKTLVKNCIVVVDAAPFKLWYAKHYGIDLDAAKSKKTAQSTTEKKKSKKTSHAMTEKYDVKKASDELK |
| | | RKWMLRRENHKIEKAVADQLKEGRLLARITSRPGQTARADGALLEGAELQFYLKKLEKKKR (SEQ ID NO: 7) |
| | | ATGGGTATCGTTCGCAGCCGCCTGCATAAGCGCAAGATCACCGGTGGAAAGACGAAGATCCACCGGAAGCGCATGAAGGC |
| | | CGAACTCGGCCGTCTTCCCGCGCACACGAAACTTGGCGCCCGCCGCGTGAGTCCCGTCCGCGCCCGCGGTGGGAACTTCA |
| | | AGCTCCGCGGTCTTCGCCTGGACACCGGCAATTTTGCGTGGGGCACAGAAGCCATTGCTCAGCGGGCCCGTATCCTCGAC |
| | | GTCGTGTACAACGCCACTTCTAACGAGCTGGTGCGCACGAAGACGCTTGTGAAGAACTGCATTGTTGTGGTGGACGCCGC |
| | | GCCCTTCAAGTTATGGTACGCGAAGCACTACGGTATCGATCTTGACGCCGCGAAGAGCAAGAAGACGGCGCAGAGCACGA |
| | | CGGAGAAGAAGAAGTCGAAGAAGACCTCACACGCCATGACTGAGAAGTACGACGTCAAGAAGGCCTCCGACGAGCTGAAG |
| | | CGCAAGTGGATGCTCCGCCGCGAGAACCACAAGATTGAGAAGGCAGTCGCTGATCAGCTCAAGGAGGGCCGTCTGCTCGC |
| | | CCGCATCACCAGCCGCCCTGGCCAGACAGCCCGCGCCGATGGTGCACTGCTGGAGGGCGCCGAACTGCAGTTCTATCTGA |
| | | AGAAGCTCGAGAAGAAGAAGCGGTAG (SEQ ID NO: 29) |
| TcBrA4_0028230 | hypothetical protein, | MGAPQIVYSALITNTTTIAVTVVVTYTMPNEMPPETLELLIQPGEEMLAPQKLVEDGIVTWTGYISKVAIQGGPSMSEPF |
| | conserved | PGVECPTRRYDFEVFMHAGVLRLFALGPAESSSD (SEQ ID NO: 8) |
| | | ATGGGGGCTCCTCAGATCGTGTACTCCGCCCTCATAACGAACACCACCACAATTGCTGTGACGGTGGTTGTCACCTACAC |
| | | CATGCCGAACGAAATGCCCCCGGAGACTCTGGAATTGCTCATTCAACCAGGCGAAGAAATGTTAGCGCCGCAGAAATTGG |
| | | TGGAGGACGGTATAGTAACCTGGACAGGCTATATTAGCAAGGTTGCCATTCAGGGTGGGCCGTCTATGAGTGAACCTTTC |
| | | CCGGGAGTGGAGTGTCCTACGAGAAGATACGACTTTGAAGTTTTCATGCATGCCGGCGTGCTGCGGCTATTCGCATTGGG |
| | | CCCTGCCGAATCAAGCAGTGATTGA (SEQ ID NO: 30) |
| TcYC6_0097920 | hypothetical protein, | MGSPKIVYSALIRNTTTISVTVLVTYSMPSEMPQETVQLLIPPGEEKEAPQKLVEEDTVTWTGFISKVAVEGGQSMSAPF |
| | conserved | LGVESPTRRYGFEVYMQAGMLRLLALGPVESSSD (SEQ ID NO: 9) |
| | | ATGGGGTCTCCTAAGATCGTGTACTCCGCCCTCATAAGGAACACCACCACGATTTCTGTGACGGTGCTTGTCACCTATTC |
| | | CATGCCGAGCGAAATGCCCCAGGAAACTGTGCAATTGCTCATTCCACCAGGCGAAGAAAAGGAAGCGCCCCAGAAATTGG |
| | | TGGAGGAAGATACAGTAACCTGGACAGGCTTTATTAGCAAGGTTGCCGTTGAGGGTGGGCAGTCTATGAGTGCTCCTTTC |
| | | CTGGGAGTGGAATCTCCTACGAGAAGATACGGTTTTGAAGTTTACATGCAAGCCGGCATGCTGCGGCTATTAGCATTGGG |
| | | CCCTGTCGAATCAAGCAGTGATTGA (SEQ ID NO: 31) |
| TcBrA4_0122270 | ubiquitin- | MPSTPTPQCVRRLQKELSALCREAESFFFTRPSAKSILVWYFVIKGPADTPYEGGRYFGKLNFPPDYPMKPPEIIILTPN |
| | conjugating enzyme | GRFETNKSICLTMSNYHPENWSPLWGVRTILTGLLSFMVGDELTTGCMTSSDELRRKYARESRRFNAEKMSVYKELFPEE |
| | E2, putative | YQKDLEELKREDSEKNGRTSGSAGCGANTKGGGVMESQEKEQWRGLFPALLGLFAVLMGAYFWPW (SEQ ID NO: 10) |
| | | ATGCCAAGCACACCCACCCCGCAGTGTGTGCGGCGGCTGCAAAAGGAGCTTTCCGCCCTATGCCGAGAGGCCGAGTCGTT |
| | | TTTTTTCACCCGTCCCTCAGCAAAGAGTATTCTGGTTTGGTATTTCGTCATCAAGGGTCCTGCGGATACCCCTTATGAAG |
| | | GCGGTCGCTACTTTGGCAAGCTGAATTTTCCCCCCGACTATCCAATGAAACCGCCTGAGATTATCATTTTGACGCCAAAT |
| | | GGACGTTTTGAGACCAACAAGAGCATTTGTCTCACCATGAGCAATTATCATCCGGAGAATTGGAGCCCTTTGTGGGGGGT |
| | | CCGCACCATTCTTACGGGGCTGCTCTCATTCATGGTGGGAGACGAACTCACTACTGGGTGCATGACGAGCAGCGATGAGT |
| | | TGCGGAGGAAGTATGCTCGTGAGAGCCGTCGTTTCAATGCAGAGAAAATGTCAGTATACAAGGAACTGTTTCCTGAGGAG |
| | | TATCAAAAGGATTTGGAGGAATTGAAGCGAGAGGACAGTGAGAAAAACGGTCGTACTTCTGGAAGTGCTGGTTGTGGTGC |
| | | GAATACGAAAGGAGGAGGTGTGATGGAATCGCAAGAAAAAGAACAATGGCGTGGGTTATTCCCGGCACTTTTGGGACTTT |
| | | TTGCTGTGTTAATGGGAGCCTACTTTTGGCCATGGTAA (SEQ ID NO: 32) |
| TcYC6_0088050 | ubiquitin- | MPSTPTPQCVRRLQKELSALCREAESFFFTRPSAKSILVWYFVIKGPADTPYEGGRYFGKLNFPPDYPMKPPEIIILTPN |
| | conjugating enzyme | GRFETNKSICLTMSNYHPENWSPLWGVRTILTGLLSFMVGDELTTGCMTSSDELRRKYARESRRFNAEKMPVYKELFPEE |
| | E2, putative | YQKDLEELKREDNEKNGRISGSAGCGANTKGGGVMESQEKEQWRGLFPALLGLFAVLMGAYFWPW (SEQ ID NO: 11) |
| | | ATGCCAAGCACACCCACCCCGCAGTGTGTGCGGCGGTTGCAAAAGGAGCTTTCCGCCCTATGCCGAGAGGCCGAGTCGTT |
| | | TTTTTTCACCCGTCCCTCAGCAAAGAGTATTCTGGTTTGGTATTTCGTCATCAAGGGTCCTGCGGATACCCCTTATGAAG |
| | | GCGGTCGCTACTTTGGCAAGCTGAATTTCCCCCCCGACTATCCAATGAAACCGCCTGAGATTATCATTTTGACGCCAAAT |
| | | GGACGTTTTGAGACCAACAAGAGCATTTGTCTCACCATGAGCAATTATCATCCGGAGAATTGGAGCCCTTTGTGGGGGGT |
| | | CCGCACCATTCTTACGGGGTTGCTCTCTTTCATGGTGGGAGACGAACTCACTACTGGGTGCATGACGAGCAGCGATGAGT |
| | | TGCGGAGGAAGTACGCTCGTGAGAGCCGTCGTTTCAATGCAGAGAAAATGCCAGTATACAAGGAACTGTTTCCAGAGGAG |
| | | TATCAGAAGGACTTGGAGGAATTGAAGCGAGAGGACAATGAGAAAAACGGTCGTATTTCTGGAAGTGCTGGCTGTGGTGC |
| | | GAATACGAAAGGAGGAGGTGTGATGGAATCGCAAGAAAAAGAGCAATGGCGTGGGTTATTCCCGGCACTTTTGGGACTTT |
| | | TTGCTGTGTTAATGGGAGCCTACTTTTGGCCATGGTAA (SEQ ID NO: 33) |
| TcYC6_0028190 | 60S acidic ribosomal | MSMKYLAAYALASLNKPTPGAADVEAICKACGIEVESDALSFVMESIAGRSVATLVAEGAAKMSAVAVSAAPAAGGAAAP |
| | protein P2 putative | AAAAGGAAAPAAADAKKEEEEEDDDMGFGLFD (SEQ ID NO: 12) |
| | | ATGTCCATGAAGTACCTCGCCGCATACGCTCTTGCTTCGCTGAACAAACCAACGCCAGGCGCCGCCGATGTGGAGGCCAT |
| | | CTGCAAGGCCTGCGGTATCGAAGTTGAGAGCGACGCACTCTCGTTTGTCATGGAATCCATTGCCGGCCGGAGCGTTGCCA |
| | | CTCTCGTGGCGGAGGGCGCGGCGAAGATGAGCGCTGTTGCCGTCTCCGCTGCTCCTGCTGCCGGTGGTGCAGCCGCTCCT |
| | | GCTGCTGCTGCTGGCGGTGCCGCCGCCCCTGCCGCTGCTGACGCCAAGAAGGAAGAAGAGGAGGAGGACGATGACATGGG |
| | | CTTTGGTCTGTTTGACTAA (SEQ ID NO: 34) |
| TcBrA4_0101960 | surface protein TolT | MAPPADMRGALREVLGAMQKAQEYADEANRHCVQARMSAESAREHEEGAKNALRKLGSEATRMSRALQQADEAVKLADAA |
| | | VAECKAAEEAAQAAGIMTLDAVGEVLKHLKDEKTKVGSGPELLKRAAEQTVLSLEKAKEAEAEAEKAAAAAQKTREAAEK |
| | | AAAARTLAQDVAATASALLRQREKEEERRRARDQEVAEAAKKAAVAEVMKKFAAKGNDTAPGRNSTSTRFQRTRPRVDGG |
| | | GIPLLLRAPLLMLAAVASVFGFLSC (SEQ ID NO: 13) |
| | | ATGGCGCCACCGGCTGATATGAGGGGGGCGTTGAGAGAGGTGTTGGGAGCCATGCAGAAGGCGCAGGAGTATGCTGACGA |
| | | GGCTAACCGGCACTGCGTGCAGGCAAGAATGAGCGCTGAGAGTGCGCGGGAGCATGAAGAGGGGGCTAAGAATGCTTTGA |
| | | GGAAGCTCGGCTCTGAGGCTACAAGGATGAGCAGGGCGCTGCAGCAAGCGGACGAGGCTGTGAAATTGGCCGATGCTGCC |
| | | GTGGCCGAATGCAAGGCGGCGGAGGAGGCTGCACAGGCGGCGGGGATAATGACGCTTGATGCCGTTGGGGAGGTGCTGAA |
| | | GCATCTGAAGGACGAGAAGACCAAGGTTGGAAGTGGACCGGAGCTGTTGAAGAGGGCGGCGGAGCAGACTGTGCTTTCTC |
| | | TGGAGAAGGCAAAGGAGGCGGAGGCGGAGGCTGAGAAGGCGGCAGCGGCGGCGCAGAAAACCCGGGAAGCAGCAGAGAAG |
| | | GCAGCAGCGGCGCGGACCTTGGCACAAGATGTTGCCGCAACGGCCAGTGCGCTGCTGCGGCAGCGGGAGAAGGAGGAGGA |
| | | GAGGCGAAGAGCGAGGGACCAGGAGGTGGCGGAGGCCGCGAAGAAGGCTGCCGTTGCTGAGGTGATGAAAAAATTTGCTG |
| | | CGAAGGGGAATGACACAGCGCCTGGCAGGAATTCCACATCCACCCGCTTTCAAAGGACGAGGCCACGGGTGGATGGCGGC |
| | | GGCATCCCATTGCTTTTGCGTGCACCGCTTCTGATGCTTGCTGCCGTGGCATCCGTTTTCGGCTTCTTATCGTGCTAG |
| | | (SEQ ID NO: 35) |
| TcBrA4_0101980 | mucin-associated | MTRNRLFFPLLLLLSCSVIVGANATEKKASTPRKAEGVQPQSVSPSSSFPGDGTGVPLKLELGELRDKALLAAKDAFGNT |
| | surface protein | TGAAMQCMQAKTDVEETKKYAEEAKKLFDKIGGDYVSKSAALADAVKASTDAEEALKSCVEAEKAAVDADTAVLAAVLEV |
| | (MASP), | LQHSKFWRRDTAVSTEKLANVSKHSANATNEAQKAGIQASKAAEAAKRAAESKKKAAAALDTVKEVVAMAEMLKEKFFEN |
| | syntenic/homologous | ERLQKEKHEAQLEAERQFIQEEVQKKEAEAEKALNRAAAADKRVAELELARQKQSKEQGNEGRGHRRVRRSGSDSSSNYA |
| | with Surface protein | PAYEPRLLLLPLLSFTLFCFVAWC (SEQ ID NO: 14) |
| | TolT, group C | ATGACGCGTAATAGGCTTTTTTTCCCTCTGCTTCTTCTACTCTCCTGCAGCGTAATTGTCGGCGCAAATGCAACAGAAAA |
| | | GAAAGCGTCAACGCCAAGGAAAGCAGAGGGAGTGCAGCCGCAATCGGTCTCACCGTCTTCGTCGTTTCCAGGGGATGGGA |
| | | CGGGTGTGCCGCTCAAATTGGAACTGGGGGAACTGAGGGACAAAGCATTGCTGGCAGCAAAGGATGCTTTTGGCAATACG |
| | | ACAGGGGCGGCAATGCAATGCATGCAGGCCAAGACGGATGTCGAAGAGACCAAGAAATACGCCGAAGAGGCGAAAAAGCT |
| | | TTTTGATAAGATTGGCGGGGACTATGTGTCAAAAAGTGCTGCTCTGGCGGATGCAGTGAAAGCTAGCACCGACGCCGAAG |
| | | AGGCGCTGAAAAGCTGTGTGGAGGCGGAAAAGGCCGCTGTTGATGCTGATACCGCGGTTTTAGCTGCTGTCCTGGAGGTG |
| | | CTGCAACATTCCAAGTTTTGGCGAAGGGACACTGCAGTTTCGACTGAAAAATTGGCGAATGTCAGTAAACATTCGGCGAA |
| | | CGCCACAAATGAGGCGCAAAAGGCAGGGATTCAAGCGTCGAAGGCGGCAGAAGCGGCGAAGAGGGCAGCGGAGTCGAAAA |
| | | AAAAAGCTGCAGCAGCTCTGGATACGGTCAAGGAAGTCGTTGCGATGGCCGAGATGTTGAAGGAAAAGTTTTTCGAGAAT |
| | | GAGAGGCTGCAAAAGGAAAAACATGAGGCTCAATTGGAAGCCGAAAGACAGTTCATTCAGGAAGAGGTACAGAAGAAGGA |
| | | GGCGGAGGCCGAAAAGGCACTCAATCGCGCTGCTGCGGCTGATAAACGTGTCGCCGAGTTGGAACTTGCCAGACAAAAGC |
| | | AGAGCAAAGAGCAGGGGAATGAAGGAAGAGGCCATAGGCGAGTCAGACGCAGTGGGAGTGACAGCAGCAGCAACTATGCG |
| | | CCTGCATATGAACCACGGCTACTGTTACTGCCTCTGCTTTCTTTCACACTGTTCTGTTTTGTTGCATGGTGCTAG (SEQ |
| | | ID NO: 36) |
| TcBrA4_0088260 | 60S ribosomal | MPAKTAVSKAAAPKKAAAPKKAAAPQKAAAPKKAAAPKKAAAPQKAAVAKKAVREAPKKGVKKTAKKGAPAAMTKVVKVT |
| | protein L23a | KRKAYTRPQFRRPHTYRRPSIPKPSNNMSAIPNKWDAFRVIRYPLTTDKAMKKIEENNTLTFIVDSNANKTEIKKAMRKL |
| | putative | YQVKAVKVNTLIRPDGLKKAYIRLSASYDALETANKMGLL (SEQ ID NO: 15) |
| | | ATGCCTGCCAAAACCGCCGTTTCGAAGGCTGCTGCGCCCAAAAAGGCCGCTGCGCCCAAGAAGGCCGCTGCACCACAAAA |
| | | GGCTGCTGCGCCCAAGAAGGCTGCTGCGCCCAAGAAGGCTGCTGCACCCCAAAAGGCTGCTGTCGCCAAGAAGGCCGTCA |
| | | GGGAGGCCCCCAAAAAGGGTGTCAAGAAGACCGCCAAGAAGGGCGCGCCGGCCGCTATGACGAAGGTGGTGAAGGTCACG |
| | | AAGCGCAAGGCGTACACCCGCCCGCAGTTCCGTCGTCCGCACACGTACCGGAGGCCGTCGATCCCCAAGCCGAGCAACAA |
| | | CATGAGTGCGATTCCCAACAAGTGGGATGCGTTTCGTGTGATCCGCTACCCGCTGACCACCGACAAGGCGATGAAGAAGA |
| | | TTGAGGAGAACAATACGCTGACCTTCATTGTGGACTCGAACGCCAACAAGACGGAAATCAAGAAGGCCATGCGCAAGCTC |
| | | TACCAGGTGAAGGCCGTGAAGGTGAACACCCTCATCCGACCGGACGGCCTTAAGAAGGCGTACATCCGCCTCTCCGCCTC |
| | | GTACGACGCCCTCGAGACAGCCAACAAGATGGGTCTGCTGTAG (SEQ ID NO: 37) |
| TcBrA4_0074300 | 40S ribosomal | MTKKHLKRLYAPKDWMLSKLTGVFAPRPRAGPHKLRECMTLMIIIRNRLKYALNAAEAQMILRQGLVCVDGKPRKDTKYP |
| | protein S4 putative | VGFMDVVEIPRTGDRFRILYDVKGRFALVKVGEAEGNIKLLKVENVYTSTGRIPVAMTHDGHRIRYPDPRTHRGDTLVYN |
| | | LKEKKVVDLIKSSNGKVVMVTGGANRGRIGEIMSIERHPGAFDIARLKDAAGHEFATRASNIFVIGKDMQSVPVTLPKQQ |
| | | GLRINVIQEREEKLIAAEARKNMQTRGVRKARK (SEQ ID NO: 16) |
| | | ATGACCAAGAAGCACCTGAAGCGCCTTTATGCCCCCAAGGACTGGATGCTGAGCAAGCTCACGGGCGTGTTCGCTCCACG |
| | | TCCCCGTGCTGGACCCCACAAGCTGCGTGAGTGCATGACTCTTATGATCATCATCCGCAATCGTCTGAAGTATGCGCTGA |
| | | ACGCCGCCGAGGCTCAGATGATTCTCCGTCAGGGCCTTGTGTGCGTGGACGGTAAGCCCCGCAAGGACACCAAGTATCCG |
| | | GTTGGCTTCATGGACGTTGTGGAGATCCCACGGACCGGGGATCGTTTCCGCATTCTGTACGACGTGAAGGGCCGCTTTGC |
| | | CCTCGTGAAGGTTGGCGAGGCTGAGGGGAACATCAAGCTCCTGAAGGTGGAGAACGTCTACACAAGCACTGGTCGCATTC |
| | | CTGTTGCCATGACACACGACGGTCACCGCATTCGTTACCCCGACCCCCGCACCCACCGTGGCGACACCCTGGTGTACAAC |
| | | CTGAAGGAGAAGAAGGTGGTGGACCTCATCAAGTCCAGCAACGGCAAGGTGGTGATGGTCACCGGCGGCGCGAACCGCGG |
| | | CCGTATTGGCGAGATCATGTCGATTGAGCGCCACCCTGGTGCGTTCGACATTGCACGCCTGAAGGATGCGGCGGGACACG |
| | | AGTTTGCTACCCGAGCGTCCAACATTTTTGTGATTGGCAAGGACATGCAGAGCGTTCCTGTGACGCTGCCGAAGCAACAG |
| | | GGTCTCCGCATCAACGTGATTCAGGAGCGTGAGGAGAAGCTTATCGCTGCTGAGGCACGCAAGAATATGCAGACTCGCGG |
| | | CGTACGCAAGGCCCGCAAATAG (SEQ ID NO: 38) |
| TcYC6_0122760 | hypothetical protein | MMRFTRFLVVAAKRSATSAKLGKSVGLTAALSPKQRSLPRVSVTKLMKPSGSGKHVTSSFLLKDKKKVATAKVAVPPKKK |
| | conserved | RALKVRKGRSSGKKAAALYVRFYHALKKSGLVKGKRRMQKTGELWRATKKAKDFKKRVEAAMRLAKKGQKSRARKLKAQK |
| | | KAKGKKSAKGVRRVYRRVSRKKTVTSTVPPLP(SEQ ID NO: 17) |
| | | ATGATGCGTTTTACCCGGTTCCTTGTCGTTGCAGCAAAGCGGAGTGCCACCAGCGCCAAACTCGGTAAGAGTGTTGGACT |
| | | CACCGCGGCGCTGAGTCCCAAGCAAAGGTCCCTTCCCCGCGTCTCAGTGACGAAGTTGATGAAGCCCAGCGGGAGCGGGA |
| | | AACACGTTACGTCGTCATTCTTGTTGAAGGACAAGAAGAAGGTGGCCACCGCAAAAGTTGCTGTGCCGCCGAAAAAGAAG |
| | | AGGGCTTTAAAGGTGAGGAAGGGCCGCAGCAGCGGCAAAAAGGCCGCGGCTCTCTATGTGCGCTTTTATCACGCCTTGAA |
| | | GAAGAGCGGACTTGTGAAGGGGAAGCGACGCATGCAGAAAACGGGTGAGCTGTGGCGTGCCACAAAGAAGGCGAAGGACT |
| | | TCAAGAAGCGCGTTGAGGCGGCGATGAGGCTTGCAAAGAAGGGACAAAAAAGCAGGGCTCGTAAGCTGAAGGCGCAGAAG |
| | | AAGGCAAAGGGCAAAAAGTCGGCGAAGGGCGTCAGGAGGGTCTACCGGAGGGTCAGCAGGAAGAAGACTGTCACGAGCAC |
| | | CGTGCCGCCTCTCCCTTAA (SEQ ID NO: 39) |
| TcBrA4_0130080 | 60S ribosomal | MPKGKNAIPHVHQRKHWNPCSSQKGNVKVFLNQPAQKLRRRRLRLLKAKKTFPRPLKALRPQVNCPTVRHNMKKRLGRGF |
| | protein L13 putative | TVEELKAAGINPRFAPTIGIRVDRRRKNKSEEGMSINIQRLKTYMSKLVLFPMSYKNVQKGEATEEEVKSATQDRTRFGT |
| | | AAVGGFVTPAPEAPRKVTEEERTKNVYKFLKKNHSAVRFFGIRRARQERREAKENEKK (SEQ ID NO: 18) |
| | | ATGCCGAAGGGAAAAAACGCGATCCCCCACGTGCACCAGAGGAAGCACTGGAACCCGTGCTCTTCCCAGAAGGGTAATGT |
| | | GAAGGTTTTCCTCAACCAGCCCGCACAGAAGCTGCGCCGTCGCCGCCTACGTCTTTTGAAGGCGAAGAAGACGTTCCCAC |
| | | GCCCACTCAAGGCGCTGCGCCCGCAGGTGAATTGCCCCACGGTGCGTCACAACATGAAGAAGCGCCTGGGCCGTGGCTTT |
| | | ACCGTTGAGGAGCTGAAGGCTGCCGGCATCAACCCTCGTTTTGCCCCGACGATTGGCATCCGTGTGGATCGTCGCCGCAA |
| | | GAACAAGAGCGAGGAGGGCATGAGCATCAACATCCAGCGCCTGAAGACGTACATGAGCAAGCTGGTGCTCTTCCCCATGA |
| | | GCTACAAGAACGTGCAGAAGGGCGAGGCCACTGAGGAGGAGGTGAAGTCTGCCACTCAGGACCGCACACGCTTTGGTACT |
| | | GCGGCTGTTGGTGGTTTTGTGACGCCTGCTCCCGAGGCACCACGCAAGGTGACAGAGGAGGAGCGCACAAAGAACGTGTA |
| | | CAAGTTCCTCAAGAAGAACCACAGCGCTGTTCGCTTCTTTGGCATTCGCAGGGCACGTCAGGAACGCAGGGAGGCCAAGG |
| | | AGAACGAGAAGAAGTAA (SEQ ID NO: 40) |
| TcBrA4_0029760 | calcium-binding | MDTTLYSEVNRLERGDFLLFHCVQLSQHERDVQRYFFGCYFPRWRGFYLEEVRDMPGPLGYKVQRHFPAYPFDVYLKDNG |
| | protein, putative | EHFLTDDFQEGSIFTLGASQNQRDGDSKRYKVVHCDDSRLRTRTGTTLADIGNDITTKLNQTHRVPGEVIDLLREIRDAY |
| | | VVYAGNGIPEIGIKAMGRHFRHVSEDGKRWMSLENIGKLVRDSRAFSNTLSFEDTQRTNSTISNNARSIHEAFPQNEEGC |
| | | IDYDLFMDYVRGPMSQKRKDAVWEIFRKLDFDGDGYLNILDIQARYNAQQHPVVAVERLFSADKLLKGFLTVWDENKQYG |
| | | LIPYAEFIDYYNGVSAVIADDYIFFDILRNQWKVMRDWGGTVGTRGGNCEFPTM (SEQ ID NO: 19) |
| | | ATGGATACGACGCTTTACAGTGAGGTGAATCGTCTCGAACGCGGTGACTTTCTTCTTTTTCACTGTGTGCAGCTCTCACA |
| | | ACACGAGCGTGACGTGCAGCGGTACTTCTTTGGATGCTACTTTCCGCGCTGGCGTGGGTTCTACCTGGAGGAGGTGAGGG |
| | | ATATGCCGGGCCCTCTAGGCTACAAGGTGCAGCGACACTTTCCTGCGTATCCCTTTGACGTGTATCTGAAGGACAATGGT |
| | | GAACACTTTCTCACGGATGACTTCCAGGAGGGTTCTATATTCACTTTGGGAGCCTCGCAAAATCAGCGTGACGGCGACTC |
| | | GAAGCGATATAAAGTAGTGCACTGCGACGATAGTCGTTTGCGCACGCGCACGGGTACGACTCTTGCAGACATTGGCAATG |
| | | ACATCACGACGAAGTTGAATCAAACACACCGTGTCCCTGGCGAGGTGATAGATCTCCTGCGTGAGATTAGAGATGCGTAT |
| | | GTTGTGTATGCCGGCAATGGCATTCCTGAGATTGGTATCAAGGCAATGGGACGTCACTTTCGCCACGTCAGCGAGGATGG |
| | | AAAGCGGTGGATGTCGTTGGAGAACATTGGAAAGCTTGTTCGTGACTCTCGTGCCTTTTCCAACACATTGTCATTTGAGG |
| | | ACACGCAGAGGACGAATTCCACGATTAGCAATAATGCAAGGAGCATTCATGAAGCCTTTCCGCAGAATGAAGAAGGCTGC |
| | | ATTGACTATGATTTATTCATGGACTACGTTCGTGGACCGATGAGCCAAAAAAGGAAGGATGCCGTCTGGGAAATATTCCG |
| | | CAAGCTTGACTTTGATGGAGACGGCTACCTCAACATCTTAGACATTCAGGCCCGCTACAATGCGCAGCAGCACCCTGTGG |
| | | TGGCGGTGGAGAGACTCTTCTCCGCGGACAAACTGCTCAAGGGCTTCCTCACCGTTTGGGATGAGAACAAACAATACGGG |
| | | TTGATCCCATACGCCGAGTTTATCGACTACTACAACGGCGTCAGCGCGGTAATTGCGGACGACTACATCTTTTTTGATAT |
| | | TCTCCGGAATCAATGGAAGGTCATGCGTGACTGGGGAGGGACGGTGGGGACGAGGGGAGGGAATTGTGAGTTCCCGACGA |
| | | TGTAA (SEQ ID NO: 41) |
| TcYC6_0096240 | calcium-binding | MDTTLYSEVNRLERGDFLFFHCVQLSQHERDVQRYFFGCYFPRWRGFYLEEVRDMPGPLGYKVQRHFPAYPFDVYLKDNG |
| | protein, putative | EHFLTDDFQEGSIFTLGASQNQRDGESKRYKVVHCDDSRLRTRTGTTLADIGNDITTRLNQTHRVPGEVIDLLREIRDAY |
| | | VVYAGNGIPEIGIKAMGRHFRHVSEDGKRWMSLENIGKLVRDSRAFSTTLSFEDTQKTNSTISNNARSIHEAFPQNEEGC |
| | | IDYDLFMDYVRGPMSQKRKDAVWEIFRKLDFDGDGYLNILDIQARYNAQQHPVVAVERLFSADKLLKGFLTVWDENKQYG |
| | | LIPYAEFIDYYNGVSAVIADDYIFFDILRNQWKVMRDWGGTVGTRRGKSEVSTM (SEQ ID NO: 20) |
| | | ATGGATACGACGCTTTACAGTGAGGTGAATCGTCTCGAACGCGGTGACTTTCTTTTTTTTCACTGTGTGCAGCTCTCACA |
| | | ACACGAGCGTGACGTGCAGCGGTACTTCTTTGGATGCTACTTTCCGCGCTGGCGTGGGTTCTACCTGGAGGAGGTGAGGG |
| | | ATATGCCAGGCCCTCTAGGCTACAAGGTGCAGCGACACTTTCCTGCGTATCCCTTTGACGTGTATCTGAAGGACAATGGT |
| | | GAACACTTTCTCACGGATGACTTCCAGGAGGGTTCTATATTCACTTTGGGAGCCTCGCAAAATCAGCGTGACGGCGAGTC |
| | | GAAGCGATATAAAGTAGTGCACTGCGACGATAGTCGTCTGCGCACGCGCACGGGCACGACTCTTGCAGACATTGGCAATG |
| | | ACATCACGACGAGGTTGAATCAAACACACCGTGTCCCTGGCGAGGTGATAGATCTCCTGCGTGAGATTAGAGATGCGTAT |
| | | GTTGTGTATGCCGGCAATGGCATTCCTGAGATTGGTATCAAGGCAATGGGACGTCACTTTCGCCACGTCAGCGAGGATGG |
| | | AAAGCGGTGGATGTCGTTGGAGAACATTGGAAAGCTTGTTCGTGACTCTCGTGCCTTTTCCACCACATTGTCATTTGAGG |
| | | ACACGCAGAAGACGAATTCCACGATTAGCAATAATGCAAGGAGCATTCATGAAGCCTTTCCGCAGAATGAAGAAGGCTGC |
| | | ATTGACTATGATTTATTCATGGACTACGTTCGTGGGCCGATGAGCCAAAAACGGAAGGATGCCGTCTGGGAAATATTCCG |
| | | CAAGCTTGACTTTGATGGAGACGGCTACCTCAACATCTTAGACATTCAGGCCCGCTACAATGCGCAGCAGCACCCTGTGG |
| | | TGGCGGTGGAGAGACTCTTCTCCGCGGACAAACTGCTTAAGGGCTTCCTCACCGTTTGGGATGAGAACAAACAATACGGG |
| | | TTGATCCCATACGCCGAGTTTATCGACTACTACAACGGCGTCAGCGCGGTAATTGCGGACGACTACATCTTTTTTGATAT |
| | | TCTCCGGAATCAATGGAAGGTCATGCGTGACTGGGGAGGGACGGTGGGGACGAGGAGAGGGAAGAGTGAGGTTTCGACGA |
| | | TGTAA (SEQ ID NO: 42) |
| TcBrA4_0131050 | 60S acidic ribosomal | MADKVEANDTLACTYAALMLSDAGLPITAEGIEAACVAAGLKVRNTLPVIFARFLEKKPLETLFAAAAATAPAEGAAAAP |
| | protein P2, putative | AAGSAAPAAAAAGAAPEKDTKEEEEDDDMGFGLFD (SEQ ID NO: 21) |
| | | ATGGCCGATAAGGTTGAAGCGAACGACACGCTGGCGTGCACCTACGCCGCCCTCATGCTCAGCGACGCGGGTCTGCCCAT |
| | | CACGGCGGAGGGCATTGAGGCCGCGTGTGTGGCTGCCGGTCTGAAGGTGCGCAACACCCTGCCCGTTATTTTTGCTCGCT |
| | | TTCTCGAAAAGAAGCCGCTGGAGACTCTCTTTGCCGCTGCCGCTGCTACGGCACCTGCAGAGGGCGCCGCTGCTGCTCCT |
| | | GCCGCTGGCAGTGCCGCCCCTGCCGCCGCAGCTGCCGGTGCTGCGCCAGAAAAGGACACAAAGGAGGAGGAGGAAGACGA |
| | | CGATATGGGTTTTGGCTTGTTTGACTAG (SEQ ID NO: 43) |
| TcYC6_0111870 | 60S acidic ribosomal | MADKVEANDTLACTYAALMLSDAGLPITAEGIEAACVAAGLKVRNTLPVIFARFLEKKPLESLFAAAAATAPAEGAAAVP |
| | protein P2, putative | AAGSAAPAAAAAAAAPAKDTKEEEEDDDMGFGLFD (SEQ ID NO: 22) |
| | | ATGGCCGATAAGGTTGAAGCGAACGACACGCTGGCGTGCACCTACGCCGCCCTCATGCTTAGCGACGCGGGTCTGCCCAT |
| | | CACGGCGGAGGGCATTGAGGCCGCGTGTGTGGCTGCCGGTCTGAAGGTGCGCAACACCCTGCCCGTTATTTTTGCTCGCT |
| | | TTCTTGAAAAGAAGCCGCTGGAGAGTCTCTTCGCTGCTGCCGCTGCTACGGCTCCTGCAGAGGGCGCCGCTGCTGTTCCT |
| | | GCCGCTGGCAGTGCCGCCCCTGCTGCCGCAGCTGCCGCTGCTGCGCCAGCAAAGGACACAAAGGAGGAGGAGGAAGACGA |
| | | CGATATGGGTTTTGGCTTGTTTGACTAG (SEQ ID NO: 44) |
| TcBrA4_0056330 | hypothetical protein | MYKFGGEAKDLRNIYNFGDMSQRETEPPKDLSLAENKAYLVDVEVHSDNNEEEMGNRESQQPNSRVSPTAHGVPQSSAFF |
| | | PEFSHSSGPDVPRKPSMESTSEQKNSKEKQKENSKVKIAKEVLGINKKNTSGMSPEEKERVLLEERWKRAMAEENRLNAL |
| | | EEQVTHREQATNSSGLLPNFPPKFLCIKPLVHHDISSVPEVRRQFVRFNFINWIATCVLLLVNMIIVIAVVFASHKEDAK |
| | | KFHTSQNTVLAILYLMGAPLSFIVWYWQIYSACSTGRHTKHLLALSGLVIALAFDIFMIVGRTNYAACGVSLAIDISKTK |
| | | SKLAVLPVIVVLFFWVVEAVILCYCIAKQWMYYRLDVNAQEEVRRQMRNVIGI(SEQ ID NO: 53) |
| | | ATGTATAAGTTTGGAGGTGAGGCGAAGGATCTTAGAAACATTTATAATTTTGGCGATATGAGCCAACGAGAAACGGAGCC |
| | | ACCGAAGGACTTATCATTAGCAGAAAATAAAGCTTATTTGGTGGATGTAGAGGTGCATTCTGATAATAATGAAGAGGAAA |
| | | TGGGGAATCGTGAGAGCCAACAACCCAATTCCAGGGTCTCACCGACGGCTCATGGAGTTCCTCAATCCTCCGCGTTTTTT |
| | | CCGGAATTTTCACACTCTTCTGGACCTGATGTTCCTCGAAAACCCTCAATGGAAAGTACTTCGGAACAAAAAAACTCAAA |
| | | GGAAAAACAAAAGGAGAATAGTAAAGTAAAGATTGCAAAAGAAGTTTTAGGAATAAACAAGAAAAATACCTCTGGGATGT |
| | | CACCTGAAGAGAAGGAGCGTGTATTACTTGAAGAAAGGTGGAAAAGAGCCATGGCAGAGGAGAATCGTTTGAACGCACTC |
| | | GAAGAGCAAGTAACTCATCGTGAGCAAGCGACTAATTCTTCAGGCCTTCTTCCAAACTTCCCTCCCAAGTTCTTATGTAT |
| | | TAAGCCACTTGTACACCATGATATTTCGAGTGTTCCCGAGGTGAGAAGACAATTTGTCAGGTTTAATTTTATAAATTGGA |
| | | TTGCCACATGTGTTTTGCTCCTTGTCAATATGATTATTGTTATTGCTGTGGTATTTGCATCTCATAAAGAAGATGCAAAA |
| | | AAATTCCATACTTCTCAAAACACTGTTTTAGCCATTTTGTACCTGATGGGAGCCCCTTTAAGCTTTATTGTTTGGTATTG |
| | | GCAGATTTATTCTGCTTGTTCCACAGGACGTCATACTAAACATCTTTTGGCTCTAAGTGGGTTGGTTATAGCTCTTGCCT |
| | | TTGATATATTTATGATTGTTGGTCGGACAAACTATGCTGCATGCGGTGTATCTCTTGCAATAGATATATCGAAAACGAAA |
| | | AGTAAGCTTGCCGTATTGCCCGTGATCGTTGTTCTTTTTTTCTGGGTTGTAGAGGCTGTTATATTGTGTTACTGTATCGC |
| | | AAAACAGTGGATGTACTATCGGTTGGATGTGAACGCGCAAGAAGAAGTGAGACGCCAGATGCGGAATGTGATTGGAATTT |
| | | AG (SEQ ID NO: 60) |
| TcBrA4_0033670 | kinetoplastid- | MGKKYAQLETLHNVNGRVVIVGDIHGCLAQLEDILSVTDFARGRDQLITAGDMVNKGPDSFGVVRLLKSLGARGVIGNHD |
| | specific phospho- | AKLLKLRKKIRKHGTLHGTNSQSSLAPLAMSLPQDVEEYLLQLPHILRIPAHNILVVHAGLHVQHPLERQLVKEVTTMRN |
| | protein phosphatase, | LILQDDGLYRASEDTTDGVPWASLWQGPETVVFGHDARRGLQRHPHAIGLDTRCVYGGELTALVCPGEHLVSVPGWTSNR |
| | putative | SKV (SEQ ID NO: 54) |
| | | ATGGGAAAAAAATACGCACAGTTAGAGACTCTCCACAACGTGAATGGGCGGGTTGTCATTGTGGGCGACATTCATGGCTG |
| | | CCTTGCCCAACTGGAGGACATTTTATCAGTCACAGACTTTGCGAGGGGAAGGGATCAGTTAATCACCGCTGGGGACATGG |
| | | TGAACAAAGGGCCAGACTCGTTTGGCGTTGTGCGTCTGCTGAAGAGCCTTGGAGCACGCGGTGTGATTGGCAATCATGAC |
| | | GCCAAGCTTCTCAAACTTCGGAAAAAGATACGAAAACATGGGACGCTGCACGGGACGAATAGCCAATCGAGTTTGGCCCC |
| | | GCTTGCCATGTCGCTACCGCAGGATGTTGAAGAGTATTTATTACAACTGCCGCATATTCTCCGCATTCCTGCACACAACA |
| | | TTCTGGTGGTACATGCGGGCCTTCACGTTCAACACCCACTCGAGCGGCAATTGGTTAAGGAGGTCACTACGATGCGCAAC |
| | | CTCATTTTGCAGGATGACGGGCTGTACAGGGCATCTGAGGATACAACGGACGGTGTGCCCTGGGCATCGCTGTGGCAGGG |
| | | TCCGGAGACTGTTGTCTTTGGCCACGACGCCAGACGAGGCCTCCAACGCCACCCTCATGCGATCGGGTTGGACACTCGGT |
| | | GTGTGTATGGCGGGGAGCTCACTGCTCTTGTGTGTCCCGGTGAACACCTCGTTTCCGTGCCTGGATGGACTTCCAATAGA |
| | | TCGAAGGTGTGA (SEQ ID NO: 61) |
| TcYC6_0074990 | hypothetical protein | MYKFGGEAKDLRNIYNFGDMSQRETEPQKELSLAENRAYLVDVEVHSDNNEEEMGHRESQQPNSRVSPTAQGVPQSSAFF |
| | | SEFSHSSGIDFPQKPSMENTSDQKNSNEKPKENSKVKIAKEVLGINKKNTSGMSPEEKERVLLEERWKRAMAEENRLNAL |
| | | EEQVTHREQATNSSGLLPNFPPKFLFIKPLVHHDISSVPEVRRQFVRFNFINWIATCVLLLVNMIIVIAVVFASHKEDAK |
| | | KFNTSQNTVLAILYLVGAPLSFIVWYWQIYSACSTGRHTKHLLALSGLVIALAFVIFMIVGRTNYAACGVSLAIDISKTK |
| | | SKFAVLPVIIVLFFWVVEAVILCYCIVKQWIYYRLDVNAQEEVRRQMRNVIGI(SEQ ID NO: 55) |
| | | ATGTATAAGTTTGGAGGTGAGGCGAAGGATCTTCGAAACATTTATAATTTTGGCGATATGAGCCAACGAGAAACGGAGCC |
| | | ACAGAAGGAATTATCATTGGCAGAAAATAGAGCTTATTTGGTGGATGTAGAGGTGCATTCTGATAATAATGAAGAGGAAA |
| | | TGGGGCATCGTGAGAGCCAACAACCCAACTCCAGAGTCTCACCGACGGCTCAGGGAGTTCCTCAGTCCTCCGCGTTTTTT |
| | | TCGGAATTTTCACACTCTTCTGGAATTGATTTTCCTCAAAAACCCTCAATGGAAAATACTTCGGACCAAAAAAACTCAAA |
| | | CGAAAAACCAAAGGAGAATAGTAAAGTAAAGATCGCAAAAGAAGTTTTAGGAATAAATAAGAAAAATACCTCTGGGATGT |
| | | CACCTGAAGAGAAGGAGCGTGTATTACTTGAAGAAAGATGGAAAAGAGCCATGGCAGAGGAGAATCGTTTGAACGCACTC |
| | | GAAGAGCAAGTAACTCATCGTGAGCAAGCGACTAATTCTTCAGGTCTTCTTCCCAACTTCCCTCCCAAGTTCTTATTTAT |
| | | TAAGCCACTTGTACACCATGATATTTCGAGTGTTCCCGAGGTCAGAAGACAATTTGTCAGGTTTAATTTTATAAATTGGA |
| | | TCGCCACATGTGTTTTGCTCCTTGTCAATATGATTATTGTTATTGCTGTGGTATTTGCATCTCATAAAGAAGATGCAAAA |
| | | AAATTCAATACTTCTCAAAACACTGTTTTAGCCATTTTGTACCTGGTGGGAGCCCCTTTAAGCTTTATTGTTTGGTATTG |
| | | GCAGATTTATTCTGCTTGTTCCACAGGACGTCATACCAAACATCTTTTGGCTCTAAGTGGGTTGGTTATAGCACTTGCCT |
| | | TTGTTATATTTATGATTGTTGGTCGGACAAACTATGCTGCATGCGGTGTATCTCTTGCAATAGATATATCGAAAACGAAA |
| | | AGCAAGTTTGCCGTATTGCCCGTGATCATTGTTCTTTTTTTCTGGGTTGTAGAGGCTGTTATATTGTGTTACTGTATCGT |
| | | AAAACAGTGGATCTACTATCGGTTGGATGTGAACGCGCAAGAAGAAGTGAGGCGCCAGATGCGGAATGTGATTGGAATTT |
| | | AG (SEQ ID NO: 62) |
| TcYC6_0106870 | kinetoplastid- | MGKKYAQLETLHNVNGRVVIVGDIHGCLAQLEDILSVTEFARGRDQLITAGDMVNKGPDSFGVVRLLKSLGARGVIGNHD |
| | specific phospho- | AKLLKLRKKIRKHGALHGKNSQSSLAPLAMSLPQDVEEYLSQLPHILRIPAHNILVVHAGLHVQHPLERQLVKEVTTMRN |
| | protein phosphatase, | LILQDDGLYRASEDTTDGVPWASLWQGPETVVFGHDARRGLQRYPHAIGLDTRCVYGGELTALVCPGEHLVSVPGWTSNR |
| | putative | SKV (SEQ ID NO: 56) |
| | | ATGGGAAAAAAATACGCACAGTTGGAGACTCTCCACAACGTGAATGGGCGGGTTGTGATTGTAGGCGACATTCATGGCTG |
| | | CCTTGCCCAACTGGAGGACATTTTATCAGTCACAGAATTTGCGAGGGGAAGGGATCAGTTAATCACCGCTGGGGACATGG |
| | | TGAACAAAGGGCCAGACTCGTTTGGCGTTGTGCGTCTGCTGAAGAGCCTTGGAGCACGCGGTGTGATTGGCAATCATGAC |
| | | GCCAAGCTTCTCAAACTTCGGAAAAAGATACGAAAACATGGGGCGCTGCACGGGAAGAATAGCCAATCGAGTTTAGCCCC |
| | | GCTTGCCATGTCGCTACCGCAGGATGTTGAAGAGTATTTATCACAACTGCCGCATATTCTCCGCATTCCCGCACACAACA |
| | | TTCTGGTGGTACATGCGGGCCTTCACGTTCAACACCCGCTTGAGCGGCAATTGGTTAAGGAGGTCACTACGATGCGCAAC |
| | | CTCATTTTGCAGGATGACGGGCTGTACAGGGCATCTGAGGATACAACGGACGGTGTGCCGTGGGCATCGCTGTGGCAGGG |
| | | TCCGGAGACTGTTGTCTTTGGCCACGACGCCAGACGAGGCCTCCAACGCTACCCTCATGCGATCGGATTGGACACTCGGT |
| | | GTGTGTATGGCGGGGAGCTCACTGCTCTTGTGTGTCCCGGTGAACACCTCGTTTCCGTGCCTGGATGGACTTCCAATAGA |
| | | TCGAAGGTGTGA (SEQ ID NO: 63) |
| TcBrA4_0101970 | surface protein | MLAVMVMRPFLCALLFFALCRCFPNSACAASATANNATENASAMAPPADMRGALREVLGAMQKAQEYADEANRHCVQARM |
| | TolT, putative | SAESAREHEEGAKNALRKLGSEATRMSRALQQADEAVKLADAAVAECKAAEEAAQAAGIMTLDAVGEVLKHLKDEKTKVG |
| | | SGPELLKRAAEQTVLSLEKAKEAEAEAEKAAAAAQKTRDAAEKAAAARTLAQDVAATASALLRQREKEEERRRARDRVRA |
| | | YVGNERAENAMRVAWLDWVECCVAVLVNEGAEGGNGVFFPIF (SEQ ID NO: 57) |
| | | ATGCTGGCGGTGATGGTGATGCGCCCTTTCTTGTGTGCCCTGCTATTTTTTGCGCTCTGCCGCTGCTTTCCTAATTCCGC |
| | | GTGTGCGGCTTCTGCCACAGCCAATAATGCCACTGAGAATGCCAGTGCTATGGCGCCACCGGCTGATATGAGGGGGGCGT |
| | | TGAGAGAGGTGTTGGGAGCCATGCAGAAGGCGCAGGAGTATGCTGACGAGGCTAACCGGCACTGCGTGCAGGCAAGAATG |
| | | AGCGCTGAGAGTGCGCGGGAGCATGAAGAGGGGGCTAAGAATGCTTTGAGGAAGCTCGGCTCTGAGGCTACAAGGATGAG |
| | | CAGGGCGCTGCAGCAAGCGGACGAGGCTGTGAAATTGGCCGATGCTGCCGTGGCCGAATGCAAGGCGGCGGAGGAGGCTG |
| | | CACAGGCGGCGGGGATAATGACGCTTGATGCCGTTGGGGAGGTGCTGAAGCATCTGAAGGACGAGAAGACCAAGGTTGGA |
| | | AGTGGACCGGAGCTGTTGAAGAGGGCGGCAGAGCAGACTGTGCTTTCTCTGGAGAAGGCAAAGGAGGCGGAGGCGGAGGC |
| | | TGAGAAGGCGGCAGCGGCGGCGCAGAAAACCCGGGACGCAGCAGAGAAGGCAGCAGCGGCGCGGACCTTGGCACAAGATG |
| | | TTGCCGCAACGGCCAGTGCGCTGCTGCGGCAGCGGGAGAAGGAGGAGGAGAGGCGAAGAGCGAGGGACAGGGTGAGGGCT |
| | | TACGTTGGAAATGAACGCGCCGAGAATGCCATGAGGGTTGCGTGGCTGGACTGGGTGGAGTGTTGTGTTGCAGTTCTTGT |
| | | CAATGAAGGAGCTGAAGGTGGCAATGGCGTTTTTTTTCCTATATTTTAA (SEQ ID NO: 64) |
| TcYC6_0077100 | surface protein TolT | MLAVMVMRPFLCALLFFALCCCFPNSVCAADDTAANTTEDVNASAIPTNMKEAFDWAFKAMFKAREEVDEASQHCVQAKL |
| | | SAAKAAGLEREAEMALKKLGAEAMTLSKALRDARGANSEAKAAVTECEAAEEAAQQAEIATLDAAYEVLNHVKTDRRSKN |
| | | SKTEGLLDEAAKHTAIAVKKAKEAEAESEKAAAAARKTLEAAEKAAAARTLAQDVAATASALLRQREREEERRRAKDREA |
| | | AEAAKKAAVAEVMKKFAAKKGNDAASGRNSTTTRIQRTRPRVDGGGIPLLLRAPLLMLAAVASVFGFLSC (SEQ ID |
| | | NO: 58) |
| | | ATGCTGGCGGTGATGGTGATGCGCCCTTTCTTGTGTGCCCTGCTGTTTTTTGCGCTCTGCTGCTGCTTTCCTAATTCCGT |
| | | GTGTGCGGCGGATGATACAGCTGCTAATACCACTGAGGATGTCAATGCTTCGGCGATACCGACTAATATGAAAGAAGCTT |
| | | TTGACTGGGCATTCAAGGCGATGTTTAAGGCGCGGGAGGAGGTGGATGAGGCCAGCCAGCATTGTGTGCAAGCTAAACTA |
| | | AGTGCGGCGAAGGCAGCTGGGCTTGAGAGAGAAGCAGAGATGGCTTTAAAAAAGCTTGGCGCGGAGGCTATGACACTGAG |
| | | CAAGGCATTACGAGATGCGAGGGGAGCCAACAGTGAAGCTAAGGCTGCTGTGACCGAATGCGAGGCTGCAGAGGAGGCTG |
| | | CACAACAAGCAGAGATTGCGACGCTCGATGCCGCATACGAGGTGCTGAACCATGTTAAGACCGATAGGAGGAGCAAGAAT |
| | | TCCAAAACAGAGGGTCTTTTGGACGAGGCGGCAAAGCATACTGCAATTGCCGTAAAGAAGGCAAAGGAGGCGGAGGCGGA |
| | | GTCTGAGAAGGCGGCAGCGGCGGCGCGGAAAACCCTGGAAGCAGCAGAGAAGGCAGCAGCGGCGCGGACCTTGGCACAAG |
| | | ACGTTGCCGCAACGGCCAGTGCGCTGCTGCGGCAGCGGGAGAGGGAGGAGGAGAGACGAAGAGCGAAGGACCGGGAGGCG |
| | | GCGGAGGCCGCGAAAAAGGCTGCCGTTGCTGAGGTGATGAAGAAATTTGCTGCGAAGAAGGGGAATGACGCGGCGTCTGG |
| | | CAGGAATTCCACGACCACCCGCATTCAAAGGACGAGACCGCGGGTGGATGGCGGCGGCATTCCATTGCTTTTGCGTGCAC |
| | | CGCTTCTGATGCTTGCTGCCGTGGCATCCGTTTTCGGCTTCTTATCGTGCTAG (SEQ ID NO: 65) |
| TcYC6_0078140 | surface protein | MLAVMVKRPFLCAPLFFALCCCFPNSVCAASATANNATENVSSMAPPADMRGALREVLGAMQKAQEYADEANRHCVQAGM |
| | TolT, putative | SAKNAREHEEGAKNALRKLGSEATRMSRALQQAEEAVKLADAAVAECKAAEEAAQAAGIMTLDAVGEVLKHVKDEKTKVG |
| | | SGPELLKRAAEQTVLSLEKAKEAEAETEKAAAAAQKTREAAEKAAAAQTLAQDVAATAIALLRQREKEEERRRARDREEA |
| | | EAAKKAAVAEVMNKFAAKKGNDAAPGRNSTATRIQRTRPRVDGGGIPLLLRAPLLMLAAVASVFGFLSC (SEQ ID |
| | | NO: 59) |
| | | ATGCTGGCGGTGATGGTGAAGCGCCCTTTCTTGTGTGCCCCGCTTTTTTTTGCGCTCTGCTGCTGCTTTCCTAATTCCGT |
| | | GTGTGCGGCTTCTGCCACAGCCAATAATGCCACTGAGAATGTCAGTTCTATGGCGCCACCGGCTGATATGAGGGGGGCGT |
| | | TGAGAGAGGTGTTGGGAGCCATGCAGAAGGCGCAGGAGTATGCTGATGAGGCTAACCGGCACTGCGTGCAAGCAGGAATG |
| | | AGCGCTAAGAATGCGCGGGAGCATGAAGAGGGGGCTAAGAATGCTTTGAGGAAGCTCGGCTCTGAGGCTACAAGGATGAG |
| | | CAGGGCGCTGCAGCAAGCGGAGGAGGCTGTGAAATTGGCCGATGCTGCCGTGGCCGAATGCAAGGCGGCAGAGGAGGCTG |
| | | CACAGGCGGCGGGGATAATGACGCTTGATGCCGTTGGGGAGGTGCTGAAGCATGTGAAGGACGAGAAGACCAAGGTTGGA |
| | | AGTGGACCGGAGCTGTTGAAGAGGGCGGCGGAGCAGACTGTGCTTTCTCTGGAGAAGGCAAAGGAGGCGGAGGCGGAGAC |
| | | TGAGAAGGCGGCAGCGGCGGCGCAGAAAACCCGGGAAGCAGCAGAGAAGGCAGCAGCGGCGCAGACCTTGGCACAAGATG |
| | | TTGCCGCAACGGCCATTGCGCTGCTGCGGCAGCGGGAGAAGGAGGAGGAGAGGCGAAGAGCGAGGGACCGGGAGGAGGCG |
| | | GAGGCCGCGAAGAAGGCTGCCGTTGCTGAGGTGATGAATAAATTTGCTGCGAAGAAGGGGAATGACGCAGCGCCTGGCAG |
| | | GAATTCCACAGCCACCCGCATTCAAAGGACGAGACCACGGGTGGATGGCGGCGGCATTCCATTGCTTTTGCGTGCACCGC |
| | | TTCTGATGCTTGCTGCCGTGGCATCCGTTTTCGGCTTCTTATCGTGCTAG (SEQ ID NO: 66) |

2. Linker Domains

In some embodiments, to fusion protein includes one or more linkers or spacers. Peptide linker sequences are typically two or more amino acids in length. In some embodiments linker or spacer is one or more polypeptides. In some embodiments, the linker includes a glycine-glutamic acid di-amino acid sequence. The linkers can be used to link or connect two domains, regions, or sequences of the fusion protein.

Suitable peptide/polypeptide linker domains include naturally occurring or non-naturally occurring peptides or polypeptides. Preferably the peptide or polypeptide domains are flexible peptides or polypeptides. A “flexible linker” herein refers to a peptide or polypeptide containing two or more amino acid residues joined by peptide bond(s) that provides increased rotational freedom for two polypeptides linked thereby than the two linked polypeptides would have in the absence of the flexible linker. Such rotational freedom allows two or more antigen binding sites joined by the flexible linker to each access target antigen(s) more efficiently. Exemplary flexible peptides/polypeptides include, but are not limited to, the amino acid sequences Gly-Ser, Gly-Ser-Gly-Ser (SEQ ID NO:45), Ala-Ser, Gly-Gly-Gly-Ser (SEQ ID NO:46), (Gly₄-Ser)₃ (SEQ ID NO:47), (Gly₄-Ser)₄ (SEQ ID NO:48), and Gly-Gly-Gly-Gly-Ser (SEQ ID NO:109). Additional flexible peptide/polypeptide sequences are well known in the art.

3. Dimerization and Multimerization Domains

The fusion proteins disclosed herein can optionally contain a dimerization or multimerization domain that functions to dimerize or multimerize two or more fusion proteins. The domain that functions to dimerize or multimerize the fusion proteins can either be a separate domain, or alternatively can be contained within one of the other domains of the fusion protein.

a. Dimerization Domains

A “dimerization domain” is formed by the association of at least two amino acid residues or of at least two peptides or polypeptides (which may have the same, or different, amino acid sequences). The peptides or polypeptides may interact with each other through covalent and/or non-covalent association(s). Preferred dimerization domains contain at least one cysteine that is capable of forming an intermolecular disulfide bond with a cysteine on the partner fusion protein. The dimerization domain can contain one or more cysteine residues such that disulfide bond(s) can form between the partner fusion proteins. In one embodiment, dimerization domains contain one, two or three to about ten cysteine residues. An exemplary dimerization domain is the hinge region of an immunoglobulin. Such a dimerization domain can be contained within the linker peptide/polypeptide of the fusion protein.

Additional exemplary dimerization domain can be any known in the art and include, but not limited to, coiled coils, acid patches, zinc fingers, calcium hands, a C_{H}1-C_{L} pair, an “interface” with an engineered “knob” and/or “protruberance” as described in U.S. Pat. No. 5,821,333, leucine zippers (e.g., from jun and/or fos) (U.S. Pat. No. 5,932,448), SH2 (src homology 2), SH3 (src Homology 3) (Vidal, et al., Biochemistry, 43, 7336-44 ((2004)), phosphotyrosine binding (PTB) (Zhou, et al., Nature, 378:584-592 (1995)), WW (Sudol, Prog. Biochys. Mol. Bio., 65:113-132 (1996)), PDZ (Kim, et al., Nature, 378: 85-88 (1995); Komau, et al., Science, 269:1737-1740 (1995)) 14-3-3, WD40 (Hu, et al., J Biol Chem., 273, 33489-33494 (1998)) EH, Lim, an isoleucine zipper, a receptor dimer pair (e.g., interleukin-8 receptor (IL-8R); and integrin heterodimers such as LFA-1 and GPIIIb/IIIa), or the dimerization region(s) thereof, dimeric ligand polypeptides (e.g. nerve growth factor (NGF), neurotrophin-3 (NT-3), interleukin-8 (IL-8), vascular endothelial growth factor (VEGF), VEGF-C, VEGF-D, PDGF members, and brain-derived neurotrophic factor (BDNF) (Arakawa, et al., J. Biol. Chem., 269(45): 27833-27839 (1994) and Radziejewski, et al., Biochem., 32(48): 1350 (1993)) and can also be variants of these domains in which the affinity is altered. The polypeptide pairs can be identified by methods known in the art, including yeast two hybrid screens. Yeast two hybrid screens are described in U.S. Pat. Nos. 5,283,173 and 6,562,576, both of which are herein incorporated by reference in their entireties. Affinities between a pair of interacting domains can be determined using methods known in the art, including as described in Katahira, et al., J. Biol. Chem., 277, 9242-9246 (2002)). Alternatively, a library of peptide sequences can be screened for heterodimerization, for example, using the methods described in WO 01/00814. Useful methods for protein-protein interactions are also described in U.S. Pat. No. 6,790,624.

b. Multimerization Domains

A “multimerization domain” is a domain that causes three or more peptides or polypeptides to interact with each other through covalent and/or non-covalent association(s). Suitable multimerization domains include, but are not limited to, coiled-coil domains. A coiled-coil is a peptide sequence with a contiguous pattern of mainly hydrophobic residues spaced 3 and 4 residues apart, usually in a sequence of seven amino acids (heptad repeat) or eleven amino acids (undecad repeat), which assembles (folds) to form a multimeric bundle of helices. Coiled-coils with sequences including some irregular distribution of the 3 and 4 residues spacing are also contemplated. Hydrophobic residues are in particular the hydrophobic amino acids Val, Ile, Leu, Met, Tyr, Phe and Trp. Mainly hydrophobic means that at least 50% of the residues must be selected from the mentioned hydrophobic amino acids.

The coiled coil domain may be derived from laminin. In the extracellular space, the heterotrimeric coiled coil protein laminin plays an important role in the formation of basement membranes. Apparently, the multifunctional oligomeric structure is required for laminin function. Coiled coil domains may also be derived from the thrombospondins in which three (TSP-1 and TSP-2) or five (TSP-3, TSP-4 and TSP-5) chains are connected, or from COMP (COMPcc) (Guo, et al., EMBO J., 1998, 17: 5265-5272) which folds into a parallel five-stranded coiled coil (Malashkevich, et al., Science, 274: 761-765 (1996)).

Additional coiled-coil domains derived from other proteins, and other domains that mediate polypeptide multimerization are known in the art and are suitable for use in the disclosed fusion proteins.

4. Other Domains

The fusion protein can optionally include additional sequences or moieties, including, but not limited to purification tags and/or solubility enhancers.

In some embodiments the purification tag is a polypeptide. Polypeptide purification tags are known in the art and include, but are not limited to His tags which typically include six or more, typically consecutive, histidine residues; green fluorescent protein (GFP), glutathione S-transferase (GST), polyhistidine, c-myc, hemagglutinin, Flag™ tag (Kodak, New Haven, CT), maltose E binding protein and protein A. More specific examples include FLAG tags including the sequence DYKDDDDK (SEQ ID NO:49); haemagglutinin (HA) tags including the sequence YPYDVP (SEQ ID NO:50); or MYC tags including the sequence ILKKATAYIL (SEQ ID NO:51) or EQKLISEEDL (SEQ ID NO:52). Methods of using purification tags to facilitate protein purification are known in the art and include, for example, a chromatography step wherein the tag reversibly binds to a chromatography resin.

Although many recombinant proteins can be produced by recombinant organisms, the yield and quality of the expressed protein are variable due to many factors. For example, heterologous protein expression by genetically engineered organisms can be affected by the size and source of the protein to be expressed, the presence of an affinity tag linked to the protein to be expressed, codon biasing, the strain of the microorganism, the culture conditions of microorganism, and the in vivo degradation of the expressed protein. In some embodiments, the fusion proteins and other polypeptide are designed so they are reasonably small and do not require a solubilizing polypeptide to enhance solubility. In other embodiments, expression problems can be mitigated by fusing the protein of interest to an expression or solubility enhancing amino acid sequence. Exemplary expression or solubility enhancing amino acid sequences include maltose-binding protein (MBP), glutathione S-transferase (GST), thioredoxin (TRX), NUS A, ubiquitin (Ub), and a small ubiquitin-related modifier (SUMO).

In some embodiments, the compositions disclosed herein include expression or solubility enhancing amino acid sequence. In some embodiments, the expression or solubility enhancing amino acid sequence is cleaved prior to use. The expression or solubility enhancing amino acid sequence can be cleaved in the recombinant expression system, or after the expressed protein in purified. In some embodiments, the expression or solubility enhancing is a ULP1 or SUMO sequence. Recombinant protein expression systems that incorporate the SUMO protein (“SUMO fusion systems”) have been shown to increase efficiency and reduce defective expression of recombinant proteins in E. coli., see for example Malakhov, et al., J. Struct. Funct. Genomics, 5: 75-86 (2004), U.S. Pat. Nos. 7,060,461, and 6,872,551. SUMO fusion systems enhance expression and solubility of certain proteins, including severe acute respiratory syndrome coronavirus (SARS-CoV) 3CL protease, nucleocapsid, and membrane proteins (Zuo et al., J. Struct. Funct. Genomics, 6:103-111 (2005)).

Purifications tags and solubility enhancers can be inserted anywhere in the fusions, including, but not limited, N-terminal or C-terminal or internal relative to other domains (e.g., T. cruzi polypeptides) of the fusion protein.

5. Exemplary Fusion Protein Constructs

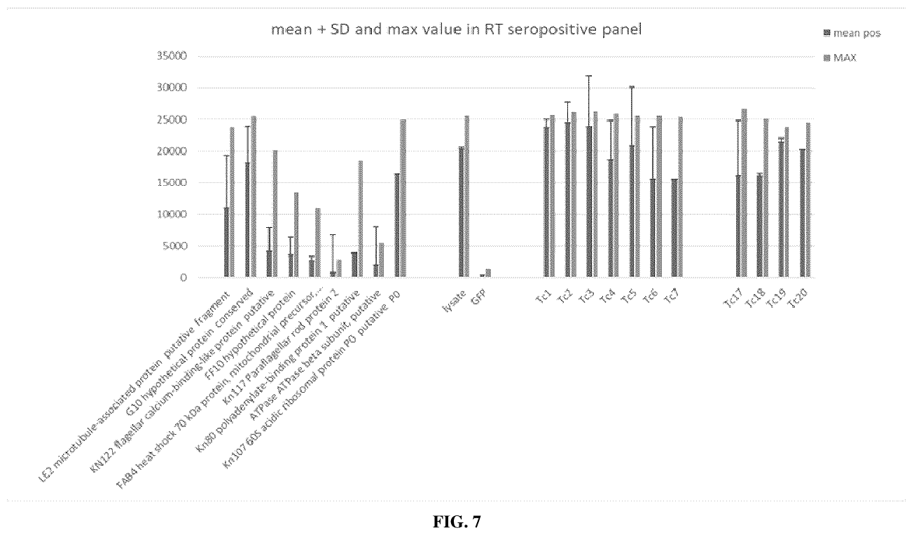

Table 2 provides preferred polypeptide combinations. These combinations, when presented as fusion proteins for testing against a panel of 17 seropositive sera, demonstrated that in comparison to previously used single T. cruzi recombinant proteins, were superior in consistently detecting anti-T. cruzi antibodies. See, e.g., Example 1 below, and FIG. 7.

TABLE 2
Exemplary Fusion Protein Combinations

| Designation | Gene ID 1* | Gene ID 2* | Gene name 1 | Gene name 2 |
|---|---|---|---|---|
| Tc1 | TcBrA4_0116860 | TcYC6_0028190 | 60S acidic | 60S acidic ribosomal |
| | | | ribosomal protein | protein P2 putative |
| | | | putative | |
| Tc2 | TcBrA4_0088420 | TcBrA4_0101960 | 60S ribosomal | surface protein TolT |
| | | | protein L19 putative | |
| Tc3 | TcBrA4_0104680 | TcBrA4_0101980 | RNA-binding | mucin-associated |
| | | | protein putative | surface protein |
| | | | | (MASP), |
| | | | | syntenic/homologous |
| | | | | with Surface protein |
| | | | | TolT, group C |
| Tc4 | TcBrA4_0028480 | TcBrA4_0088260 | reticulon domain | 60S ribosomal protein |
| | | | protein putative | L23a putative |
| Tc5 | TcYC6_0100010 | TcBrA4_0074300 | 60S ribosomal | 40S ribosomal protein |
| | | | protein L7a putative | S4 putative |
| Tc6 | TcYC6_0043560 | TcYC6_0122760 | 40S ribosomal | hypothetical protein |
| | | | protein S21 putative | conserved |
| Tc7 | TcYC6_0083710 | TcBrA4_0130080 | 40S ribosomal | 60S ribosomal protein |
| | | | protein S8 putative | L13 putative |
| Tc15 | TcBrA4_0056330 | TcBrA4_0033670 | hypothetical protein | kinetoplastid-specific |
| | | | | phospho-protein |
| | | | | phosphatase, putative |
| Tc16 | TcYC6_0074990 | TcYC6_0106870 | hypothetical protein | kinetoplastid-specific |
| | | | | phospho-protein |
| | | | | phosphatase, putative |
| Tc17 | TcBrA4_0028230 | TcBrA4_0029760 | hypothetical protein, | calcium-binding |
| | | | conserved | protein, putative |
| Tc18 | TcYC6_0097920 | TcYC6_0096240 | hypothetical protein, | calcium-binding |
| | | | conserved | protein, putative |
| Tc19 | TcBrA4_0122270 | TcBrA4_0131050 | ubiquitin- | 60S acidic ribosomal |
| | | | conjugating enzyme | protein P2, putative |
| | | | E2, putative | |
| Tc20 | TcYC6_0088050 | TcYC6_0111870 | ubiquitin- | 60S acidic ribosomal |
| | | | conjugating enzyme | protein P2, putative |
| | | | E2, putative | |

*Gene ID's and names are from the TriTrypDB database TriTrypDB.org

In another example, the combination is of TcBrA4_0101970, TcYC6_0077100, and TcYC6_0078140, or fragment(s) or variant(s) thereof. In a specific example the fusion protein is a fusion of aa 150-260 for TcBrA4_0101970, TcYC6_0077100, and TcYC6_0078140 with linkers in between (also referred to herein as 3TolT). Tolt3 is a fusion of 3 variants of the same gene present in different genetic types of parasites (i.e., this single protein should detect infections by multiple different parasite lineages/strains). This is often a concern with T. cruzi—that the genetic variation in the population makes detection of all infections difficult.

TABLE 3
Exemplary Fusion Constructs

| | | | size | Size | | | |
|---|---|---|---|---|---|---|---|
| | Gene Id | DNA Sequence | (bp) | (aa) | | Optimized DNA sequence | Amino Acid sequence |
| Tc1 | TcBrA4_ | ATGTCCTCCAAACAGCAGCTTGCCT | 678 | 226 | 60S | ATGAGCAGCAAGCAGCAACTGGCGT | MSSKQQLACTYAALILADSGKTDMDSL |
| | 0116860 + | GCACCTACGCCGCCCTGATTCTTGC | | | acidic | GCACCTATGCGGCGCTGATCCTGGC | LKVTKAAGVDVSKGMASAFASILKNVD |
| | TcYC6_ | CGATAGCGGCAAGACGGATATGGAC | | | rib & | GGACAGCGGTAAAACCGACATGGAT | INDVLSKVSFGGVAPAAGGATAAPAAA |
| | 0028190 | AGCCTGTTGAAAGTGACAAAGGCCG | | | 60S | AGCCTGCTGAAGGTGACCAAAGCGG | AAAAAPAAAAAKKEEEEEDDDMGFGLF |
| | | CCGGTGTTGACGTCAGCAAAGGGAT | | | acidic | CGGGTGTGGATGTTAGCAAGGGCAT | DGGGGSMSMKYLAAYALASLNKPTPGA |
| | | GGCCTCGGCGTTTGCCAGCATCCTC | | | rib P2 | GGCGAGCGCGTTCGCGAGCATCCTG | ADVEAICKACGIEVESDALSFVMESIA |
| | | AAGAACGTTGACATCAACGACGTGC | | | | AAGAACGTGGACATTAACGATGTGC | GRSVATLVAEGAAKMSAVAVSAAPAAG |
| | | TCTCCAAAGTGAGCTTTGGTGGTGT | | | | TGAGCAAAGTTAGCTTTGGTGGCGT | GAAAPAAAAGGAAAPAAADAKKEEEEE |
| | | TGCTCCTGCTGCCGGTGGTGCCACC | | | | TGCGCCGGCGGCGGGTGGCGCGACC | DDDMGFGLED (SEQ ID NO: 67) |
| | | GCTGCTCCTGCTGCTGCTGCTGCTG | | | | GCGGCGCCGGCTGCTGCTGCGGCGG | |
| | | CCGCCGCCCCTGCCGCCGCCGCCGC | | | | CGGCGGCGCCGGCGGCGGCGGCGGC | |
| | | AAAGAAGGAAGAGGAAGAGGAAGAC | | | | GAAGAAAGAGGAAGAGGAAGAGGAC | |
| | | GACGATATGGGCTTTGGTCTGTTTG | | | | GATGACATGGGTTTCGGCCTGTTTG | |
| | | ACGGAGGTGGTGGCTCTATGTCCAT | | | | ACGGTGGCGGTGGCAGCATGAGCAT | |
| | | GAAGTACCTCGCCGCATACGCTCTT | | | | GAAGTACCTGGCGGCGTATGCGCTG | |
| | | GCTTCGCTGAACAAACCAACGCCAG | | | | GCGAGCCTGAACAAACCGACCCCGG | |
| | | GCGCCGCCGATGTGGAGGCCATCTG | | | | GTGCGGCGGATGTTGAGGCGATCTG | |
| | | CAAGGCCTGCGGTATCGAAGTTGAG | | | | CAAGGCGTGCGGCATTGAGGTGGAA | |
| | | AGCGACGCACTCTCGTTTGTCATGG | | | | AGCGATGCGCTGAGCTTCGTTATGG | |
| | | AATCCATTGCCGGCCGGAGCGTTGC | | | | AGAGCATTGCGGGTCGTAGCGTGGC | |
| | | CACTCTCGTGGCGGAGGGCGCGGCG | | | | GACCCTGGTTGCGGAGGGTGCGGCG | |
| | | AAGATGAGCGCTGTTGCCGTCTCCG | | | | AAAATGAGCGCGGTGGCGGTTAGCG | |
| | | CTGCTCCTGCTGCCGGTGGTGCAGC | | | | CGGCTCCTGCGGCGGGTGGCGCGGC | |
| | | CGCTCCTGCTGCTGCTGCTGGCGGT | | | | TGCTCCGGCGGCGGCGGCGGGTGGC | |
| | | GCCGCCGCCCCTGCCGCTGCTGACG | | | | GCGGCCGCTCCGGCGGCGGCGGACG | |
| | | CCAAGAAGGAAGAAGAGGAGGAGGA | | | | CGAAGAAAGAAGAGGAAGAGGAAGA | |
| | | CGATGACATGGGCTTTGGTCTGTTT | | | | TGACGATATGGGCTTTGGCCTGTTT | |
| | | GAC (SEQ ID NO: 81) | | | | GAT (SEQ ID NO: 82) | |
| Tc2 | TcBrA4_ | ATGGTGTCGCTGAAGCTGCAGGCTC | 1818 | 606 | 60S | ATGGTGAGCCTGAAGCTGCAAGCGC | MVSLKLQARLAADILRCGRHRVWLDPN |
| | 0088420 + | GTTTGGCGGCGGACATTCTCCGCTG | | | ribosomal | GTCTGGCGGCGGACATCCTGCGTTG | EASEISNANSRKSVRKLIKDGLIIRKP |
| | TcBrA4_ | CGGTCGCCACCGTGTGTGGCTGGAC | | | protein | CGGCCGTCACCGTGTGTGGCTGGAT | VKVHSRSRWRHMKEAKSMGRHEGAGRR |
| | 0101960 | CCTAATGAGGCCTCTGAGATTTCCA | | | L19, | CCGAACGAAGCGAGCGAGATCAGCA | EGTREARMPSKELWMRRLRILRRLLRK |
| | | ATGCAAACTCGCGCAAGAGCGTGCG | | | surface | ACGCGAACAGCCGTAAAAGCGTTCG | YREEKKIDRHIYRELYVKAKGNVFRNK |
| | | CAAGTTGATCAAGGATGGTCTGATT | | | protein | TAAACTGATTAAGGACGGTCTGATC | RNLMEHIHKVKNEKKKERQLAEQLAAK |
| | | ATTCGCAAGCCTGTCAAGGTGCACT | | | TolT | ATTCGTAAACCGGTGAAGGTTCACA | RLKDEQHRHKARKQELRKREKDRERAR |
| | | CGCGCTCCCGCTGGCGCCACATGAA | | | | GCCGTAGCCGTTGGCGTCACATGAA | REDAAAAAAAKQKAAAKKAAAPSGKKS |
| | | GGAGGCGAAGAGCATGGGCCGCCAC | | | | AGAGGCGAAAAGCATGGGTCGTCAT | AKASAPAKAATAPAKATAAPAKAAAAP |
| | | GAGGGCGCTGGGCGCCGCGAGGGTA | | | | GAGGGTGCGGGTCGTCGTGAAGGCA | AKATAAPAKATAAPAKAAAAPAKATAA |
| | | CCCGCGAAGCCCGCATGCCGAGCAA | | | | CCCGTGAGGCGCGTATGCCGAGCAA | PAKAAAAPAKAAAAPAKAAAAPAKAAA |
| | | GGAGCTGTGGATGCGCCGTCTGCGC | | | | GGAACTGTGGATGCGTCGTCTGCGT | APAKATAAPAKAAAAPAKVAAAPAKAA |
| | | ATTCTCCGCCGCCTGCTGCGCAAGT | | | | ATCCTGCGTCGTCTGCTGCGCAAAT | AAPVGKKAGGKKGGGGSMAPPADMRGA |
| | | ACCGCGAGGAGAAGAAGATTGACCG | | | | ACCGTGAGGAAAAGAAAATCGACCG | LREVLGAMQKAQEYADEANRHCVQARM |
| | | CCACATTTACCGCGAGCTGTACGTG | | | | TCACATTTACCGTGAACTGTATGTG | SAESAREHEEGAKNALRKLGSEATRMS |
| | | AAGGCGAAGGGGAACGTGTTTCGCA | | | | AAAGCGAAGGGCAACGTTTTCCGTA | RALQQADEAVKLADAAVAECKAAEEAA |
| | | ACAAGCGTAACCTCATGGAGCACAT | | | | ACAAGCGTAACCTGATGGAGCACAT | QAAGIMTLDAVGEVLKHLKDEKTKVGS |
| | | CCACAAGGTGAAGAACGAGAAGAAG | | | | CCACAAGGTTAAGAACGAAAAGAAA | GPELLKRAAEQTVLSLEKAKEAEAEAE |
| | | AAGGAAAGGCAGCTGGCTGAGCAGC | | | | AAGGAGCGTCAGCTGGCGGAGCAAC | KAAAAAQKTREAAEKAAAARTLAQDVA |
| | | TCGCGGCGAAGCGCCTGAAGGATGA | | | | TGGCGGCGAAACGTCTGAAGGATGA | ATASALLRQREKEEERRRARDQEVAEA |
| | | GCAGCACCGTCACAAGGCCCGCAAG | | | | ACAGCACCGTCACAAAGCGCGTAAG | AKKAAVAEVMKKFAAKGNDTAPGRNST |
| | | CAGGAGCTGCGTAAGCGCGAGAAGG | | | | CAAGAGCTGCGTAAACGTGAAAAGG | STRFQRTRPRVDGGGIPLLLRAPLLML |
| | | ACCGCGAGCGTGCGCGTCGCGAAGA | | | | ACCGTGAGCGTGCGCGTCGTGAGGA | AAVASVFGFLSC (SEQ ID NO: 68) |
| | | TGCTGCTGCTGCCGCCGCCGCGAAG | | | | TGCGGCTGCTGCGGCGGCGGCGAAA | |
| | | CAGAAGGCAGCTGCGAAGAAGGCCG | | | | CAGAAAGCGGCGGCGAAAAAGGCGG | |
| | | CTGCTCCCTCTGGCAAGAAGTCCGC | | | | CGGCGCCGAGCGGTAAAAAGAGCGC | |
| | | GAAGGCTTCTGCACCTGCCAAGGCC | | | | GAAAGCGAGCGCGCCGGCGAAGGCG | |
| | | GCTACTGCACCCGCGAAGGCCACTG | | | | GCGACCGCGCCGGCGAAAGCGACCG | |
| | | CCGCACCCGCGAAGGCTGCTGCTGC | | | | CGGCGCCGGCGAAAGCGGCGGCGGC | |
| | | ACCCGCGAAGGCCACTGCTGCACCC | | | | GCCGGCGAAGGCTACTGCGGCGCCG | |
| | | GCGAAGGCCACTGCTGCACCCGCGA | | | | GCGAAGGCCACTGCTGCTCCGGCGA | |
| | | AGGCTGCTGCTGCACCCGCGAAGGC | | | | AGGCTGCTGCTGCTCCTGCTAAAGC | |
| | | CACTGCTGCACCCGCGAAGGCCGCT | | | | TACTGCTGCTCCTGCCAAGGCTGCT | |
| | | GCTGCACCCGCGAAGGCTGCTGCTG | | | | GCTGCTCCCGCTAAAGCTGCTGCTG | |
| | | CACCCGCGAAGGCCGCTGCTGCACC | | | | CTCCTGCCAAAGCTGCTGCTGCTCC | |
| | | CGCGAAGGCTGCTGCTGCACCCGCG | | | | CGCCAAAGCTGCTGCTGCCCCGGCG | |
| | | AAGGCCACTGCTGCACCCGCGAAGG | | | | AAGGCTACAGCGGCTCCTGCGAAGG | |
| | | CCGCTGCTGCACCTGCGAAGGTCGC | | | | CTGCTGCGGCGCCGGCGAAAGTTGC | |
| | | TGCTGCACCCGCGAAGGCTGCTGCC | | | | GGCTGCTCCTGCTAAAGCCGCTGCG | |
| | | GCTCCCGTTGGAAAGAAGGCTGGTG | | | | GCGCCGGTTGGCAAAAAGGCGGGTG | |
| | | GCAAGAAGGGAGGTGGTGGCTCTAT | | | | GCAAAAAGGGTGGCGGTGGCAGCAT | |
| | | GGCGCCACCGGCTGATATGAGGGGG | | | | GGCTCCGCCGGCGGACATGCGTGGC | |
| | | GCGTTGAGAGAGGTGTTGGGAGCCA | | | | GCGCTGCGTGAAGTGCTGGGTGCGA | |
| | | TGCAGAAGGCGCAGGAGTATGCTGA | | | | TGCAGAAGGCGCAAGAATATGCGGA | |
| | | CGAGGCTAACCGGCACTGCGTGCAG | | | | TGAGGCGAACCGTCACTGCGTTCAG | |
| | | GCAAGAATGAGCGCTGAGAGTGCGC | | | | GCGCGTATGAGCGCGGAAAGCGCGC | |
| | | GGGAGCATGAAGAGGGGGCTAAGAA | | | | GTGAGCACGAGGAAGGCGCGAAAAA | |
| | | TGCTTTGAGGAAGCTCGGCTCTGAG | | | | CGCGCTGCGTAAGCTGGGTAGCGAG | |
| | | GCTACAAGGATGAGCAGGGCGCTGC | | | | GCGACCCGTATGAGCCGTGCGCTGC | |
| | | AGCAAGCGGACGAGGCTGTGAAATT | | | | AGCAAGCGGATGAAGCGGTGAAGCT | |
| | | GGCCGATGCTGCCGTGGCCGAATGC | | | | GGCGGATGCGGCGGTTGCGGAGTGC | |
| | | AAGGCGGCGGAGGAGGCTGCACAGG | | | | AAGGCGGCGGAGGAAGCGGCGCAAG | |
| | | CGGCGGGGATAATGACGCTTGATGC | | | | CGGCGGGTATCATGACCCTGGATGC | |
| | | CGTTGGGGAGGTGCTGAAGCATCTG | | | | GGTGGGCGAGGTTCTGAAACACCTG | |
| | | AAGGACGAGAAGACCAAGGTTGGAA | | | | AAGGATGAAAAAACCAAAGTGGGCA | |
| | | GTGGACCGGAGCTGTTGAAGAGGGC | | | | GCGGTCCGGAACTGCTGAAACGTGC | |
| | | GGCGGAGCAGACTGTGCTTTCTCTG | | | | GGCGGAGCAGACCGTTCTGAGCCTG | |
| | | GAGAAGGCAAAGGAGGCGGAGGCGG | | | | GAAAAAGCGAAGGAAGCGGAAGCGG | |
| | | AGGCTGAGAAGGCGGCAGCGGCGGC | | | | AAGCGGAAAAGGCGGCGGCGGCGGC | |
| | | GCAGAAAACCCGGGAAGCAGCAGAG | | | | GCAAAAGACCCGTGAAGCGGCGGAG | |
| | | AAGGCAGCAGCGGCGCGGACCTTGG | | | | AAAGCGGCGGCGGCGCGTACCCTGG | |
| | | CACAAGATGTTGCCGCAACGGCCAG | | | | CGCAGGACGTGGCGGCGACCGCGAG | |
| | | TGCGCTGCTGCGGCAGCGGGAGAAG | | | | CGCGCTGCTGCGTCAACGTGAAAAG | |
| | | GAGGAGGAGAGGCGAAGAGCGAGGG | | | | GAAGAGGAGCGTCGTCGTGCGCGTG | |
| | | ACCAGGAGGTGGCGGAGGCCGCGAA | | | | ATCAGGAAGTTGCGGAAGCTGCGAA | |
| | | GAAGGCTGCCGTTGCTGAGGTGATG | | | | AAAGGCGGCTGTGGCGGAGGTTATG | |
| | | AAAAAATTTGCTGCGAAGGGGAATG | | | | AAAAAGTTTGCGGCGAAGGGTAACG | |
| | | ACACAGCGCCTGGCAGGAATTCCAC | | | | ACACCGCGCCGGGTCGTAACAGCAC | |
| | | ATCCACCCGCTTTCAAAGGACGAGG | | | | CAGCACCCGTTTTCAACGTACCCGT | |
| | | CCACGGGTGGATGGCGGCGGCATCC | | | | CCGCGTGTGGATGGTGGCGGTATTC | |
| | | CATTGCTTTTGCGTGCACCGCTTCT | | | | CGCTGCTGCTGCGTGCGCCGCTGCT | |
| | | GATGCTTGCTGCCGTGGCATCCGTT | | | | GATGCTGGCGGCGGTGGCGAGCGTT | |
| | | TTCGGCTTCTTATCGTGC (SEQ | | | | TTCGGTTTTCTGAGCTGC (SEQ | |
| | | ID NO: 83) | | | | ID NO: 84) | |
| Tc3 | TcBrA4_ | ATGCCCGCCAAGTCTGCCAACAAGC | 1767 | 589 | RNA- | ATGCCGGCGAAAAGCGCGAACAAAC | MPAKSANKPASKPAAKPAAKPAAKAPA |
| | 0104680 + | CTGCATCCAAGCCTGCCGCCAAGCC | | | binding | CGGCGAGCAAACCGGCGGCGAAGCC | PKAAAPAPKAAAAAPKPAVRDAKQRSD |
| | TcBrA4_ | CGCTGCGAAGCCTGCCGCCAAGGCT | | | protein, | GGCGGCGAAACCGGCGGCGAAAGCG | AANHNGLYVKNWGQGSVDDARALFGTA |
| | 0101980 | CCCGCACCCAAAGCTGCTGCCCCTG | | | mucin- | CCGGCGCCGAAAGCGGCGGCGCCGG | GKVVGVRVRRRRYAIIFFENAAAVKKA |
| | | CTCCCAAGGCTGCTGCGGCTGCGCC | | | associated | CGCCGAAGGCGGCGGCGGCGGCGCC | IDLFNGKEFMGNVLSVVPAKTTPKPDP |
| | | CAAGCCAGCTGTGAGGGACGCAAAG | | | surface | GAAACCGGCGGTGCGTGATGCGAAG | HANSSVVFVSPIFRASTTKKQILELFS |
| | | CAGCGCTCTGATGCCGCCAATCACA | | | protein | CAGCGTAGCGATGCGGCGAACCACA | GMKVLRLRTYRNNYAYVYLDTPAAAQR |
| | | ACGGCTTGTACGTGAAGAACTGGGG | | | (MASP) | ACGGCCTGTATGTGAAAAACTGGGG | AVKEKNGAEFRGKQLRVALSTRSLAKD |
| | | CCAGGGTTCTGTGGACGACGCCAGG | | | | TCAAGGCAGCGTTGATGATGCGCGT | RARAERARLLIAAQKFNKRKNHTKGGG |
| | | GCGCTTTTTGGCACTGCTGGGAAGG | | | | GCGCTGTTTGGCACCGCGGGCAAGG | GSMTRNRLFFPLLLLLSCSVIVGANAT |
| | | TTGTGGGTGTGAGAGTGCGTCGTCG | | | | TGGTTGGTGTGCGTGTGCGTCGTCG | EKKASTPRKAEGVQPQSVSPSSSFPGD |
| | | CCGTTACGCCATTATCTTCTTTGAG | | | | TCGTTACGCGATCATTTTCTTTGAA | GTGVPLKLELGELRDKALLAAKDAFGN |
| | | AACGCAGCGGCTGTGAAGAAGGCCA | | | | AACGCGGCGGCGGTGAAGAAAGCGA | TTGAAMQCMQAKTDVEETKKYAEEAKK |
| | | TTGATCTTTTCAACGGGAAAGAATT | | | | TCGACCTGTTCAACGGCAAAGAGTT | LFDKIGGDYVSKSAALADAVKASTDAE |
| | | TATGGGCAATGTTTTGTCCGTTGTT | | | | TATGGGTAACGTTCTGAGCGTGGTT | EALKSCVEAEKAAVDADTAVLAAVLEV |
| | | CCCGCCAAGACGACTCCGAAGCCGG | | | | CCGGCGAAAACCACCCCGAAGCCGG | LQHSKFWRRDTAVSTEKLANVSKHSAN |
| | | ATCCGCATGCGAACTCCTCTGTTGT | | | | ATCCGCACGCGAACAGCAGCGTGGT | ATNEAQKAGIQASKAAEAAKRAAESKK |
| | | GTTTGTTTCCCCGATATTCCGCGCG | | | | TTTCGTGAGCCCGATCTTTCGTGCG | KAAAALDTVKEVVAMAEMLKEKFFENE |
| | | TCGACTACAAAGAAGCAGATTCTTG | | | | AGCACCACCAAGAAACAGATTCTGG | RLQKEKHEAQLEAERQFIQEEVQKKEA |
| | | AGCTTTTTTCAGGCATGAAGGTACT | | | | AACTGTTCAGCGGTATGAAGGTTCT | EAEKALNRAAAADKRVAELELARQKQS |
| | | GCGCCTGCGCACGTACCGCAACAAC | | | | GCGTCTGCGTACCTATCGTAACAAC | KEQGNEGRGHRRVRRSGSDSSSNYAPA |
| | | TACGCATACGTCTATCTGGACACCC | | | | TACGCGTATGTGTATCTGGACACCC | YEPRLLLLPLLSFTLFCFVAWC (SEQ |
| | | CAGCTGCCGCGCAAAGGGCTGTGAA | | | | CGGCGGCGGCGCAACGTGCGGTTAA | ID NO: 69) |
| | | GGAGAAGAACGGTGCAGAGTTCCGT | | | | AGAAAAGAACGGCGCGGAGTTCCGT | |
| | | GGCAAGCAACTCAGAGTTGCCCTCT | | | | GGTAAACAACTGCGTGTTGCGCTGA | |
| | | CGACTCGTTCTCTTGCGAAGGACAG | | | | GCACCCGTAGCCTGGCGAAGGATCG | |
| | | GGCTCGTGCGGAGCGTGCAAGACTT | | | | TGCGCGTGCGGAGCGTGCGCGTCTG | |
| | | CTTATAGCCGCCCAAAAGTTCAACA | | | | CTGATTGCGGCGCAAAAATTTAACA | |
| | | AGAGAAAGAACCACACGAAGGGAGG | | | | AGCGTAAGAACCACACCAAGGGTGG | |
| | | TGGTGGCTCTATGACGCGTAATAGG | | | | CGGTGGCAGCATGACCCGTAACCGT | |
| | | CTTTTTTTCCCTCTGCTTCTTCTAC | | | | CTGTTCTTTCCGCTGCTGCTGCTGC | |
| | | TCTCCTGCAGCGTAATTGTCGGCGC | | | | TGAGCTGCAGCGTGATTGTTGGCGC | |
| | | AAATGCAACAGAAAAGAAAGCGTCA | | | | GAACGCGACCGAAAAGAAAGCGAGC | |
| | | ACGCCAAGGAAAGCAGAGGGAGTGC | | | | ACCCCGCGTAAAGCGGAGGGTGTGC | |
| | | AGCCGCAATCGGTCTCACCGTCTTC | | | | AGCCGCAAAGCGTTAGCCCGAGCAG | |
| | | GTCGTTTCCAGGGGATGGGACGGGT | | | | CAGCTTTCCGGGTGATGGCACCGGT | |
| | | GTGCCGCTCAAATTGGAACTGGGGG | | | | GTGCCGCTGAAGCTGGAACTGGGCG | |
| | | AACTGAGGGACAAAGCATTGCTGGC | | | | AGCTGCGTGACAAAGCGCTGCTGGC | |
| | | AGCAAAGGATGCTTTTGGCAATACG | | | | GGCGAAGGATGCGTTTGGCAACACC | |
| | | ACAGGGGCGGCAATGCAATGCATGC | | | | ACCGGTGCGGCGATGCAGTGCATGC | |
| | | AGGCCAAGACGGATGTCGAAGAGAC | | | | AAGCGAAAACCGATGTTGAGGAAAC | |
| | | CAAGAAATACGCCGAAGAGGCGAAA | | | | CAAGAAATATGCGGAGGAAGCGAAG | |
| | | AAGCTTTTTGATAAGATTGGCGGGG | | | | AAACTGTTCGACAAAATTGGTGGCG | |
| | | ACTATGTGTCAAAAAGTGCTGCTCT | | | | ATTATGTGAGCAAAAGCGCGGCGCT | |
| | | GGCGGATGCAGTGAAAGCTAGCACC | | | | GGCGGATGCGGTTAAGGCGAGCACC | |
| | | GACGCCGAAGAGGCGCTGAAAAGCT | | | | GATGCGGAGGAAGCGCTGAAAAGCT | |
| | | GTGTGGAGGCGGAAAAGGCCGCTGT | | | | GCGTGGAGGCGGAAAAAGCGGCGGT | |
| | | TGATGCTGATACCGCGGTTTTAGCT | | | | GGATGCGGATACCGCGGTTCTGGCG | |
| | | GCTGTCCTGGAGGTGCTGCAACATT | | | | GCGGTGCTGGAAGTTCTGCAGCACA | |
| | | CCAAGTTTTGGCGAAGGGACACTGC | | | | GCAAATTTTGGCGTCGTGACACCGC | |
| | | AGTTTCGACTGAAAAATTGGCGAAT | | | | GGTGAGCACCGAAAAACTGGCGAAC | |
| | | GTCAGTAAACATTCGGCGAACGCCA | | | | GTTAGCAAGCACAGCGCGAACGCGA | |
| | | CAAATGAGGCGCAAAAGGCAGGGAT | | | | CCAACGAGGCGCAGAAGGCGGGTAT | |
| | | TCAAGCGTCGAAGGCGGCAGAAGCG | | | | CCAAGCGAGCAAAGCGGCGGAAGCG | |
| | | GCGAAGAGGGCAGCGGAGTCGAAAA | | | | GCGAAGCGTGCGGCGGAGAGCAAGA | |
| | | AAAAAGCTGCAGCAGCTCTGGATAC | | | | AAAAGGCGGCGGCGGCGCTGGATAC | |
| | | GGTCAAGGAAGTCGTTGCGATGGCC | | | | CGTTAAAGAGGTGGTTGCGATGGCG | |
| | | GAGATGTTGAAGGAAAAGTTTTTCG | | | | GAAATGCTGAAAGAGAAGTTCTTTG | |
| | | AGAATGAGAGGCTGCAAAAGGAAAA | | | | AGAACGAACGTCTGCAGAAAGAAAA | |
| | | ACATGAGGCTCAATTGGAAGCCGAA | | | | GCACGAGGCGCAACTGGAGGCGGAA | |
| | | AGACAGTTCATTCAGGAAGAGGTAC | | | | CGTCAGTTCATTCAAGAGGAAGTGC | |
| | | AGAAGAAGGAGGCGGAGGCCGAAAA | | | | AGAAGAAAGAGGCGGAAGCGGAGAA | |
| | | GGCACTCAATCGCGCTGCTGCGGCT | | | | AGCGCTGAACCGTGCGGCGGCGGCG | |
| | | GATAAACGTGTCGCCGAGTTGGAAC | | | | GATAAGCGTGTTGCGGAACTGGAGC | |
| | | TTGCCAGACAAAAGCAGAGCAAAGA | | | | TGGCGCGTCAGAAACAAAGCAAGGA | |
| | | GCAGGGGAATGAAGGAAGAGGCCAT | | | | ACAAGGCAACGAGGGTCGTGGTCAC | |
| | | AGGCGAGTCAGACGCAGTGGGAGTG | | | | CGTCGTGTGCGTCGTAGCGGTAGCG | |
| | | ACAGCAGCAGCAACTATGCGCCTGC | | | | ACAGCAGCAGCAACTATGCGCCGGC | |
| | | ATATGAACCACGGCTACTGTTACTG | | | | GTATGAACCGCGTCTGCTGCTGCTG | |
| | | CCTCTGCTTTCTTTCACACTGTTCT | | | | CCGCTGCTGAGCTTTACCCTGTTCT | |
| | | GTTTTGTTGCATGGTGC (SEQ ID | | | | GCTTTGTTGCGTGGTGC (SEQ ID | |
| | | NO: 85) | | | | NO: 86) | |
| Tc4 | TcBrA4_ | ATGGCGTTTTGTATCATTTCTGAGA | 113 | 391 | reticulon | ATGGCGTTCTGCATCATTAGCGAAA | MAFCIISESRGMSLWDMLAWHRPKVTG |
| | 0028480 + | GCAGGGGCATGTCTCTGTGGGATAT | | | domain | GCCGTGGTATGAGCCTGTGGGACAT | VLLGTVLSVLTFFCLMKYTMVTFLCRI |
| | TcBrA4_ | GCTAGCGTGGCACCGCCCAAAAGTT | | | protein, | GCTGGCGTGGCACCGTCCGAAAGTG | LQLVLLAGVLLGFTNRWHLTSDDIHEA |
| | 0088260 | ACGGGTGTACTTCTTGGAACCGTAC | | | 60S | ACCGGTGTTCTGCTGGGCACCGTGC | VNRLVDCATPRLVTALESMHQLVTWRD |
| | | TTTCCGTCCTGACGTTTTTTTGCCT | | | ribosomal | TGAGCGTTCTGACCTTCTTTTGCCT | YRRSGLVTLVSFVVALLGNLVSDAAFL |
| | | TATGAAATACACAATGGTGACGTTC | | | protein | GATGAAGTACACCATGGTGACCTTC | TFFLLLAFTVPAVYEKKKDLIDKWISA |
| | | CTCTGCCGCATCCTGCAGTTGGTCC | | | L23a | CTGTGCCGTATTCTGCAGCTGGTGC | ATAQVEKYMGKIKTKVEEATKKKEGGG |
| | | TATTGGCCGGCGTTCTGTTGGGCTT | | | | TGCTGGCGGGTGTTCTGCTGGGCTT | GSMPAKTAVSKAAAPKKAAAPKKAAAP |
| | | CACGAATCGATGGCACCTCACCTCC | | | | TACCAACCGTTGGCACCTGACCAGC | QKAAAPKKAAAPKKAAAPQKAAVAKKA |
| | | GACGACATCCACGAGGCCGTCAACC | | | | GACGATATCCACGAGGCGGTGAACC | VREAPKKGVKKTAKKGAPAAMTKVVKV |
| | | GCCTTGTGGACTGCGCCACGCCCCG | | | | GTCTGGTTGACTGCGCGACCCCGCG | TKRKAYTRPQFRRPHTYRRPSIPKPSN |
| | | GCTGGTGACGGCCCTTGAGTCCATG | | | | TCTGGTGACCGCGCTGGAAAGCATG | NMSAIPNKWDAFRVIRYPLTTDKAMKK |
| | | CACCAACTCGTGACGTGGCGTGACT | | | | CACCAACTGGTTACCTGGCGTGATT | IEENNTLTFIVDSNANKTEIKKAMRKL |
| | | ACCGCCGCTCCGGGCTCGTCACGCT | | | | ATCGTCGTAGCGGTCTGGTGACCCT | YQVKAVKVNTLIRPDGLKKAYIRLSAS |
| | | GGTGAGCTTCGTGGTTGCTCTTCTC | | | | GGTTAGCTTTGTGGTTGCGCTGCTG | YDALETANKMGLL (SEQ ID |
| | | GGCAACCTCGTCTCCGACGCCGCCT | | | | GGCAACCTGGTGAGCGATGCGGCGT | NO: 70) |
| | | TTCTCACGTTTTTTCTTTTGTTGGC | | | | TCCTGACCTTCTTTCTGCTGCTGGC | |
| | | CTTCACCGTTCCTGCGGTGTACGAG | | | | GTTTACCGTGCCGGCGGTTTACGAG | |
| | | AAGAAGAAGGATTTGATCGACAAGT | | | | AAGAAAAAGGACCTGATCGATAAAT | |
| | | GGATCAGCGCTGCCACGGCTCAGGT | | | | GGATTAGCGCGGCGACCGCGCAGGT | |
| | | GGAGAAGTACATGGGGAAGATCAAA | | | | GGAAAAGTATATGGGCAAGATTAAA | |
| | | ACAAAGGTGGAAGAGGCGACCAAGA | | | | ACCAAGGTTGAGGAAGCGACCAAAA | |
| | | AGAAAGAG | | | | AGAAAGAGGGTGGCGGTGGCAGCAT | |
| | | GGAGGTGGTGGCTCTATGCCTGCCA | | | | GCCGGCGAAAACCGCGGTTAGCAAA | |
| | | AAACCGCCGTTTCGAAGGCTGCTGC | | | | GCGGCGGCGCCGAAGAAAGCGGCTG | |
| | | GCCCAAAAAGGCCGCTGCGCCCAAG | | | | CTCCGAAGAAAGCGGCGGCGCCGCA | |
| | | AAGGCCGCTGCACCACAAAAGGCTG | | | | GAAGGCTGCTGCGCCGAAGAAAGCG | |
| | | CTGCGCCCAAGAAGGCTGCTGCGCC | | | | GCCGCTCCGAAGAAAGCGGCTGCGC | |
| | | CAAGAAGGCTGCTGCACCCCAAAAG | | | | CGCAAAAGGCGGCGGTGGCGAAGAA | |
| | | GCTGCTGTCGCCAAGAAGGCCGTCA | | | | AGCGGTTCGTGAGGCGCCGAAGAAA | |
| | | GGGAGGCCCCCAAAAAGGGTGTCAA | | | | GGTGTGAAGAAAACCGCGAAGAAAG | |
| | | GAAGACCGCCAAGAAGGGCGCGCCG | | | | GCGCGCCGGCGGCGATGACCAAAGT | |
| | | GCCGCTATGACGAAGGTGGTGAAGG | | | | GGTTAAGGTTACCAAGCGTAAGGCG | |
| | | TCACGAAGCGCAAGGCGTACACCCG | | | | TACACCCGTCCGCAGTTCCGTCGTC | |
| | | CCCGCAGTTCCGTCGTCCGCACACG | | | | CGCACACCTATCGTCGTCCGAGCAT | |
| | | TACCGGAGGCCGTCGATCCCCAAGC | | | | CCCGAAACCGAGCAACAACATGAGC | |
| | | CGAGCAACAACATGAGTGCGATTCC | | | | GCGATTCCGAACAAGTGGGACGCGT | |
| | | CAACAAGTGGGATGCGTTTCGTGTG | | | | TCCGTGTTATCCGTTACCCGCTGAC | |
| | | ATCCGCTACCCGCTGACCACCGACA | | | | CACCGATAAAGCGATGAAGAAAATC | |
| | | AGGCGATGAAGAAGATTGAGGAGAA | | | | GAGGAAAACAACACCCTGACCTTTA | |
| | | CAATACGCTGACCTTCATTGTGGAC | | | | TTGTGGACAGCAACGCGAACAAAAC | |
| | | TCGAACGCCAACAAGACGGAAATCA | | | | CGAAATCAAGAAAGCGATGCGTAAG | |
| | | AGAAGGCCATGCGCAAGCTCTACCA | | | | CTGTATCAAGTTAAAGCGGTGAAGG | |
| | | GGTGAAGGCCGTGAAGGTGAACACC | | | | TTAACACCCTGATTCGTCCGGACGG | |
| | | CTCATCCGACCGGACGGCCTTAAGA | | | | TCTGAAGAAAGCGTACATCCGTCTG | |
| | | AGGCGTACATCCGCCTCTCCGCCTC | | | | AGCGCGAGCTATGATGCGCTGGAAA | |
| | | GTACGACGCCCTCGAGACAGCCAAC | | | | CCGCGAACAAGATGGGCCTGCTG | |
| | | AAGATGGGTCTGCTG (SEQ ID | | | | (SEQ ID NO: 88) | |
| | | NO: 87) | | | | | |
| Tc5 | TcYC6_ | ATGCCCGGCAAGGAAGTGAAAAAGG | 1779 | 593 | 60S | ATGCCGGGTAAAGAGGTGAAGAAGG | MPGKEVKKAAKPAAKTAAKPAAKSAAK |
| | 0100010 + | CCGCCAAGCCCGCTGCCAAGACTGC | | | ribosomal | CGGCGAAGCCGGCGGCGAAAACCGC | PAAKPAAKPAAKTAAKPAAKTAAKPAK |
| | TcBrA4_ | TGCAAAGCCTGCTGCCAAGTCTGCT | | | protein | GGCGAAACCGGCGGCGAAAAGCGCT | KPAVKPTVKPAAKAAAPYKKPAAISPF |
| | 0074300 | GCCAAGCCAGCTGCCAAGCCAGCTG | | | L7a, | GCTAAGCCGGCTGCTAAACCGGCTG | VARPKNFGIGHDVPYARDLSRFMRWPT |
| | | CCAAGCCAGCCGCGAAGACCGCTGC | | | 40S | CTAAACCGGCGGCGAAGACCGCTGC | FVTMQRKKRVLQRRLKVPPALHQFTKV |
| | | GAAGCCGGCCGCGAAGACTGCTGCC | | | ribosomal | TAAGCCTGCTGCGAAAACCGCGGCG | LDRSSRNELLKLVKKYPSETRRARRQR |
| | | AAGCCCGCTAAGAAGCCCGCTGTGA | | | protein | AAGCCGGCGAAGAAACCGGCGGTGA | LFDVATEKKKNPEAASKKAPLSVVTGL |
| | | AGCCCACTGTCAAGCCTGCTGCCAA | | | S4 | AACCGACCGTTAAGCCGGCGGCGAA | QEVTRTIEKKTARLVMIANNVDPIELV |
| | | GGCAGCCGCGCCCTACAAGAAGCCT | | | | AGCGGCGGCGCCGTACAAGAAGCCG | LWMPTLCRANKVPYAIVKDKARLGDAV |
| | | GCGGCCATCTCACCTTTTGTGGCGC | | | | GCGGCGATCAGCCCGTTTGTTGCGC | GRKTATCVAITDVNAEDEAALKNLIRS |
| | | GGCCGAAAAACTTTGGTATTGGCCA | | | | GTCCGAAGAACTTTGGTATTGGCCA | VNARFLARSDVIRRQWGGLQLSLRSRA |
| | | CGATGTTCCGTACGCCCGTGATCTT | | | | CGACGTGCCGTATGCGCGTGATCTG | ELRKKRARNAGKDAAAVMGGGGSMTKK |
| | | TCTCGCTTTATGCGGTGGCCCACGT | | | | AGCCGTTTCATGCGTTGGCCGACCT | HLKRLYAPKDWMLSKLTGVFAPRPRAG |
| | | TTGTGACGATGCAGCGGAAGAAGCG | | | | TTGTTACCATGCAGCGTAAGAAACG | PHKLRECMTLMIIIRNRLKYALNAAEA |
| | | TGTACTGCAGCGCCGTCTGAAGGTG | | | | TGTTCTGCAACGTCGTCTGAAAGTG | QMILRQGLVCVDGKPRKDTKYPVGFMD |
| | | CCGCCCGCGCTCCACCAATTTACGA | | | | CCGCCGGCGCTGCACCAGTTCACCA | VVEIPRTGDRFRILYDVKGRFALVKVG |
| | | AGGTGCTTGACCGCTCCAGTCGCAA | | | | AGGTTCTGGACCGTAGCAGCCGTAA | EAEGNIKLLKVENVYTSTGRIPVAMTH |
| | | CGAGCTGCTGAAGCTGGTGAAGAAG | | | | CGAGCTGCTGAAACTGGTGAAGAAA | DGHRIRYPDPRTHRGDTLVYNLKEKKV |
| | | TATCCTTCCGAGACGCGCAGGGCCC | | | | TACCCGAGCGAAACCCGTCGTGCGC | VDLIKSSNGKVVMVTGGANRGRIGEIM |
| | | GCAGGCAGCGCCTGTTTGACGTGGC | | | | GTCGTCAGCGTCTGTTTGATGTGGC | SIERHPGAFDIARLKDAAGHEFATRAS |
| | | GACTGAGAAAAAGAAGAATCCAGAG | | | | GACCGAGAAGAAAAAGAACCCGGAA | NIFVIGKDMQSVPVTLPKQQGLRINVI |
| | | GCGGCGTCCAAGAAGGCCCCGCTCA | | | | GCGGCGAGCAAAAAGGCGCCGCTGA | QEREEKLIAAEARKNMQTRGVRKARK |
| | | GCGTCGTTACCGGTCTGCAGGAGGT | | | | GCGTGGTTACCGGCCTGCAAGAGGT | (SEQ ID NO: 71) |
| | | AACCCGCACCATTGAGAAGAAGACC | | | | TACCCGTACCATCGAGAAGAAGACC | |
| | | GCACGCCTTGTGATGATCGCGAACA | | | | GCGCGTCTGGTGATGATCGCGAACA | |
| | | ATGTGGACCCCATTGAGCTGGTGCT | | | | ACGTTGACCCGATTGAGCTGGTGCT | |
| | | GTGGATGCCGACTTTGTGCCGTGCC | | | | GTGGATGCCGACCCTGTGCCGTGCG | |
| | | AACAAAGTCCCATACGCGATTGTGA | | | | AACAAAGTGCCGTATGCGATCGTTA | |
| | | AGGACAAGGCACGTCTCGGCGACGC | | | | AAGATAAAGCGCGTCTGGGTGATGC | |
| | | GGTGGGCCGGAAGACCGCCACGTGC | | | | GGTGGGTCGTAAGACCGCGACCTGC | |
| | | GTTGCAATCACCGATGTGAATGCCG | | | | GTGGCGATTACCGACGTTAACGCGG | |
| | | AGGACGAGGCCGCTTTGAAGAATCT | | | | AGGATGAAGCGGCGCTGAAAAACCT | |
| | | CATCCGCTCTGTGAATGCACGCTTC | | | | GATCCGTAGCGTTAACGCGCGTTTC | |
| | | CTGGCCCGTAGCGATGTTATCCGTC | | | | CTGGCGCGTAGCGATGTGATTCGTC | |
| | | GCCAATGGGGAGGCCTGCAGCTCTC | | | | GTCAGTGGGGTGGCCTGCAACTGAG | |
| | | ACTGCGTTCTCGAGCCGAGCTGCGC | | | | CCTGCGTAGCCGTGCGGAACTGCGT | |
| | | AAGAAGCGTGCCCGCAACGCCGGCA | | | | AAAAAGCGTGCGCGTAACGCGGGTA | |
| | | AGGATGCTGCCGCCGTAATGGGAGG | | | | AAGATGCGGCGGCGGTGATGGGTGG | |
| | | TGGTGGCTCTATGACCAAGAAGCAC | | | | CGGTGGCAGCATGACCAAAAAGCAC | |
| | | CTGAAGCGCCTTTATGCCCCCAAGG | | | | CTGAAGCGTCTGTACGCGCCGAAAG | |
| | | ACTGGATGCTGAGCAAGCTCACGGG | | | | ATTGGATGCTGAGCAAACTGACCGG | |
| | | CGTGTTCGCTCCACGTCCCCGTGCT | | | | TGTTTTTGCGCCGCGTCCGCGTGCG | |
| | | GGACCCCACAAGCTGCGTGAGTGCA | | | | GGTCCGCACAAACTGCGTGAGTGCA | |
| | | TGACTCTTATGATCATCATCCGCAA | | | | TGACCCTGATGATCATTATCCGTAA | |
| | | TCGTCTGAAGTATGCGCTGAACGCC | | | | CCGTCTGAAGTATGCGCTGAACGCG | |
| | | GCCGAGGCTCAGATGATTCTCCGTC | | | | GCGGAAGCGCAGATGATCCTGCGTC | |
| | | AGGGCCTTGTGTGCGTGGACGGTAA | | | | AAGGTCTGGTGTGCGTTGACGGCAA | |
| | | GCCCCGCAAGGACACCAAGTATCCG | | | | ACCGCGTAAGGATACCAAATACCCG | |
| | | GTTGGCTTCATGGACGTTGTGGAGA | | | | GTTGGTTTTATGGACGTGGTTGAGA | |
| | | TCCCACGGACCGGGGATCGTTTCCG | | | | TCCCGCGTACCGGCGACCGTTTCCG | |
| | | CATTCTGTACGACGTGAAGGGCCGC | | | | TATTCTGTATGATGTGAAAGGTCGT | |
| | | TTTGCCCTCGTGAAGGTTGGCGAGG | | | | TTTGCGCTGGTGAAGGTTGGCGAGG | |
| | | CTGAGGGGAACATCAAGCTCCTGAA | | | | CGGAAGGCAACATTAAGCTGCTGAA | |
| | | GGTGGAGAACGTCTACACAAGCACT | | | | AGTGGAAAACGTTTACACCAGCACC | |
| | | GGTCGCATTCCTGTTGCCATGACAC | | | | GGTCGTATTCCGGTTGCGATGACCC | |
| | | ACGACGGTCACCGCATTCGTTACCC | | | | ATGATGGTCACCGTATTCGTTACCC | |
| | | CGACCCCCGCACCCACCGTGGCGAC | | | | GGACCCGCGTACCCACCGTGGCGAT | |
| | | ACCCTGGTGTACAACCTGAAGGAGA | | | | ACCCTGGTTTATAACCTGAAGGAGA | |
| | | AGAAGGTGGTGGACCTCATCAAGTC | | | | AAAAGGTGGTTGATCTGATCAAGAG | |
| | | CAGCAACGGCAAGGTGGTGATGGTC | | | | CAGCAACGGTAAAGTGGTTATGGTG | |
| | | ACCGGCGGCGCGAACCGCGGCCGTA | | | | ACCGGTGGCGCGAACCGTGGTCGTA | |
| | | TTGGCGAGATCATGTCGATTGAGCG | | | | TTGGCGAGATCATGAGCATTGAACG | |
| | | CCACCCTGGTGCGTTCGACATTGCA | | | | TCACCCGGGCGCGTTTGACATTGCG | |
| | | CGCCTGAAGGATGCGGCGGGACACG | | | | CGTCTGAAAGATGCGGCGGGTCATG | |
| | | AGTTTGCTACCCGAGCGTCCAACAT | | | | AATTTGCGACCCGTGCGAGCAACAT | |
| | | TTTTGTGATTGGCAAGGACATGCAG | | | | CTTTGTTATTGGCAAAGATATGCAA | |
| | | AGCGTTCCTGTGACGCTGCCGAAGC | | | | AGCGTGCCGGTTACCCTGCCGAAGC | |
| | | AACAGGGTCTCCGCATCAACGTGAT | | | | AGCAAGGTCTGCGTATCAACGTGAT | |
| | | TCAGGAGCGTGAGGAGAAGCTTATC | | | | TCAGGAGCGTGAGGAAAAACTGATC | |
| | | GCTGCTGAGGCACGCAAGAATATGC | | | | GCGGCGGAAGCGCGTAAGAACATGC | |
| | | AGACTCGCGGCGTACGCAAGGCCCG | | | | AAACCCGTGGTGTTCGTAAGGCGCG | |
| | | CAAA (SEQ ID NO: 89) | | | | TAAA (SEQ ID NO: 90) | |
| Tc6 | TcYC6_ | ATGACGACAATCGGTACGTACAACG | 1311 | 437 | 40S | ATGACCACCATCGGCACCTACAACG | MTTIGTYNEEGVNVDLYIPRKCHATNN |
| | 0043560 + | AGGAGGGTGTTAACGTGGACCTGTA | | | ribosomal | AGGAAGGCGTGAACGTTGACCTGTA | LITSYDHSAVQIAIANVDANGVLNGTT |
| | TcYC6_ | CATCCCACGCAAGTGCCACGCGACA | | | protein | TATCCCGCGTAAGTGCCACGCGACC | TTFCIAGYLRRQAESDHAINHLAISKG |
| | 0122760 | AACAACCTTATCACGTCATACGACC | | | S21, | AACAACCTGATTACCAGCTACGACC | IIRIKTGKKPRAKKLKNVKGLGVRGLP |
| | | ACTCCGCCGTGCAGATTGCCATTGC | | | hypothetical | ACAGCGCGGTGCAGATCGCGATTGC | RGALQQRGARVLPTQRGVAQRGGAQKG |
| | | GAATGTGGACGCCAACGGTGTGCTA | | | protein | GAACGTGGATGCGAACGGTGTTCTG | NVRKLQPQPQKQRSQLNQRSQQQHGAR |
| | | AACGGCACGACGACAACCTTCTGCA | | | | AACGGCACCACCACCACCTTCTGCA | PTRKEEGGRTQRGGRDAPQARKQQGRN |
| | | TTGCTGGCTATCTTCGTCGCCAGGC | | | | TCGCGGGTTATCTGCGTCGTCAGGC | EPQARRQQGRNEPQARRQQGRNEPQAR |
| | | TGAGTCTGACCACGCAATCAACCAC | | | | GGAAAGCGATCACGCGATCAACCAC | KQQGRDAPQARKQQGRNAPRSQKAGGG |
| | | CTGGCGATTTCGAAGGGCATTATCC | | | | CTGGCGATTAGCAAGGGTATCATTC | GSMMRFTRFLVVAAKRSATSAKLGKSV |
| | | GCATCAAGACCGGCAAGAAGCCTCG | | | | GTATTAAAACCGGCAAGAAACCGCG | GLTAALSPKQRSLPRVSVTKLMKPSGS |
| | | CGCGAAGAAGCTTAAGAATGTGAAG | | | | TGCGAAGAAACTGAAGAACGTGAAA | GKHVTSSFLLKDKKKVATAKVAVPPKK |
| | | GGCCTTGGCGTACGCGGCTTACCAA | | | | GGTCTGGGTGTTCGTGGTCTGCCGC | KRALKVRKGRSSGKKAAALYVRFYHAL |
| | | GGGGTGCTCTGCAACAGAGGGGAGC | | | | GTGGCGCGCTGCAGCAACGTGGTGC | KKSGLVKGKRRMQKTGELWRATKKAKD |
| | | TCGTGTCCTCCCAACCCAGAGGGGT | | | | GCGTGTGCTGCCGACCCAGCGTGGC | FKKRVEAAMRLAKKGQKSRARKLKAQK |
| | | GTCGCGCAGCGTGGCGGCGCTCAGA | | | | GTTGCGCAGCGTGGTGGCGCGCAAA | KAKGKKSAKGVRRVYRRVSRKKTVTST |
| | | AGGGCAACGTCCGCAAGCTGCAGCC | | | | AGGGCAACGTTCGTAAACTGCAACC | VPPLP(SEQ ID NO: 72) |
| | | ACAGCCGCAGAAGCAAAGGTCACAG | | | | GCAGCCGCAAAAACAGCGTAGCCAA | |
| | | CTGAATCAAAGGTCACAGCAGCAGC | | | | CTGAACCAGCGTAGCCAGCAACAGC | |
| | | ACGGCGCCCGGCCGACCCGGAAGGA | | | | ATGGTGCGCGTCCGACCCGTAAGGA | |
| | | AGAGGGCGGTCGCACGCAGCGTGGT | | | | AGAGGGTGGTCGTACCCAACGTGGT | |
| | | GGCAGGGATGCGCCTCAAGCTCGCA | | | | GGCCGTGATGCGCCGCAAGCGCGTA | |
| | | AGCAGCAAGGCAGGAACGAGCCTCA | | | | AACAACAGGGTCGTAACGAGCCGCA | |
| | | AGCTCGCAGGCAGCAAGGCAGGAAC | | | | GGCGCGTCGTCAACAGGGTCGTAAC | |
| | | GAGCCTCAAGCTCGCAGGCAGCAAG | | | | GAACCGCAAGCGCGTCGTCAGCAAG | |
| | | GCAGGAACGAGCCTCAAGCTCGCAA | | | | GCCGCAATGAACCGCAGGCGCGTAA | |
| | | GCAGCAAGGCAGGGATGCGCCTCAA | | | | ACAACAGGGCCGCGACGCGCCGCAA | |
| | | GCTCGTAAGCAGCAAGGCAGGAATG | | | | GCGCGTAAGCAACAGGGTCGTAACG | |
| | | CACCTCGTTCCCAGAAGGCAGGAGG | | | | CGCCGCGTAGCCAGAAAGCGGGTGG | |
| | | TGGTGGCTCTATGATGCGTTTTACC | | | | CGGTGGCAGCATGATGCGTTTCACC | |
| | | CGGTTCCTTGTCGTTGCAGCAAAGC | | | | CGTTTTCTGGTTGTTGCGGCGAAGC | |
| | | GGAGTGCCACCAGCGCCAAACTCGG | | | | GTAGCGCGACCAGCGCGAAGCTGGG | |
| | | TAAGAGTGTTGGACTCACCGCGGCG | | | | TAAAAGCGTGGGCCTGACCGCGGCG | |
| | | CTGAGTCCCAAGCAAAGGTCCCTTC | | | | CTGAGCCCGAAACAGCGTAGCCTGC | |
| | | CCCGCGTCTCAGTGACGAAGTTGAT | | | | CGCGTGTGAGCGTTACCAAGCTGAT | |
| | | GAAGCCCAGCGGGAGCGGGAAACAC | | | | GAAACCGAGCGGTAGCGGCAAGCAC | |
| | | GTTACGTCGTCATTCTTGTTGAAGG | | | | GTGACCAGCAGCTTTCTGCTGAAAG | |
| | | ACAAGAAGAAGGTGGCCACCGCAAA | | | | ACAAGAAAAAGGTTGCGACCGCGAA | |
| | | AGTTGCTGTGCCGCCGAAAAAGAAG | | | | GGTGGCGGTTCCGCCGAAAAAGAAA | |
| | | AGGGCTTTAAAGGTGAGGAAGGGCC | | | | CGTGCGCTGAAGGTTCGTAAAGGTC | |
| | | GCAGCAGCGGCAAAAAGGCCGCGGC | | | | GTAGCAGCGGCAAGAAAGCGGCGGC | |
| | | TCTCTATGTGCGCTTTTATCACGCC | | | | GCTGTACGTGCGTTTCTATCACGCG | |
| | | TTGAAGAAGAGCGGACTTGTGAAGG | | | | CTGAAGAAAAGCGGTCTGGTTAAGG | |
| | | GGAAGCGACGCATGCAGAAAACGGG | | | | GCAAACGTCGTATGCAGAAGACCGG | |
| | | TGAGCTGTGGCGTGCCACAAAGAAG | | | | TGAACTGTGGCGTGCGACCAAGAAA | |
| | | GCGAAGGACTTCAAGAAGCGCGTTG | | | | GCGAAAGATTTTAAGAAACGTGTGG | |
| | | AGGCGGCGATGAGGCTTGCAAAGAA | | | | AGGCGGCGATGCGTCTGGCGAAGAA | |
| | | GGGACAAAAAAGCAGGGCTCGTAAG | | | | AGGTCAGAAGAGCCGTGCGCGTAAG | |
| | | CTGAAGGCGCAGAAGAAGGCAAAGG | | | | CTGAAAGCGCAAAAGAAAGCGAAGG | |
| | | GCAAAAAGTCGGCGAAGGGCGTCAG | | | | GTAAGAAAAGCGCGAAAGGCGTGCG | |
| | | GAGGGTCTACCGGAGGGTCAGCAGG | | | | TCGTGTTTACCGTCGTGTTAGCCGT | |
| | | AAGAAGACTGTCACGAGCACCGTGC | | | | AAGAAAACCGTGACCAGCACCGTTC | |
| | | CGCCTCTCCCT (SEQ ID | | | | CGCCGCTGCCG (SEQ ID | |
| | | NO: 91) | | | | NO: 92) | |
| Tc7 | TcYC6_ | ATGGGTATCGTTCGCAGCCGCCTGC | 1332 | 444 | 40S | ATGGGTATCGTTCGTAGCCGTCTGC | MGIVRSRLHKRKITGGKTKIHRKRMKA |
| | 0083710 + | ATAAGCGCAAGATCACCGGTGGAAA | | | ribosomal | ACAAGCGTAAAATCACCGGTGGCAA | ELGRLPAHTKLGARRVSPVRARGGNFK |
| | TcBrA4_ | GACGAAGATCCACCGGAAGCGCATG | | | protein | GACCAAAATTCACCGTAAGCGTATG | LRGLRLDTGNFAWGTEAIAQRARILDV |
| | 0130080 | AAGGCCGAACTCGGCCGTCTTCCCG | | | S8, | AAAGCGGAGCTGGGTCGTCTGCCGG | VYNATSNELVRTKTLVKNCIVVVDAAP |
| | | CGCACACGAAACTTGGCGCCCGCCG | | | 60S | CGCACACCAAACTGGGTGCGCGTCG | FKLWYAKHYGIDLDAAKSKKTAQSTTE |
| | | CGTGAGTCCCGTCCGCGCCCGCGGT | | | ribosomal | TGTTAGCCCGGTGCGTGCGCGTGGT | KKKSKKTSHAMTEKYDVKKASDELKRK |
| | | GGGAACTTCAAGCTCCGCGGTCTTC | | | protein | GGCAACTTCAAGCTGCGTGGCCTGC | WMLRRENHKIEKAVADQLKEGRLLARI |
| | | GCCTGGACACCGGCAATTTTGCGTG | | | L13 | GTCTGGACACCGGTAACTTTGCGTG | TSRPGQTARADGALLEGAELQFYLKKL |
| | | GGGCACAGAAGCCATTGCTCAGCGG | | | | GGGCACCGAGGCGATTGCGCAGCGT | EKKKRGGGGSMPKGKNAIPHVHQRKHW |
| | | GCCCGTATCCTCGACGTCGTGTACA | | | | GCGCGTATTCTGGATGTGGTTTACA | NPCSSQKGNVKVFLNQPAQKLRRRRLR |
| | | ACGCCACTTCTAACGAGCTGGTGCG | | | | ACGCGACCAGCAACGAACTGGTTCG | LLKAKKTFPRPLKALRPQVNCPTVRHN |
| | | CACGAAGACGCTTGTGAAGAACTGC | | | | TACCAAGACCCTGGTGAAAAACTGC | MKKRLGRGFTVEELKAAGINPRFAPTI |
| | | ATTGTTGTGGTGGACGCCGCGCCCT | | | | ATCGTGGTTGTGGACGCGGCGCCGT | GIRVDRRRKNKSEEGMSINIQRLKTYM |
| | | TCAAGTTATGGTACGCGAAGCACTA | | | | TCAAGCTGTGGTACGCGAAACACTA | SKLVLFPMSYKNVQKGEATEEEVKSAT |
| | | CGGTATCGATCTTGACGCCGCGAAG | | | | TGGTATTGACCTGGATGCGGCGAAA | QDRTRFGTAAVGGFVTPAPEAPRKVTE |
| | | AGCAAGAAGACGGCGCAGAGCACGA | | | | AGCAAGAAAACCGCGCAGAGCACCA | EERTKNVYKFLKKNHSAVRFFGIRRAR |
| | | CGGAGAAGAAGAAGTCGAAGAAGAC | | | | CCGAAAAGAAAAAGAGCAAAAAGAC | QERREAKENEKK (SEQ ID NO: 73) |
| | | CTCACACGCCATGACTGAGAAGTAC | | | | CAGCCACGCGATGACCGAGAAGTAC | |
| | | GACGTCAAGAAGGCCTCCGACGAGC | | | | GACGTTAAAAAGGCGAGCGATGAAC | |
| | | TGAAGCGCAAGTGGATGCTCCGCCG | | | | TGAAGCGTAAATGGATGCTGCGTCG | |
| | | CGAGAACCACAAGATTGAGAAGGCA | | | | TGAGAACCACAAGATCGAAAAAGCG | |
| | | GTCGCTGATCAGCTCAAGGAGGGCC | | | | GTGGCGGACCAACTGAAAGAGGGTC | |
| | | GTCTGCTCGCCCGCATCACCAGCCG | | | | GTCTGCTGGCGCGTATTACCAGCCG | |
| | | CCCTGGCCAGACAGCCCGCGCCGAT | | | | TCCGGGTCAGACCGCGCGTGCGGAT | |
| | | GGTGCACTGCTGGAGGGCGCCGAAC | | | | GGTGCGCTGCTGGAGGGCGCGGAAC | |
| | | TGCAGTTCTATCTGAAGAAGCTCGA | | | | TGCAATTTTATCTGAAAAAGCTGGA | |
| | | GAAGAAGAAGCGGGGAGGTGGTGGC | | | | GAAGAAGAAGCGTGGTGGTGGTGGT | |
| | | TCTATGCCGAAGGGAAAAAACGCGA | | | | AGCATGCCGAAGGGTAAAAACGCGA | |
| | | TCCCCCACGTGCACCAGAGGAAGCA | | | | TCCCGCACGTGCACCAGCGTAAGCA | |
| | | CTGGAACCCGTGCTCTTCCCAGAAG | | | | CTGGAACCCGTGCAGCAGCCAAAAG | |
| | | GGTAATGTGAAGGTTTTCCTCAACC | | | | GGCAACGTTAAAGTGTTCCTGAACC | |
| | | AGCCCGCACAGAAGCTGCGCCGTCG | | | | AGCCGGCGCAAAAGCTGCGTCGTCG | |
| | | CCGCCTACGTCTTTTGAAGGCGAAG | | | | TCGTCTGCGTCTGCTGAAAGCGAAG | |
| | | AAGACGTTCCCACGCCCACTCAAGG | | | | AAAACCTTTCCGCGTCCGCTGAAGG | |
| | | CGCTGCGCCCGCAGGTGAATTGCCC | | | | CGCTGCGTCCGCAGGTTAACTGCCC | |
| | | CACGGTGCGTCACAACATGAAGAAG | | | | GACCGTGCGTCACAACATGAAGAAA | |
| | | CGCCTGGGCCGTGGCTTTACCGTTG | | | | CGTCTGGGTCGTGGCTTCACCGTTG | |
| | | AGGAGCTGAAGGCTGCCGGCATCAA | | | | AGGAACTGAAAGCGGCGGGTATTAA | |
| | | CCCTCGTTTTGCCCCGACGATTGGC | | | | CCCGCGTTTTGCGCCGACCATCGGC | |
| | | ATCCGTGTGGATCGTCGCCGCAAGA | | | | ATTCGTGTGGACCGTCGTCGTAAGA | |
| | | ACAAGAGCGAGGAGGGCATGAGCAT | | | | ACAAAAGCGAGGAAGGTATGAGCAT | |
| | | CAACATCCAGCGCCTGAAGACGTAC | | | | CAACATTCAACGTCTGAAGACCTAC | |
| | | ATGAGCAAGCTGGTGCTCTTCCCCA | | | | ATGAGCAAACTGGTTCTGTTCCCGA | |
| | | TGAGCTACAAGAACGTGCAGAAGGG | | | | TGAGCTATAAGAACGTGCAGAAAGG | |
| | | CGAGGCCACTGAGGAGGAGGTGAAG | | | | CGAGGCGACCGAGGAAGAGGTTAAA | |
| | | TCTGCCACTCAGGACCGCACACGCT | | | | AGCGCGACCCAAGATCGTACCCGTT | |
| | | TTGGTACTGCGGCTGTTGGTGGTTT | | | | TTGGCACCGCGGCGGTTGGTGGCTT | |
| | | TGTGACGCCTGCTCCCGAGGCACCA | | | | CGTGACCCCGGCGCCGGAAGCGCCG | |
| | | CGCAAGGTGACAGAGGAGGAGCGCA | | | | CGTAAGGTTACCGAAGAGGAACGTA | |
| | | CAAAGAACGTGTACAAGTTCCTCAA | | | | CCAAGAACGTGTACAAGTTCCTGAA | |
| | | GAAGAACCACAGCGCTGTTCGCTTC | | | | GAAAAACCACAGCGCGGTGCGTTTC | |
| | | TTTGGCATTCGCAGGGCACGTCAGG | | | | TTTGGTATTCGTCGTGCGCGTCAAG | |
| | | AACGCAGGGAGGCCAAGGAGAACGA | | | | AGCGTCGTGAAGCGAAGGAGAACGA | |
| | | GAAGAAG (SEQ ID NO: 93) | | | | AAAGAAA (SEQ ID NO: 94) | |
| Tc15 | TcBrA4_ | ATGTATAAGTTTGGAGGTGAGGCGA | 1863 | 621 | hypothetical | ATGTACAAGTTCGGTGGCGAAGCGA | MYKFGGEAKDLRNIYNFGDMSQRETEP |
| | 0056330 + | AGGATCTTAGAAACATTTATAATTT | | | protein, | AAGATCTGCGTAACATCTATAACTT | PKDLSLAENKAYLVDVEVHSDNNEEEM |
| | TcBrA4_ | TGGCGATATGAGCCAACGAGAAACG | | | kinetoplastid- | TGGCGACATGAGCCAGCGTGAAACC | GNRESQQPNSRVSPTAHGVPQSSAFFP |
| | 0033670 | GAGCCACCGAAGGACTTATCATTAG | | | specific | GAGCCGCCAAAGGATCTGAGCCTGG | EFSHSSGPDVPRKPSMESTSEQKNSKE |
| | | CAGAAAATAAAGCTTATTTGGTGGA | | | phospho- | CGGAAAACAAAGCGTATCTGGTGGA | KQKENSKVKIAKEVLGINKKNTSGMSP |
| | | TGTAGAGGTGCATTCTGATAATAAT | | | protein | CGTTGAGGTGCACAGCGATAACAAC | EEKERVLLEERWKRAMAEENRLNALEE |
| | | GAAGAGGAAATGGGGAATCGTGAGA | | | phosphatase | GAGGAAGAGATGGGTAACCGTGAAA | QVTHREQATNSSGLLPNFPPKFLCIKP |
| | | GCCAACAACCCAATTCCAGGGTCTC | | | | GCCAGCAACCGAACAGCCGTGTTAG | LVHHDISSVPEVRRQFVRFNFINWIAT |
| | | ACCGACGGCTCATGGAGTTCCTCAA | | | | CCCGACCGCGCATGGCGTGCCGCAA | CVLLLVNMIIVIAVVFASHKEDAKKFH |
| | | TCCTCCGCGTTTTTTCCGGAATTTT | | | | AGCAGCGCGTTCTTTCCGGAGTTTA | TSQNTVLAILYLMGAPLSFIVWYWQIY |
| | | CACACTCTTCTGGACCTGATGTTCC | | | | GCCACAGCAGCGGTCCGGATGTTCC | SACSTGRHTKHLLALSGLVIALAFDIF |
| | | TCGAAAACCCTCAATGGAAAGTACT | | | | GCGTAAGCCGAGCATGGAAAGCACC | MIVGRTNYAACGVSLAIDISKTKSKLA |
| | | TCGGAACAAAAAAACTCAAAGGAAA | | | | AGCGAGCAGAAAAACAGCAAGGAAA | VLPVIVVLFFWVVEAVILCYCIAKQWM |
| | | AACAAAAGGAGAATAGTAAAGTAAA | | | | AACAAAAGGAGAACAGCAAAGTTAA | YYRLDVNAQEEVRRQMRNVIGIGGGGS |
| | | GATTGCAAAAGAAGTTTTAGGAATA | | | | GATCGCGAAGGAAGTGCTGGGCATT | MGKKYAQLETLHNVNGRVVIVGDIHGC |
| | | AACAAGAAAAATACCTCTGGGATGT | | | | AACAAGAAAAACACCAGCGGTATGA | LAQLEDILSVTDFARGRDQLITAGDMV |
| | | CACCTGAAGAGAAGGAGCGTGTATT | | | | GCCCGGAAGAGAAGGAGCGTGTTCT | NKGPDSFGVVRLLKSLGARGVIGNHDA |
| | | ACTTGAAGAAAGGTGGAAAAGAGCC | | | | GCTGGAAGAGCGTTGGAAACGTGCG | KLLKLRKKIRKHGTLHGTNSQSSLAPL |
| | | ATGGCAGAGGAGAATCGTTTGAACG | | | | ATGGCGGAAGAGAACCGTCTGAACG | AMSLPQDVEEYLLQLPHILRIPAHNIL |
| | | CACTCGAAGAGCAAGTAACTCATCG | | | | CGCTGGAAGAGCAGGTGACCCACCG | VVHAGLHVQHPLERQLVKEVTTMRNLI |
| | | TGAGCAAGCGACTAATTCTTCAGGC | | | | TGAACAAGCGACCAACAGCAGCGGC | LQDDGLYRASEDTTDGVPWASLWQGPE |
| | | CTTCTTCCAAACTTCCCTCCCAAGT | | | | CTGCTGCCGAACTTCCCGCCGAAGT | TVVFGHDARRGLQRHPHAIGLDTRCVY |
| | | TCTTATGTATTAAGCCACTTGTACA | | | | TTCTGTGCATCAAACCGCTGGTTCA | GGELTALVCPGEHLVSVPGWTSNRSKV |
| | | CCATGATATTTCGAGTGTTCCCGAG | | | | CCACGATATTAGCAGCGTTCCGGAA | (SEQ ID NO: 74) |
| | | GTGAGAAGACAATTTGTCAGGTTTA | | | | GTGCGTCGTCAGTTCGTGCGTTTCA | |
| | | ATTTTATAAATTGGATTGCCACATG | | | | ACTTTATCAACTGGATTGCGACCTG | |
| | | TGTTTTGCTCCTTGTCAATATGATT | | | | CGTTCTGCTGCTGGTGAACATGATC | |
| | | ATTGTTATTGCTGTGGTATTTGCAT | | | | ATTGTTATCGCGGTGGTTTTCGCGA | |
| | | CTCATAAAGAAGATGCAAAAAAATT | | | | GCCACAAGGAAGACGCGAAGAAATT | |
| | | CCATACTTCTCAAAACACTGTTTTA | | | | TCACACCAGCCAGAACACCGTTCTG | |
| | | GCCATTTTGTACCTGATGGGAGCCC | | | | GCGATTCTGTACCTGATGGGTGCGC | |
| | | CTTTAAGCTTTATTGTTTGGTATTG | | | | CGCTGAGCTTTATCGTGTGGTACTG | |
| | | GCAGATTTATTCTGCTTGTTCCACA | | | | GCAAATTTATAGCGCGTGCAGCACC | |
| | | GGACGTCATACTAAACATCTTTTGG | | | | GGCCGTCACACCAAACACCTGCTGG | |
| | | CTCTAAGTGGGTTGGTTATAGCTCT | | | | CGCTGAGCGGTCTGGTGATTGCGCT | |
| | | TGCCTTTGATATATTTATGATTGTT | | | | GGCGTTCGATATCTTTATGATTGTT | |
| | | GGTCGGACAAACTATGCTGCATGCG | | | | GGCCGTACCAACTATGCGGCGTGCG | |
| | | GTGTATCTCTTGCAATAGATATATC | | | | GTGTGAGCCTGGCGATCGACATTAG | |
| | | GAAAACGAAAAGTAAGCTTGCCGTA | | | | CAAAACCAAGAGCAAACTGGCGGTT | |
| | | TTGCCCGTGATCGTTGTTCTTTTTT | | | | CTGCCGGTGATTGTGGTTCTGTTCT | |
| | | TCTGGGTTGTAGAGGCTGTTATATT | | | | TTTGGGTGGTTGAGGCGGTTATCCT | |
| | | GTGTTACTGTATCGCAAAACAGTGG | | | | GTGCTATTGCATTGCGAAACAGTGG | |
| | | ATGTACTATCGGTTGGATGTGAACG | | | | ATGTACTATCGTCTGGATGTTAACG | |
| | | CGCAAGAAGAAGTGAGACGCCAGAT | | | | CGCAGGAAGAGGTGCGTCGTCAAAT | |
| | | GCGGAATGTGATTGGAATTGGAGGT | | | | GCGTAACGTGATCGGCATTGGTGGC | |
| | | GGTGGCTCTATGGGAAAAAAATACG | | | | GGTGGCAGCATGGGTAAGAAATACG | |
| | | CACAGTTAGAGACTCTCCACAACGT | | | | CGCAACTGGAAACCCTGCACAACGT | |
| | | GAATGGGCGGGTTGTCATTGTGGGC | | | | TAACGGTCGTGTGGTTATCGTGGGC | |
| | | GACATTCATGGCTGCCTTGCCCAAC | | | | GACATTCACGGTTGCCTGGCGCAGC | |
| | | TGGAGGACATTTTATCAGTCACAGA | | | | TGGAGGACATCCTGAGCGTTACCGA | |
| | | CTTTGCGAGGGGAAGGGATCAGTTA | | | | TTTCGCGCGTGGCCGTGACCAACTG | |
| | | ATCACCGCTGGGGACATGGTGAACA | | | | ATTACCGCGGGTGATATGGTGAACA | |
| | | AAGGGCCAGACTCGTTTGGCGTTGT | | | | AGGGCCCGGACAGCTTTGGTGTGGT | |
| | | GCGTCTGCTGAAGAGCCTTGGAGCA | | | | TCGTCTGCTGAAAAGCCTGGGTGCG | |
| | | CGCGGTGTGATTGGCAATCATGACG | | | | CGTGGCGTGATCGGTAACCACGATG | |
| | | CCAAGCTTCTCAAACTTCGGAAAAA | | | | CGAAGCTGCTGAAACTGCGTAAGAA | |
| | | GATACGAAAACATGGGACGCTGCAC | | | | AATTCGTAAGCACGGCACCCTGCAT | |
| | | GGGACGAATAGCCAATCGAGTTTGG | | | | GGCACCAACAGCCAGAGCAGCCTGG | |
| | | CCCCGCTTGCCATGTCGCTACCGCA | | | | CGCCGCTGGCGATGAGCCTGCCGCA | |
| | | GGATGTTGAAGAGTATTTATTACAA | | | | GGACGTTGAAGAGTATCTGCTGCAA | |
| | | CTGCCGCATATTCTCCGCATTCCTG | | | | CTGCCGCACATCCTGCGTATTCCGG | |
| | | CACACAACATTCTGGTGGTACATGC | | | | CGCACAACATCCTGGTGGTTCATGC | |
| | | GGGCCTTCACGTTCAACACCCACTC | | | | GGGCCTGCATGTGCAGCACCCGCTG | |
| | | GAGCGGCAATTGGTTAAGGAGGTCA | | | | GAACGTCAACTGGTTAAAGAGGTGA | |
| | | CTACGATGCGCAACCTCATTTTGCA | | | | CCACCATGCGTAACCTGATTCTGCA | |
| | | GGATGACGGGCTGTACAGGGCATCT | | | | GGACGATGGTCTGTATCGTGCGAGC | |
| | | GAGGATACAACGGACGGTGTGCCCT | | | | GAAGACACCACCGATGGCGTTCCGT | |
| | | GGGCATCGCTGTGGCAGGGTCCGGA | | | | GGGCGAGCCTGTGGCAGGGTCCGGA | |
| | | GACTGTTGTCTTTGGCCACGACGCC | | | | AACCGTGGTTTTCGGCCACGATGCG | |
| | | AGACGAGGCCTCCAACGCCACCCTC | | | | CGTCGTGGTCTGCAACGTCACCCGC | |
| | | ATGCGATCGGGTTGGACACTCGGTG | | | | ACGCGATCGGTCTGGACACCCGTTG | |
| | | TGTGTATGGCGGGGAGCTCACTGCT | | | | CGTTTACGGTGGCGAACTGACCGCG | |
| | | CTTGTGTGTCCCGGTGAACACCTCG | | | | CTGGTGTGCCCGGGTGAACACCTGG | |
| | | TTTCCGTGCCTGGATGGACTTCCAA | | | | TTAGCGTGCCGGGTTGGACCAGCAA | |
| | | TAGATCGAAGGTG (SEQ ID | | | | CCGTAGCAAGGTG (SEQ ID | |
| | | NO: 95) | | | | NO: 96) | |
| Tc16 | TcYC6_ | ATGTATAAGTTTGGAGGTGAGGCGA | 1863 | 621 | hypothetical | ATGTACAAATTCGGTGGCGAAGCGA | MYKFGGEAKDLRNIYNFGDMSQRETEP |
| | 0074990 + | AGGATCTTCGAAACATTTATAATTT | | | protein, | AGGATCTGCGTAACATTTATAACTT | QKELSLAENRAYLVDVEVHSDNNEEEM |
| | TcYC6_ | TGGCGATATGAGCCAACGAGAAACG | | | kinetoplastid- | TGGCGACATGAGCCAGCGTGAAACC | GHRESQQPNSRVSPTAQGVPQSSAFFS |
| | 0106870 | GAGCCACAGAAGGAATTATCATTGG | | | specific | GAGCCGCAAAAAGAACTGAGCCTGG | EFSHSSGIDFPQKPSMENTSDQKNSNE |
| | | CAGAAAATAGAGCTTATTTGGTGGA | | | phospho- | CGGAGAACCGTGCGTATCTGGTGGA | KPKENSKVKIAKEVLGINKKNTSGMSP |
| | | TGTAGAGGTGCATTCTGATAATAAT | | | protein | CGTTGAAGTGCACAGCGATAACAAC | EEKERVLLEERWKRAMAEENRLNALEE |
| | | GAAGAGGAAATGGGGCATCGTGAGA | | | phosphatase | GAGGAAGAGATGGGTCACCGTGAGA | QVTHREQATNSSGLLPNFPPKFLFIKP |
| | | GCCAACAACCCAACTCCAGAGTCTC | | | | GCCAGCAACCGAACAGCCGTGTGAG | LVHHDISSVPEVRRQFVRFNFINWIAT |
| | | ACCGACGGCTCAGGGAGTTCCTCAG | | | | CCCGACCGCGCAGGGCGTTCCGCAA | CVLLLVNMIIVIAVVFASHKEDAKKEN |
| | | TCCTCCGCGTTTTTTTCGGAATTTT | | | | AGCAGCGCGTTCTTTAGCGAATTCA | TSQNTVLAILYLVGAPLSFIVWYWQIY |
| | | CACACTCTTCTGGAATTGATTTTCC | | | | GCCACAGCAGCGGTATCGATTTTCC | SACSTGRHTKHLLALSGLVIALAFVIF |
| | | TCAAAAACCCTCAATGGAAAATACT | | | | GCAGAAACCGAGCATGGAGAACACC | MIVGRTNYAACGVSLAIDISKTKSKFA |
| | | TCGGACCAAAAAAACTCAAACGAAA | | | | AGCGACCAAAAGAACAGCAACGAAA | VLPVIIVLFFWVVEAVILCYCIVKQWI |
| | | AACCAAAGGAGAATAGTAAAGTAAA | | | | AGCCGAAAGAGAACAGCAAGGTGAA | YYRLDVNAQEEVRRQMRNVIGIGGGGS |
| | | GATCGCAAAAGAAGTTTTAGGAATA | | | | AATTGCGAAGGAAGTTCTGGGCATC | MGKKYAQLETLHNVNGRVVIVGDIHGC |
| | | AATAAGAAAAATACCTCTGGGATGT | | | | AACAAGAAAAACACCAGCGGTATGA | LAQLEDILSVTEFARGRDQLITAGDMV |
| | | CACCTGAAGAGAAGGAGCGTGTATT | | | | GCCCGGAAGAGAAAGAGCGTGTGCT | NKGPDSFGVVRLLKSLGARGVIGNHDA |
| | | ACTTGAAGAAAGATGGAAAAGAGCC | | | | GCTGGAAGAGCGTTGGAAGCGTGCG | KLLKLRKKIRKHGALHGKNSQSSLAPL |
| | | ATGGCAGAGGAGAATCGTTTGAACG | | | | ATGGCGGAAGAGAACCGTCTGAACG | AMSLPQDVEEYLSQLPHILRIPAHNIL |
| | | CACTCGAAGAGCAAGTAACTCATCG | | | | CGCTGGAAGAGCAGGTTACCCACCG | VVHAGLHVQHPLERQLVKEVTTMRNLI |
| | | TGAGCAAGCGACTAATTCTTCAGGT | | | | TGAACAAGCGACCAACAGCAGCGGC | LQDDGLYRASEDTTDGVPWASLWQGPE |
| | | CTTCTTCCCAACTTCCCTCCCAAGT | | | | CTGCTGCCGAACTTCCCGCCGAAAT | TVVFGHDARRGLQRYPHAIGLDTRCVY |
| | | TCTTATTTATTAAGCCACTTGTACA | | | | TCCTGTTTATTAAGCCGCTGGTGCA | GGELTALVCPGEHLVSVPGWTSNRSKV |
| | | CCATGATATTTCGAGTGTTCCCGAG | | | | CCACGATATCAGCAGCGTTCCGGAA | (SEQ ID NO: 75) |
| | | GTCAGAAGACAATTTGTCAGGTTTA | | | | GTGCGTCGTCAGTTTGTTCGTTTCA | |
| | | ATTTTATAAATTGGATCGCCACATG | | | | ACTTTATCAACTGGATTGCGACCTG | |
| | | TGTTTTGCTCCTTGTCAATATGATT | | | | CGTGCTGCTGCTGGTTAACATGATC | |
| | | ATTGTTATTGCTGTGGTATTTGCAT | | | | ATTGTGATTGCGGTGGTTTTCGCGA | |
| | | CTCATAAAGAAGATGCAAAAAAATT | | | | GCCACAAAGAGGACGCGAAGAAATT | |
| | | CAATACTTCTCAAAACACTGTTTTA | | | | TAACACCAGCCAGAACACCGTGCTG | |
| | | GCCATTTTGTACCTGGTGGGAGCCC | | | | GCGATCCTGTACCTGGTTGGTGCGC | |
| | | CTTTAAGCTTTATTGTTTGGTATTG | | | | CGCTGAGCTTCATTGTTTGGTACTG | |
| | | GCAGATTTATTCTGCTTGTTCCACA | | | | GCAAATCTATAGCGCGTGCAGCACC | |
| | | GGACGTCATACCAAACATCTTTTGG | | | | GGTCGTCACACCAAGCACCTGCTGG | |
| | | CTCTAAGTGGGTTGGTTATAGCACT | | | | CGCTGAGCGGTCTGGTGATTGCGCT | |
| | | TGCCTTTGTTATATTTATGATTGTT | | | | GGCGTTCGTGATCTTTATGATTGTT | |
| | | GGTCGGACAAACTATGCTGCATGCG | | | | GGCCGTACCAACTACGCGGCGTGCG | |
| | | GTGTATCTCTTGCAATAGATATATC | | | | GTGTTAGCCTGGCGATCGATATTAG | |
| | | GAAAACGAAAAGCAAGTTTGCCGTA | | | | CAAGACCAAAAGCAAGTTTGCGGTT | |
| | | TTGCCCGTGATCATTGTTCTTTTTT | | | | CTGCCGGTGATCATTGTTCTGTTCT | |
| | | TCTGGGTTGTAGAGGCTGTTATATT | | | | TTTGGGTGGTTGAAGCGGTGATCCT | |
| | | GTGTTACTGTATCGTAAAACAGTGG | | | | GTGCTATTGCATTGTTAAACAGTGG | |
| | | ATCTACTATCGGTTGGATGTGAACG | | | | ATCTACTATCGTCTGGACGTGAACG | |
| | | CGCAAGAAGAAGTGAGGCGCCAGAT | | | | CGCAGGAAGAGGTTCGTCGTCAAAT | |
| | | GCGGAATGTGATTGGAATTGGAGGT | | | | GCGTAACGTGATCGGCATTGGTGGC | |
| | | GGTGGCTCTATGGGAAAAAAATACG | | | | GGTGGCAGCATGGGTAAGAAATACG | |
| | | CACAGTTGGAGACTCTCCACAACGT | | | | CGCAACTGGAAACCCTGCACAACGT | |
| | | GAATGGGCGGGTTGTGATTGTAGGC | | | | GAACGGTCGTGTGGTTATCGTTGGC | |
| | | GACATTCATGGCTGCCTTGCCCAAC | | | | GATATTCACGGTTGCCTGGCGCAGC | |
| | | TGGAGGACATTTTATCAGTCACAGA | | | | TGGAAGACATTCTGAGCGTGACCGA | |
| | | ATTTGCGAGGGGAAGGGATCAGTTA | | | | GTTTGCGCGTGGCCGTGACCAACTG | |
| | | ATCACCGCTGGGGACATGGTGAACA | | | | ATCACCGCGGGTGATATGGTTAACA | |
| | | AAGGGCCAGACTCGTTTGGCGTTGT | | | | AAGGCCCGGACAGCTTTGGTGTGGT | |
| | | GCGTCTGCTGAAGAGCCTTGGAGCA | | | | TCGTCTGCTGAAGAGCCTGGGTGCG | |
| | | CGCGGTGTGATTGGCAATCATGACG | | | | CGTGGCGTTATTGGTAACCACGATG | |
| | | CCAAGCTTCTCAAACTTCGGAAAAA | | | | CGAAACTGCTGAAGCTGCGTAAGAA | |
| | | GATACGAAAACATGGGGCGCTGCAC | | | | AATCCGTAAACACGGCGCGCTGCAC | |
| | | GGGAAGAATAGCCAATCGAGTTTAG | | | | GGCAAGAACAGCCAGAGCAGCCTGG | |
| | | CCCCGCTTGCCATGTCGCTACCGCA | | | | CGCCGCTGGCGATGAGCCTGCCGCA | |
| | | GGATGTTGAAGAGTATTTATCACAA | | | | GGACGTGGAAGAGTATCTGAGCCAA | |
| | | CTGCCGCATATTCTCCGCATTCCCG | | | | CTGCCGCACATCCTGCGTATTCCGG | |
| | | CACACAACATTCTGGTGGTACATGC | | | | CGCACAACATTCTGGTGGTTCATGC | |
| | | GGGCCTTCACGTTCAACACCCGCTT | | | | GGGCCTGCATGTTCAGCACCCGCTG | |
| | | GAGCGGCAATTGGTTAAGGAGGTCA | | | | GAACGTCAACTGGTGAAAGAGGTTA | |
| | | CTACGATGCGCAACCTCATTTTGCA | | | | CCACCATGCGTAACCTGATCCTGCA | |
| | | GGATGACGGGCTGTACAGGGCATCT | | | | GGACGATGGTCTGTACCGTGCGAGC | |
| | | GAGGATACAACGGACGGTGTGCCGT | | | | GAAGACACCACCGATGGCGTGCCGT | |
| | | GGGCATCGCTGTGGCAGGGTCCGGA | | | | GGGCGAGCCTGTGGCAGGGTCCGGA | |
| | | GACTGTTGTCTTTGGCCACGACGCC | | | | AACCGTGGTTTTCGGCCACGATGCG | |
| | | AGACGAGGCCTCCAACGCTACCCTC | | | | CGTCGTGGTCTGCAACGTTACCCGC | |
| | | ATGCGATCGGATTGGACACTCGGTG | | | | ACGCGATCGGTCTGGACACCCGTTG | |
| | | TGTGTATGGCGGGGAGCTCACTGCT | | | | CGTGTATGGTGGCGAACTGACCGCG | |
| | | CTTGTGTGTCCCGGTGAACACCTCG | | | | CTGGTTTGCCCGGGCGAGCACCTGG | |
| | | TTTCCGTGCCTGGATGGACTTCCAA | | | | TTAGCGTGCCGGGTTGGACCAGCAA | |
| | | TAGATCGAAGGTG (SEQ ID | | | | CCGTAGCAAGGTT (SEQ ID | |
| | | NO: 97) | | | | NO: 98) | |
| Tc17 | TcBrA4_ | ATGGGGGCTCCTCAGATCGTGTACT | 1479 | 493 | hypothetical | ATGGGTGCGCCGCAGATCGTGTACA | MGAPQIVYSALITNTTTIAVTVVVTYT |
| | 0028230 + | CCGCCCTCATAACGAACACCACCAC | | | protein, | GCGCGCTGATTACCAACACCACCAC | MPNEMPPETLELLIQPGEEMLAPQKLV |
| | TcBrA4_ | AATTGCTGTGACGGTGGTTGTCACC | | | calcium- | CATCGCGGTTACCGTGGTTGTGACC | EDGIVTWTGYISKVAIQGGPSMSEPFP |
| | 0029760 | TACACCATGCCGAACGAAATGCCCC | | | binding | TATACCATGCCGAACGAGATGCCGC | GVECPTRRYDFEVFMHAGVLRLFALGP |
| | | CGGAGACTCTGGAATTGCTCATTCA | | | protein | CAGAAACCCTGGAACTGCTGATTCA | AESSSDGGGGSMDTTLYSEVNRLERGD |
| | | ACCAGGCGAAGAAATGTTAGCGCCG | | | | GCCGGGCGAGGAAATGCTGGCGCCG | FLLFHCVQLSQHERDVQRYFFGCYFPR |
| | | CAGAAATTGGTGGAGGACGGTATAG | | | | CAAAAGCTGGTTGAAGATGGTATCG | WRGFYLEEVRDMPGPLGYKVQRHFPAY |
| | | TAACCTGGACAGGCTATATTAGCAA | | | | TGACCTGGACCGGCTACATCAGCAA | PFDVYLKDNGEHFLTDDFQEGSIFTLG |
| | | GGTTGCCATTCAGGGTGGGCCGTCT | | | | AGTGGCGATTCAAGGTGGCCCGAGC | ASQNQRDGDSKRYKVVHCDDSRLRTRT |
| | | ATGAGTGAACCTTTCCCGGGAGTGG | | | | ATGAGCGAACCGTTCCCGGGTGTTG | GTTLADIGNDITTKLNQTHRVPGEVID |
| | | AGTGTCCTACGAGAAGATACGACTT | | | | AATGCCCGACCCGTCGTTATGACTT | LLREIRDAYVVYAGNGIPEIGIKAMGR |
| | | TGAAGTTTTCATGCATGCCGGCGTG | | | | CGAGGTTTTTATGCACGCGGGTGTT | HFRHVSEDGKRWMSLENIGKLVRDSRA |
| | | CTGCGGCTATTCGCATTGGGCCCTG | | | | CTGCGTCTGTTTGCGCTGGGTCCGG | FSNTLSFEDTQRTNSTISNNARSIHEA |
| | | CCGAATCAAGCAGTGATGGAGGTGG | | | | CGGAAAGCAGCAGCGATGGTGGCGG | FPQNEEGCIDYDLFMDYVRGPMSQKRK |
| | | TGGCTCTATGGATACGACGCTTTAC | | | | TGGCAGCATGGACACCACCCTGTAC | DAVWEIFRKLDFDGDGYLNILDIQARY |
| | | AGTGAGGTGAATCGTCTCGAACGCG | | | | AGCGAGGTGAACCGTCTGGAACGTG | NAQQHPVVAVERLFSADKLLKGFLTVW |
| | | GTGACTTTCTTCTTTTTCACTGTGT | | | | GTGATTTCCTGCTGTTTCACTGCGT | DENKQYGLIPYAEFIDYYNGVSAVIAD |
| | | GCAGCTCTCACAACACGAGCGTGAC | | | | TCAGCTGAGCCAACACGAGCGTGAC | DYIFFDILRNQWKVMRDWGGTVGTRGG |
| | | GTGCAGCGGTACTTCTTTGGATGCT | | | | GTGCAGCGTTACTTCTTTGGTTGCT | NCEFPTM (SEQ ID NO: 76) |
| | | ACTTTCCGCGCTGGCGTGGGTTCTA | | | | ATTTCCCGCGTTGGCGTGGCTTTTA | |
| | | CCTGGAGGAGGTGAGGGATATGCCG | | | | CCTGGAGGAAGTTCGTGATATGCCG | |
| | | GGCCCTCTAGGCTACAAGGTGCAGC | | | | GGTCCGCTGGGCTATAAGGTGCAGC | |
| | | GACACTTTCCTGCGTATCCCTTTGA | | | | GTCACTTCCCGGCGTACCCGTTTGA | |
| | | CGTGTATCTGAAGGACAATGGTGAA | | | | TGTTTATCTGAAAGACAACGGCGAG | |
| | | CACTTTCTCACGGATGACTTCCAGG | | | | CACTTCCTGACCGACGATTTTCAAG | |
| | | AGGGTTCTATATTCACTTTGGGAGC | | | | AAGGTAGCATCTTCACCCTGGGCGC | |
| | | CTCGCAAAATCAGCGTGACGGCGAC | | | | GAGCCAGAACCAACGTGACGGCGAT | |
| | | TCGAAGCGATATAAAGTAGTGCACT | | | | AGCAAGCGTTACAAAGTTGTGCACT | |
| | | GCGACGATAGTCGTTTGCGCACGCG | | | | GCGACGATAGCCGTCTGCGTACCCG | |
| | | CACGGGTACGACTCTTGCAGACATT | | | | TACCGGCACCACCCTGGCGGATATC | |
| | | GGCAATGACATCACGACGAAGTTGA | | | | GGCAACGACATTACCACCAAGCTGA | |
| | | ATCAAACACACCGTGTCCCTGGCGA | | | | ACCAGACCCACCGTGTTCCGGGCGA | |
| | | GGTGATAGATCTCCTGCGTGAGATT | | | | AGTGATTGATCTGCTGCGTGAAATC | |
| | | AGAGATGCGTATGTTGTGTATGCCG | | | | CGTGACGCGTACGTTGTGTATGCGG | |
| | | GCAATGGCATTCCTGAGATTGGTAT | | | | GTAACGGCATTCCGGAAATCGGTAT | |
| | | CAAGGCAATGGGACGTCACTTTCGC | | | | TAAAGCGATGGGCCGTCACTTCCGT | |
| | | CACGTCAGCGAGGATGGAAAGCGGT | | | | CACGTGAGCGAGGACGGCAAGCGTT | |
| | | GGATGTCGTTGGAGAACATTGGAAA | | | | GGATGAGCCTGGAAAACATCGGCAA | |
| | | GCTTGTTCGTGACTCTCGTGCCTTT | | | | ACTGGTTCGTGATAGCCGTGCGTTC | |
| | | TCCAACACATTGTCATTTGAGGACA | | | | AGCAACACCCTGAGCTTTGAGGACA | |
| | | CGCAGAGGACGAATTCCACGATTAG | | | | CCCAGCGTACCAACAGCACCATTAG | |
| | | CAATAATGCAAGGAGCATTCATGAA | | | | CAACAACGCGCGTAGCATCCACGAA | |
| | | GCCTTTCCGCAGAATGAAGAAGGCT | | | | GCGTTCCCGCAAAACGAGGAAGGTT | |
| | | GCATTGACTATGATTTATTCATGGA | | | | GCATTGACTACGATCTGTTTATGGA | |
| | | CTACGTTCGTGGACCGATGAGCCAA | | | | TTATGTGCGTGGCCCGATGAGCCAG | |
| | | AAAAGGAAGGATGCCGTCTGGGAAA | | | | AAGCGTAAAGACGCGGTTTGGGAGA | |
| | | TATTCCGCAAGCTTGACTTTGATGG | | | | TCTTCCGTAAGCTGGACTTTGATGG | |
| | | AGACGGCTACCTCAACATCTTAGAC | | | | TGACGGCTACCTGAACATCCTGGAT | |
| | | ATTCAGGCCCGCTACAATGCGCAGC | | | | ATTCAAGCGCGTTATAACGCGCAGC | |
| | | AGCACCCTGTGGTGGCGGTGGAGAG | | | | AACACCCGGTTGTGGCGGTGGAACG | |
| | | ACTCTTCTCCGCGGACAAACTGCTC | | | | TCTGTTCAGCGCGGATAAGCTGCTG | |
| | | AAGGGCTTCCTCACCGTTTGGGATG | | | | AAAGGTTTTCTGACCGTTTGGGACG | |
| | | AGAACAAACAATACGGGTTGATCCC | | | | AGAACAAGCAGTACGGCCTGATTCC | |
| | | ATACGCCGAGTTTATCGACTACTAC | | | | GTATGCGGAATTCATCGACTACTAT | |
| | | AACGGCGTCAGCGCGGTAATTGCGG | | | | AACGGTGTTAGCGCGGTGATCGCGG | |
| | | ACGACTACATCTTTTTTGATATTCT | | | | ACGATTACATCTTCTTTGATATTCT | |
| | | CCGGAATCAATGGAAGGTCATGCGT | | | | GCGTAACCAATGGAAAGTTATGCGT | |
| | | GACTGGGGAGGGACGGTGGGGACGA | | | | GACTGGGGTGGCACCGTGGGCACCC | |
| | | GGGGAGGGAATTGTGAGTTCCCGAC | | | | GTGGTGGCAACTGCGAGTTTCCGAC | |
| | | GATG (SEQ ID NO: 99) | | | | CATG (SEQ ID NO: 100) | |
| Tc18 | TcYC6_ | ATGGGGTCTCCTAAGATCGTGTACT | 1479 | 493 | hypothetical | ATGGGTAGCCCGAAGATCGTGTACA | MGSPKIVYSALIRNTTTISVTVLVTYS |
| | 0097920 + | CCGCCCTCATAAGGAACACCACCAC | | | protein, | GCGCGCTGATTCGTAACACCACCAC | MPSEMPQETVQLLIPPGEEKEAPQKLV |
| | TcYC6_ | GATTTCTGTGACGGTGCTTGTCACC | | | calcium- | CATCAGCGTGACCGTTCTGGTGACC | EEDTVTWTGFISKVAVEGGQSMSAPFL |
| | 0096240 | TATTCCATGCCGAGCGAAATGCCCC | | | binding | TATAGCATGCCGAGCGAGATGCCGC | GVESPTRRYGFEVYMQAGMLRLLALGP |
| | | AGGAAACTGTGCAATTGCTCATTCC | | | protein | AGGAAACCGTTCAACTGCTGATTCC | VESSSDGGGGSMDTTLYSEVNRLERGD |
| | | ACCAGGCGAAGAAAAGGAAGCGCCC | | | | GCCGGGTGAAGAAAAAGAGGCGCCG | FLFFHCVQLSQHERDVQRYFFGCYFPR |
| | | CAGAAATTGGTGGAGGAAGATACAG | | | | CAGAAACTGGTTGAGGAAGATACCG | WRGFYLEEVRDMPGPLGYKVQRHFPAY |
| | | TAACCTGGACAGGCTTTATTAGCAA | | | | TGACCTGGACCGGTTTCATCAGCAA | PFDVYLKDNGEHFLTDDFQEGSIFTLG |
| | | GGTTGCCGTTGAGGGTGGGCAGTCT | | | | AGTTGCGGTGGAAGGTGGCCAAAGC | ASQNQRDGESKRYKVVHCDDSRLRTRT |
| | | ATGAGTGCTCCTTTCCTGGGAGTGG | | | | ATGAGCGCGCCGTTCCTGGGTGTTG | GTTLADIGNDITTRLNQTHRVPGEVID |
| | | AATCTCCTACGAGAAGATACGGTTT | | | | AGAGCCCGACCCGTCGTTACGGTTT | LLREIRDAYVVYAGNGIPEIGIKAMGR |
| | | TGAAGTTTACATGCAAGCCGGCATG | | | | TGAAGTGTATATGCAAGCGGGTATG | HFRHVSEDGKRWMSLENIGKLVRDSRA |
| | | CTGCGGCTATTAGCATTGGGCCCTG | | | | CTGCGTCTGCTGGCGCTGGGTCCGG | FSTTLSFEDTQKTNSTISNNARSIHEA |
| | | TCGAATCAAGCAGTGATGGAGGTGG | | | | TGGAGAGCAGCAGCGATGGTGGCGG | FPQNEEGCIDYDLFMDYVRGPMSQKRK |
| | | TGGCTCTATGGATACGACGCTTTAC | | | | TGGCAGCATGGACACCACCCTGTAC | DAVWEIFRKLDFDGDGYLNILDIQARY |
| | | AGTGAGGTGAATCGTCTCGAACGCG | | | | AGCGAGGTTAACCGTCTGGAACGTG | NAQQHPVVAVERLFSADKLLKGFLTVW |
| | | GTGACTTTCTTTTTTTTCACTGTGT | | | | GTGATTTTCTGTTCTTTCACTGCGT | DENKQYGLIPYAEFIDYYNGVSAVIAD |
| | | GCAGCTCTCACAACACGAGCGTGAC | | | | TCAGCTGAGCCAACACGAACGTGAC | DYIFFDILRNQWKVMRDWGGTVGTRRG |
| | | GTGCAGCGGTACTTCTTTGGATGCT | | | | GTGCAACGTTACTTCTTTGGCTGCT | KSEVSTM (SEQ ID NO: 77) |
| | | ACTTTCCGCGCTGGCGTGGGTTCTA | | | | ATTTCCCGCGTTGGCGTGGTTTTTA | |
| | | CCTGGAGGAGGTGAGGGATATGCCA | | | | CCTGGAGGAAGTTCGTGATATGCCG | |
| | | GGCCCTCTAGGCTACAAGGTGCAGC | | | | GGTCCGCTGGGCTATAAGGTGCAAC | |
| | | GACACTTTCCTGCGTATCCCTTTGA | | | | GTCACTTCCCGGCGTACCCGTTTGA | |
| | | CGTGTATCTGAAGGACAATGGTGAA | | | | TGTTTATCTGAAAGACAACGGCGAG | |
| | | CACTTTCTCACGGATGACTTCCAGG | | | | CACTTCCTGACCGACGATTTTCAGG | |
| | | AGGGTTCTATATTCACTTTGGGAGC | | | | AAGGTAGCATTTTCACCCTGGGCGC | |
| | | CTCGCAAAATCAGCGTGACGGCGAG | | | | GAGCCAGAACCAACGTGACGGCGAG | |
| | | TCGAAGCGATATAAAGTAGTGCACT | | | | AGCAAGCGTTACAAAGTGGTTCACT | |
| | | GCGACGATAGTCGTCTGCGCACGCG | | | | GCGACGATAGCCGTCTGCGTACCCG | |
| | | CACGGGCACGACTCTTGCAGACATT | | | | TACCGGCACCACCCTGGCGGATATC | |
| | | GGCAATGACATCACGACGAGGTTGA | | | | GGCAACGACATTACCACCCGTCTGA | |
| | | ATCAAACACACCGTGTCCCTGGCGA | | | | ACCAGACCCACCGTGTTCCGGGCGA | |
| | | GGTGATAGATCTCCTGCGTGAGATT | | | | AGTGATTGATCTGCTGCGTGAAATC | |
| | | AGAGATGCGTATGTTGTGTATGCCG | | | | CGTGACGCGTACGTGGTTTATGCGG | |
| | | GCAATGGCATTCCTGAGATTGGTAT | | | | GTAACGGCATTCCGGAAATCGGTAT | |
| | | CAAGGCAATGGGACGTCACTTTCGC | | | | TAAGGCGATGGGCCGTCACTTTCGT | |
| | | CACGTCAGCGAGGATGGAAAGCGGT | | | | CACGTGAGCGAGGACGGCAAGCGTT | |
| | | GGATGTCGTTGGAGAACATTGGAAA | | | | GGATGAGCCTGGAAAACATCGGTAA | |
| | | GCTTGTTCGTGACTCTCGTGCCTTT | | | | ACTGGTTCGTGATAGCCGTGCGTTC | |
| | | TCCACCACATTGTCATTTGAGGACA | | | | AGCACCACCCTGAGCTTTGAGGACA | |
| | | CGCAGAAGACGAATTCCACGATTAG | | | | CCCAGAAAACCAACAGCACCATTAG | |
| | | CAATAATGCAAGGAGCATTCATGAA | | | | CAACAACGCGCGTAGCATCCACGAA | |
| | | GCCTTTCCGCAGAATGAAGAAGGCT | | | | GCGTTCCCGCAAAACGAGGAAGGCT | |
| | | GCATTGACTATGATTTATTCATGGA | | | | GCATTGACTACGATCTGTTTATGGA | |
| | | CTACGTTCGTGGGCCGATGAGCCAA | | | | TTATGTGCGTGGTCCGATGAGCCAA | |
| | | AAACGGAAGGATGCCGTCTGGGAAA | | | | AAGCGTAAAGACGCGGTTTGGGAGA | |
| | | TATTCCGCAAGCTTGACTTTGATGG | | | | TCTTCCGTAAGCTGGACTTTGATGG | |
| | | AGACGGCTACCTCAACATCTTAGAC | | | | TGACGGCTACCTGAACATCCTGGAT | |
| | | ATTCAGGCCCGCTACAATGCGCAGC | | | | ATTCAGGCGCGTTATAACGCGCAGC | |
| | | AGCACCCTGTGGTGGCGGTGGAGAG | | | | AACACCCGGTGGTTGCGGTGGAACG | |
| | | ACTCTTCTCCGCGGACAAACTGCTT | | | | TCTGTTCAGCGCGGATAAGCTGCTG | |
| | | AAGGGCTTCCTCACCGTTTGGGATG | | | | AAAGGCTTTCTGACCGTTTGGGACG | |
| | | AGAACAAACAATACGGGTTGATCCC | | | | AGAACAAACAATACGGTCTGATTCC | |
| | | ATACGCCGAGTTTATCGACTACTAC | | | | GTATGCGGAATTCATCGACTACTAT | |
| | | AACGGCGTCAGCGCGGTAATTGCGG | | | | AACGGTGTTAGCGCGGTGATCGCGG | |
| | | ACGACTACATCTTTTTTGATATTCT | | | | ACGATTACATCTTCTTTGATATTCT | |
| | | CCGGAATCAATGGAAGGTCATGCGT | | | | GCGTAACCAGTGGAAGGTTATGCGT | |
| | | GACTGGGGAGGGACGGTGGGGACGA | | | | GACTGGGGTGGCACCGTGGGCACCC | |
| | | GGAGAGGGAAGAGTGAGGTTTCGAC | | | | GTCGTGGCAAAAGCGAGGTGAGCAC | |
| | | GATG (SEQ ID NO: 101) | | | | CATG (SEQ ID NO: 102) | |
| Tc19 | TcBrA4_ | ATGCCAAGCACACCCACCCCGCAGT | 1035 | 345 | ubiquitin- | ATGCCGAGCACCCCGACCCCGCAGT | MPSTPTPQCVRRLQKELSALCREAESF |
| | 0122270 + | GTGTGCGGCGGCTGCAAAAGGAGCT | | | conjugating | GCGTTCGTCGTCTGCAAAAAGAACT | FFTRPSAKSILVWYFVIKGPADTPYEG |
| | TcBrA4_ | TTCCGCCCTATGCCGAGAGGCCGAG | | | enzyme E2, | GAGCGCGCTGTGCCGTGAGGCGGAA | GRYFGKLNFPPDYPMKPPEIIILTPNG |
| | 0131050 | TCGTTTTTTTTCACCCGTCCCTCAG | | | 60S | AGCTTCTTTTTCACCCGTCCGAGCG | RFETNKSICLTMSNYHPENWSPLWGVR |
| | | CAAAGAGTATTCTGGTTTGGTATTT | | | acidic | CGAAAAGCATTCTGGTGTGGTACTT | TILTGLLSFMVGDELTTGCMTSSDELR |
| | | CGTCATCAAGGGTCCTGCGGATACC | | | ribosomal | TGTTATCAAAGGTCCGGCGGATACC | RKYARESRRFNAEKMSVYKELFPEEYQ |
| | | CCTTATGAAGGCGGTCGCTACTTTG | | | protein | CCGTATGAGGGTGGCCGTTATTTTG | KDLEELKREDSEKNGRTSGSAGCGANT |
| | | GCAAGCTGAATTTTCCCCCCGACTA | | | P2 | GCAAACTGAACTTCCCGCCGGACTA | KGGGVMESQEKEQWRGLFPALLGLFAV |
| | | TCCAATGAAACCGCCTGAGATTATC | | | | TCCGATGAAGCCGCCAGAAATCATT | LMGAYFWPWGGGGSMADKVEANDTLAC |
| | | ATTTTGACGCCAAATGGACGTTTTG | | | | ATCCTGACCCCGAACGGTCGTTTTG | TYAALMLSDAGLPITAEGIEAACVAAG |
| | | AGACCAACAAGAGCATTTGTCTCAC | | | | AAACCAACAAAAGCATTTGCCTGAC | LKVRNTLPVIFARFLEKKPLETLFAAA |
| | | CATGAGCAATTATCATCCGGAGAAT | | | | CATGAGCAACTACCACCCGGAAAAC | AATAPAEGAAAAPAAGSAAPAAAAAGA |
| | | TGGAGCCCTTTGTGGGGGGTCCGCA | | | | TGGAGCCCGCTGTGGGGCGTTCGTA | APEKDTKEEEEDDDMGFGLFD (SEQ |
| | | CCATTCTTACGGGGCTGCTCTCATT | | | | CCATCCTGACCGGTCTGCTGAGCTT | ID NO: 78) |
| | | CATGGTGGGAGACGAACTCACTACT | | | | CATGGTGGGCGATGAACTGACCACC | |
| | | GGGTGCATGACGAGCAGCGATGAGT | | | | GGTTGCATGACCAGCAGCGACGAGC | |
| | | TGCGGAGGAAGTATGCTCGTGAGAG | | | | TGCGTCGTAAGTATGCGCGTGAGAG | |
| | | CCGTCGTTTCAATGCAGAGAAAATG | | | | CCGTCGTTTTAACGCGGAAAAGATG | |
| | | TCAGTATACAAGGAACTGTTTCCTG | | | | AGCGTTTACAAAGAGCTGTTCCCGG | |
| | | AGGAGTATCAAAAGGATTTGGAGGA | | | | AGGAATATCAGAAGGATCTGGAGGA | |
| | | ATTGAAGCGAGAGGACAGTGAGAAA | | | | ACTGAAACGTGAGGACAGCGAAAAG | |
| | | AACGGTCGTACTTCTGGAAGTGCTG | | | | AACGGTCGTACCAGCGGTAGCGCGG | |
| | | GTTGTGGTGCGAATACGAAAGGAGG | | | | GTTGCGGTGCGAACACCAAAGGTGG | |
| | | AGGTGTGATGGAATCGCAAGAAAAA | | | | CGGTGTGATGGAAAGCCAGGAGAAG | |
| | | GAACAATGGCGTGGGTTATTCCCGG | | | | GAACAATGGCGTGGCCTGTTTCCGG | |
| | | CACTTTTGGGACTTTTTGCTGTGTT | | | | CGCTGCTGGGTCTGTTCGCGGTTCT | |
| | | AATGGGAGCCTACTTTTGGCCATGG | | | | GATGGGTGCGTACTTTTGGCCGTGG | |
| | | GGAGGTGGTGGCTCTATGGCCGATA | | | | GGCGGTGGCGGTAGCATGGCGGATA | |
| | | AGGTTGAAGCGAACGACACGCTGGC | | | | AAGTGGAGGCGAACGACACCCTGGC | |
| | | GTGCACCTACGCCGCCCTCATGCTC | | | | GTGCACCTATGCGGCGCTGATGCTG | |
| | | AGCGACGCGGGTCTGCCCATCACGG | | | | AGCGATGCGGGTCTGCCGATTACCG | |
| | | CGGAGGGCATTGAGGCCGCGTGTGT | | | | CGGAAGGTATTGAAGCGGCGTGCGT | |
| | | GGCTGCCGGTCTGAAGGTGCGCAAC | | | | GGCGGCGGGTCTGAAGGTTCGTAAC | |
| | | ACCCTGCCCGTTATTTTTGCTCGCT | | | | ACCCTGCCGGTGATTTTTGCGCGTT | |
| | | TTCTCGAAAAGAAGCCGCTGGAGAC | | | | TCCTGGAGAAGAAACCGCTGGAAAC | |
| | | TCTCTTTGCCGCTGCCGCTGCTACG | | | | CCTGTTCGCGGCGGCGGCGGCGACC | |
| | | GCACCTGCAGAGGGCGCCGCTGCTG | | | | GCGCCGGCGGAGGGTGCGGCGGCGG | |
| | | CTCCTGCCGCTGGCAGTGCCGCCCC | | | | CGCCGGCGGCGGGTAGCGCGGCGCC | |
| | | TGCCGCCGCAGCTGCCGGTGCTGCG | | | | GGCGGCGGCGGCGGCGGGTGCGGCG | |
| | | CCAGAAAAGGACACAAAGGAGGAGG | | | | CCGGAGAAGGATACCAAAGAGGAAG | |
| | | AGGAAGACGACGATATGGGTTTTGG | | | | AGGAAGACGATGACATGGGCTTTGG | |
| | | CTTGTTTGAC (SEQ ID | | | | TCTGTTCGAC (SEQ ID | |
| | | NO: 103) | | | | NO: 104) | |
| Tc20 | TcYC6_ | ATGCCAAGCACACCCACCCCGCAGT | 1035 | 345 | ubiquitin- | ATGCCGAGCACCCCGACCCCGCAGT | MPSTPTPQCVRRLQKELSALCREAESF |
| | 0088050 + | GTGTGCGGCGGTTGCAAAAGGAGCT | | | conjugating | GCGTTCGTCGTCTGCAAAAAGAACT | FFTRPSAKSILVWYFVIKGPADTPYEG |
| | TcYC6_ | TTCCGCCCTATGCCGAGAGGCCGAG | | | enzyme E2, | GAGCGCGCTGTGCCGTGAGGCGGAA | GRYFGKLNFPPDYPMKPPEIIILTPNG |
| | 0111870 | TCGTTTTTTTTCACCCGTCCCTCAG | | | 60S | AGCTTCTTTTTCACCCGTCCGAGCG | RFETNKSICLTMSNYHPENWSPLWGVR |
| | | CAAAGAGTATTCTGGTTTGGTATTT | | | acidic | CGAAAAGCATTCTGGTGTGGTACTT | TILTGLLSFMVGDELTTGCMTSSDELR |
| | | CGTCATCAAGGGTCCTGCGGATACC | | | ribosomal | TGTTATCAAAGGTCCGGCGGATACC | RKYARESRRFNAEKMPVYKELFPEEYQ |
| | | CCTTATGAAGGCGGTCGCTACTTTG | | | protein | CCGTATGAGGGTGGCCGTTATTTTG | KDLEELKREDNEKNGRISGSAGCGANT |
| | | GCAAGCTGAATTTCCCCCCCGACTA | | | P2 | GCAAACTGAACTTCCCGCCGGACTA | KGGGVMESQEKEQWRGLFPALLGLFAV |
| | | TCCAATGAAACCGCCTGAGATTATC | | | | TCCGATGAAGCCGCCAGAAATCATT | LMGAYFWPWGGGGSMADKVEANDTLAC |
| | | ATTTTGACGCCAAATGGACGTTTTG | | | | ATCCTGACCCCGAACGGTCGTTTTG | TYAALMLSDAGLPITAEGIEAACVAAG |
| | | AGACCAACAAGAGCATTTGTCTCAC | | | | AAACCAACAAAAGCATTTGCCTGAC | LKVRNTLPVIFARFLEKKPLESLFAAA |
| | | CATGAGCAATTATCATCCGGAGAAT | | | | CATGAGCAACTACCACCCGGAAAAC | AATAPAEGAAAVPAAGSAAPAAAAAAA |
| | | TGGAGCCCTTTGTGGGGGGTCCGCA | | | | TGGAGCCCGCTGTGGGGCGTTCGTA | APAKDTKEEEEDDDMGFGLFD (SEQ |
| | | CCATTCTTACGGGGTTGCTCTCTTT | | | | CCATCCTGACCGGTCTGCTGAGCTT | ID NO: 79) |
| | | CATGGTGGGAGACGAACTCACTACT | | | | CATGGTGGGCGATGAACTGACCACC | |
| | | GGGTGCATGACGAGCAGCGATGAGT | | | | GGTTGCATGACCAGCAGCGACGAGC | |
| | | TGCGGAGGAAGTACGCTCGTGAGAG | | | | TGCGTCGTAAGTATGCGCGTGAGAG | |
| | | CCGTCGTTTCAATGCAGAGAAAATG | | | | CCGTCGTTTTAACGCGGAAAAGATG | |
| | | CCAGTATACAAGGAACTGTTTCCAG | | | | CCGGTTTACAAAGAGCTGTTCCCGG | |
| | | AGGAGTATCAGAAGGACTTGGAGGA | | | | AGGAATATCAGAAGGATCTGGAGGA | |
| | | ATTGAAGCGAGAGGACAATGAGAAA | | | | ACTGAAACGTGAGGACAACGAAAAG | |
| | | AACGGTCGTATTTCTGGAAGTGCTG | | | | AACGGTCGTATTAGCGGTAGCGCGG | |
| | | GCTGTGGTGCGAATACGAAAGGAGG | | | | GTTGCGGTGCGAACACCAAAGGTGG | |
| | | AGGTGTGATGGAATCGCAAGAAAAA | | | | CGGTGTGATGGAAAGCCAGGAGAAG | |
| | | GAGCAATGGCGTGGGTTATTCCCGG | | | | GAACAATGGCGTGGCCTGTTTCCGG | |
| | | CACTTTTGGGACTTTTTGCTGTGTT | | | | CGCTGCTGGGTCTGTTCGCGGTTCT | |
| | | AATGGGAGCCTACTTTTGGCCATGG | | | | GATGGGTGCGTACTTTTGGCCGTGG | |
| | | GGAGGTGGTGGCTCTATGGCCGATA | | | | GGCGGTGGCGGTAGCATGGCGGATA | |
| | | AGGTTGAAGCGAACGACACGCTGGC | | | | AAGTGGAGGCGAACGACACCCTGGC | |
| | | GTGCACCTACGCCGCCCTCATGCTT | | | | GTGCACCTATGCGGCGCTGATGCTG | |
| | | AGCGACGCGGGTCTGCCCATCACGG | | | | AGCGATGCGGGTCTGCCGATTACCG | |
| | | CGGAGGGCATTGAGGCCGCGTGTGT | | | | CGGAAGGTATTGAAGCGGCGTGCGT | |
| | | GGCTGCCGGTCTGAAGGTGCGCAAC | | | | GGCGGCGGGTCTGAAGGTTCGTAAC | |
| | | ACCCTGCCCGTTATTTTTGCTCGCT | | | | ACCCTGCCGGTGATCTTTGCGCGTT | |
| | | TTCTTGAAAAGAAGCCGCTGGAGAG | | | | TCCTGGAGAAGAAACCGCTGGAAAG | |
| | | TCTCTTCGCTGCTGCCGCTGCTACG | | | | CCTGTTTGCGGCGGCGGCGGCGACC | |
| | | GCTCCTGCAGAGGGCGCCGCTGCTG | | | | GCGCCGGCGGAGGGTGCGGCGGCGG | |
| | | TTCCTGCCGCTGGCAGTGCCGCCCC | | | | TGCCGGCGGCGGGCAGCGCGGCGCC | |
| | | TGCTGCCGCAGCTGCCGCTGCTGCG | | | | GGCTGCTGCGGCGGCGGCGGCGGCG | |
| | | CCAGCAAAGGACACAAAGGAGGAGG | | | | CCGGCGAAGGATACCAAAGAGGAAG | |
| | | AGGAAGACGACGATATGGGTTTTGG | | | | AGGAAGACGATGACATGGGCTTTGG | |
| | | CTTGTTTGAC (SEQ ID | | | | TCTGTTCGAC (SEQ ID | |
| | | NO: 105) | | | | NO: 106) | |
| 3TolT | TcBrA4_ | AAGCATCTGAAGGACGAGAAGACCA | 1029 | 343 | a | AAGCACCTAAAAGATGAAAAAACAA | KHLKDEKTKVGSGPELLKRAAEQTVLS |
| | 0101970, | AGGTTGGAAGTGGACCGGAGCTGTT | | | fusion | AAGTAGGCTCTGGTCCGGAATTGCT | LEKAKEAEAEAEKAAAAAQKTRDAAEK |
| | TcYC6_ | GAAGAGGGCGGCAGAGCAGACTGTG | | | of aa | GAAACGTGCTGCGGAGCAGACCGTG | AAAARTLAQDVAATASALLRQREKEEE |
| | 0077100, | CTTTCTCTGGAGAAGGCAAAGGAGG | | | 150- | CTGAGCCTGGAAAAGGCAAAAGAGG | RRRARDRVRAYVGNERAENAMRVAWLD |
| | TcYC6_ | CGGAGGCGGAGGCTGAGAAGGCGGC | | | 260 | CGGAGGCAGAGGCGGAGAAGGCCGC | WVEGGGGSNHVKTDRRSKNSKTEGLLD |
| | 0078140 | AGCGGCGGCGCAGAAAACCCGGGAC | | | for | AGCCGCCGCACAAAAAACTCGCGAC | EAAKHTAIAVKKAKEAEAESEKAAAAA |
| | | GCAGCAGAGAAGGCAGCAGCGGCGC | | | TcBrA4_ | GCAGCCGAGAAGGCGGCGGCGGCGC | RKTLEAAEKAAAARTLAQDVAATASAL |
| | | GGACCTTGGCACAAGATGTTGCCGC | | | 0101970, | GTACCCTGGCTCAAGATGTTGCTGC | LRQREREEERRRAKDREAAEAAKKAAV |
| | | AACGGCCAGTGCGCTGCTGCGGCAG | | | TcYC6_ | GACCGCGAGCGCACTGTTGCGTCAG | AEVMKKFAAKKGGGGSKHVKDEKTKVG |
| | | CGGGAGAAGGAGGAGGAGAGGCGAA | | | 0077100, | CGTGAAAAAGAAGAAGAGCGTCGTC | SGPELLKRAAEQTVLSLEKAKEAEAET |
| | | GAGCGAGGGACAGGGTGAGGGCTTA | | | and | GGGCGCGTGACCGTGTTCGTGCATA | EKAAAAAQKTREAAEKAAAAQTLAQDV |
| | | CGTTGGAAATGAACGCGCCGAGAAT | | | TcYC6_ | CGTGGGCAACGAAAGAGCCGAGAAC | AATAIALLRQREKEEERRRARDREEAE |
| | | GCCATGAGGGTTGCGTGGCTGGACT | | | 0078140 | GCAATGCGTGTCGCGTGGCTGGATT | AAKKAAVAEVMNKFAAKKG (SEQ ID |
| | | GGGTGGAGGGAGGTGGTGGCTCTAA | | | with | GGGTTGAAGGTGGTGGTGGCTCTAA | NO: 80) |
| | | CCATGTTAAGACCGATAGGAGGAGC | | | linkers | TCATGTGAAGACCGATCGCCGTAGC | |
| | | AAGAATTCCAAAACAGAGGGTCTTT | | | in | AAAAACAGCAAAACGGAAGGCCTGT | |
| | | TGGACGAGGCGGCAAAGCATACTGC | | | between | TAGATGAAGCGGCGAAGCACACCGC | |
| | | AATTGCCGTAAAGAAGGCAAAGGAG | | | | GATCGCTGTGAAAAAAGCGAAAGAA | |
| | | GCGGAGGCGGAGTCTGAGAAGGCGG | | | | GCTGAGGCTGAGAGCGAAAAGGCCG | |
| | | CAGCGGCGGCGCGGAAAACCCTGGA | | | | CGGCGGCGGCTCGTAAGACCTTGGA | |
| | | AGCAGCAGAGAAGGCAGCAGCGGCG | | | | GGCGGCGGAGAAAGCGGCAGCGGCT | |
| | | CGGACCTTGGCACAAGACGTTGCCG | | | | CGCACCTTGGCTCAAGATGTGGCCG | |
| | | CAACGGCCAGTGCGCTGCTGCGGCA | | | | CCACGGCTTCGGCACTGTTGCGTCA | |
| | | GCGGGAGAGGGAGGAGGAGAGACGA | | | | GCGTGAGCGCGAGGAAGAGCGCCGT | |
| | | AGAGCGAAGGACCGGGAGGCGGCGG | | | | AGAGCTAAGGACAGAGAAGCGGCGG | |
| | | AGGCCGCGAAAAAGGCTGCCGTTGC | | | | AGGCGGCAAAGAAGGCCGCTGTGGC | |
| | | TGAGGTGATGAAGAAATTTGCTGCG | | | | AGAGGTAATGAAGAAATTCGCAGCG | |
| | | AAGAAGGGAGGTGGTGGCTCTAAGC | | | | AAGAAAGGTGGCGGTGGCAGCAAAC | |
| | | ATGTGAAGGACGAGAAGACCAAGGT | | | | ACGTTAAGGACGAAAAAACAAAAGT | |
| | | TGGAAGTGGACCGGAGCTGTTGAAG | | | | TGGTTCCGGTCCGGAACTGCTGAAG | |
| | | AGGGCGGCGGAGCAGACTGTGCTTT | | | | CGCGCCGCAGAACAGACTGTTCTGT | |
| | | CTCTGGAGAAGGCAAAGGAGGCGGA | | | | CCCTGGAGAAAGCGAAAGAAGCGGA | |
| | | GGCGGAGACTGAGAAGGCGGCAGCG | | | | GGCGGAAACCGAGAAGGCCGCTGCG | |
| | | GCGGCGCAGAAAACCCGGGAAGCAG | | | | GCGGCGCAGAAAACGCGTGAGGCGG | |
| | | CAGAGAAGGCAGCAGCGGCGCAGAC | | | | CGGAAAAGGCAGCAGCAGCGCAAAC | |
| | | CTTGGCACAAGATGTTGCCGCAACG | | | | CCTTGCCCAGGACGTGGCGGCTACC | |
| | | GCCATTGCGCTGCTGCGGCAGCGGG | | | | GCGATTGCACTGCTCCGTCAACGTG | |
| | | AGAAGGAGGAGGAGAGGCGAAGAGC | | | | AAAAGGAGGAAGAACGTCGCCGCGC | |
| | | GAGGGACCGGGAGGAGGCGGAGGCC | | | | TCGCGACCGCGAAGAGGCAGAGGCG | |
| | | GCGAAGAAGGCTGCCGTTGCTGAGG | | | | GCCAAGAAGGCCGCGGTCGCAGAGG | |
| | | TGATGAATAAATTTGCTGCGAAGAA | | | | TCATGAATAAATTTGCAGCGAAAAA | |
| | | GGGG (SEQ ID NO: 107) | | | | GGGC (SEQ ID NO: 108) | |

### B. Nucleic Acids Molecules, Vectors, and Host Cells

1. Isolated Nucleic Acid Molecules

Encoding Fusion Proteins

Isolated nucleic acid sequences encoding antigenic polypeptides and fusion proteins are also provided. As used herein, “isolated nucleic acid” refers to a nucleic acid that is separated from other nucleic acid molecules that are present in a mammalian genome, including nucleic acids that normally flank one or both sides of the nucleic acid in a mammalian genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. Thus, nucleic acids encoding the disclosed T. cruzi fusion proteins are isolated nucleic acids. A nucleic acid existing among hundreds to millions of other nucleic acids within, for example, a cDNA library or a genomic library, or a gel slice containing a genomic DNA restriction digest, is not to be considered an isolated nucleic acid.

Nucleic acids can be in sense or antisense orientation, or can be complementary to a reference sequence encoding the fusion protein. Reference sequences include, but are not limited to, nucleotide sequences which are known in the art and the specific sequences provided discussed above.

Nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid. Modifications at the base moiety can include deoxyuridine for deoxythymidine, and 5-methyl-2′-deoxycytidine or 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7:187-195; and Hyrup et al. (1996) Bioorgan. Med. Chem. 4:5-23. In addition, the deoxyphosphate backbone can be replaced with, for example, a phosphorothioate or phosphorodithioate backbone, a phosphoroamidite, or an alkyl phosphotriester backbone.

2. Vectors and Host Cells Expressing Fusion Proteins

Nucleic acids, such as those described above, can be inserted into vectors for expression in cells. As used herein, a “vector” is a replicon, such as a plasmid, phage, or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, WI), Clontech (Palo Alto, CA), Stratagene (La Jolla, CA), and Invitrogen Life Technologies (Carlsbad, CA).

An expression vector can include a tag sequence. As introduced above, tag sequences, are typically expressed as a fusion with the encoded polypeptide, an such tags can be inserted anywhere within the polypeptide including at either the carboxyl or amino terminus.

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell such as a CHO cell) can be used to, for example, produce the fusion proteins described herein.

C. Substrates

Substrates and other compositions including one or more of the disclosed polypeptide combinations, preferably as fusion proteins are also provided.

In some embodiments, one or more of polypeptide combinations, preferably as fusion proteins, are immobilized on a substrate such as a surface or support.

Exemplary surfaces include slides, plates (e.g., microplate, culture dish, etc.), paper, and beads.

Exemplary supports include those utilized to immobilize enzymes. See, e.g., Mohamad, et al., “An overview of technologies for immobilization of enzymes and surface analysis techniques for immobilized enzymes” Biotechnol Equip. 29(2): 205-220 (2015), which is specifically incorporated by reference herein in its entirety. The most commonly used supports are carboxymethyl-cellulose, starch, collagen, modified sepharose, ion exchange resins, active charcoal, silica, clay, aluminum oxide, titanium, diatomaceous earth, hydroxyapatite, ceramic, celite, agarose, treated porous glass (which is an organic material) and certain polymers. In some embodiments, the support material is a mesoporous material where a large surface areas and greater number of pores will lead to higher protein loading per unit mass. Porous supports are generally preferred as the high surface area permits a higher protein loading and the immobilized protein receives better protection from the environment. Nanostructured forms such as nanoparticles, nanofibres, nanotubes and nanocomposites are preferred to be used as carrier for protein immobilization and stabilization. These nanoscaffolds can be excellent support materials for immobilization as they can have the characteristics that balance large surface area and high mechanical properties.
The polypeptides and fusion proteins can be attached by interactions ranging from reversible physical adsorption, ionic linkages and affinity binding, to the irreversible but stable covalent bonds that are present through ether, thio-ether, amide or carbamate bonds.

Although there is no universal support that is appropriately suited for all proteins and applications, when selecting a support, certain characteristics of the support material can be considered such as having high affinity for protein, availability of reactive functional group, mechanical stability, rigidity, feasibility of regeneration, non-toxicity, biodegradability, and cost.

In some embodiments, the fusion proteins form part of a Bio-Plex suspension array system such as Luminex. Some such systems include a flow-based 96-well fluorescent microplate assay reader integrated with specialized software, automated validation and calibration protocols, and assay kits. The multiplex analysis system utilizes up to hundreds of fluorescent color-coded bead sets, each of which can be conjugated with a different specific fusion protein. The term “multiplexing” refers to the ability to analyze many different fusion proteins essentially simultaneously. To perform a multiplexed assay, sample and reporter antibodies are allowed to react with the conjugated bead mixture in microplate wells. The constituents of each well are drawn up into the flow-based Bio-Plex array reader, which identifies each specific reaction based on bead color and quantitates it. The magnitude of the reaction is measured using fluorescently labeled reporter antibodies specific for each antibody that may associate with the antigen being tested.

The Bio-Plex suspension array system uses a liquid suspension array of up to 500 sets of micrometer-sized beads, each internally dyed with different ratios of two spectrally distinct fluorophores to assign it a unique spectral address. For example, analyte such as fusion protein can be bound to a microsphere bead by, for example, a histidine tag. The fusion protein is then contacted with a sample of sera containing an antibody; for example, an anti-T. cruzi antibody. This antibody, in turn, is contacted with a fluorescently labeled reporter antibody to form a microsphere-antigen-antibody complex. Since the microsphere beads provide a large variety of different colors, and the microsphere beads were earlier attached only to specific fusion proteins, a number of microsphere-antigen-antibody complexes may be present in a microplate well. The complexes can then then run through a flow cytometry apparatus that includes a classifying laser and a reporting laser. The reporting laser can determine the amount of a particular fusion protein present, based on the amount of fluorescently labeled reporter antibody. The classifying laser, on the other hand, can determine the frequency of fluorescence provided by the microsphere bead, and based on this frequency, the identity of the fusion protein can be determined.

In some embodiments, the Bio-Plex (or another solid phase array) assay utilizes beads to capture the tagged T. cruzi fusion proteins. Each spectrally addressed bead captures a different protein. The protein-conjugated beads are allowed to react with a sample, and biomolecules in the sample (typically antibodies) bind to the bound protein antigens as further described elsewhere herein.

### III. Methods of Use

The disclosed polypeptide combinations and fusions proteins can be utilized in methods of detecting, diagnosing, and treating T. cruzi infections, and monitoring of the efficacy of treatment.

A. Detection, Diagnosis, and Treatment

1. Methods of Detection

Methods of detecting T. cruzi antibodies and other immune system components in a sample, e.g., a biological sample from a subject, are also provided. In preferred embodiments, the antibodies or other immune system components is an antibody that specifically binds to a T. cruzi antigen.

The methods typically include contacting one or more combinations of two more T. cruzi polypeptides (including, but not limited to, a combination of two or more polypeptides of Table 1, and/or one or combinations of Tables 2 and/or 3) with a sample. T. cruzi polypeptides can be fused or unfused. In preferred embodiments, two or more T. cruzi polypeptides are fused. Thus, in preferred embodiments, the methods include contacting one or more of the T. cruzi fusion proteins with a sample. For example, in some embodiments, such methods include contacting one or more of the disclosed T. cruzi unfused polypeptide combinations or T. cruzi fusion proteins with a biological sample under conditions suitable for antibodies therein capable of specifically binding to one or more of the disclosed unfused T. cruzi polypeptides or fusion proteins to bind thereto, and detecting the bound antibodies. Biological samples can be fluids, for example a body fluid. The body fluid can be any fluid found within the body of an organism that is capable of containing components of T. cruzi or immune system components prepared in response to exposure to T. cruzi. Such body fluids include, for example, whole blood, plasma, serum, urine, saliva, tears, lymphatic fluid, and the like.

Detection of bound antibodies can take any convenient form, including that of traditional immunoassays. For example, standard immunoassays such as indirect immunofluorescence assays (IFA), enzyme-linked immunosorbent assay (ELISA), radioimmunoassay (RIA), fluorescent bead technology and Western blots can be employed. Detection can be by way of an enzyme label, radiolabel, chemical label, fluorescent label, chemiluminescent label, a change in spectroscopic or electrical property, and the like. Most typically, detection includes incubation with and subsequent detection of a detection antibody (i.e., a secondary antibody) or other binding protein that binds to the fusion protein-bound T. cruzi antibodies.

In some embodiments, the method of detecting T. cruzi antibodies or other immune system components in a sample is a multiplexed assay. In a multiplexed assay, multiple analytes are simultaneously measured. The analytes typically include at least unfused T. cruzi polypeptide combination or one T. cruzi fusion protein and one more additional analytes, which can be, for example, one or more additional unfused T. cruzi polypeptide combinations or T. cruzi fusion proteins, one or more T. cruzi antigenic proteins (see, e.g., U.S. Published Application No. 2010/0323909), or a combination thereof. A collection of two or more analytes can also be referred to as a panel.

The panel may contain a number of antigenic unfused T. cruzi polypeptide combinations and/or T. cruzi fusion proteins, wherein said number is between 2 and 50 or even more, depending on the embodiment and the intended application. For example, the panel may contain 5, 8, 10, 12, 15, 18, 20, 25, 30, 40 or more antigenic unfused T. cruzi polypeptide combinations and/or T. cruzi fusion proteins. A typical multicomponent panel may contain 5 to 20 T. cruzi analytes. Preferably, some or all of the antigenic unfused T. cruzi polypeptide combinations and/or T. cruzi fusion proteins used in the multicomponent panel are derived or selected from those listed in one or more of Tables 1, 2, and/or 3. For example, in some embodiments, the panel includes between 2 and 100 unfused T. cruzi polypeptide combinations and/or fusion proteins formed of two polypeptides of Table 1, or fragments or variants thereof. In some embodiments, the panel includes 2, 3, 4, 5, 6, 7, 8, 9, 10, 11, 12, 13, or 14 of the fusion proteins of Table 2 or 3.

A panel-based detection assay can be used to assess the presence of an immune response (e.g., the presence of antibodies or reactive T cells) in a subject to multiple T. cruzi antigens.

In some embodiments, each analyte is positioned such that it is individually addressable. For example, the fusion protein antigens can be immobilized in a substrate. In a preferred embodiment, a multiplexed assay is performed using a bioassay such as the Luminex system (Luminex Corporation, Austin, Tex.). The Luminex system, which utilizes fluorescently labeled microspheres, allows up to 100 analytes to be simultaneously measured in a single microplate well, using very small sample volumes. However, other multiplex platforms such as protein microarrays can also be used, and the invention is not intended to be limited by the type of multiplex platform selected.

Unfused T. cruzi polypeptide combinations, fusion proteins, and other analytes, either singularly or part of panel can be assembled on any convenient substrate, for example on a microtiter plate, on beads, or in a microarray on a microchip. A microarray format is advantageous because it is inexpensive and easy to read using a standard fluorescence microscope. In this format, one can use the total number of spots (proteins) positive for each test patient to make a positive or negative detection. In addition, such tests are well-suited to adaptation for use with commercially available high-throughput devices and immunoassay protocols, for example those available from Abbott Laboratories and Applied Biosystems, Inc.

In some embodiments, the detection methods take the form of a serodetection or serodiagnostic assay, which detects a humoral (antibody) immune response in the subject. The binding of an antibody that is present in a biological fluid, such as a serum antibody, to a polypeptide or fusion protein is determined. The serodetection/diagnostic assays can take any convenient form, e.g., an immunoassay, etc., as introduced above. The assays can also take the form of an immunochromatographic test, in the form of a test strip loaded with the individual fusions proteins or panel components. The sample fluid can be wicked up onto the test strip and the binding pattern of antibodies from the fluid can be evaluated.

In another embodiment, the detection methods take the form of a cellular assay. In this embodiment, one or more antigenic polypeptides or fusion proteins are used to assess T cell responses in a mammalian subject, thereby providing another method for evaluating the presence or absence (or stage, etc.) of T. cruzi infection. Individuals are known who are serologically negative (based upon conventional tests) but who have T cells reactive with parasite antigens (usually a lysate of trypomastigotes and epimastigotes—but in some cases also against specific T. cruzi polypeptides). This indicates that T cell responses may be a sensitive way to assess infection, or to determine the stage of infection or exposure.

T cell responses to the unfused polypeptide and fusions proteins adding one or more antigenic fusion proteins to a blood fraction containing peripheral blood lymphocytes (e.g., a peripheral blood mononuclear cell, PBMC, fraction). The ability of the T cells to make IFN-gamma is then assessed, for example using an ELISPOT assay (e.g., Laucella et al., J Infect Dis. 2004 Mar. 1; 189(5):909-18). As another example, antigenic T. cruzi polypeptides and fusion proteins, can be bound to major histocompatibility complex (MHC) tetramers and presented to T cells, for example in a composition of peripheral blood lymphocytes, in a microarray format. In this assay, smaller polypeptides are preferred as they are more readily bound to the MHC tetramers and recognized by the T cells. Antigenic subunits of antigenic T. cruzi polypeptides and fusion proteins can be predicted using various computer algorithms, and are amenable to chemical synthesis. Binding of T cells to the spots containing MHC-polypeptide complexes indicates recognition and hence T. cruzi infection. See, for example, Stone at al (Proc. Nat'l. Acad. Sci. USA, 2005, 102:3744) and Soen et al. (PLoS. Biol, 2003, 1:429) for a description of the general technique.

2. Methods of Diagnosis

The methods of detection can be utilized in diagnosis of T. cruzi infection. Typically, the diagnostic methods include a detection assay, and further includes a diagnostic step. For example, where the sample is derived from a subject, and the assay detects T. cruzi antibodies, the subject can be diagnosed as positive for a T. cruzi infection. Conversely, in some embodiments where no T. cruzi antibodies are detected, the subject may be diagnosed as negative for a T. cruzi infection. In some embodiments, a negative antibody test is not sufficient for a negative diagnosis.

The threshold for a diagnosis of T. cruzi infection can be readily determined by the scientist, medical personnel, or clinician, for example based upon the response of known infected and control sera to the particular individual antigenic fusion protein(s) or panel being used. For example, diagnosis criteria can be based on a quantitative determination, for example, on the intensity of binding and optional subtraction of background. Optionally, the diagnostic test could be further refined to set quantitative cutoffs for positive and negative based upon the background response to singular fusion protein or unfused polypeptide combination or individual panel components. So, for example, the response to each fusion protein could be set to be >2 standard deviations above the response of “pooled normal,” sera.

Additionally or alternatively, where a panel is use, the diagnosis can be based on the number of “hits” (i.e., positive binding events). As an illustrative example, a multicomponent panel could contain 15 to 20 antigenic fusion proteins or combinations of two or more antigen polypeptides a positive diagnosis could be interpreted as, say, 5 or more positive responses.

A positive diagnosis for T. cruzi infection may lead to, or otherwise be accompanied by, a positive diagnosis for Chagas disease. Chagas disease, also known as American trypanosomiasis, is a tropical parasitic disease caused by T. cruzi. It is spread mostly by insects known as Triatominae, or “kissing bugs”. A diagnosis of Chagas disease may include a positive detection of one or more anti-T. cruzi antibodies alone or in combination with one or symptoms of Chagas disease. The symptoms change over the course of the infection. For example, in the early stage, symptoms are typically either not present or mild, and may include fever, swollen lymph nodes, headaches, or swelling at the site of the bite. After four to eight weeks, untreated individuals enter the chronic phase of disease, which in most cases does not result in further symptoms. Up to 45% of people with chronic infection develop heart disease 10-30 years after the initial illness, which can lead to heart failure. Digestive complications, including an enlarged esophagus or an enlarged colon, may also occur in up to 21% of people, and up to 10% of people may experience nerve damage.

Signs and symptoms differ for people infected with T. cruzi through less common routes. People infected through ingestion of parasites tend to develop severe disease within three weeks of consumption, with symptoms including fever, vomiting, shortness of breath, cough, and pain in the chest, abdomen, and muscles. Those infected congenitally typically have few to no symptoms, but can have mild non-specific symptoms, or severe symptoms such as jaundice, respiratory distress, and heart problems. People infected through organ transplant or blood transfusion tend to have symptoms similar to those of vector-borne disease, but the symptoms may not manifest for anywhere from a week to five months. Chronically infected individuals who become immunosuppressed due to HIV infection can suffer particularly severe and distinct disease, most commonly characterized by inflammation in the brain and surrounding tissue or brain abscesses. Symptoms vary widely based on the size and location of brain abscesses, but typically include fever, headaches, seizures, loss of sensation, or other neurological issues that indicate particular sites of nervous system damage. Occasionally, these individuals also experience acute heart inflammation, skin lesions, and disease of the stomach, intestine, or peritoneum.

a. Detection of T. cruzi Infection in Infants

In some embodiments, the methods of detection and diagnosis are utilized in a method for determining whether an infant has a T. cruzi infection. In situations where the infant's mother is infected with T. cruzi, the method facilitates early detection of a maternally transmitted infection. A maternally transmitted infection can be transmitted prior to or during birth (a congenital infection), or it may be transmitted after birth, as through breastfeeding.

At birth and for a period shortly thereafter, the antibody response of an infant born to an infected mother mirrors the antibody response of the infant's infected mother, reflecting the presence of maternal antibodies in the baby's fluids. With time, however, if the infant is infected, the infant will begin producing its own antibodies, and the pattern of response will begin to differentiate from that of the mother. Eventually, typically by about six months after birth, the antibody response of the infant will either diminish to near background levels (if the infant is not infected), or will appear distinct from that of the mother, indicating possible infection.

The method for detecting T. cruzi infection in an infant, particularly an infant born to a mother with a known or suspected T. cruzi infection, therefore can include analyzing at least one biological sample obtained from the infant. Preferably the biological sample is a body fluid such as blood, plasma or serum. The sample is obtained at a time after birth by which the infant's antibody response to the antigentic T. cruzi fusion protein or unfused combinations of two or more antigen polypeptides, if the infant is infected, is detectably different from the mother's antibody response. Preferably the sample is obtained from the infant at about 6 months of age, but the sample can be obtained earlier, for example at about 5 months, 4 months, 3 months or 2 months. Likewise, the sample can be obtained later since after 6 months the baby is expected to be producing its own antibodies at a detectable level. Analysis is preferably performed using the multiplexed assay of the invention.

An infant that exhibits a background level antibody response to T. cruzi antigens in the detection assay is unlikely to be infected with T. cruzi. However, an antibody response that exceeds background levels indicates possible infection. Optionally, the method therefore also includes administering a therapeutic agent an infant suspected of having a T. cruzi infection.

In a preferred embodiment of the method, the infant's antibody response is first analyzed shortly after birth. When a neonate's antibody response is measured shortly after birth (preferably no later than one month after birth, more preferably no later then two months after birth), the neonate's antibody response will parallel that of its mother, due to the presence of maternal antibodies. Optionally, the mother's antibody response to the antigen panel is thus also analyzed. The infant's antibody response, measured at the later time point (when its own antibodies have begun to be produced), is compared to the antibody response of the mother, and/or to its own antibody response at a time shortly after birth. Comparison of the antibody response of the later infant sample with the antibody response of the earlier neonate sample and/or with the antibody response of the mother (preferably using a sample obtained from the mother at about the same time as the sample or samples are obtained from the infant, although the sample from the mother can be obtained at any convenient time as it is expected to be fairly stable) is preferred, as it facilitates the determination as to whether the infant's own antibody response is sufficiently different from the mother's to support the diagnosis of T. cruzi infection.

It should be understood that in this method, as in all methods involving the use of the singular and multiplexed assays described herein, that the serodetection/diagnostic targets can include any one or more of the antigenic fusion proteins described herein. The methods of detecting T. cruzi infection in an infant can take the form of either a serodiagnostic method, wherein the sample components that interact with an antigenic T. cruzi fusion proteins are antibodies, or a cellular assay method, and wherein the sample components that interact with the antigenic T. cruzi fusion proteins are T cells.

b. Blood Supply Screening

In some embodiments, the methods of detection and diagnosis are utilized to detect the presence of T. cruzi infection in blood and blood products or fractions include whole blood as well as such as cellular blood components, including red blood cell concentrates, leukocyte concentrates, and platelet concentrates and extracts; liquid blood components such as plasma and serum; and blood proteins such as clotting factors, enzymes, albumin, plasminogen, and immunoglobulins, or mixtures of cellular, protein and/or liquid blood components. Details regarding the make-up of blood, the usefulness of blood transfusions, cell-types found in blood and proteins found in blood are set forth in U.S. Pat. No. 5,232,844. Techniques regarding blood plasma fractionation are generally well known to those of ordinary skill in the art and an excellent survey of blood fractionation also appears in Kirk-Othmer's Encyclopedia of Chemical Technology, Third Edition, Interscience Publishers, Volume 4.

B. Methods of Treatment

Any of the disclosed methods, including the methods of detection and diagnosis and include a further method of treatment. Chagas disease is managed using antiparasitic drugs to eliminate T. cruzi from the body and symptomatic treatment to address the effects of the infection. Benznidazole and nifurtimox are the antiparasitic drugs of choice for treating Chagas disease. For either drug, treatment typically consists of two to three oral doses per day for 60 to 90 days. Elimination of T. cruzi does not cure the cardiac and gastrointestinal damage caused by chronic Chagas disease, so these conditions must be treated separately.

Benznidazole is usually considered the first-line treatment because it has milder adverse effects than nifurtimox and its efficacy is better understood. Both benznidazole and nifurtimox have common side effects that can result in treatment being discontinued. The most common side effects of benznidazole are skin rash, digestive problems, decreased appetite, weakness, headache, and sleeping problems. These side effects can sometimes be treated with antihistamines or corticosteroids, and are generally reversed when treatment is stopped.

Thus, in some embodiments, a method of treatment includes administering to a subject positive for T. cruzi infection and/or Chagas disease an antiparasitic such as benznidazole or nifurtimox, alone or in combination with an antihistamine or corticosteroid and/or one or more agents to treat a symptom(s) of the infection.

Additional treatments are discussed in the Examples below are incorporate here by reference thereto, for use in the disclosed methods of treatment.

C. Methods of Monitoring Treatment

The disclosed methods of detection can also be used to monitor treatment of a subject for a T. cruzi infection and/or Chagas disease. Direct detection of parasites or parasite antigens is difficult and following treatment (even when not fully effective) is difficult using conventional techniques. Using the disclosed compositions and methods, declining antibody levels over time, preferably to multiple of these antigens, is indicative of treatment efficacy.

For example, the therapeutic efficacy of a treatment for a T. cruzi infection and/or Chagas disease can be assessed by quantifying the level of T. cruzi antibodies in an individual's biological sample over the course of treatment. Levels of T. cruzi antibodies present in a biological sample from the individual can be determined prior to treatment and subsequently at various time intervals during treatment. The levels of T. cruzi antibodies present in the biological sample of the individual undergoing treatment can be compared to the levels of T. cruzi antibodies present in biological samples from the same individual prior to treatment to determine the efficacy of the treatment in treating or resolving the T. cruzi infection and/or Chagas disease. The levels of T. cruzi antibodies in biological samples of the individual undergoing treatment can additionally or alternatively be compared to amounts of T. cruzi antibodies indicative of different stages or severity of a T. cruzi infection and/or Chagas disease.

For example, method for determining the efficacy of a treatment for a T. cruzi infection and/or Chagas disease in a subject can include determining the level of T. cruzi antibodies from one or more biological samples obtained from the subject before or during the course of the treatment, wherein a decrease in the level of T. cruzi antibodies in samples obtained from the subject over time is indicative that the treatment is efficacious.

A method for determining the efficacy of a treatment for a T. cruzi infection and/or Chagas disease in a subject can also include determining the levels of T. cruzi antibodies in a first biological sample and a second biological sample taken after the first sample wherein the samples are obtained from the subject over the course of the treatment, and wherein a decrease in the level of T. cruzi antibodies in the second sample compared to the first sample is indicative that the treatment is efficacious.

### Examples

### Example 1: T. cruzi Fusion Antigens Improved Ability to Detect Antibodies in the Blood of these Infected Hosts

Previous results have shown that multiplexed antigen arrays, like that made possible with Luminex bead arrays, can reveal substantial drops in serum antibodies to certain individual antigens much more rapidly that with antigen mixtures. In a previous study, pools of recombinant proteins were used to select antigens for T. cruzi Luminex assay (Cooley, et al., “High Throughput Selection of Effective Serodiagnostics for Trypanosoma cruzi infection,” PLOS (2008), doi.org/10.1371/journal.pntd.0000316). However even though this assay used or more parasite antigens, since antibody responses to T. cruzi are highly variable between individuals some individuals mounted antibody responses to only one of these select antigens.

Using a slide-based protein array platform, and sera from hundreds of humans who acquired T. cruzi infection in geographically diverse sites, and as well as from dogs and nonhuman primates with naturally acquired T. cruzi infection, >1000 T. cruzi proteins were evaluated for the ability to detect antibodies in the blood of these infected hosts. Next, fusions of the genes encoding the proteins having the broadest and strongest pattern of recognition by the large set of sera, were engineered, creating the 11 constructs shown in Table 2 (above) and produced the fusion proteins in E. coli.

Testing of a panel of 17 seropositive sera demonstrated that in comparison to previously used single T. cruzi recombinant proteins, the new fusions were superior in consistently detecting antibodies (FIG. 7).

These new fusions will next be tested with a broad array of both seropositive and seronegative serum samples from multiple species and will be utilized for monitoring post-treatment changes in anti-T. cruzi antibodies levels as and indicator of treatment efficacy. Additionally, fusions sets Tc17/Tc18 and Tc19/Tc20 are composed of genetic variants that may allow determination of the parasite genetic types responsible for the infections.

### Example 2: An Orally Active Benzoxaborole Prodrug Effective in the Treatment of Chagas Disease in Non-Human Primates

Padilla, et al., Nat Microbiol. 2022 October; 7(10):1536-1546. doi: 10.1038/s41564-022-01211-y. Epub 2022 Sep. 5. PMID: 36065062; PMCID: PMC9519446, is specifically incorporated by reference herein in its entirety, including all supplemental materials associated therewith.

Introduction

Chagas disease, caused by the protozoan parasite Trypanosoma cruzi, remains the highest impact parasitic disease in Latin America and one of the major causes of infection-induced myocarditis worldwide (Bonney & Engman, Curr Mol Med 8, 510-518 (2008)). For more than 5 decades, two nitroheterocyclic compounds, benznidazole and nifurtimox, have been available for treatment of the infection, but are relatively rarely used due to their inconsistent efficacy and high frequency of side effects. Recent trials of potential new therapies have yielded disappointing results (Torrico, et al., Lancet Infect Dis 18, 419-430 (2018)), Molina, et al., The New England journal of medicine 370, 1899-1908 (2014)). Among the challenges for drug development in T. cruzi infection is the parasite's predominantly intracytoplasmic location in mammals and its ability to invade a wide variety of host cell types and tissues, although it shows a clear preference for muscle cells, including cardiac, skeletal and smooth muscle of the gut. The recent discovery of arrested “dormant” intracellular forms of T. cruzi that are relatively and transiently resistant to otherwise highly effective trypanocidal compounds (Sanchez-Valdez, et al., eLife 7 (2018)) may partially explain why these therapeutics must be given for extended periods of time (60 days is common) but nevertheless still have a high failure rate. Previous work has identified a class of boron-containing molecules, the benzoxaboroles (Bustamante, et al., The Journal of infectious diseases 209, 150-162 (2014)), Baker, et al., Future Med Chem 1, 1275-1288 (2009)) as having potent activity against protozoans including Trypanosoma brucei (Jacobs, et al., PLoS neglected tropical diseases 5, e1151 (2011)), Leishmania donovani (Mowbray, et al., J Med Chem 64, 16159-16176 (2021)) and Plasmodium falciparum: (Zhang, et al., J Med Chem 60, 5889-5908 (2017)). Screening of the Anacor benzoxaborole compound library against T. cruzi revealed several hits, but initial assessment of structure-activity relationships (SAR) suggested limited opportunity for improvement of potency and/or selectivity, particularly in those sub-classes previously found to have activity against T. brucei and L. donovani. This work takes advantage of this benzoxaborole scaffold and the multiple natural host species for T. cruzi, to move rapidly from in vitro detection of trypanocidal activity in lead compounds into facile in vivo tests of efficacy in mice and ultimately in naturally infected non-human primates (NHPs). The result is identification of a class of benzoxaboroles that provide high rates of parasitological cure of T. cruzi infection. AN15368 from this class is the first, extensively validated and safe potential clinical candidate in over 50 years for the prevention/treatment of Chagas disease.

Materials and Methods

Compound Synthesis

All compounds used in this study were prepared as described in U.S. Pat. No. 10,882,272, granted Jan. 5, 2021, which is specifically incorporated by reference herein in its entirety. The syntheses of representative compounds AN14353 and AN15368 is described.

Parasites and Mice

C57BL/6J mice were purchased from The Jackson Laboratory (Bar Harbor, ME), and B6.129S7-Ifngtm1Ts/J (IFN-gamma deficient) were bred in-house at the University of Georgia Animal Facility. The SKH-1 “hairless” mice backcrossed to C57BL/6 were a gift from Dr. Lisa DeLouise (University of Rochester). All the animals were maintained in the University of Georgia Animal Facility under specific pathogen-free conditions at 22° C., 50% humidity and in a 12:12 hs light:dark cycle. Male and female mice of 6 to 9 weeks of age were used. All mouse experiments were carried out in strict accordance with the Public Health Service Policy on Humane Care and Use of Laboratory Animals and Association for Assessment and Accreditation of Laboratory Animal Care accreditation guidelines. The protocol was approved by the University of Georgia Institutional Animal Care and Use Committee. T. cruzi tissue culture trypomastigotes of the wild-type Brazil strain, Colombiana strain coexpressing firefly luciferase and tdTomato reporter proteins (Sanchez-Valdez, et al., eLife 7 (2018)), and CL strain expressing the fluorescent protein tdTomato (Canavaci, et al., PLoS neglected tropical diseases 4, e740 (2010)) were maintained through passage in Vero cells (American Type Culture Collection) cultured in RPMI 1640 medium with 10% fetal bovine serum at 37° C. in an atmosphere of 5% CO2. Parasites genotypes were determined as previously described (Padilla, et al., (PLoS neglected tropical diseases 15, e0009141 (2021)).

In Vitro Amastigote Growth Inhibition and Killing Assays

The in vitro anti-T. cruzi amastiogote activity assay was performed and optimized based on the protocol described previously (Canavaci, et al., PLoS neglected tropical diseases 4, e740 (2010)). The change in tdTomato fluorescence intensity was determined as a measurement of growth over 72 hours of culture. For assaying drug effects on extracellular amastigotes, trypomastigotes were collected from infected Vero cell cultures and converted in acidic media as previously described (Tomlinson, et al., Parasitology 110 (Pt 5), 547-554 (1995)). Amastigotes (50,000/well) were incubated with 2-fold serial dilutions of compounds for 48 hours. The ATP production was used as an indication of growth in this case, and was measured by ATPlite Luminescence ATP Detection Assay System (PerkinElmer). Both fluorescence and luminescence were read using a BioTek Synergy Hybrid Multi-Mode reader equipped with the software Gene5 v 2.0 (BioTek). The dose-response curve was generated by linear regression analysis with GraphPad Prism v9.4.0 (GraphPad Software). IC50 was determined as the drug concentration that was required to inhibit 50% of growth compared to that of parasites with no drug exposure.

In Vivo Compound Screens

Rapid assays: C57BL/6 mice were injected in the hind footpads with 2.5×10⁵ tdTomato expressing T. cruzi (CL strain) and orally treated with a single dose of the compounds (50 mg/kg) at 2 dpi. Fluorescent intensity of the feet was measured at 2 dpi before compound administration and at 4 dpi in the Maestro in vivo imaging system equipped with the software Maestro 2.1.0 (CRi) as previously described (Fleau, et al., J Med Chem 62, 10362-10375 (2019)). The proliferation index was estimated as PI=[(T4d−T2d)/(mUnt4d−mUnt2d)]*100; where T4d and T2d are the fluorescence intensity of the feet of the treated animals at days 4 and 2 post-infection respectively; mUnt4d and mUnt2d are the average fluorescence intensity of the feet of the untreated animals at 4 and 2 dpi respectively.

Cure assays: Male or female C57BL/6 mice were intraperitoneally infected with 10⁴ trypomastigotes of the Brazil strain. Mouse infection was confirmed at 25-30 dpi by detection of CD8⁺ T cells specific against the T. cruzi TSKb20 peptide in blood (Martin, et al., PLoS pathogens 2, e77 (2006)). Compounds resuspended in 1% carboxymethyl cellulose and 0.1% Tween 80, were administered daily by gavage at the specified concentrations. In order to optimally detect persistent infection, immune responses in the mice were suppressed by intraperitoneal injection of four doses of cyclophosphamide every 2-3 days (200 mg/kg/day), beginning one week after the end of therapeutic treatment. At the end of the immunosuppression regimen, peripheral blood was checked under light microscope for parasites and cultured in LDNT media (Bustamante, et al., The Journal of infectious diseases 209, 150-162 (2014)). Mouse skeletal muscle samples were obtained at the end of the immunosuppression and processed for T. cruzi-DNA detection by qPCR as previously described (Bustamante, et al., Sci Transl Med 12 (2020)). In vivo killing time assays: C57BL/6 mice were infected with 2.5×10⁵ Luciferase-expressing T. cruzi (Brazil strain) in the foot pads, and two days later one oral dose of AN14353 (25 mg/kg) was administered. The bioluminescent signal in the feet after i.p. injection of D-luciferin (PerkinElmer; 250 mg/kg) was measured in a Lumina II IVIS imager (PerkinElmer). Low dose short treatment: Hairless mice (SKH-1) were infected intraperitoneally with 5×10⁴ Luciferase expressing T. cruzi (Colombiana strain) and orally treated from 12 to 22 dpi with 1, 2.5 or 5 mg/kg of AN16109 or AN15368. Bioluminiscence signal of the whole body was measure after D-luciferin injection in a Lumina II IVIS in vivo imager equipped with the Living Imaging 4.0 software (PerkinElmer).

Generation of CBPs Knockout

The CBPs knockout was produced using ribonucleoprotein (RNP) complexes as previously described (Soares Medeiros, et al., mBio 8 (2017)). Briefly, Brazil tdTomato strain epimastigotes were electroporated with RNP complexes containing SaCas9 and an sgRNA targeting CBP gene TcBrA4_0048170, plus a repair template containing stop codons in all three reading frames and the M13 sequence for use as a PCR anchor. The sgRNA targeted the ‘GATTTACGTTGACCAGCCTGC’ (SEQ ID NO:110) sequence. After 2 days of recovery, single-cell clones were derived by depositing epimastigotes into a 96-well plate at a density of 0.5 cell/well by using a MoFlo Astrios EQ cell sorter (Beckman Coulter). DNA isolation was performed for clones, and primer pair of 5′-ACGTTGACCAGCCTGCAG-3′ (SEQ ID NO:111) and 5′-TGTGTATGGGTCTGTGAG-3′ (SEQ ID NO:112) was used for amplifying the wild type allele, while primer pair of M13-F: 5′-TGTAAAACGACGGCCAGT-3′ (SEQ ID NO:113) and 5′-TGTGTATGGGTCTGTGAG-3′ (SEQ ID NO:114) was used for amplifying the mutant allele.

Western Blot

A total of 5×10⁷ epimastigotes were harvested at 4° C. and washed once with cold PBS. Pellets were suspended in RIPA buffer (150 mM NaCl, 20 mM Tris.HCl pH7.5, 1 mM EDTA, 1% SDS, 0.1% Triton X-100) with 1% Protease Inhibitor Cocktail (Thermo Scientific), and incubated 1 hour on ice. Then the suspension was sonicated (Sonics & Materials, model 501) for 10 sec using microtip probe at 25 amplitude, and the sonicate centrifuged at 16,000 g for 10 min to remove the pellets and obtain total protein. Western Blot was performed according to the general established protocol. TcCBP-specific antibody (the gift of Drs. Juan José Cazzulo and Gabriela Niemirowicz at Instituto de Investigaciones Biotecnológicas, Buenos Aires, Argentina) was diluted at 1:500 and β-tubulin antibody was diluted at 1:1000. The IRDye 800CW Donkey anti-Rabbit IgG (Li-COR) was used as secondary antibody for both TcCBP and tubulin at 1:10,000 dilution. Images were taken in the BioRad ChemiDoc imaging system with the software ImageLab Touch 2.4.03.

Generation and Confirmation of the CPSF3 Overexpression Line

The CPSF3 gene was amplified using primers ‘ATGCTCCCTGCGGCAGCAGCAGTAA’ (SEQ ID NO:115) and ‘TTACACAGCCTCCTCTGGCAAAGGCT’ (SEQ ID NO:116), and integrated into the pTrex vector (Vazquez & Levin, Gene 239, 217-225 (1999)) by NEBuilder HiFi DNA assembly (New England Biolabs). The construct of pTrex-CPSF was then transfected into Brazil tdTomato epimastigotes and selected by 60 ug/ml blasticidin. To confirm the CPSF overexpression in the selected transfectants, RNA was extracted as previously described (Wang, et al., PLoS pathogens 17, e1009254 (2021)) and converted to cDNA using SuperScript Reverse Transcriptase (Invitrogen). Quantitative PCR reactions were performed in triplicate on the C1000 Touch Bio-Rad CFX96 real-time PCR detection system for CPSF using primer sets CPSF-1 (5′-TGAAACAGCAGCATGCCAAC-3′ (SEQ ID NO:117) and 5′-CGCGTCTGTCTACCATCAGA-3′ (SEQ ID NO:118)) and CPSF-2 (5′-CGGCTCATTCTGATGGTAGACA-3′ (SEQ ID NO:119) and 5′-TGTGCGTTGCACACTGAATG-3′ (SEQ ID NO:120) in both control and CPSF over-expressing parasites, and the expression level was normalized to tubulin which was amplified using primers: ‘AAGTGCGGCATCAACTACCA’ (SEQ ID NO:121) and ‘ACCCTCCTCCATACCCTCA’ (SEQ ID NO:122).

Generation of CPSF3 Mutant

To generate CPSF3 mutant, a RNP complex was transfected into Brazil tdTomato strain epimastigotes to target the CPSF3 gene (TcBrA4_0124800), together with a repair template that contained the mutation of Asn²³¹ to His²¹. The sgRNA targeted the ‘TCTGATTGCGGAAAGCACAA’ (SEQ ID NO:123) site. After 24 hours of recovery, 20 uM AN15368 was added to the parasites to select CPSF3 mutants that were resistant to drug treatment. The ultimate resistant parasites were validated to have acquired the Asn²³′His mutation in CPSF3 via sequencing.

RNA-Seq Sample Preparation, Sequencing and Analysis

Vero cells (10⁶) were infected with 10⁷ CL strain trypomastigotes of T. cruzi for 2 days before treating with either 5 uM benznidazole or 30 nM AN14353. The drug concentration used for treatment was set at 5 times the IC50. Samples were collected at several time points for RNA extraction as previously described (Minning, et al., BMC genomics 10, 370 (2009)). rRNA-depleted RNA library construction and RNA sequencing using Illumina Nextseq 75PE was carried out by Georgia Genomics and Bioinformatics Core (GGBC, University of Georgia, Georgia). Illumina reads with mean quality lower than 30 (Phred Score based) were removed from analysis, then mapped to CL Brener genome (TritrypDB release-33) and African green monkey genome (Osada, et al., DNA Res 21, 673-683 (2014)) using the HiSAT software package v0.1.6⁵⁰ with default parameters. The mapping rate was quantified by HTseq v0.6.1 (Anders, et al., Bioinformatics 31, 166-169 (2015)).

LC-MS/MS analysis of intracellular AN15368 and AN14667 Wild-type and peptidase knockout T. cruzi epimastigotes (5×10⁸) were treated with AN15368 (10 uM) or with DMSO vehicle control for 6 hours. The cells were then pelleted and resuspended in 100 μL of PBS. The cell suspension was mixed with 200 μl of acetonitrile and centrifuged at 735 g for 10 minutes at room temperature. After extraction, the supernatant was further diluted with methanol:water 30:70 (v/v) containing 0.4 nM internal standard (IS) AN14817 to a concentration within the calibration range. Each sample was diluted in triplicate to provide technical replicates and the diluted sample (10 μL) was injected for subsequent LC-MS/MS analysis.

LC-MS/MS analysis was performed on a Waters ACQUITY I-Class UPLC system coupled to a Xevo TQ-S triple quadrupole mass spectrometer. An ACQUITY UPLC BEH C18 column (130 Å, 1.7 m, 2.1 mm×50 mm) was used for chromatographic separation, and the column temperature was 40° C. The mobile phase consisted of water (A) and methanol (B), both containing 0.1% (v/v) formic acid. The following gradient elution was performed at a flow rate of 0.4 mL/min: 0-0.5 min, 30% B; 0.5-3 min, 30-95% B; 3-4 min, 95% B; 4-4.1 min, 95-30% B; and 4.1-5 min, 30% B. The MS ionization was carried out in the positive electrospray ionization (ESI) mode with following conditions: capillary voltage=1.50 kV; desolvation temperature=500° C.; desolvation gas flow=1000 L/h; and nebulizer gas pressure=7.0 bar. The MS/MS transitions used for detection and quantification were 390.1->174.9 for AN15368, 292.0->174.9 for AN14667, and 416.1->109.0 for AN14817. Data were processed using TargetLynx v4.1 software (Waters).

NHP Resource and Facilities

All NHP utilized for these studies were acquired from the approximately 1000-animal, Rhesus Macaque (Macaca mulatta) Breeding and Research Resource housed at the AAALAC accredited, Michale E. Keeling Center for Comparative Medicine and Research (KCCMR) of The University of Texas MD Anderson Cancer Center, in Bastrop. TX. This is a closed colony, which is specific pathogen free (SPF) for Macacine herpesvirus-1 (Herpes B), Simian retroviruses (SRV-1, SRV-2, SIV, and STLV-1), and Mycobacterium tuberculosis complex. All animals are socially housed in shaded, temperature-regulated indoor-outdoor enclosures with numerous barrels, perches, swings, and various feeding puzzles and substrates to mimic natural foraging and feeding behaviors. Standard monkey chow, ad libitum water, and food enrichment items are provided daily. Study animals that were seropositive for T. cruzi had acquired the infection naturally through exposure to the insect vector of the parasite while in their indoor-outdoor housing facilities. The NHP experiments were performed at the KCCMR and all protocols were approved by the MD Anderson Cancer Center's IACUC, and followed the NIH standards established by the Guide for the Care and Use of Laboratory Animals (Animals, National Academies Press: Washington, D C, 2011)).

Pharmacokinetic (PK) Analysis in NHP

Pre-treatment PK analysis of AN15368 distribution and clearance was performed to assist in determining the treatment dosing regimen. While under sedation/general anesthesia AN15368 was administered at various dosing levels either intravenously (i.v.) or via oral gavage to a T. cruzi-seronegative rhesus macaque, and 500 uL blood samples were collected prior to dosing and at 2, 5, 15, 30 and 60 minutes post-dose administration at which time the animal was recovered from general anesthesia. Additional 500 uL blood samples were collected at 3, 6, 9, and 24-hours post-dose administration under light-anesthesia/sedation. After the initial IV/PO PK assessment, a pre-regimen PK assessment of oral dosing was conducted with administration of a single dose of AN15368 in 3 animals over 3 dosing periods, with AN15368 (30 or 50 mg/kg dose) administer in food treats. For this pre-regimen phase, blood samples were collected at pre-dose, then at 0.25, 0.5, 1, 3, 6, 9 and 24 hours post-dose.

Mid- and end-regimen PK assessments were also performed. A “peak and trough” mid-regimen (day 30) PK assessment was performed on 3 treated animals: 1) blood was collected prior to drug dosing; 2) the animals were gavage-dosed with AN15368 in pumpkin slurry; and 3) a second blood sample was collected three hours post-dosing. The end-regimen PK analysis was performed on the 60th (and final) day of AN15368 dosing, with 18 of the 19 treated animals utilizing a non-serial, sparse sampling design. For this study 3 animals had blood collected prior to being provided the AN15368 in food treats. The other 15 animals were provided AN15368 in food treats and then blood was collected from 3 separate animals at 0.5, 1, 3, 6 and 9 hours post-dosing. Only 1 blood sample was collected at a single time point from each of the 18 animals. The composite plasma PK profile on Day 60 was obtained using the mean concentrations (n=3) at each sampling timepoint (predose, 0.5, 1, 3, 6 and 9 hr). All blood samples (500 uL) for PK analysis (pre-, mid- and end-regimen) were collected into EDTA microtainers and plasma harvested for the determination of AN15368. The plasma samples were provided to Pharmout Labs (Fremont, California) for analysis using LC-MS/MS. The mean pre-dose concentration was also depicted as the 24 hr post-dose concentration and used for the calculation of post-treatment AUC0-24 value on Day 60.

For calculation of PK parameters, the Cmax (maximum concentrations) and tmax (time to maximum concentrations) were determined by visual inspection of the plasma concentration vs. time curves from the pre-regimen and end-regimen periods. PK calculation was not performed for the mid-regimen PK samples since only 2 time points were collected. The AUC values for the pre- and end-regimen were calculated using the linear-trapezoidal rule with the following equation: AUC(t1−t2)=[(Ct2+Ct1)×(t2−t1)]/2 where t1 and t2 are consecutive sampling time points, AUC(t1−t2) is the fractional area-under-the-curve over time intervals t1 and t2, Ct2 is the concentration at time t2, Ct1 is the concentration at time t1. The total AUC (AUC0-24) over the dosing interval (24 hr) was calculated by summation of all fractional AUC values over the intervals between 0 (pre-dose) and 24 hours post-dose. When a terminal elimination phase was apparent in the plasma concentration vs. time curve, the terminal half-life (t1/2) was estimated using the equation t1/2=0.693/λz, where λz was the elimination rate constant estimated from the slope of the terminal elimination phase. AUC0-∞ was estimated using the following equation: AUC0-∞=AUClast+Clast/λz Where AUC0-∞ was AUC from zero to infinity, AUClast was the AUC from zero to the last measurable time point, Clast was the concentration at the last measurable time point. AUC0-∞ and t1/2 were not estimated when the terminal elimination phase was not defined. Plasma clearance (CLp) after the IV dose was estimated using the following: CLp=Dose/AUC0-∞. Bioavailability (% F) after a single oral dose was estimated using the following: % F=(AUC0-∞, PO/Dose PO)/(AUC0-∞, IV/Dose IV) Mean AN15368 plasma concentrations and PK parameters after a single IV or PO dose and mean AN15368 (total and free) plasma concentrations and PK parameters in the pre- and end-regimen periods are depicted.

NHP Treatment Study

A total of 22 rhesus macaques that had been confirmed to be serologically- and PCR-positive for T. cruzi were utilized in these studies. Using 19 animals in the treatment group provided 85% power of detecting 100% efficacy. The 19 animals were treated with a 30 mg/kg dose of AN15368 delivered in food treats once a day for 60 days. The remaining three animals on the study were maintained as untreated control animals and received food treats but were not dosed with AN15368.

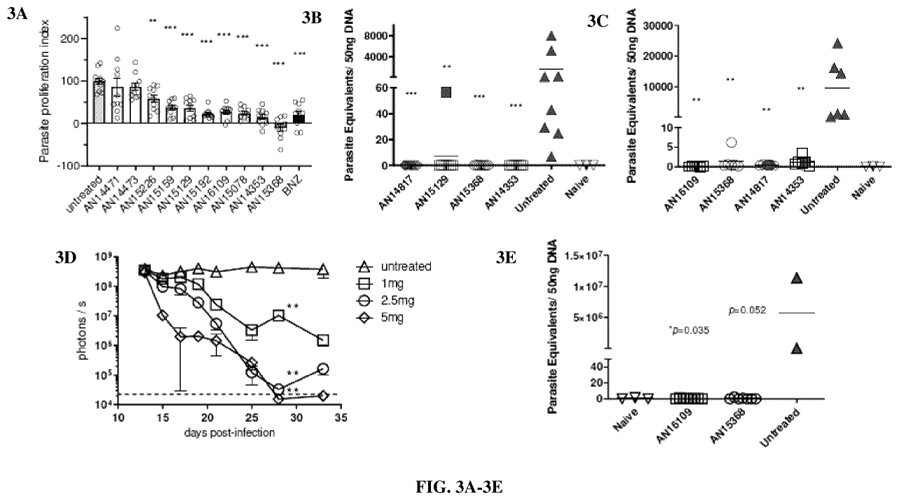

The selected dose of 30 mg/kg in NHP was determined based upon the following rationale. The minimal efficacious dose in mice was determined to be 2.5 mg/kg (FIG. 3E) and pharmacokinetic (PK) studies in mice at a 10 mg/kg dose yielded an exposure of 17.5 (AUC0-last (μg·hr/kg);). Assuming linear exposure, a minimal curative exposure in mice was estimated at 4.375 μg·hr/kg (i.e. 17.5/4). PK analysis of a 30 mg/kg dose in NHP indicated an exposure of 3.5-4.7·μg·hr/kg. Based on allometric scaling¹⁵ the NOAEL of 120 mg/kg in rats translates to a NOAEL dose of 60.5 mg/kg in monkeys and 19.4 mg/kg in humans. Thus, the 30 mg/kg NHP dose was projected to achieve an efficacious level based upon PK comparisons of mouse and NHP and be safe as it is well below the estimated NOAEL dose determined in rats. Under light-anesthesia/sedation, peripheral blood samples were collected from each animal prior to treatment and at 7 time points following. Blood analysis (CBC and serum chemistry assays) and physical exams to evaluate the health of the study animals were performed on each animal prior to the beginning of the study and also at three time points during the treatment protocol. At the termination of the study, 9 treated and 2 control animals were euthanized, necropsied, and blood and tissues were evaluated histologically and using PCR and hemoculture for evidence of active T. cruzi infection. The remaining 10 treated and 1 control animals from the study were returned to the breeding colony at the Keeling Center.

NHP Blood and Tissue PCR for T. cruzi DNA and Hemoculture

Blood samples from each macaque were collected at various time points and processed for quantification of T. cruzi DNA by real-time qPCR. Between 8-ml of whole blood collected in EDTA anticoagulant tubes was subjected to DNA extraction using the Omega E.Z.N.A. Blood DNA Maxi Kit (Omega Bio-Tek), following the manufacturer's instructions for up to 10 ml whole blood and using a total of 650 μl of elution buffer. Each round of extractions included a negative (no-template) control comprised of 10 ml PBS. The concentration of DNA in the eluted solution was quantified after each extraction using an Epoch microplate spectrophotometer (BioTek).

DNA from each sample was then subjected to a series of two qPCR assays for detection of T. cruzi satellite DNA. The first qPCR used the cruzi 1, 2 primer set and cruzi 3 TaqMan probe as previously described (Piron, et al., (Acta tropica 103, 195-200 (2007)), Duffy, et al., (PLoS neglected tropical diseases 7, e2000 (2013)), using BioRad iTaq Universal Probes Supermix (Bio-Rad). This qPCR amplifies a 166-bp region of a repetitive satellite DNA sequence and is sensitive and specific for T. cruzi when compared to other PCR techniques (Schijman, et al., (PLoS neglected tropical diseases 5, e931 (2011)). In order to rule out false negative PCR results due to inhibition, an internal amplification control (IAC) was added to the second qPCR reaction, which was run as a multiplex as previously described, with the cruzi 1/2/3 primers and probe and the IAC primers and probe (Duffy, et al., PLoS neglected tropical diseases 7, e2000 (2013)), except that the IAC sequence was synthesized as a gene fragment by a commercial laboratory (gBlocks Gene Fragments, Integrated DNA Technologies), and was added at the time of PCR, rather than before extraction. Positive (DNA extracted from T. cruzi Sylvio X10 clone 4, American Type Culture Collection, ATCC #50800, known concentration 1.7×10⁻³ parasite equivalents) and negative (water) controls were included in each PCR plate for both assays. C1000 Touch Bio-Rad CFX96 real-time PCR detection system was used for both assays under the following cycling conditions: (i) initial denaturation, 95°, 3 min; (ii) denaturation, 95° C., 15 s; (iii) annealing, 58° C., 1 min; (iv)×50 cycles. FAM and VIC channels were selected for each read.

Frozen tissues were screened for T. cruzi DNA using 8 mm biopsies (Sklar instruments #96-1130), collecting 3 to 10 individual ˜100 μl tissue punches for each tissue type and one of more pooled sample consisting of five punches from different areas of the tissue, totaling ˜500 μl per pool. The tissues sampled included liver, heart, fat, esophagus, quadricep, bicep, large intestine, and brain, as well as tongue and spleen in a few instances. Two individual and one pooled sample of tissues from an uninfected macaque was collected for each sampling batch. DNA from macaque tissue was extracted and analyzed as previously described (Bustamante, et al., Sci Transl Med 12 (2020)) with the exceptions that the purification was scaled up to accommodate the larger amount of tissue in the pooled samples and the range for the standards was 2.6×10²-2.6×10⁻³ parasite equivalents using the kDNA minicircle S35 and S36 primers. The Biorad CFX manager software version 3.1 was used to analyze PCR data. For samples to be considered positive, both replicates per sample must show a product (cQ value) of ≤40 and less than that of the included naïve sample and melt curves had to be in the same temperature range as the standards for each plate.

For hemoculture determinations, peripheral blood from macaques was collected and cultured at 26° C. in supplemented liver digest neutralized tryptose medium as described previously (Padilla, et al., PLoS neglected tropical diseases 15, e0009141 (2021)). The presence of T. cruzi parasites was assessed every week for 3 months under an inverted microscope. T. cruzi DTU of the macaque isolates was determined as previously described (Padilla, et al., PLoS neglected tropical diseases 15, e0009141 (2021)).

Multiplex Serological Analysis

Luminex-based multiplex serological assays were performed as previously described (Cooley, et al., PLoS neglected tropical diseases 2, e316 (2008), Padilla, et al., PLoS neglected tropical diseases 15, e0009141 (2021)). For a number of smaller proteins, fusions of up to 2 individual genes are employed for some target proteins in order to expand the array of antibodies being detected while controlling costs and complexity of the assay. (TritrypDb.org identifiers: Tc1=fusion of TcBrA4_0116860 and TcYC6_0028190; Tc2=fusion of TcBrA4_0088420 and TcBrA4_0101960; Tc3=fusion of TcBrA4_0104680 and TcBrA4_0101980; Tc4=fusion of TcBrA4_0028480 and TcBrA4_0088260; Tc5=fusion of TcYC6_0100010 and TcBrA4_0074300; Tc7=fusion of TcYC6_0083710 and TcBrA4_0130080; Tc8=TcYC6_0037170; Tc11=TcYC6_0124160; Tc17=fusion of TcBrA4_0028230 and TcBrA4_0029760; Kn107=TcCLB.508355.250; G10=TcCLB.504199.20). Macaque antibody binding to individual beads in the Multiplex assays was detected with donkey anti-human IgG (H+L) conjugated to phycoerythrin (Jackson ImmunoResearch) in a 1:200 dilution.

Statistics and Reproducibility

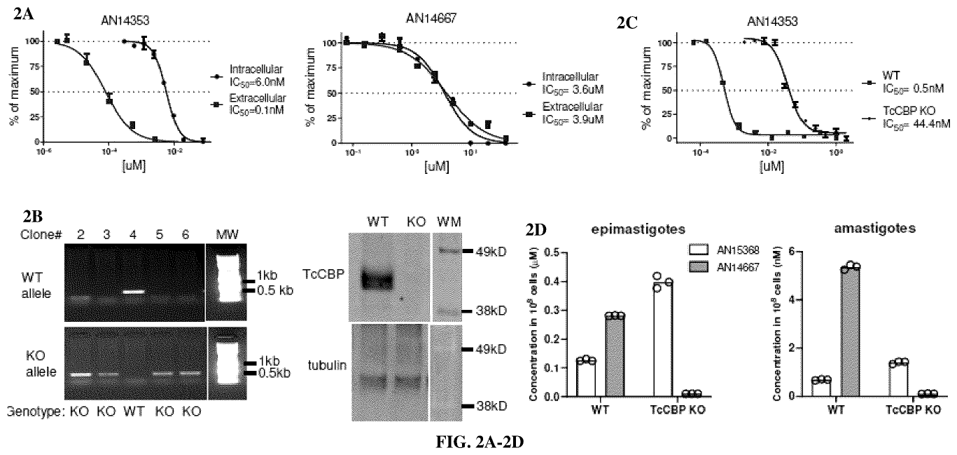

The non-parametric Mann-Whitney U test and the unpaired t-test from the software GraphPad Prism v9.4.0 were used. Values are expressed as mean±SEM. Statistical significance of P values was considered as *=p<0.05; **=p<0.01; ***=p<0.001. All mouse experiments were performed at least twice with similar results. All in vitro parasite proliferation assays were repeated at least one time with similar results. PCR and Western blot assays depicted as representative microphotographs in FIG. 2B were repeated three and one time respectively with similar results. The quantitative liquid chromatography tandem mass spectrometry assay described in FIG. 2D was performed once. Due to cost and complexity, the NHP trial was performed once.

Results

In Vitro Activity and SAR.

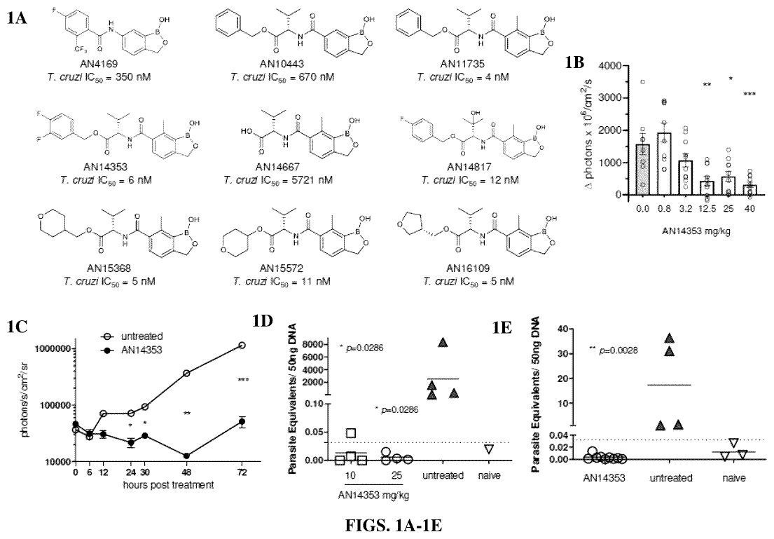

The initial lead benzoxaborole 6-carboxamide AN4169 (FIG. 1A) provided 100% cure of mice infected with the T. cruzi Brazil strain (Bustamante, et al., The Journal of infectious diseases 209, 150-162 (2014)), however rodent tolerability studies suggested that an insufficient therapeutic margin existed for further progression of this compound. Profiting from a concurrent project evaluating analogues of AN4169 against Trypanosoma congolense (Akama, et al., Bioorg Med Chem Lett 28, 6-10 (2018)), several compounds with submicromolar activity against T. cruzi in vitro and good metabolic stability in an in vitro mouse S9 liver fraction assay were identified, among these an ester of the 6-valine “transposed” carboxamide AN10443 (FIGS. 1A-1E). Further manipulation of this compound, in particular inclusion of a methyl group at C(7) of the benzoxaborole ring as in AN11735, drastically increased in vitro activity against T. cruzi (IC50<10 nM) whereas substituents larger than methyl at C(7) ablated activity (below, Table 4). Structure activity relationship (SAR) development of the ester region in the C(7)-methyl series showed that potency was not significantly impacted by substituents on the benzyl ester, with the exception of the 4-(3-pyrrolidinylethyloxy) analog AN14502 and the 4-methylsulfonyl analog AN14561, which were significantly less potent.

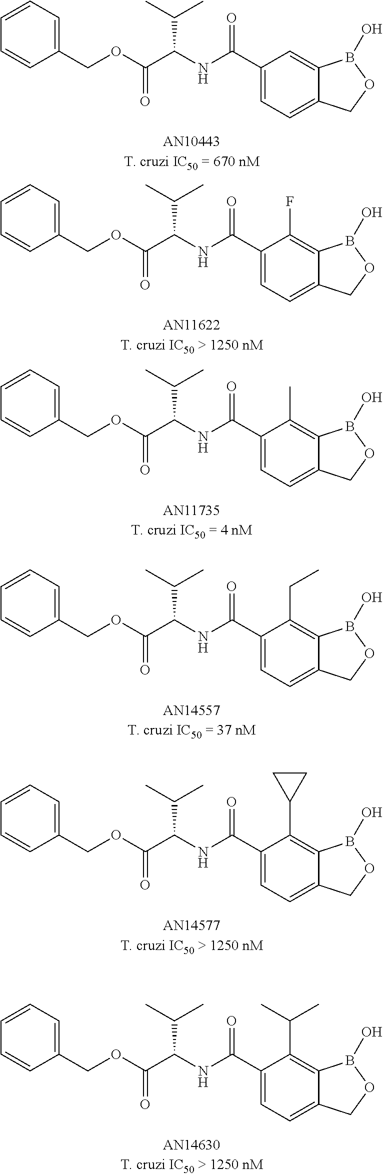 [SMILES: CC(C)[C@H](NC(=O)c1ccc2c(c1)B(O)OC2)C(=O)OCc1ccccc1 ; CC(C)[C@H](NC(=O)c1ccc2c(c1F)B(O)OC2)C(=O)OCc1ccccc1 ; Cc1c(C(=O)N[C@H](C(=O)OCc2ccccc2)C(C)C)ccc2c1B(O)OC2 ; CCc1c(C(=O)N[C@H](C(=O)OCc2ccccc2)C(C)C)ccc2c1B(O)OC2 ; CC(C)[C@H](NC(=O)c1ccc2c(c1C1CC1)B(O)OC2)C(=O)OCc1ccccc1 ; CC(C)c1c(C(=O)N[C@H](C(=O)OCc2ccccc2)C(C)C)ccc2c1B(O)OC2]

TABLE 4

AN14353

| Strains | DTU | IC₅₀ (nM) | IC₉₀ (nM) |
|---|---|---|---|
| Colombiana | TcI | 7 | 10 |
| Montalvania | TcI | 1 | 2 |
| 20290 | TcI | 3 | 5 |
| ARC0704 | TcI | 3 | 7 |
| Tul8 | TcII | 1 | 2 |
| M5631 | TcIII | 1 | 3 |
| 20392 | TcIV | 5 | 10 |
| CL | TcVI | 6 | 18 |

Physicochemical properties were more significantly affected, with most simple halogenated analogs being poorly soluble in aqueous media. Metabolic stability, as estimated by incubation with the mouse S9 liver fraction, was variable, and roughly tracked with lipophilicity (c Log D). These observations prompted a more substantially modify the ester region of the molecule through preparation of aliphatic and heterocyclic esters that would be thought to be less lipophilic, more water soluble and less susceptible to metabolism. Several interesting SARs emerged from this group of analogs: (1) esters containing basic amines (e.g. AN15143, AN15144, AN15658, AN15678, AN15129, AN15192, AN15078, AN14504 and AN15159) were less active than neutral compounds and (2) small aliphatic esters were quite potent except for the t-butyl ester (AN15134). The relationship between lipophilicity and solubility or metabolic stability continued to exist for these compounds and provided reasonably wide latitude for modulation of such properties by choice of ester substituent.

In Vivo Activities.

In addition to being very potent in vitro, the valine esters were also of generally good stability, including in mouse and human S9 liver fraction assays. Several also exhibited low clearance (<20% hepatic blood flow) following intravenous dosing and good bioavailability following oral administration to mice, achieving good to excellent exposure (AUC >10 μg·hr/mL) with low mg/kg doses. Concurrent testing of these compounds in vivo for the ability of a single oral dose to reduce an established focal infection in the footpad of mice over 3 days (Canavaci, et al., PLoS neglected tropical diseases 4, e740 (2010), Bustamante & Tarleton, (Expert Opin Drug Discov 6, 653-661 (2011)) were very encouraging, with AN14353 emerging as the lead based on activity at reduced doses (FIG. 1B). Notably, AN14353 demonstrated rapid in vivo trypanocidal activity (FIG. 1C), had high in vitro potency for a range of T. cruzi isolates for different genetic lineages (discrete typing units, DTUs; Table 4) and could consistently resolve established T. cruzi infections at a dose of 25 mg/kg in a standard (Bustamante, et al., (The Journal of infectious diseases 209, 150-162 (2014), Bustamante, et al., (Nature medicine 14, 542-550 (2008)) 40 day treatment protocol in wild-type mice (FIG. 1D) as well as infections in immunodeficient mice (FIG. 1E). Lead benzoxaboroles are prodrugs activated by a parasite serine carboxypeptidase Experiments were next designed to understand the importance of the ester group for activity, as this functional group carries liabilities for hydrolytic and metabolic instability. An early indication of the importance of the ester function to anti-T. cruzi activity was evident from the already noted lack of potency of the t-butyl ester AN15134. Amide, N-methyl amide, ketone, ether and acylsulfonamide analogs of AN11735, as well the 1,2,4-oxadiazole ester bioisostere AN14562 all lacked activity. Furthermore, the carboxylic acid metabolite, AN14667 had ˜1000-fold reduced activity on both intra- and extracellular amastigotes (FIGS. 1A and 2A). Thus, the ester functionality is important for anti-T. cruzi activity. It was believed that the esters might be pro-drugs to the carboxylic acid and were cleaved within either the host cell or by a parasite enzyme. The high sensitivity of extracellular amastigotes to AN14353, but not to the carboxylic acid AN14667 (FIG. 2A) virtually eliminated the requirement for a host peptidase in pro-drug activation. Several candidate enzymes with potential ester cleavage activity in T. cruzi were identified, including a serine carboxypeptidase (CBP; TcCLB.508671.20) present at 2-18 copies in different T. cruzi strains (Wang, et al., PLoS pathogens 17, e1009254 (2021)) and a metallocarboxypeptidase 2 (TcCLB.504045.60). CRISPR-Cas9-driven disruption of all copies of the serine carboxypeptidases (FIGS. 2B and 2C) but not the metallocarboxypeptidase decreased in vitro sensitivity of T. cruzi amastigotes by up to 100-fold. Accumulation of the AN14353 analogue AN15368, and its conversion to the cleaved product in wild-type T. cruzi epimastigotes and amastigotes but not in the KO line, was documented by quantitative liquid chromatography tandem mass spectrometry (FIG. 2D), thus confirming the serine carboxypeptidase-dependent prodrug to drug conversion within T. cruzi.

Mouse Test of Cure Studies.

Evaluation of dose proportionality of exposure with AN14353 and generation of the carboxylic acid metabolite AN14667 in vivo suggested solubility-limited absorption of this compound, prompting an attempt to further optimize aqueous solubility. Focusing on the ester region, a variety of more polar, non-basic substituents such as aliphatic and cyclic ethers as well as hydroxyvaline analogs (predicted to be less hydrophilic) of the lead compounds were evaluated for both solubility and in the in vitro trypanocidal assay. The highest in vitro-active compounds in this set were then evaluated and found to have anti-T. cruzi activity in the 2 day in vivo assay (FIG. 3A). Extensive in vitro and in vivo pharmacokinetic analysis and metabolic stability assays in mouse and human liver S9 fraction and both mouse and human plasma and in vivo pharmacokinetics studies ultimately identified three additional compounds of particular interest—AN14817, AN15368, and AN16109. Based on these aggregate data, these and related compounds were progressed into a series of “test of cure” assays in mice with a terminal immunosuppression period to reveal residual infection (Bustamante, et al., The Journal of infectious diseases 209, 150-162 (2014)). An initial screening using 10 mg/kg for 40 days identified several compounds for which the treated animals showed no parasite recovery by hemoculture and no parasite DNA detection in skeletal muscle using PCR (FIG. 3B). A more stringent test using only 20 days of treatment (a treatment period for which benznidazole is only partially effective in generating cure; (Bustamante, et al., The Journal of infectious diseases 209, 150-162 (2014)) further distinguished the highest potency compounds from less potent ones (FIG. 3C). Based on these results, lower doses of the candidates were evaluated in a short-term (non-cure) treatment course of intraperitoneal infection with luciferase-expressing parasites (FIG. 3D) from which a dose of 2.5 mg/kg was identified and shown effective in a 40 day-treatment cure assay (FIG. 3E).

Non-Human Primate Test of Cure Study.

AN14353, AN14817 and AN15368 were progressed to an array of preliminary safety pharmacology, genotoxicity and toxicology studies and all three compounds were found to exhibit little to no affinity for a broad array of mammalian enzymes, receptors and ion channels, were non-genotoxic in standard Ames and in vitro micronucleus studies, and did not demonstrate significant inhibition of representative cytochrome P450 enzymes at 10 uM. High dose 7-day toxicology studies did not distinguish the three compounds from each other, but the non-dose proportional exposure noted previously with AN14353 was also seen with the benzylic ester AN14817, likely a consequence of solubility limited oral absorption. In contrast, the more hydrophilic analog AN15368 exhibited good dose proportional exposure in rats and modest effects on hematology and clinical chemistry at 150 mg/kg, but none at 120 mg/kg/day or lower. Total plasma exposure (AUC0-24 h) in rats at 120 mg/kg/day was approximately 30,000 ng*hr/mL, with no evidence of drug accumulation between the first and seventh days of the study. Based on these observations, AN15368 was selected as a pre-clinical candidate for the treatment of Chagas disease and for evaluation in rhesus macaques (Macca mulatta) infected with T. cruzi via natural exposure in the U.S.

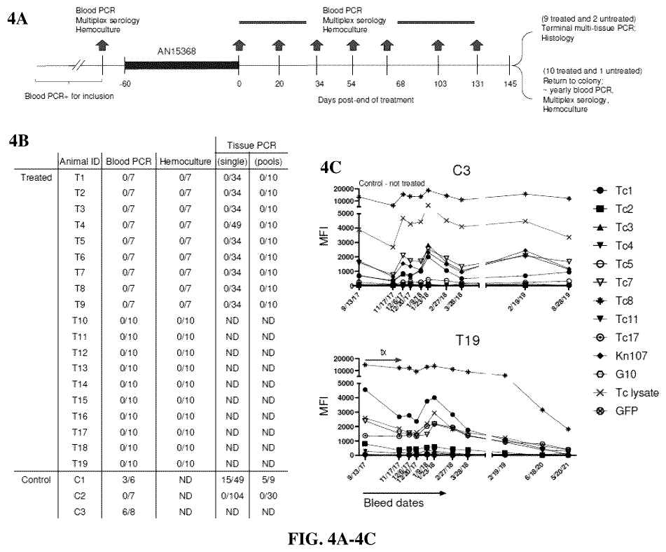

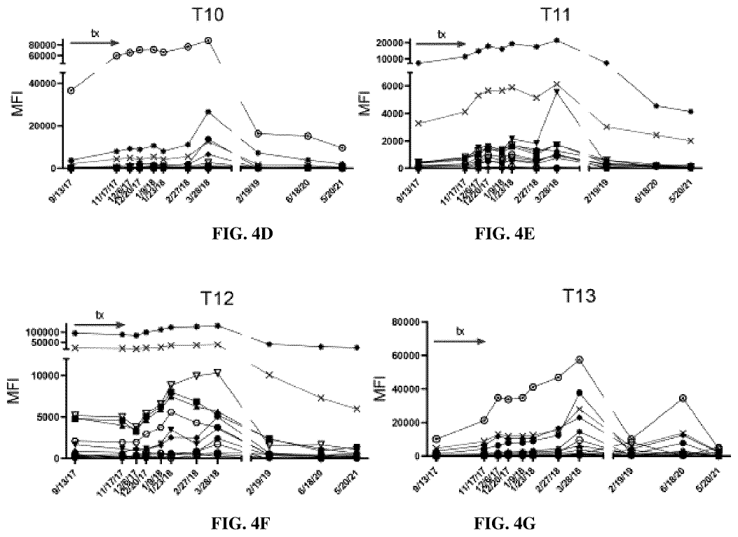

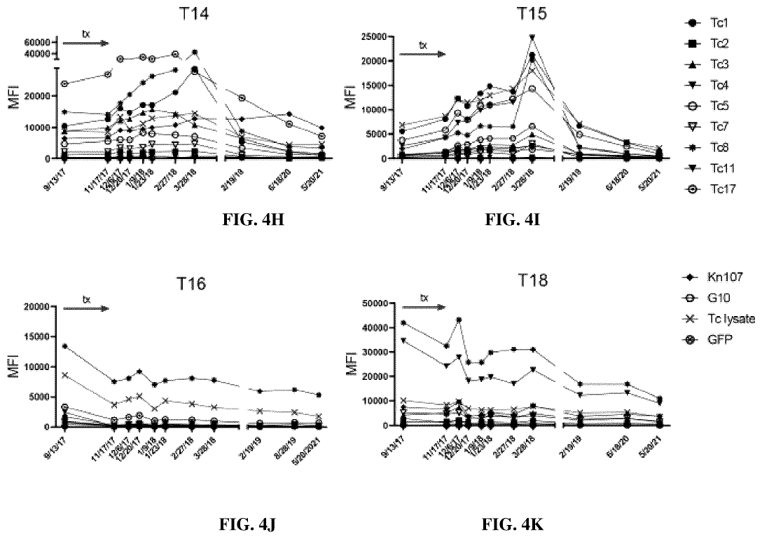

NHP were treated for 60 days as this is the standard length of treatment employed for human infections and in previous clinical trials (Torrico, et al., Lancet Infect Dis 18, 419-430 (2018), Molina, et al., The New England journal of medicine 370, 1899-1908 (2014)) (FIG. 4A). Based on pharmacokinetic studies in NHPs and allometric scaling (FDA Guidance for Industry for Estimating the Maximum Safe Starting Dose in Initial Clinical Trials for Therapeutics in Adult Healthy Volunteers (2005)) (see Methods for additional details) a dose of 30 mg/kg was chosen for NHP to provide high possible rate of cure without compromising safety. To maximize the power of detecting a high rate of cure, 19 animals were enrolled in the single arm treatment study and 3 animals served as untreated controls. Pre-treatment data on the animals is shown in Sup table 8; the mean age of the animals in the treatment group was 19.4 years and the average minimal length of infection was 5.7 yrs. All animals in the trial were positive by PCR for T. cruzi DNA in blood at one or more time points pre-treatment and were seropositive for anti-T. cruzi antibodies by standard facility screening tests and via a Luminex-based multiplex serological assay (Cooley, et al., PLoS neglected tropical diseases 2, e316 (2008), Padilla, et al., PLoS neglected tropical diseases 15, e0009141 (2021)) (Table 5, and FIGS. 4A-4K). Parasites were also isolated from hemocultures of pre-treatment blood samples from 18 of the 19 animals in the treatment group and 2 of 3 control animals. Importantly, the isolated parasite lines showed a variability in genetic types (DTU), indicating a diversity of parasite lineages in these animals, despite the fact they acquire infection in the relatively restricted geographic footprint of the primate colony (Table 5). These latter characteristics are similar to those found in a previous U.S.-based study in cynomolgus macaques with naturally-acquired T. cruzi infection (Padilla, et al., PLoS neglected tropical diseases 15, e0009141 (2021)).

The primary endpoints of the trial were detection of parasite DNA in blood and culture of parasites from blood, for which all animals in the study were assayed a minimum of 7 times at 2-4.5-week intervals following the end of treatment (FIG. 4A). All AN15368-treated animals were negative by both assays at all time points (FIG. 4B). In contrast, 2 of the 3 untreated animals were positive by one or both hemoculture and PCR at multiple time points.

A secondary determinant of treatment efficacy was the detection of T. cruzi DNA by PCR in post-necropsy tissues. For this purpose, 9 of the 19 treated animals and 2 of the untreated controls were euthanized and tissues harvested. DNA was extracted and analyzed by PCR for T. cruzi kDNA using both individual and pooled tissue samples (FIGS. 4A and 4B). For control animal C1, 30 of 68 (39.4%) of the single or pooled tissues samples from multiple tissues yielded positive PCR results. In contrast, for the 9 treated animals, tissue samples assayed singly or in pools (range 84-99 total sample sites/animal) from heart, quadriceps, biceps, small and large intestine, esophagus, tongue, liver, spleen, abdominal fat and brain all were negative. Interestingly, parasite DNA was note detected in non-treated control animal C2 by blood or tissue PCR despite it being positive by blood PCR and hemoculture, in sampling done pre-treatment.

A third measure of treatment efficacy was declining antibody levels to a set of recombinant T. cruzi proteins in the multiplex serological assay (Cooley, et al., PLoS neglected tropical diseases 2, e316 (2008), Padilla, et al., PLoS neglected tropical diseases 15, e0009141 (2021)). Monitoring for decreases in anti-T. cruzi antibodies has been useful for assessing treatment efficacy in humans and conversion to seronegative is considered the standard for determining infection cure—but in many cases can take years post-treatment to achieve (Albareda, et al., Journal of immunology 183, 4103-4108 (2009), Olivera, et al., Microbes Infect 12, 359-363 (2010), Viotti, et al., PLoS neglected tropical diseases 5, e1314 (2011)). Nine of the remaining 10 treated macaques not terminated at the end of treatment were returned to the breeding colony and thus were available for continued periodic monitoring over >3 years (FIG. 4A). All animals showed declines in antibody levels to multiple recombinant proteins from T. cruzi over the 42 months after the end of treatment (FIGS. 4C-4K). Importantly, blood PCR and hemoculture samples at these additional post-treatment time points also remained negative in all treated macaques (Table 5). Thus, the exhaustive examination of blood and tissues for parasite DNA and long-term monitoring of anti-T. cruzi antibodies conclusively establish a 100% efficacy of AN15368 in a population of time-variable, naturally infected NHPs harboring genetically diverse populations of T. cruzi.

Throughout the dosing period, macaques readily accepted food treats containing compound and no post-dose nausea or other interruption of normal activity was observed. No adverse events were noted in any of the 19 treated macaques during the 60 day treatment period and repeated physical examinations revealed no clinical signs that could be associated with drug administration. Per the blood-based health screening performed throughout the study, the mean values of the liver enzymes alanine aminotransferase (ALT) and alkaline phosphatase (ALP) and the levels of lymphocytes and monocytes in the blood were mildly elevated during the drug-treatment phase of the study and returned to pre-study values by the final blood draw at the end of the study. The 9 female animals that returned to the breeding colony have shown no abnormalities in the yearly examinations and have produced 13 healthy and T. cruzi-seronegative infants, in the first two years following treatment. These latter numbers are wholly consistent with the fecundity rate of the colony in general. At necropsy, 3 of 11 euthanized animals (2 treated animals and 1 control) were identified on gross examination to have pale areas in the heart that could be consistent with myocardial damage associated with Chagas disease. Histological examination revealed inflammation in several cases but did not differentiate the treated from untreated (control) study animals and no T. cruzi amastigotes were detected in any tissues from the study animals. Thus, AN15368 is both highly effective in curing long-standing T. cruzi infections and presents no overt safety or reproductive health concerns in a 60 day course of treatment in NHPs.

Target Identification.

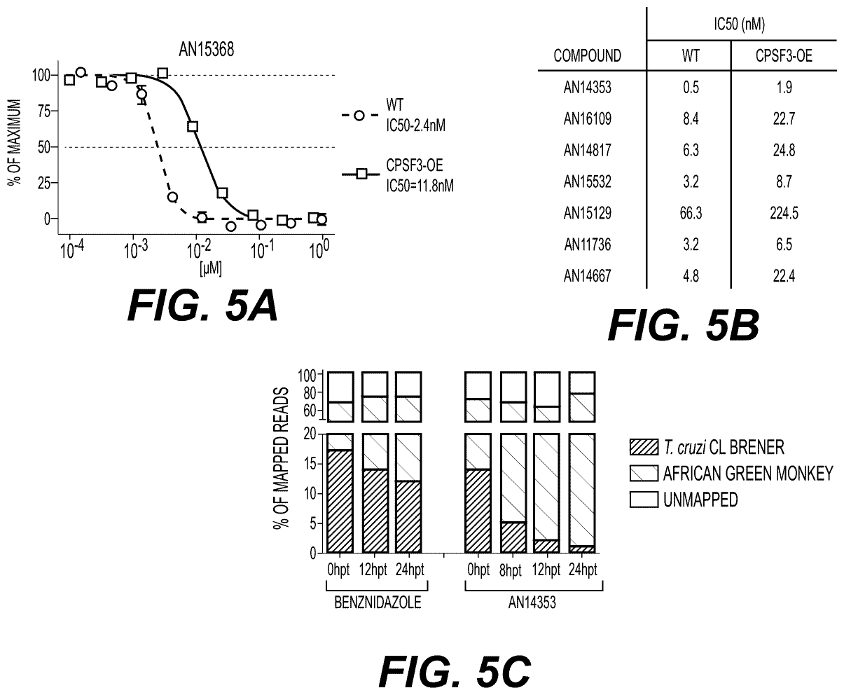

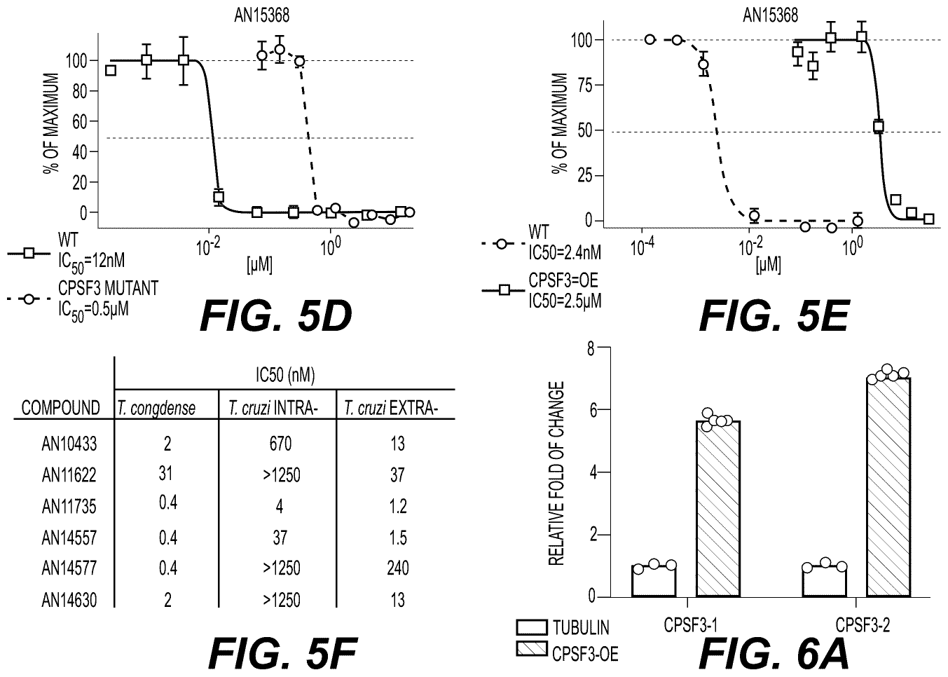

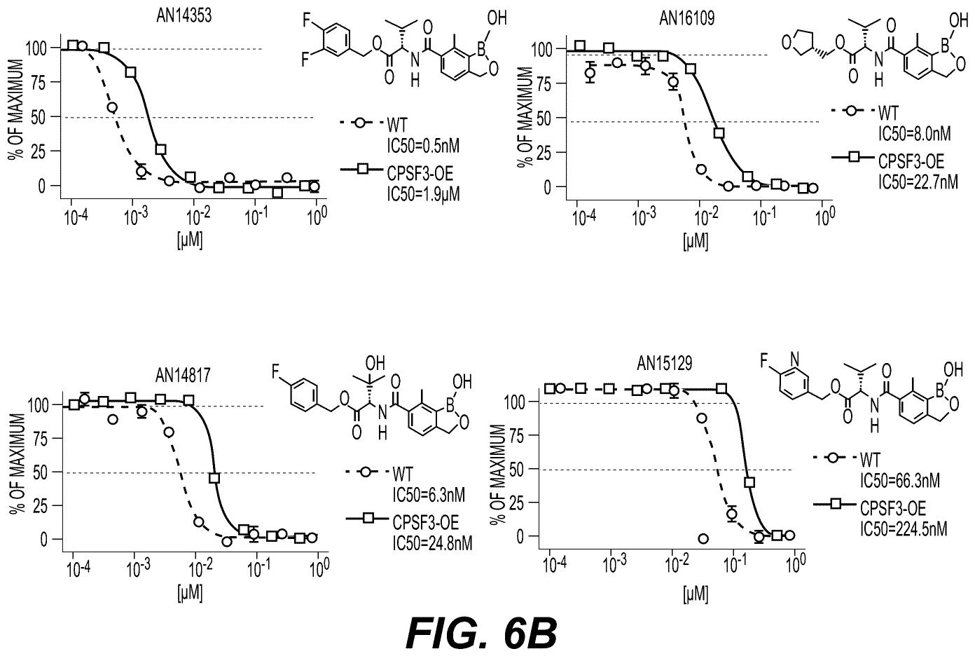

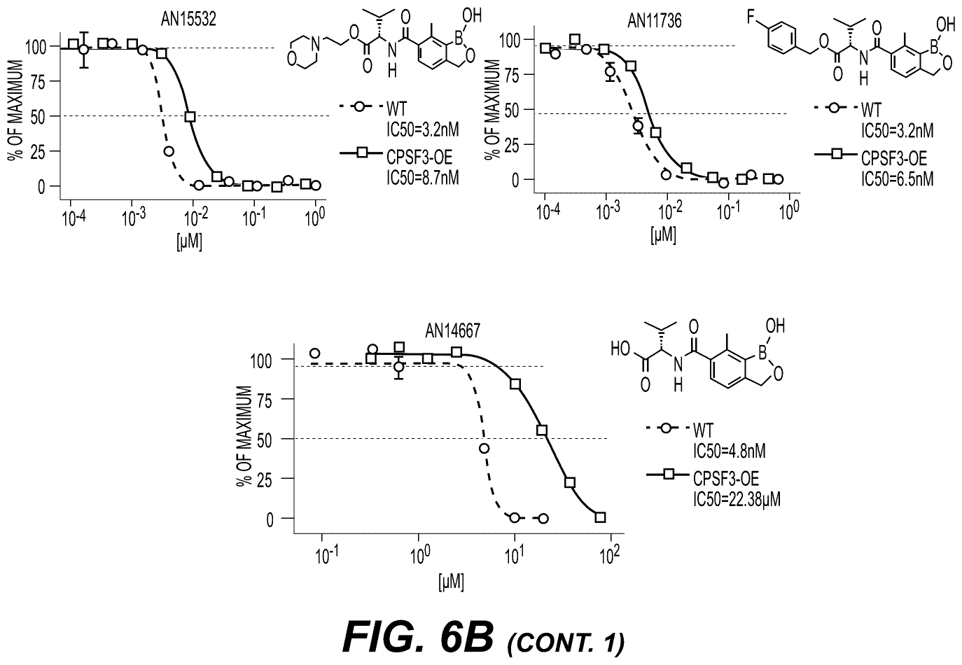

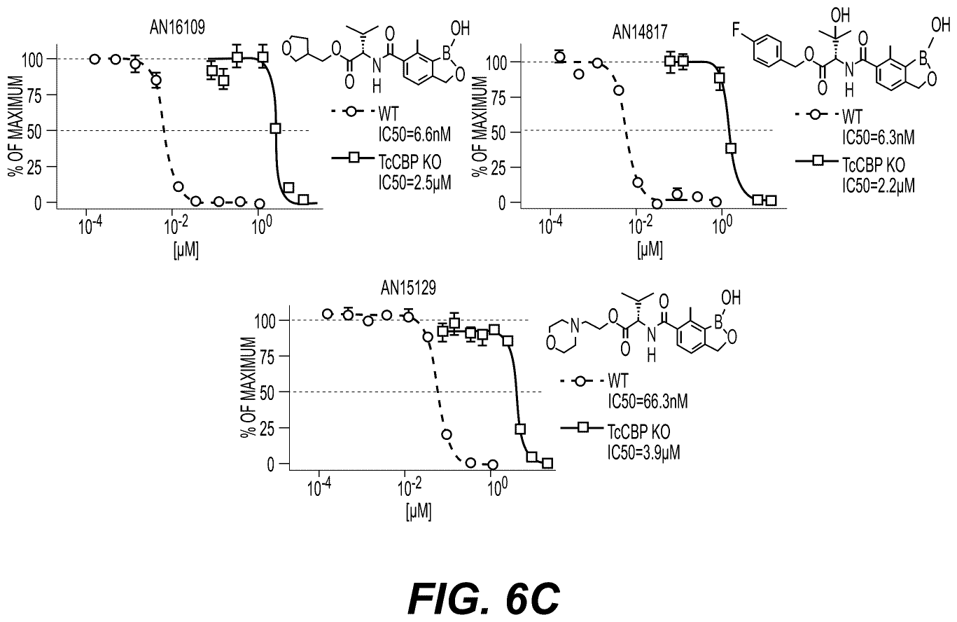

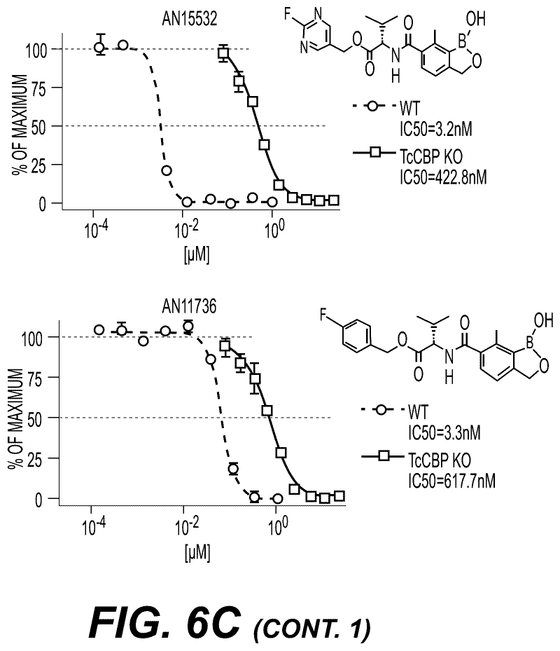

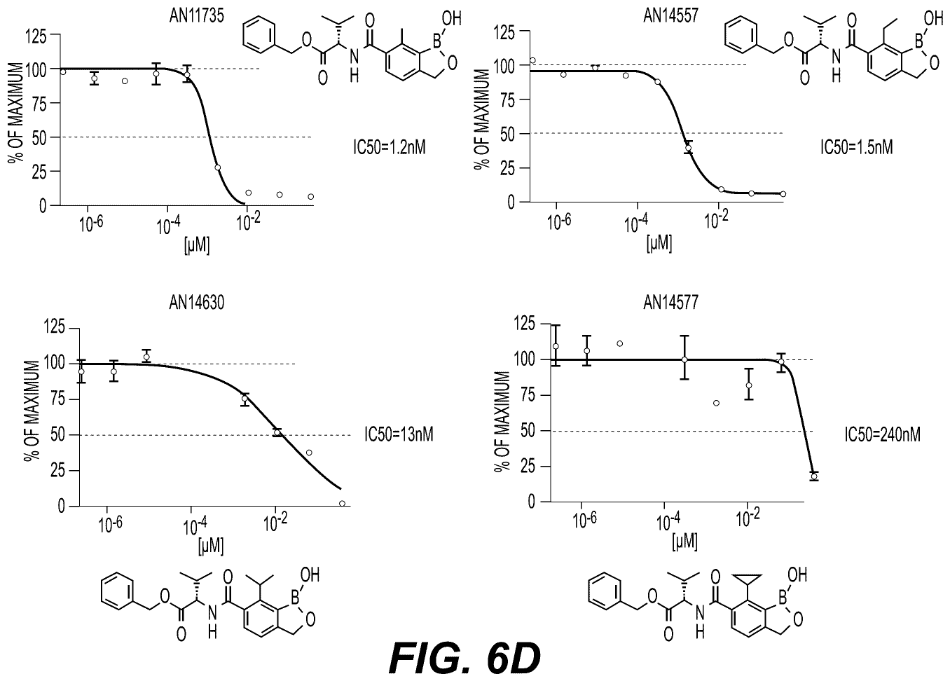

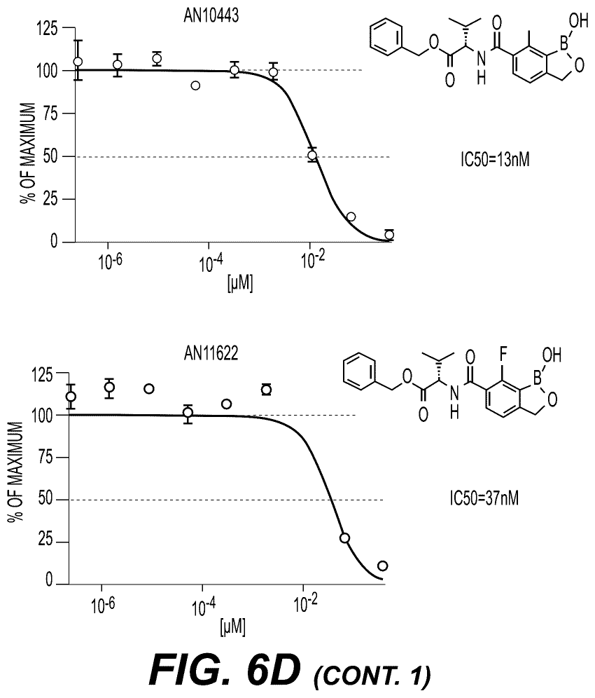

During the course of this work, several benzoxaborole analogues of AN15368 with efficacy in the treatment of African trypanosomiasis in cattle were shown to target the Cleavage and Polyadenylation Specificity Factor (CPSF3), an important factor in mRNA processing (Wall, et al., Proceedings of the National Academy of Sciences of the United States of America 115, 9616-9621 (2018), Begolo, et al., PLoS pathogens 14, e1007315 (2018)). Similar to as reported in these studies, the overexpression of CPSF3 in T. cruzi (FIGS. 6A-6D) also resulted in a 3-5 fold increase in resistance to AN15368 (FIGS. 5A-5F) as well as cross resistance to other benzoxaborole analogues (FIG. 5B and FIG. 6A-6D), indicating CPSF3 as at least one of the targets of AN15368 in T. cruzi. This conclusion is also supported by the marked and continuing reduction in parasite mRNA as early as 6 hrs following addition of AN14353 to T. cruzi-infected host cells, but not in benznidazole-treated cultures (FIG. 5C). Furthermore, introduction into T. cruzi of the Asn232 mutation to His in CPSF3, reported by Wall, et al (Wall, et al., Proceedings of the National Academy of Sciences of the United States of America 115, 9616-9621 (2018)) to disrupt binding of benzoxaborole compounds in T. brucei, also conferred resistance to AN15368 (FIG. 5D). Lastly, and as noted above for AN14353 (FIG. 2A-2D), AN15368 and other related analogues all function as prodrugs and require activation by the T. cruzi CBP in order to efficiently kill intracellular T. cruzi (FIG. 5E and FIG. 6C), as is the case for the benzoxaboroles effective against African trypanosomes (Giordani, et al., PLoS pathogens 16, e1008932 (2020)). Thus, the highly effective benzoxaborole AN15368 is a prodrug that enters host cells and then T. cruzi, wherein it is cleaved by a T. cruzi peptidase. The product of this cleavage selectively targets CPSF3-mediated mRNA maturation in intracellular amastigotes. Although the target of these benzoxaboroles in T. cruzi and in African trypanosomes appear to be the same, a number of the compounds with previously reported potent activity in vitro to T. congolense, had activity on extracellular amastigotes but not on T. cruzi amastigotes in host cells (FIG. 5E and FIG. 6D). Reasoning that this differential effect could be due to variable or limited entry into or metabolism by T. cruzi host cells, activity of these compounds was screened on extracellular amastigotes of T. cruzi. With one possible exception, all of these compounds have low nanomolar activity on extracellular amastigotes, indicating an additional selectivity of benzoxaborole activity on T. cruzi due to this parasite's intracellular lifestyle.

TABLE 5

| | | | | Years | | | | | | | | | | | | | | |
|---|---|---|---|---|---|---|---|---|---|---|---|---|---|---|---|---|---|---|
| | | | | Sero- | Pre- | | | | | | | | | | | | | |
| | | | Year | positive | trest | Pre-treat | | | | | | | | | | | | |
| | | Age | Sero- | at study | blood | hemo- | T. cruzi | days post treatment (blood PCR/hemoculture) | | | | | | | | | | |
| Treatment | Male | (yr) | positive | start | PCR | culture | genotype | 0 | 20 | 34 | 54 | 68 | 103 | 131 | 145 | 460 | 945 | 1281 |
| T1 | − | 20 | 2012 | 6 | + | + | TCI | −/− | −/− | −/− | −/− | −/− | −/− | −/− | euth/Hem− | | | |
| T2 | − | 20 | 2014 | 4 | + | + | TCI | −/− | −/− | −/− | −/− | −/− | −/− | −/− | euth/Hem− | | | |
| T3 | − | 21 | 2013 | 5 | + | + | TCIV | −/− | −/− | −/− | −/− | −/− | −/− | −/− | euth/Hem− | | | |
| T4 | 1 | 19 | 2011 | 7 | + | + | TCI | −/− | −/− | −/− | −/− | −/− | −/− | −/− | euth/Hem− | | | |
| T5 | − | 19 | 2010 | 8 | + | + | TCI | −/− | −/− | −/− | −/− | −/− | −/− | −/− | euth/Hem− | | | |
| T6 | − | 16 | 2014 | 4 | + | + | TCI | −/− | −/− | −/− | −/− | −/− | −/− | −/− | euth/Hem− | | | |
| T7 | − | 22 | 2013 | 5 | + | + | TCI | −/− | −/− | −/− | −/− | −/− | −/− | −/− | euth/Hem− | | | |
| T8 | − | 19 | 2013 | 5 | + | − | | −/− | −/− | −/− | −/− | −/− | −/− | −/− | euth/Hem− | | | |
| T9 | − | 19 | 2008 | 11 | + | + | TCIV | −/− | −/− | −/− | −/− | −/− | −/− | −/− | | −/− | −/− | −/− |
| T10 | − | 5 | 2016 | 2 | + | + | TCIV | −/− | −/− | −/− | −/− | −/− | −/− | −/− | | −/− | −/− | −/− |
| T11 | − | 12 | 3014 | 4 | + | + | TCIV | −/− | −/− | −/− | −/− | −/− | −/− | −/− | | −/− | −/− | −/− |
| T12 | − | 11 | 2015 | 3 | + | + | TCI | −/− | −/− | −/− | −/− | −/− | −/− | −/− | | −/− | −/− | −/− |
| T13 | − | 11 | 2015 | 3 | + | + | TCI | −/− | −/− | −/− | −/− | −/− | −/− | −/− | | −/− | −/− | −/− |
| T14 | − | 9 | 2014 | 4 | + | + | TCI | −/− | −/− | −/− | −/− | −/− | −/− | −/− | | −/− | −/− | −/− |
| T15 | − | 16 | 2011 | 7 | + | + | TCTV | −/− | −/− | −/− | −/− | −/− | −/− | −/− | | −/− | −/− | −/− |
| T16 | − | 20 | 2010 | 8 | + | + | TCTV | −/− | −/− | −/− | −/− | −/− | −/− | −/− | | −/− | −/− | −/− |
| T17 | − | 21 | 2010 | 8 | + | + | TCIV | −/− | −/− | −/− | −/− | −/− | −/− | −/− | LTF | | | |
| T18 | − | 22 | 2010 | 8 | + | + | TCI | −/− | −/− | −/− | −/− | −/− | −/− | −/− | | −/− | −/− | −/− |
| T19 | − | 23 | 2012 | 6 | + | + | TCI | −/− | −/− | −/− | −/− | −/− | −/− | −/− | | −/− | −/− | −/− |
| Totals/ | 1 | 19.4 | | 5.7 | 19 | | | | | | | | | | | | | |
| Means | | | | | | | | | | | | | | | | | | |
| untreated | | Age | Year | | | | | | | | | | | | | | | |
| controls | Male | (yr) | positive | | | | | | | | | | | | | | | |
| C1 | − | 3 | 2017 | | + | + | TCIV | −/− | +/+ | +/+ | −/+ | +/+ | +/− | −/− | euth/Hem+ | | | |
| C2 | − | 22 | 2012 | | + | + | TCIV | −/− | −/− | −/− | −/− | −/− | −/− | −/− | euth/Hem− | | | |
| C3 | 1 | 5 | 2016 | | + | − | | +/− | +/− | +/− | +/− | −/− | +/− | +/+ | | | | |
| Totals/ | 1 | 5 | | 3 | 3 | | | | | | | | | | | | | |
| Means | | | | | | | | | | | | | | | | | | |

### DISCUSSION

The benzoxaborale AN15368 is the first, highly effective compound for the treatment of T. cruzi infection discovered in >50 years, and the only compound to date shown to achieve unequivocal and apparently uniform cure of infection in NHP with long-term, naturally acquired infections of diverse T. cruzi genetic types. AN15368 is orally bioactive and exhibited no overt toxicity in a 60 day course of treatment in NHP. Thus, AN15368 is a very strong candidate for ultimately progressing into human clinical trials.

Despite some successful vector control efforts, risk of infection with T. cruzi remains significant for human and other animals from the southern U.S. to southern South American. The currently available drugs benznidazole and nifurtimox suffer from variable efficacy and high rates of adverse events. Consequently, these drugs are not routinely used despite their relatively wide availability. The absence of highly effective treatments undermines the use of widespread and routine screening that would detect the usually asymptomatic early infection before irreversible damage is done (Tarleton, (Trends in molecular medicine (2016)). A relatively large number of potential candidates have been targeted for development, some for decades (reviewed in Mazzeti, et al., J Exp Pharmacol 13, 409-432 (2021))), but those that have been progressed to human clinical trials (Torrico, et al., Lancet Infect Dis 18, 419-430 (2018), Molina, et al., The New England journal of medicine 370, 1899-1908 (2014), Morillo, Journal of the American College of Cardiology 69, 939-947 (2017)) have performed significantly worse than currently available drugs.

The benzoxaboroles have become a rich source of development candidates for treatment of protozoal infections, with an apparent target of all being the mRNA-processing endonuclease, CPSF3 (Akama, et al., Bioorg Med Chem Lett 28, 6-10 (2018), Wall, et al., Proceedings of the National Academy of Sciences of the United States of America 115, 9616-9621 (2018), Begolo, et al., PLoS pathogens 14, e1007315 (2018), Giordani, et al., PLoS pathogens 16, e1008932 (2020), Sonoiki, et al., Nature communications 8, 14574 (2017), Palencia, et al., EMBO Mol Med 9, 385-394 (2017), Swale, et al., Sci Transl Med 11 (2019), Bellini, et al., iScience 23, 101871 (2020), Van den Kerkhof, et al., Microorganisms 9 (2021). Although it was initially hypothesized that AN15368 and the analogue AN11736 may not target CPSF3, based upon their limited effect on mRNA processing (Begolo, et al., PLoS pathogens 14, e1007315 (2018)), subsequent work demonstrated that overexpression of CPSF3 in T. b. brucei induced resistance to killing by AN11736 (Wall, et al., Proceedings of the National Academy of Sciences of the United States of America 115, 9616-9621 (2018)) and a similar conclusion with respect to the activity of AN15368 in T. cruzi, indicating CPSF3 as one likely target is arrived at here. Also, like a number of benzoxaboroles highly effective versus the African trypanosomes, AN15368 requires processing into its carboxylate form in order to achieve full potency and this activation is shown to be mediated by a parasite serine carboxypeptidase. However, unlike the case in the extracellular African trypanosomes, AN15368 must traffic unprocessed through both the host and the parasite plasma membranes in order to reach these activating enzymes. This requirement appears to account for the differential activity of a number of highly similar benzoxaboroles on African trypanosomes and T. cruzi and emphasizes the need to tailor drugs to match the specific biology of the pathogen, even when the processing/activation requirements and the target of the compounds are the same. Likewise, this outcome highlights the challenge of designing compounds with cross-species activity for genetically-related but biologically-diverse pathogens like the kinetoplastids.

The differential dosing requirements for the benzoxaboroles in T. cruzi infection, where 20 or more days of treatment is necessary for sterile cure, as compared to African trypanosomes—where a single dose effects cure in cattle (Akama, et al., Bioorg Med Chem Lett 28, 6-10 (2018))—is remarkable and further underscores the difficulties of drug discovery for T. cruzi. T. cruzi can invade diverse host cells types in tissues throughout the body, presenting a challenge for any one drug to reach effective levels in all tissues. Furthermore, T. cruzi amastigotes have recently been shown to assume a non-dividing, apparently low-metabolic state that provides substantial resistance to drugs (Sanchez-Valdez, et al., eLife 7 (2018), Bustamante, et al., Sci Transl Med 12 (2020)). Fortunately, these properties do not prevent drug-induced sterile cure, but appear to make necessary an extended treatment course, as observed herein, and previously for other anti-T. cruzi drugs (Bustamante, et al., Sci Transl Med 12 (2020)). The NHP trial conducted as part of this study was initiated before knowledge of this dormancy property in T. cruzi and thus utilized daily dosing for 60 days, as is common for the currently used benznidazole and nifurtimox. Despite these lengthy treatment periods, drug-induced resistance in T. cruzi has not been reported with respect to the benznidazole and nifurtimox nor were resistance during the extended treatment courses using benzoxaboroles observed, despite the fact that all three drugs are produgs. Nevertheless, shortened or modified treatment regimens may be possible with the benzoxaboroles (FIG. 3C) as is the case for benznidazole (Bustamante, et al., The Journal of infectious diseases 209, 150-162 (2014), Bustamante, et al., Sci Transl Med 12 (2020), Alvarez, et al., Antimicrobial agents and chemotherapy 64 (2020)), to further reduce this possibility. As a prodrug, AN15368 has the liability of potential selection of resistance via loss of the processing carboxypeptidase (Giordani, et al., PLoS pathogens 16, e1008932 (2020)), although deletion of the CBP array in T. cruzi substantially reduces, but does not totally abolish, susceptibility to AN15368.

One significant advantage of drug discovery in T. cruzi is the very wide natural host range of the parasite, including most wild and domesticated mammals as well as mice, canines, and NHP, in addition to humans. In all these hosts, T. cruzi appears to behave similarly, infecting the same host cell types, being controlled (but rarely eliminated) by similar immune effector mechanisms, generating analogous pathologies, and being affected correspondingly by the same drugs. Animals in T. cruzi endemic areas, including the southern U.S., are at risk of acquiring T. cruzi infection and in some areas, this risk is severe, leading to 20-30%/yr new infection in some populations (Busselman, et al., (bioRxiv, 2021.2006.2024.449798 (2021)) as well as infections in zoo animals (Huckins, et al., J Vet Diagn Invest 31, 752-755 (2019), Minuzzi-Souza, et al., Parasit Vectors 9, 39 (2016)). The commonly used indoor/outdoor housing of NHPs in T. cruzi-endemic regions also results in naturally acquired T. cruzi infection in animals in these facilities, despite vector control and other preventative measures (Hodo, et al., Ecohealth 15, 426-436 (2018)). Infections in these NHPs mirror that in humans, initiating at different points in life and extending for decades in some cases, involving genetically and phenotypically diverse parasite populations, and leading to a diversity of immune responses and disease outcomes (Padilla, et al., PLoS neglected tropical diseases 15, e0009141 (2021)). All these characteristics make these NHPs incredibly valuable resources for trialing anti-T. cruzi drugs prior to human clinical trials. The observed 100% cure with AN15368 in macaques harboring long-term infections with genetically diverse parasite lineages and without any apparent drug toxicity, bodes well for the potential safety and efficacy of AN15368 in humans. It is noteworthy that the only other documented treatment trials in T. cruzi-infected NHPs recorded a high degree of failures for posaconazole (100%) (Cox, et al., ILAR J 58, 235-250 (2017)) and benznidazole (>60%), in agreement with the high failure rate of these drugs in human clinical trials (Torrico, et al., Lancet Infect Dis 18, 419-430 (2018), Molina, et al., The New England journal of medicine 370, 1899-1908 (2014), Morillo, et al., Journal of the American College of Cardiology 69, 939-947 (2017)). The same methods used for monitoring treatment efficacy in humans were validated, specifically serial blood PCR for T. cruzi DNA and changes in immune profiles, for use in NHPs, and reinforce these metrics with extensive tissue PCR in a subset of necropsied animals. Lastly, the ability to return infection-cured macaques to the breeding colony extends the utility of this resources, providing the potential to monitor disease development and the susceptibility to reinfection, for example, in hosts previously cured of T. cruzi infections.

One apparent self-cure among the three untreated NHPs was observed. Although documentation of spontaneous cures are relatively rare (Francolino, et al., Revista da Sociedade Brasileira de Medicina Tropical 36, 103-107 (2003), Pinto Dias, et al., Revista da Sociedade Brasileira de Medicina Tropical 41, 505-506 (2008), Tarleton, Revista Española de Salud Pdblica 87, 33-39 (2013)) they are not without precedent and may occur more frequently than currently appreciated (Buss, et al., PLoS neglected tropical diseases 14, e0008787 (2020)) but have not been previously observed in this NHP colony.

The success of this project provides insights into some best practices for drug discovery in T. cruzi and perhaps related organisms. In addition to taking advantage of the potency of the oxaboroles in general as anti-infectives, and the med-chem knowledgebase in this class of compounds, this project also benefited substantially from the accessibility of infection systems for screening and testing compounds. Specifically, the power of the mouse-based assays to quickly, easily and quantitatively assess in vitro-active compounds for in vivo activity was instrumental in rapidly identifying the compounds with the highest potential. The availability of substantial numbers of NHPs coupled with naturally acquired T. cruzi infections for pre-clinical validation, should help avoid the clinical trial failures that have accompanied previous drug discovery efforts in Chagas Disease.

### Example 3: Prophylactic Low-Dose, Bi-Weekly Benznidazole Treatment Fails to Prevent Trypanosoma cruzi Infection in Dogs Under Intense Transmission Pressure

Bustamante, et al., PLoS Negl Trop Dis. 2022 Oct. 31; 16(10):e0010688. doi: 10.1371/journal.pntd.0010688. PMID: 36315597; PMCID: PMC9648846, is specifically incorporated by reference herein in its entirety, including all supplemental materials associated therewith.

Introduction

Chagas disease, caused by the protozoan parasite Trypanosoma cruzi, is a problem for human and animal health across the Americas where triatomine vectors are endemic. T. cruzi is predominantly transmitted in the feces of infected triatomines through contact with wounds or mucous membranes or ingestion of infected insects or fecal material (Ber, et al., Clin Microbiol Rev. 24(4):655-81 (2011)). Oral transmission is thought to be the most important route in domestic dogs and is a highly efficient mode of transmission (Roellig, et al., The Journal of parasitology 95(2):360-4 (2009), Barr, Vet Clin North Am Small Anim Pract 39(6):1055-64 (2009), Velasquez-Ortiz & Ramirez (Res Vet Sci. 132:448-61(2020), Montenegro, et al., Memorias do Instituto Oswaldo Cruz 97:491-4 (2002)).

Dogs are important to study in the context of Chagas disease for at least three reasons: i) they experience similar disease progression to humans, so are useful to study as a model for human disease especially in considering treatments; ii) they share exposure to vectors with humans in domestic and peridomestic environments and therefore may serve as sentinels for human health risk Gurtler & Cardinal, Acta tropica (2015), Estrada-Franco, et al., Emerging infectious diseases 12(4):624-30 (2006), Jaimes-Duenez, et al., Prev Vet Med. 141:1-6 (2017); and iii) canine Chagas disease is an increasingly recognized problem in veterinary medicine, especially in the southern U.S. Busselman, et al., Annu Rev Anim Biosci. 10:325-48 (2022), leading to dog mortality (Meyers, et al., Vet Parasitol Reg Stud Reports. 24:100545 (2021)). Large multi-dog kennels in central and south Texas have emerged as settings with particularly high transmission risk, with a recent study documenting incidence of over 30 infections per 100 dogs per year (Busselman, et al., PLoS neglected tropical diseases 15(11):e0009935 (2021)), despite variable vector control efforts in and around the kennels. Although some therapeutic regimens have shown promise in reducing disease impact in dogs infected with T. cruzi (Madigan, et al., Journal of the American Veterinary Medical Association 255(3):317-29 (2019)), no therapeutics or vaccines have been evaluated as potential preventatives of T. cruzi infection in dogs.

In the Example, an approach for preventing new infections in dogs within multi-dog kennel environments with a history of triatomine occurrence and canine Chagas disease was tested. Disappointingly, prophylaxis during the period of peak adult vector activity (Curtis-Robles, et al., PLoS neglected tropical diseases 9(12):e0004235 (2015)) with benznidazole (BNZ), an FDA-approved drug used in the treatment of human T. cruzi infection, had no impact on the incidence of new infections in this setting, However the long-term impact of prophylaxis on clinical disease was not monitored. Further, this study design provides a model for future evaluation of different prophylactic regimens (higher dose; treatments earlier in the season) in high transmission settings.

Material and Methods

Study Design

For the studies in mice, female mice, 8-12 weeks old were used for infections throughout the study. Sample sizes were determined based upon knowledge of heterogeneity in parasite burden during T. cruzi infection and published reports using similar experimental strategies. Data collected were included if productive T. cruzi infection was established (visualized by luciferase imaging or flow cytometry by detection of T. cruzi-tetramer specific CD8⁺ T cells). The investigators were not blinded during the collection or analysis of data and mice were randomly assigned to treatment groups prior to the start of each experiment.

For studies in dogs, a small network of kennels was formed in central and south Texas with a history of triatomine vector occurrence and canine Chagas disease with owners who were willing to participate in the study. Kennels are identified through (i) canine patients with Chagas disease presenting to the cardiology unit at the TAMU VMTH and (ii) the TAMU Kissing Bug Community Science program, in which many dog owners submit triatomines collected from large kennel environments. At these large kennels, dogs are primarily bred and trained to aid hunting parties. Approximately 40-80 dogs reside within each kennel, and the predominant breeds include Belgian Malinois, Brittany spaniels, English pointers, German shorthaired pointers, Labrador retrievers, and hound dogs. Dogs >3 months of age, including males and females, were eligible for enrollment. Dogs identified after blood screening as seronegative based on multiplex serology and PCR-negative were randomly assigned to treatment groups. Neither the investigators, private veterinarian providing care to the dogs in the field, nor the dog owners were blinded to the treatment groups and no placebo was given to control dogs. Informed consent was obtained from dog owners prior to their participation, and this study was approved by the Texas A&M University Institutional Committee on Animal Use and Care and the Clinical Research Review Committee.

Mice, Parasites and Infections

C57BL/6J (Stock No:000664) mice (C57BL/6 wild-type) were purchased from The Jackson Laboratory (Bar Harbor, ME) and C57BL/6J-IFN-gamma knockout mice (also known as B6.129S7-Ifngtm1 Ts/J; The Jackson Laboratory stock No 002287) were bred in-house at the University of Georgia Animal Facility. All the animals were maintained in the University of Georgia Animal Facility under specific pathogen-free conditions. T. cruzi tissue culture trypomastigotes of the Colombiana strain co-expressing firefly luciferase and tdTomato reporter proteins, generated as described previously (Sanchez-Valdez, et al., eLife 7 (2018), Bustamante, et al., Sci Transl Med. 12(567) 2020)), were maintained through passage in Vero cells (American Type Culture Collection (Manassas, VA)) cultured in RPMI 1640 medium with 10% fetal bovine serum at 37° C. in an atmosphere of 5% CO₂. Mice were infected intraperitoneally with 10³ tissue culture trypomastigotes of T. cruzi. This study was carried out in strict accordance with the Public Health Service Policy on Humane Care and Use of Laboratory Animals and Association for Assessment and Accreditation of Laboratory Animal Care accreditation guidelines. The protocol was approved by the University of Georgia Institutional Animal Care and Use Committee.

Drug Treatment and In Vivo Imaging

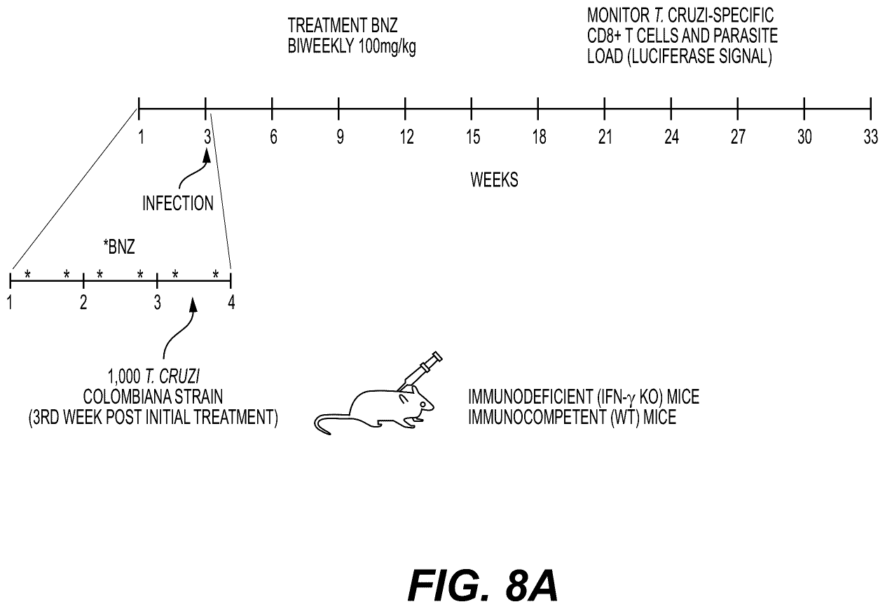

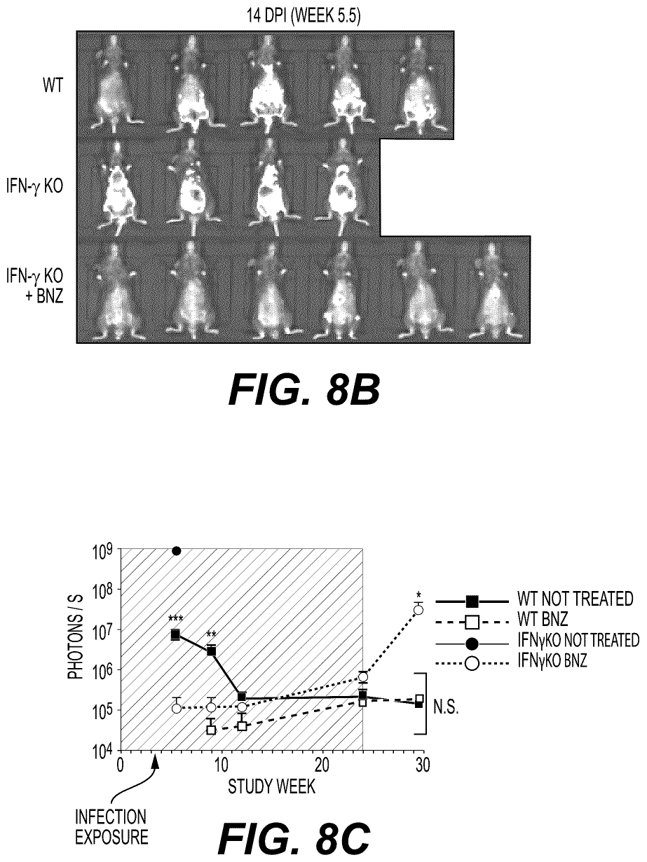

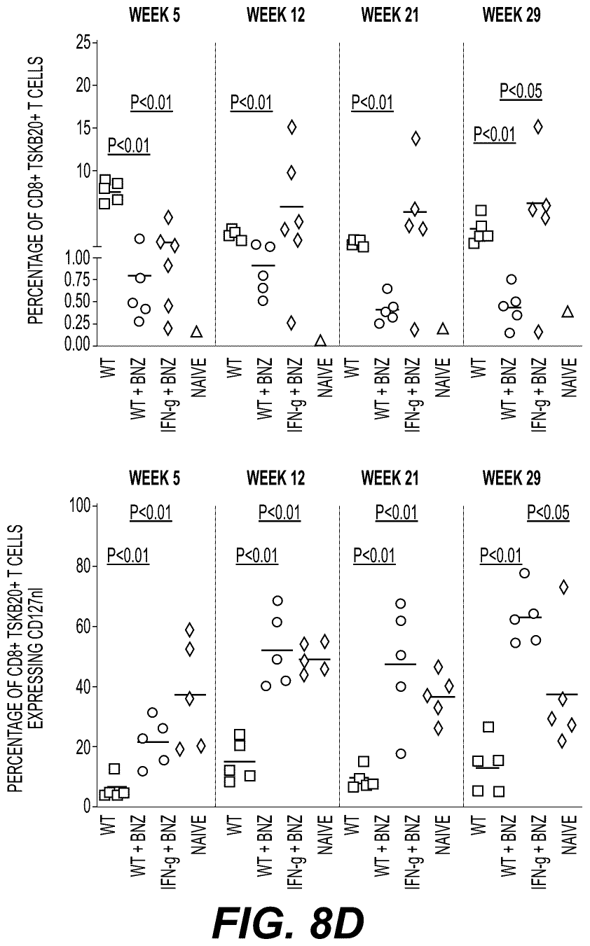

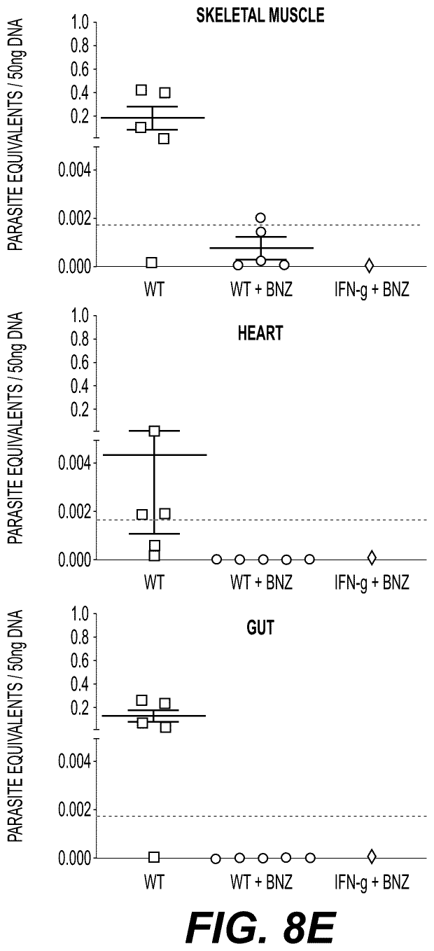

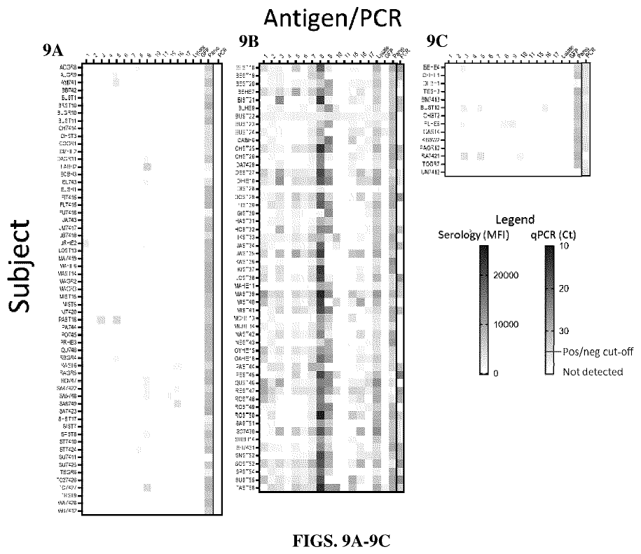

Mice were treated with benznidazole twice weekly at a 100 mg/kg/day and infected midway during the 3^{rd} week of treatment (FIG. 8A). Benznidazole (BNZ—Elea Phoenix, Buenos Aires, Argentina) was prepared by pulverization of tablets followed by suspension in an aqueous solution of 1% sodium carboxymethylcellulose with 0.1% Tween 80 and delivered orally by gavage at a concentration dosage of 100 mg/kg of body weight. Each mouse received 0.2 ml of this suspension. Luciferase-expressing parasites were quantified in mice by bioluminescent detection. Mice were injected intraperitoneally with D-luciferin (150 mg/kg; PerkinElmer, Waltham, MA) and anesthetized using 2.5% (vol/vol) gaseous isofluorane in oxygen prior to imaging on an IVIS Lumina II imager (Xenogen, Alameda, CA) for an exposure time of 5 minutes, as previously described (Canavaci, et al., PLoS neglected tropical diseases 4(7):e740 (2010)). Quantification of bioluminescence and data analysis was performed using Aura Imaging Software version 4.0.7 (Spectral Instruments Imaging, Tucson, AZ).

T-Cell Phenotyping

Mouse peripheral blood was obtained, processed and analyzed as previously described (Bustamante, et al., Sci Transl Med. 12(567) (2020), Bustamante, et al., The Journal of infectious diseases 209(1):150-62 (2014)). Whole blood was incubated with a major histocompatibility complex I (MHC I) tetramer containing the T. cruzi transialidase TSKB20 peptide (ANYKFTLV (SEQ ID NO:124)/Kb) labeled with BV421 (Tetramer Core Facility at Emory University, Atlanta, GA) and the following labeled antibodies: anti-CD8 FITC, anti-CD4 APC EF780, anti-CD127 PE (BD Bioscience, San Jose, CA). At least 500,000 cells were acquired using a CyAn ADP flow cytometer (Beckman Coulter, Hialeah, Florida) and analyzed with FlowJo software v10.6.1 (Treestar, Inc., Ashland, OR).

Quantitative Polymerase Chain Reaction

For determination of tissue parasite load in mice, samples of skeletal muscle, heart and intestine were collected at necropsy and processed for quantification of T. cruzi DNA by real-time polymerase chain reaction (qPCR) as previously described (Bustamante, et al., Sci Transl Med. 12(567) (2020), Bustamante, et al., The Journal of infectious diseases 209(1):150-62 (2014), Cummings & Tarleton, Molecular and biochemical parasitology 129(1):53-9 (2003)). The limit of detection was set at the lowest standard of 0.0017 parasite equivalents per 50 ng of DNA. For detection of T. cruzi infection in dogs, DNA was extracted from 250 uL of the buffy coat fraction of EDTA-treated blood using the E.Z.N.A Tissue DNA kit (Omega Bio-Tek, Norcross, GA, USA) according to the manufacturer's protocol except 50 μL of elution buffer was used. T. cruzi-negative controls (phosphate buffered saline) were included in the DNA extractions. Samples were tested using qPCR for the presence of T. cruzi satellite DNA using the Cruzi 1/2 primer set and Cruzi 3 probe in a real-time assay, which amplifies a 166-bp segment of a repetitive nuclear DNA (Piron, et al., Acta tropica. 103(3):195-200 (2007), Cummings & Tarleton, Molecular and biochemical parasitology 129(1):53-9 (2003)) as previously described (Meyers, et al., PLoS neglected tropical diseases 11(8) 2017)). A sample was considered T. cruzi-positive if the Ct value was <35 (Curtis-Robles, et al., Vet Parasitol Reg Stud Reports 12:85-8 (2018)).

Multiplex Serology

Luminex-based multiplex serological assays were performed as previously described (Cooley, et al., PLoS neglected tropical diseases 2(10) (2008), Hartley, et al., Vet Res. 45:6 (2014)). For a number of smaller proteins, fusions of up to 2 individual genes are employed in order to expand the array of antibodies being detected. (TritrypDb.org identifiers: Antigen 1 (Tc1)=fusion of TcBrA4_0116860 and TcYC6_0028190; 2 (Tc2)=fusion of TcBrA4_0088420 and TcBrA4_0101960; 3 (Tc3)=fusion of TcBrA4_0104680 and TcBrA4_0101980; 4 (Tc4)=fusion of TcBrA4_0028480 and TcBrA4_0088260; 5 (Tc5)=fusion of TcYC6_0100010 and TcBrA4_0074300; 6 (Tc6)=fusion of TcYC6_0043560 and TcYC6_0122760; 7 (Tc7)=fusion of TcYC6_0083710 and TcBrA4_0130080; 8 (Tc8)=TcYC6_0037170; 9 (Tc11)=TcYC6_0124160; 10 (Tc17)=fusion of TcBrA4_0028230 and TcBrA4_0029760; 11 (Tc19)=TcBrA4_0122270 and TcBrA4_0131050; 12 (tc2−tol2)=TcBrA4_0101960; 13 (3tolt)=portions of TcBrA4_0101970, TcYC6_0077100 and TcYC6_0078140; 14 (beta-tubulin)=TcYC6_0010960; 15 (G10)=TcCLB.504199.20; 16 (Kn107)=TcCLB.508355.250; 17 (LE2)=TcCLB.507447.19; Parvo=Recombinant Canine Parvovirus VP2 (MyBioSource.com).

Cardiac Troponin

Cardiac troponin (cTnI) analysis was performed using the ADVIA Centaur CP® immunoassay (Ultra-TnI, Siemens Medical Solutions USA, Inc., Malvern, PA) validated in dogs and with a reported range of 0.006 to 50.0 ng/mL (Winter, et al., J Vet Cardiol. 16(2):81-9 (2014)).

Results

Studies show that weekly (Bustamante, et al., Sci Transl Med. 12(567) (2020)) or twice weekly administration of high dose (2.5-5× the normal daily dose) BNZ could cure established infections with T. cruzi in mice. To determine if BNZ might also prevent the initial establishment of T. cruzi in naïve mice, a pilot study (FIG. 8A) was conducted using twice weekly delivery of low dose BNZ (100 mg/kg oral, the standard daily dosage for continuous treatment in mice (Bustamante, et al., Nature medicine. 14(5):542-50 (2008)). At 3.5 weeks (between the 5^{th} and 6^{th} BNZ dose) all mice were injected i.p. with 1000 typomastigotes of the luciferase transgenic Colombiana strain of T. cruzi. Groups of both wild-type (C57BL/6J) and IFNγ deficient mice were included in the study; the IFNγ deficient (KO) mice are very highly susceptible to T. cruzi infection (Bustamante, et al., Sci Transl Med. 12(567) (2020), Marinho, et al., Scand J Immunol. 66(2-3):297-308 (2007)) and were expected to reveal even very low levels of persistent infection. Untreated mice in both groups exhibited evidence of active infection at 14 days post-exposure and beyond and as expected, all mice in the IFNγ deficient group not receiving prophylaxis experienced uncontrolled infections and had to be euthanized by ˜21 days post-exposure. In contrast, none of the mice in the BNZ-treated WT group had a detectable luciferase signal above background at day 14 post-exposure or beyond while some BNZ-prophylaxed IFNγ KO mice showed low level signal at day 14 post-exposure but not at 12 weeks (FIGS. 8B and 8C).

Infection exposure was also confirmed in mice by monitoring the generation of T. cruzi-induced CD8⁺ T cells specific for the immunodominant TSKb20 epitope (Bustamante, et al., Nature medicine. 14(5):542-50 (2008)). With the exception of one prophylaxed IFNγ KO mouse, all mice exhibited T. cruzi-specific CD8⁺ T cell responses with comparable numbers in the untreated WT and prophylaxed IFNγ KO mice and lower levels in the prophylaxed WT mice. Results that the expression of the T cell central memory marker CD127 on these parasite-specific T cells is a useful surrogate for parasite load (Bustamante, et al., Sci Transl Med. 12(567) (2020), Bustamante, et al., Nature medicine. 14(5):542-50 (2008)) and the 2 groups under prophylaxis had a substantially higher fraction of CD127+ cells in the TSKb20-specific population relative to mice not receiving prophylaxis, consistent with the expected low/absent parasite burden through the 21^{st} week of prophylaxis (FIG. 8D).

Twice weekly BNZ was continued for a total of 24 weeks, simulating a seasonal period of vector activity typical of south Texas (Curtis-Robles, et al., PLoS neglected tropical diseases 9(12):e0004235 (2015)). In WT mice with or without prophylaxis, infection was not readily detectable by whole animal imaging through the prophylaxis period. However, in the IFNγ KO mice, although the infection was largely controlled by the prophylaxis treatment, the systemic infection was just detectable during the prophylactic treatment period and within 4 weeks of termination of BNZ prophylaxis, 4 of 5 mice in the previously treated IFNγ KO mice group were systemically parasite-positive and had to be euthanized (FIG. 8C). The remaining animals in all groups were euthanized at week 33 of the study and skeletal muscle and cardiac tissues were examined for the presence of T. cruzi using qPCR. As shown in FIG. 8E, all of the untreated WT mice were PCR positive for T. cruzi DNA in one or more of the samples from skeletal muscle, heart, or gut. In contrast, at least 3 of the 5 WT mice receiving BZN prophylaxis and the surviving IFNγ KO mouse were negative for T. cruzi DNA (the remaining 2 WT-treated mice were at or below the level of detection in this assay in skeletal muscle only). Collectively, these results indicate that the prophylaxis regimen employed here in WT mice prevented or truncated an infection in most cases. However, the protective effect of prophylaxis generally required a coincident intact immune response, as most IFNγ deficient mice became and remained infected despite prophylaxis.

Building upon these encouraging results in mice, experiments were designed to determine if BNZ prophylaxis could reduce infection in kennel and hunting dog populations in the south-central U.S. where infection pressure was high, despite other vector control efforts (Busselman, et al., PLoS neglected tropical diseases 15(11):e0009935 (2021)). An early spring (March to April) screening was conducted to identified uninfected dogs in this setting with the intent of capturing infection status prior to the window of high vector activity (Curtis-Robles, et al., PLoS neglected tropical diseases 9(12):e0004235 (2015)), using a combination of negative serological tests and negative blood PCR. The rationale for using this combination of tests is that although blood PCR can provide solid evidence of active infection, a negative blood PCR assay is not a dependable indicator of absence of infection. Additionally, serologic tests may miss very recent infections. A total of 126 dogs of previously undetermined infection status were screened using a Luminex-based assay previously employed for detection and monitoring T. cruzi infection in humans (Alvarez, et al., Antimicrobial agents and chemotherapy. 64(9) (2020), Viotti, et al., PLoS neglected tropical diseases. 5(9) (2011), Laucella, et al., Clinical infectious diseases: an official publication of the Infectious Diseases Society of America. 49(11):1675-84 (2009)) and other species (Hartley, et al., Vet Res. 45:6 (2014), Padilla, et al., PLoS neglected tropical diseases. 15(3) (2021)), a commercial Stat-Pak test (ChemBio, NY) that is validated for humans but commonly used for research purposes in dogs (Curtis-Robles, et al., PLoS neglected tropical diseases. 11(1) (2017), Nieto, et al., Veterinary parasitology. 165(3-4):241-7 (2009)) and by blood PCR. FIG. 9A displays dogs considered to be infection-negative based upon the Luminex assay and confirmed in most cases by Stat-Pak and indirect fluorescent antibody (IFA) (Texas A&M Veterinary Medical Diagnostic Laboratory, College Station, TX; (Curtis-Robles, et al., PLoS neglected tropical diseases. 11(1) (2017)) assays (n=57; 45.2% of total screened). Nearly all dogs had detectable antibodies to the parvovirus vaccine antigen, confirming the quality of the serum sample, and in all cases the negative T. cruzi serology was supported by a negative blood PCR assay.

FIG. 9B shows the antibody and/or PCR+ dogs that were excluded from the prophylaxis trial by virtue of having prior exposure and likely active infection (n=53; 42.1% of those screened). Roughly half (29/53; 54.7%) of the dogs with detectable (generally robust) serological responses also had T. cruzi DNA in their blood as determined by qPCR. It is notable that, as in other species (Hartley, et al., Vet Res. 45:6 (2014), Alvarez, et al., Antimicrobial agents and chemotherapy. 64(9) (2020), Viotti, et al., PLoS neglected tropical diseases. 5(9) (2011), Laucella, et al., Clinical infectious diseases: an official publication of the Infectious Diseases Society of America. 49(11):1675-84 (2009), Padilla, et al., PLoS neglected tropical diseases. 15(3) (2021)) the pattern of antigen specificity of antibody responses in these subjects varied extensively, although the antigen in lane 8, a polyubiquitin protein, appeared to be the most reliable. A fraction of the screened dogs (n=14; 11.1%) were seronegative by Luminex and other assays but had a weak signal in the blood PCR assay that was measurable, but above the standard cutoff for determining infection (Ct<35.0; FIG. 9C). These dogs were initially enrolled in the prophylaxis study as uninfected but 4 of these likely were already infected and these were excluded from prophylaxis study analysis (detailed further below).

The 67 animals considered infection-negative were randomly assigned to either a control (no therapy) or a prophylactic group, which received twice weekly doses of BNZ at a level of not less than 10 mg/kg. This dosage is approximately the normal daily dose previously used (mostly ineffectively) to attempt drug-cure in dogs (Guedes, et al., Acta tropica. 84(1):9-17 (2002), Santos, et al., The Journal of antimicrobial chemotherapy. 67(8):1987-95 (2012), Cunha, et al., Experimental parasitology. 204:107711 (2019)). Dogs in these groups were then re-sampled 10-12 weeks (considered the midpoint of the transmission season) and ˜24 weeks (end of season).

Apparently new infections occurred in both the control (n=6; 18.2%) and prophylaxis (n=9; 26.5%) groups, yielding a combined seasonal infection incidence of 22.4%. In all but one case, these infections occurred early in the season, prior to the 12 week screening timepoint. One newly-infected dog in each group was lost to follow up before the 24 week sampling point (one was sold and one died due to T. cruzi with severe, acute, lymphohistiocytic, necrotizing pancarditis with intralesional amastigotes). Additionally, one infected dog in each group was removed from the study after the mid-point sampling due to high serum cardiac troponin I (cTnI) levels (indicative of active heart damage) and started on a treatment protocol consisting of higher dose BNZ (to be reported on elsewhere). Thus, BNZ prophylaxis as employed in this study had no impact on the number of new infections in this high intensity transmission setting.

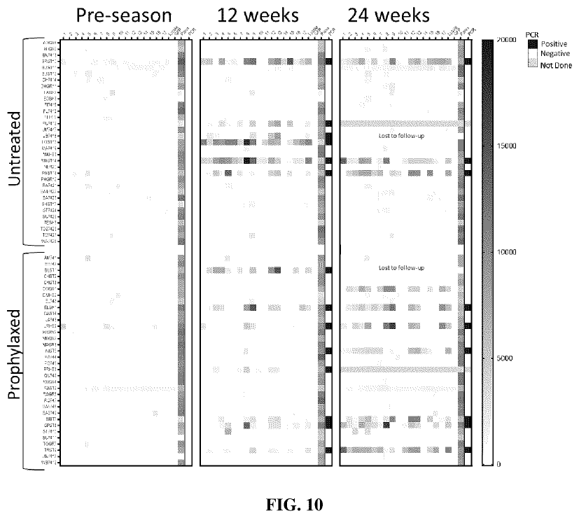

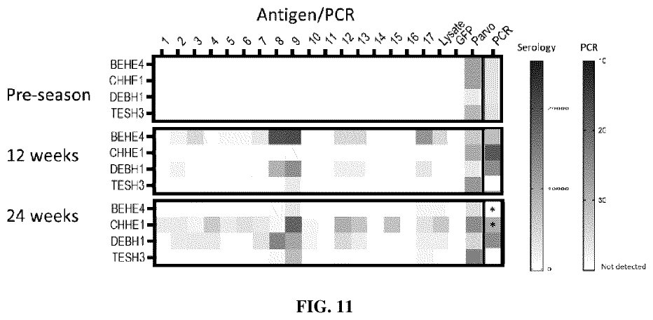

The serial sampling during a transmission season and the stable pattern of the antibody profiles in infected subjects provided the opportunity for several additional novel observations. First, 4 of the 16 seronegative dogs with blood PCR signals exceeding the positive cutoff point (FIG. 9C) developed a seropositive profile during follow-up (FIG. 11) and 3 of the 4 developed strong blood PCR signals. While these could represent new infections acquired during the follow-up period, they may also indicate dogs that were already infected at the time of the March/April survey but were so early in the infection course that antibody responses had not developed and parasites in the blood were low. The remaining 12 dogs in this subset were equally dispersed in the untreated and prophylaxis groups and none developed evidence of infection during the 24-week follow-up (FIG. 10). Looking retrospectively, one dog in each group in the prophylaxis trial was PCR negative (JRHE2, prophylaxis group and PAST16, control group) but was nonetheless also likely already infected at the pre-season survey point, as both had low reactivity to several T. cruzi antigens in the multiplex assay at this point but with a pattern that matched the more robust antigen recognition pattern in the Luminex assay that ultimately developed by the 12 and 24 week sampling points (FIG. 10).

Lastly, one dog in the control group (LOST13, FIG. 10) had developed a substantial antibody profile by the 12 week sampling point (at which he was also PCR positive), but had essentially totally lost this response by the 24 week screening point. This dog was also strongly positive by a StatPak antibody test and had detectable serum cTnI (0.021 ng/mL) at 12 weeks but was seronegative and had cTnI below lower detection limit of 0.006 ng/mL at the 24 week sample time point. The continued detection of parvovirus-specific antibodies over this same time course ruled out concerns on sample quality or immune competency. A similar pattern of cure was evident in dog BEHE4 (FIG. 11) who was switched to a BNZ treatment regimen (increasing the dosage to 30 mg/kg twice a week) after exhibiting high cTnI levels in the 12 week sample. This dog became PCR negative and seronegative by the 24 week sample point. Collectively, these data support a potential early infection spontaneous cure one dog and BNZ-induced cure in another, both resulting in a rapid decline in serological evidence of the previous infection.

Discussion

Studies have extensively documented the high risk of T. cruzi infection in dogs in south central Texas, including among kenneled working dog populations (Busselman, et al., PLoS neglected tropical diseases 15(11):e0009935 (2021), Meyers, et al., PLoS neglected tropical diseases 11(8) 2017), Curtis-Robles, et al., PLoS neglected tropical diseases. 11(1) (2017)). New infection rates of up to 30%/year put these valuable animals at high risk for morbidity and early death. It is noteworthy that this high incidence of infection is occurring despite vector awareness education for dog owners and the implementation of a variety of vector control measures. Likely, the abundance of insect vectors and wildlife reservoirs with active T. cruzi infections, the outdoor group housing conditions that expose dogs to this infection risk, and the propensity of dogs to ingest these vectors and their feces, all combine to promote this high transmission situation. The lack of interventions such as vaccines that could reduce the incidence of new infections and the high failure rates of potential therapies leave few options for reducing the impact of T. cruzi in these settings.

In this study, the potential of BNZ, a drug that has been in use for >50 years for the treatment of T. cruzi infection, was explored in a prophylactic modality to attempt to reduce the rate of new infections in dogs. Prophylactic use of trypanocidal drugs has not been previously explored for prevention of new infections with T. cruzi (Van Voorhis, Therapy and prophylaxis of systemic protozoan infections. Drugs. 40(2):176-202 (1990)), although prophylaxis has occasionally been employed to prevent potential exacerbation of infection due to immunosuppression following tissue transplantation in humans (Rossi Neto, et al., Tropical Medicine and Infectious Disease 5(3):132 (2020)). The premise behind these studies was that dosing BNZ at a standard daily dose level, but administering it twice per week instead of daily, could prevent establishment of new infections, or might rapidly terminate those infections before they could become firmly established. The twice-weekly dosing pattern is supported by the ability of BNZ to cure established T. cruzi infections in mice using this schedule, albeit with 2.5-5-fold the dose level used here for prophylaxis. Selection of this lower prophylactic dose, bi-weekly schedule was also driven by the practicality in terms of time/effort, cost, and cumulative drug toxicity over a potential 6-month transmission season.

Trials of BNZ-based prophylaxis in laboratory mice yielded promising results, demonstrating a substantial reduction in parasite burden and the ultimate resolution of infection in the majority of immunocompetent mice. Notably, this protocol had much less impact in mice compromised in the ability to produce IFNγ, an essential component of the anti-T. cruzi immune response. This latter result supports the hypothesis that a competent immune response likely works in concert with trypanocidal drugs to ultimately clear T. cruzi infection when treatment is successful.

However, in dogs in a high transmission setting, prophylaxis as applied here failed to prevent new infections during a 6-month transmission season. A number of factors could have contributed to this differential result in mice and dogs, primary among them, the likely differential parasite exposure. Mice received a single sub-lethal intraperitoneal injection of 1000 trypomastigotes, while dogs were potentially exposed repeatedly to infection, possibly at much higher doses, and likely via an oral route. The fact that one dog newly infected during the died during the acute infection and others exhibited high serum cTnI levels, prompting their transfer to a higher treatment dose regimen, supports the higher (and also variable) exposure conditions for dogs in this study. Effective prophylaxis under these scenarios might require a more aggressive dosing regimen, either more frequent or with higher dosage or both. Although not designed to explicitly address this point, the infection pattern observed in this study supports a seasonality in infection potential. The transmission season was estimated based upon cumulative vector sighting observations collected by Kissing Bug Community Science initiative (Curtis-Robles, et al., PLoS neglected tropical diseases 9(12):e0004235 (2015)), which indicates vector activity beginning in May and peaking in July. However, at least 4 dogs were borderline PCR positive but seronegative in early May, and all developed serological responses, 3 of the 4 within 10-12 weeks of the initial sampling. Additionally, two dogs that were PCR-negative, but had a suggestion of a serological response at initial screening, were PCR positive 10-12 weeks later. Collectively, these data indicate that new infections may be occurring before the early May prophylactic dosing. In combination with the fact that most new infections occurred in the first 12 weeks dosing period, it seems likely that beginning prophylaxis earlier in the year could have had a greater impact in the rate of new infections. Including these four “pre-prophylaxis” infected dogs as among those infected in 2021 year increases the yearly incidence in this setting to 28.4% (19/67).

It is believed that no previous study has sampled a susceptible, at risk, animal population at the frequency and with both PCR and multiple serology as employed in this study. In addition to the high infection pressure, this setting is also optimal for collection of novel and valuable data because new dogs are introduced each season, through either breeding or new acquisitions. Among the interesting observations from this first screening year is that 14 of the 15 (or 18 of 19 if the 4 pre-May infections are included) new infections occurred before the calculated mid-point of the estimated transmission season. The reason for this early season bias is not known, but could be due to opportunity based on location, or to behavior (e.g. aggressive bug eaters are more likely to be exposed and become infected) or a combination of the two. Given the high early season incidence, it is clear that the opportunities for infection are high even when bug activity appears low. Identifying the specific links between bug numbers, locations and infection status with the seasonality of new infections in dogs should be addressed in future seasonal studies. Active entomological surveillance at sentinel locations within the geographic region of study would be useful to establish a more precise start of the seasonal insect activity period, so that prophylaxis in future years could be initiated prior to insect activity.

The frequency of sampling applied herein also allowed for the detection of one apparent case of spontaneous cure of an acute infection. Although such spontaneous cures have been anecdotally reported in chronically infected hosts, both animal (Tarleton, et al., Revista Espanola de Salud Pública. 87:33-9 (2013)) and human (Pinto Dias, et al., Revista da Sociedade Brasileira de Medicina Tropical 41(5):505-6 (2008)), it is believed that cure during the acute infection has not been documented. The frequency of such cures is worth exploring further and as well, the protection from re-infection that might be afforded by such cases. The very rapid waning of the antibody response in this case, as well as following an apparent BNZ treatment dose-induced cure, was also surprising and would have gone undetected if screening was conducted only once or twice per year, for example.

The use of both of a multiplex serological test and blood PCR to track new infections was important to the technical success of this project and for revealing some of the less expected observations. The frequent failure of PCR to detect many chronic infections with T. cruzi is well recognized (Padilla, et al., PLoS neglected tropical diseases. 15(3) (2021)). The multiple antigen array used in this study, incorporating >25 parasite proteins, a crude lysate, and both positive and negative control proteins, provides a confidence in detection of infection that is lacking in single antigen or whole parasite assays. However, all serological assays fail infection detection very early, before anti-parasite antibodies have been formed. Thus, the combined use of PCR and multiplex serology provided insights that would have been missed using either approach alone.

Also, not addressed in this study is whether prophylaxis, while not reducing the incidence of infection, might be beneficial by ultimately reducing the severity of disease. Anecdotally it was observed that several dogs infected while under prophylaxis nonetheless experienced high serum levels of cTnI, indicating that prophylaxis in these cases was not preventing acute phase disease. A longer-term follow-up of disease development with and without early prophylaxis might be revealing. However, when infection is detected in these working dogs, they should be enrolled in an effective treatment regimen (as done for dogs BEHE4 and CHHE1) that enhances the chances of resolving the infection and thus preventing disease progression.

Although the seasonal monitoring for new infections in a population at high risk of infection provided new insights into T. cruzi transmission and development of immune responses, the prophylaxis approach applied here did not prevent infection in this setting. Future studies might apply prophylaxis earlier in the transmission season or at a more aggressive dosing level. Also, it would be of interest to determine if prophylaxis, while failing to reduce infection incidence, might be beneficial with respect to reducing infection impact either in the short-term, or long-term.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A fusion protein comprising linkage of any two, three, or more T. cruzi polypeptides, wherein each T. cruzi polypeptide is a T. cruzi protein or fragments or variants thereof.

2. The fusion protein of claim 1, wherein the T. cruzi polypeptides are selected from SEQ ID NOS:1-22 and 53-59, variants thereof with at least 70% sequence identity thereto, and fragments of the foregoing comprising at least 15 amino acids.

3. The fusion protein of claim 1, comprising the formula N—R₁-R₂-R₃-C, wherein “N” indicates the N-terminal end and “C” indicates the C-terminal end of the fusion protein;
R₁ is a first polypeptide selected from SEQ ID NOS:1-22 or 53-59, variants thereof with at least 70% sequence identity thereto, and fragments of the foregoing comprising at least 15 amino acids;
R₃ is a second polypeptide selected from SEQ ID NOS:1-22 or 53-59, variants thereof with at least 70% sequence identity thereto, and fragments of the foregoing comprising at least 15 amino acids;
and R₂ is an optional linker.

4. The fusion protein of claim 1, wherein the two, three, or more polypeptides are different polypeptides.

5. The fusion protein of claim 1, wherein the two, three, or more polypeptides are derived from different SEQ ID NOS.

6. The fusion protein of claim 1, wherein the two polypeptides are
i. SEQ ID NOS:1 and 12, variants thereof with at least 70% sequence identity thereto, or fragments of the foregoing comprising at least 15 amino acids;
ii. SEQ ID NOS:2 and 13, variants thereof with at least 70% sequence identity thereto, or fragments of the foregoing comprising at least 15 amino acids;
iii. SEQ ID NOS:3 and 14, variants thereof with at least 70% sequence identity thereto, or fragments of the foregoing comprising at least 15 amino acids;
iv. SEQ ID NOS:4 and 15, variants thereof with at least 70% sequence identity thereto, or fragments of the foregoing comprising at least 15 amino acids;
v. SEQ ID NOS:5 and 16, variants thereof with at least 70% sequence identity thereto, or fragments of the foregoing comprising at least 15 amino acids;
vi. SEQ ID NOS:6 and 17, variants thereof with at least 70% sequence identity thereto, or fragments of the foregoing comprising at least 15 amino acids;
vii. SEQ ID NOS:7 and 18, variants thereof with at least 70% sequence identity thereto, or fragments of the foregoing comprising at least 15 amino acids;
viii. SEQ ID NOS:8 and 19, variants thereof with at least 70% sequence identity thereto, or fragments of the foregoing comprising at least 15 amino acids;
ix. SEQ ID NOS:9 and 20, variants thereof with at least 70% sequence identity thereto, or fragments of the foregoing comprising at least 15 amino acids;
x. SEQ ID NOS:10 and 21, variants thereof with at least 70% sequence identity thereto, or fragments of the foregoing comprising at least 15 amino acids;
xi. SEQ ID NOS:11 and 22, variants thereof with at least 70% sequence identity thereto, or fragments of the foregoing comprising at least 15 amino acids;
xii. SEQ ID NOS:53 and 54, variants thereof with at least 70% sequence identity thereto, or fragments of the foregoing comprising at least 15 amino acids;
xiii. SEQ ID NOS:55 and 56, variants thereof with at least 70% sequence identity thereto, or fragments of the foregoing comprising at least 15 amino acids; or
xiv. SEQ ID NOS:57, 58, and 59, variants thereof with at least 70% sequence identity thereto, or fragments of the foregoing comprising at least 15 amino acids.

7. The fusion protein of claim 1, wherein the fusion protein is antigenic for one or more anti-Trypanosoma cruzi antibodies.

8. The fusion protein of claim 7, wherein the anti-Trypanosoma cruzi antibody or antibodies are from a subject or subjects infected with Trypanosoma cruzi.

9. The fusion protein of claim 8, wherein the number of antibodies, the binding affinity of the antibodies, the specificity of the antibodies, or a combination thereof for the fusion protein is higher than for
a. one of the polypeptides as single polypeptide in the absence of being linked to the other polypeptide;
b. both of the polypeptides in the absence of being linked to each other; and/or
c. the additive result of both of the polypeptides in the absence of being linked to each other.

10. A polypeptide comprising the amino acid sequence of any one of SEQ ID NOS:67-80 or a variant thereof with at least 70% sequence identity thereto.

11. A substrate comprising one or more combinations of any two, three, or more T. cruzi polypeptides immobilized thereon, wherein each T. cruzi polypeptide is a T. cruzi protein or fragments or variants thereof.

12. The substrate of claim 11, wherein the combination(s) of polypeptides comprise one or more of:
i. SEQ ID NOS:1 and 12, variants thereof with at least 70% sequence identity thereto, or fragments of the foregoing comprising at least 15 amino acids;
ii. SEQ ID NOS:2 and 13, variants thereof with at least 70% sequence identity thereto, or fragments of the foregoing comprising at least 15 amino acids;
iii. SEQ ID NOS:3 and 14, variants thereof with at least 70% sequence identity thereto, or fragments of the foregoing comprising at least 15 amino acids;
iv. SEQ ID NOS:4 and 15, variants thereof with at least 70% sequence identity thereto, or fragments of the foregoing comprising at least 15 amino acids;
v. SEQ ID NOS:5 and 16, variants thereof with at least 70% sequence identity thereto, or fragments of the foregoing comprising at least 15 amino acids;
vi. SEQ ID NOS:6 and 17, variants thereof with at least 70% sequence identity thereto, or fragments of the foregoing comprising at least 15 amino acids;
vii. SEQ ID NOS:7 and 18, variants thereof with at least 70% sequence identity thereto, or fragments of the foregoing comprising at least 15 amino acids;
viii. SEQ ID NOS:8 and 19, variants thereof with at least 70% sequence identity thereto, or fragments of the foregoing comprising at least 15 amino acids;
ix. SEQ ID NOS:9 and 20, variants thereof with at least 70% sequence identity thereto, or fragments of the foregoing comprising at least 15 amino acids;
x. SEQ ID NOS:10 and 21, variants thereof with at least 70% sequence identity thereto, or fragments of the foregoing comprising at least 15 amino acids;
xi. SEQ ID NOS:11 and 22, variants thereof with at least 70% sequence identity thereto, or fragments of the foregoing comprising at least 15 amino acids;
xii. SEQ ID NOS:53 and 54, variants thereof with at least 70% sequence identity thereto, or fragments of the foregoing comprising at least 15 amino acids;
xiii. SEQ ID NOS:55 and 56, variants thereof with at least 70% sequence identity thereto, or fragments of the foregoing comprising at least 15 amino acids; or
xiv. SEQ ID NOS:57, 58, and 59, variants thereof with at least 70% sequence identity thereto, or fragments of the foregoing comprising at least 15 amino acids.

13. The substrate of claim 11, comprising all of the combinations of (i)-(xiv).

14. The substrate of claim 11, wherein the combination(s) of T. cruzi polypeptides are linked to form a fusion protein.

15. The substrate of claim 11, wherein the substrate comprises or consists of glass, metal, or plastic.

16. A method of detecting one or more anti-Trypanosoma cruzi antibodies in a sample comprising contacting the sample with the substrate of claim 11 under conditions suitable for antibodies specific for the fusion protein or proteins to bind thereto, and detecting the bound antibodies.

17. The method of claim 16, wherein the biological sample is whole blood, plasma, serum, urine, saliva, tears, or lymphatic fluid.

18. A method of diagnosing a subject with a Trypanosoma cruzi infection comprising detecting anti-Trypanosoma cruzi antibodies according to the method of claim 16, wherein the sample is a biological sample from the subject, and the subject is diagnosed as positive for a Trypanosoma cruzi infection if anti-Trypanosoma cruzi antibodies are detected.

19. The method of claim 18, further comprising treating positive subjects for the Trypanosoma cruzi infection.

20. The method of claim 19, wherein treated comprises administering the subject an antiparasitic drug optionally selected from benznidazole and nifurtimox, and optionally in further combination with administering a subject with side effects from the antiparasitic drug an effective amount of an antihistamine or corticosteroid to reduce the side effects.


SEQUENCE LISTING (124 sequences)

SEQ ID NO: 1 - protein, 109 aa, synthetic construct
MSSKQQLACTYAALILADSGKTDMDSLLKVTKAAGVDVSKGMASAFASILKNVDINDVLSKVSFGGVAPAAGGATAAPAAAAAAAAPAAAAAKKEEEEEDDDMGFGLFD

SEQ ID NO: 2 - protein, 336 aa, synthetic construct
MVSLKLQARLAADILRCGRHRVWLDPNEASEISNANSRKSVRKLIKDGLIIRKPVKVHSRSRWRHMKEAKSMGRHEGAGRREGTREARMPSKELWMRRLRILRRLLRKYREEKKIDRHIYRELYVKAKGNVFRNKRNLMEHIHKVKNEKKKERQLAEQLAAKRLKDEQHRHKARKQELRKREKDRERARREDAAAAAAAKQKAAAKKAAAPSGKKSAKASAPAKAATAPAKATAAPAKAAAAPAKATAAPAKATAAPAKAAAAPAKATAAPAKAAAAPAKAAAAPAKAAAAPAKAAAAPAKATAAPAKAAAAPAKVAAAPAKAAAAPVGKKAGGKK

SEQ ID NO: 3 - protein, 240 aa, synthetic construct
MPAKSANKPASKPAAKPAAKPAAKAPAPKAAAPAPKAAAAAPKPAVRDAKQRSDAANHNGLYVKNWGQGSVDDARALFGTAGKVVGVRVRRRRYAIIFFENAAAVKKAIDLFNGKEFMGNVLSVVPAKTTPKPDPHANSSVVFVSPIFRASTTKKQILELFSGMKVLRLRTYRNNYAYVYLDTPAAAQRAVKEKNGAEFRGKQLRVALSTRSLAKDRARAERARLLIAAQKFNKRKNHTK

SEQ ID NO: 4 - protein, 186 aa, synthetic construct
MAFCIISESRGMSLWDMLAWHRPKVTGVLLGTVLSVLTFFCLMKYTMVTFLCRILQLVLLAGVLLGFTNRWHLTSDDIHEAVNRLVDCATPRLVTALESMHQLVTWRDYRRSGLVTLVSFVVALLGNLVSDAAFLTFFLLLAFTVPAVYEKKKDLIDKWISAATAQVEKYMGKIKTKVEEATKKKE

SEQ ID NO: 5 - protein, 315 aa, synthetic construct
MPGKEVKKAAKPAAKTAAKPAAKSAAKPAAKPAAKPAAKTAAKPAAKTAAKPAKKPAVKPTVKPAAKAAAPYKKPAAISPFVARPKNFGIGHDVPYARDLSRFMRWPTFVTMQRKKRVLQRRLKVPPALHQFTKVLDRSSRNELLKLVKKYPSETRRARRQRLFDVATEKKKNPEAASKKAPLSVVTGLQEVTRTIEKKTARLVMIANNVDPIELVLWMPTLCRANKVPYAIVKDKARLGDAVGRKTATCVAITDVNAEDEAALKNLIRSVNARFLARSDVIRRQWGGLQLSLRSRAELRKKRARNAGKDAAAVM

SEQ ID NO: 6 - protein, 240 aa, synthetic construct
MTTIGTYNEEGVNVDLYIPRKCHATNNLITSYDHSAVQIAIANVDANGVLNGTTTTFCIAGYLRRQAESDHAINHLAISKGIIRIKTGKKPRAKKLKNVKGLGVRGLPRGALQQRGARVLPTQRGVAQRGGAQKGNVRKLQPQPQKQRSQLNQRSQQQHGARPTRKEEGGRTQRGGRDAPQARKQQGRNEPQARRQQGRNEPQARRQQGRNEPQARKQQGRDAPQARKQQGRNAPRSQKA

SEQ ID NO: 7 - protein, 221 aa, synthetic construct
MGIVRSRLHKRKITGGKTKIHRKRMKAELGRLPAHTKLGARRVSPVRARGGNFKLRGLRLDTGNFAWGTEAIAQRARILDVVYNATSNELVRTKTLVKNCIVVVDAAPFKLWYAKHYGIDLDAAKSKKTAQSTTEKKKSKKTSHAMTEKYDVKKASDELKRKWMLRRENHKIEKAVADQLKEGRLLARITSRPGQTARADGALLEGAELQFYLKKLEKKKR

SEQ ID NO: 8 - protein, 114 aa, synthetic construct
MGAPQIVYSALITNTTTIAVTVVVTYTMPNEMPPETLELLIQPGEEMLAPQKLVEDGIVTWTGYISKVAIQGGPSMSEPFPGVECPTRRYDFEVFMHAGVLRLFALGPAESSSD

SEQ ID NO: 9 - protein, 114 aa, synthetic construct
MGSPKIVYSALIRNTTTISVTVLVTYSMPSEMPQETVQLLIPPGEEKEAPQKLVEEDTVTWTGFISKVAVEGGQSMSAPFLGVESPTRRYGFEVYMQAGMLRLLALGPVESSSD

SEQ ID NO: 10 - protein, 225 aa, synthetic construct
MPSTPTPQCVRRLQKELSALCREAESFFFTRPSAKSILVWYFVIKGPADTPYEGGRYFGKLNFPPDYPMKPPEIIILTPNGRFETNKSICLTMSNYHPENWSPLWGVRTILTGLLSFMVGDELTTGCMTSSDELRRKYARESRRFNAEKMSVYKELFPEEYQKDLEELKREDSEKNGRTSGSAGCGANTKGGGVMESQEKEQWRGLFPALLGLFAVLMGAYFWPW

SEQ ID NO: 11 - protein, 225 aa, synthetic construct
MPSTPTPQCVRRLQKELSALCREAESFFFTRPSAKSILVWYFVIKGPADTPYEGGRYFGKLNFPPDYPMKPPEIIILTPNGRFETNKSICLTMSNYHPENWSPLWGVRTILTGLLSFMVGDELTTGCMTSSDELRRKYARESRRFNAEKMPVYKELFPEEYQKDLEELKREDNEKNGRISGSAGCGANTKGGGVMESQEKEQWRGLFPALLGLFAVLMGAYFWPW

SEQ ID NO: 12 - protein, 112 aa, synthetic construct
MSMKYLAAYALASLNKPTPGAADVEAICKACGIEVESDALSFVMESIAGRSVATLVAEGAAKMSAVAVSAAPAAGGAAAPAAAAGGAAAPAAADAKKEEEEEDDDMGFGLFD

SEQ ID NO: 13 - protein, 265 aa, synthetic construct
MAPPADMRGALREVLGAMQKAQEYADEANRHCVQARMSAESAREHEEGAKNALRKLGSEATRMSRALQQADEAVKLADAAVAECKAAEEAAQAAGIMTLDAVGEVLKHLKDEKTKVGSGPELLKRAAEQTVLSLEKAKEAEAEAEKAAAAAQKTREAAEKAAAARTLAQDVAATASALLRQREKEEERRRARDQEVAEAAKKAAVAEVMKKFAAKGNDTAPGRNSTSTRFQRTRPRVDGGGIPLLLRAPLLMLAAVASVFGFLSC

SEQ ID NO: 14 - protein, 344 aa, synthetic construct
MTRNRLFFPLLLLLSCSVIVGANATEKKASTPRKAEGVQPQSVSPSSSFPGDGTGVPLKLELGELRDKALLAAKDAFGNTTGAAMQCMQAKTDVEETKKYAEEAKKLFDKIGGDYVSKSAALADAVKASTDAEEALKSCVEAEKAAVDADTAVLAAVLEVLQHSKFWRRDTAVSTEKLANVSKHSANATNEAQKAGIQASKAAEAAKRAAESKKKAAAALDTVKEVVAMAEMLKEKFFENERLQKEKHEAQLEAERQFIQEEVQKKEAEAEKALNRAAAADKRVAELELARQKQSKEQGNEGRGHRRVRRSGSDSSSNYAPAYEPRLLLLPLLSFTLFCFVAWC

SEQ ID NO: 15 - protein, 200 aa, synthetic construct
MPAKTAVSKAAAPKKAAAPKKAAAPQKAAAPKKAAAPKKAAAPQKAAVAKKAVREAPKKGVKKTAKKGAPAAMTKVVKVTKRKAYTRPQFRRPHTYRRPSIPKPSNNMSAIPNKWDAFRVIRYPLTTDKAMKKIEENNTLTFIVDSNANKTEIKKAMRKLYQVKAVKVNTLIRPDGLKKAYIRLSASYDALETANKMGLL

SEQ ID NO: 16 - protein, 273 aa, synthetic construct
MTKKHLKRLYAPKDWMLSKLTGVFAPRPRAGPHKLRECMTLMIIIRNRLKYALNAAEAQMILRQGLVCVDGKPRKDTKYPVGFMDVVEIPRTGDRFRILYDVKGRFALVKVGEAEGNIKLLKVENVYTSTGRIPVAMTHDGHRIRYPDPRTHRGDTLVYNLKEKKVVDLIKSSNGKVVMVTGGANRGRIGEIMSIERHPGAFDIARLKDAAGHEFATRASNIFVIGKDMQSVPVTLPKQQGLRINVIQEREEKLIAAEARKNMQTRGVRKARK

SEQ ID NO: 17 - protein, 192 aa, synthetic construct
MMRFTRFLVVAAKRSATSAKLGKSVGLTAALSPKQRSLPRVSVTKLMKPSGSGKHVTSSFLLKDKKKVATAKVAVPPKKKRALKVRKGRSSGKKAAALYVRFYHALKKSGLVKGKRRMQKTGELWRATKKAKDFKKRVEAAMRLAKKGQKSRARKLKAQKKAKGKKSAKGVRRVYRRVSRKKTVTSTVPPLP

SEQ ID NO: 18 - protein, 218 aa, synthetic construct
MPKGKNAIPHVHQRKHWNPCSSQKGNVKVFLNQPAQKLRRRRLRLLKAKKTFPRPLKALRPQVNCPTVRHNMKKRLGRGFTVEELKAAGINPRFAPTIGIRVDRRRKNKSEEGMSINIQRLKTYMSKLVLFPMSYKNVQKGEATEEEVKSATQDRTRFGTAAVGGFVTPAPEAPRKVTEEERTKNVYKFLKKNHSAVRFFGIRRARQERREAKENEKK

SEQ ID NO: 19 - protein, 214 aa, synthetic construct
VVYAGNGIPEIGIKAMGRHFRHVSEDGKRWMSLENIGKLVRDSRAFSNTLSFEDTQRTNSTISNNARSIHEAFPQNEEGCIDYDLFMDYVRGPMSQKRKDAVWEIFRKLDFDGDGYLNILDIQARYNAQQHPVVAVERLFSADKLLKGFLTVWDENKQYGLIPYAEFIDYYNGVSAVIADDYIFFDILRNQWKVMRDWGGTVGTRGGNCEFPTM

SEQ ID NO: 20 - protein, 374 aa, synthetic construct
MDTTLYSEVNRLERGDFLFFHCVQLSQHERDVQRYFFGCYFPRWRGFYLEEVRDMPGPLGYKVQRHFPAYPFDVYLKDNGEHFLTDDFQEGSIFTLGASQNQRDGESKRYKVVHCDDSRLRTRTGTTLADIGNDITTRLNQTHRVPGEVIDLLREIRDAYVVYAGNGIPEIGIKAMGRHFRHVSEDGKRWMSLENIGKLVRDSRAFSTTLSFEDTQKTNSTISNNARSIHEAFPQNEEGCIDYDLFMDYVRGPMSQKRKDAVWEIFRKLDFDGDGYLNILDIQARYNAQQHPVVAVERLFSADKLLKGFLTVWDENKQYGLIPYAEFIDYYNGVSAVIADDYIFFDILRNQWKVMRDWGGTVGTRRGKSEVSTM

SEQ ID NO: 21 - protein, 115 aa, synthetic construct
MADKVEANDTLACTYAALMLSDAGLPITAEGIEAACVAAGLKVRNTLPVIFARFLEKKPLETLFAAAAATAPAEGAAAAPAAGSAAPAAAAAGAAPEKDTKEEEEDDDMGFGLFD

SEQ ID NO: 22 - protein, 115 aa, synthetic construct
MADKVEANDTLACTYAALMLSDAGLPITAEGIEAACVAAGLKVRNTLPVIFARFLEKKPLESLFAAAAATAPAEGAAAVPAAGSAAPAAAAAAAAPAKDTKEEEEDDDMGFGLFD

SEQ ID NO: 23 - DNA, 330 bp, synthetic construct
  source 1..330: mol_type = other DNA
atgtcctccaaacagcagcttgcctgcacctacgccgccctgattcttgccgatagcggcaagacggatatggacagcctgttgaaagtgacaaaggccgccggtgttgacgtcagcaaagggatggcctcggcgtttgccagcatcctcaagaacgttgacatcaacgacgtgctctccaaagtgagctttggtggtgttgctcctgctgccggtggtgccaccgctgctcctgctgctgctgctgctgccgccgcccctgccgccgccgccgcaaagaaggaagaggaagaggaagacgacgatatgggctttggtctgtttgactag

SEQ ID NO: 24 - DNA, 1011 bp, synthetic construct
  source 1..1011: mol_type = other DNA
atggtgtcgctgaagctgcaggctcgtttggcggcggacattctccgctgcggtcgccaccgtgtgtggctggaccctaatgaggcctctgagatttccaatgcaaactcgcgcaagagcgtgcgcaagttgatcaaggatggtctgattattcgcaagcctgtcaaggtgcactcgcgctcccgctggcgccacatgaaggaggcgaagagcatgggccgccacgagggcgctgggcgccgcgagggtacccgcgaagcccgcatgccgagcaaggagctgtggatgcgccgtctgcgcattctccgccgcctgctgcgcaagtaccgcgaggagaagaagattgaccgccacatttaccgcgagctgtacgtgaaggcgaaggggaacgtgtttcgcaacaagcgtaacctcatggagcacatccacaaggtgaagaacgagaagaagaaggaaaggcagctggctgagcagctcgcggcgaagcgcctgaaggatgagcagcaccgtcacaaggcccgcaagcaggagctgcgtaagcgcgagaaggaccgcgagcgtgcgcgtcgcgaagatgctgctgctgccgccgccgcgaagcagaaggcagctgcgaagaaggccgctgctccctctggcaagaagtccgcgaaggcttctgcacctgccaaggccgctactgcacccgcgaaggccactgccgcacccgcgaaggctgctgctgcacccgcgaaggccactgctgcacccgcgaaggccactgctgcacccgcgaaggctgctgctgcacccgcgaaggccactgctgcacccgcgaaggccgctgctgcacccgcgaaggctgctgctgcacccgcgaaggccgctgctgcacccgcgaaggctgctgctgcacccgcgaaggccactgctgcacccgcgaaggccgctgctgcacctgcgaaggtcgctgctgcacccgcgaaggctgctgccgctcccgttggaaagaaggctggtggcaagaagtga

SEQ ID NO: 25 - DNA, 723 bp, synthetic construct
  source 1..723: mol_type = other DNA
atgcccgccaagtctgccaacaagcctgcatccaagcctgccgccaagcccgctgcgaagcctgccgccaaggctcccgcacccaaagctgctgcccctgctcccaaggctgctgcggctgcgcccaagccagctgtgagggacgcaaagcagcgctctgatgccgccaatcacaacggcttgtacgtgaagaactggggccagggttctgtggacgacgccagggcgctttttggcactgctgggaaggttgtgggtgtgagagtgcgtcgtcgccgttacgccattatcttctttgagaacgcagcggctgtgaagaaggccattgatcttttcaacgggaaagaatttatgggcaatgttttgtccgttgttcccgccaagacgactccgaagccggatccgcatgcgaactcctctgttgtgtttgtttccccgatattccgcgcgtcgactacaaagaagcagattcttgagcttttttcaggcatgaaggtactgcgcctgcgcacgtaccgcaacaactacgcatacgtctatctggacaccccagctgccgcgcaaagggctgtgaaggagaagaacggtgcagagttccgtggcaagcaactcagagttgccctctcgactcgttctcttgcgaaggacagggctcgtgcggagcgtgcaagacttcttatagccgcccaaaagttcaacaagagaaagaaccacacgaagtaa

SEQ ID NO: 26 - DNA, 561 bp, synthetic construct
  source 1..561: mol_type = other DNA
atggcgttttgtatcatttctgagagcaggggcatgtctctgtgggatatgctagcgtggcaccgcccaaaagttacgggtgtacttcttggaaccgtactttccgtcctgacgtttttttgccttatgaaatacacaatggtgacgttcctctgccgcatcctgcagttggtcctattggccggcgttctgttgggcttcacgaatcgatggcacctcacctccgacgacatccacgaggccgtcaaccgccttgtggactgcgccacgccccggctggtgacggcccttgagtccatgcaccaactcgtgacgtggcgtgactaccgccgctccgggctcgtcacgctggtgagcttcgtggttgctcttctcggcaacctcgtctccgacgccgcctttctcacgttttttcttttgttggccttcaccgttcctgcggtgtacgagaagaagaaggatttgatcgacaagtggatcagcgctgccacggctcaggtggagaagtacatggggaagatcaaaacaaaggtggaagaggcgaccaagaagaaagagtaa

SEQ ID NO: 27 - DNA, 948 bp, synthetic construct
  source 1..948: mol_type = other DNA
atgcccggcaaggaagtgaaaaaggccgccaagcccgctgccaagactgctgcaaagcctgctgccaagtctgctgccaagccagctgccaagccagctgccaagccagccgcgaagaccgctgcgaagccggccgcgaagactgctgccaagcccgctaagaagcccgctgtgaagcccactgtcaagcctgctgccaaggcagccgcgccctacaagaagcctgcggccatctcaccttttgtggcgcggccgaaaaactttggtattggccacgatgttccgtacgcccgtgatctttctcgctttatgcggtggcccacgtttgtgacgatgcagcggaagaagcgtgtactgcagcgccgtctgaaggtgccgcccgcgctccaccaatttacgaaggtgcttgaccgctccagtcgcaacgagctgctgaagctggtgaagaagtatccttccgagacgcgcagggcccgcaggcagcgcctgtttgacgtggcgactgagaaaaagaagaatccagaggcggcgtccaagaaggccccgctcagcgtcgttaccggtctgcaggaggtaacccgcaccattgagaagaagaccgcacgccttgtgatgatcgcgaacaatgtggaccccattgagctggtgctgtggatgccgactttgtgccgtgccaacaaagtcccatacgcgattgtgaaggacaaggcacgtctcggcgacgcggtgggccggaagaccgccacgtgcgttgcaatcaccgatgtgaatgccgaggacgaggccgctttgaagaatctcatccgctctgtgaatgcacgcttcctggcccgtagcgatgttatccgtcgccaatggggaggcctgcagctctcactgcgttctcgagccgagctgcgcaagaagcgtgcccgcaacgccggcaaggatgctgccgccgtaatgtga

SEQ ID NO: 28 - DNA, 723 bp, synthetic construct
  source 1..723: mol_type = other DNA
atgacgacaatcggtacgtacaacgaggagggtgttaacgtggacctgtacatcccacgcaagtgccacgcgacaaacaaccttatcacgtcatacgaccactccgccgtgcagattgccattgcgaatgtggacgccaacggtgtgctaaacggcacgacgacaaccttctgcattgctggctatcttcgtcgccaggctgagtctgaccacgcaatcaaccacctggcgatttcgaagggcattatccgcatcaagaccggcaagaagcctcgcgcgaagaagcttaagaatgtgaagggccttggcgtacgcggcttaccaaggggtgctctgcaacagaggggagctcgtgtcctcccaacccagaggggtgtcgcgcagcgtggcggcgctcagaagggcaacgtccgcaagctgcagccacagccgcagaagcaaaggtcacagctgaatcaaaggtcacagcagcagcacggcgcccggccgacccggaaggaagagggcggtcgcacgcagcgtggtggcagggatgcgcctcaagctcgcaagcagcaaggcaggaacgagcctcaagctcgcaggcagcaaggcaggaacgagcctcaagctcgcaggcagcaaggcaggaacgagcctcaagctcgcaagcagcaaggcagggatgcgcctcaagctcgtaagcagcaaggcaggaatgcacctcgttcccagaaggcatag

SEQ ID NO: 29 - DNA, 666 bp, synthetic construct
  source 1..666: mol_type = other DNA
atgggtatcgttcgcagccgcctgcataagcgcaagatcaccggtggaaagacgaagatccaccggaagcgcatgaaggccgaactcggccgtcttcccgcgcacacgaaacttggcgcccgccgcgtgagtcccgtccgcgcccgcggtgggaacttcaagctccgcggtcttcgcctggacaccggcaattttgcgtggggcacagaagccattgctcagcgggcccgtatcctcgacgtcgtgtacaacgccacttctaacgagctggtgcgcacgaagacgcttgtgaagaactgcattgttgtggtggacgccgcgcccttcaagttatggtacgcgaagcactacggtatcgatcttgacgccgcgaagagcaagaagacggcgcagagcacgacggagaagaagaagtcgaagaagacctcacacgccatgactgagaagtacgacgtcaagaaggcctccgacgagctgaagcgcaagtggatgctccgccgcgagaaccacaagattgagaaggcagtcgctgatcagctcaaggagggccgtctgctcgcccgcatcaccagccgccctggccagacagcccgcgccgatggtgcactgctggagggcgccgaactgcagttctatctgaagaagctcgagaagaagaagcggtag

SEQ ID NO: 30 - DNA, 345 bp, synthetic construct
  source 1..345: mol_type = other DNA
atgggggctcctcagatcgtgtactccgccctcataacgaacaccaccacaattgctgtgacggtggttgtcacctacaccatgccgaacgaaatgcccccggagactctggaattgctcattcaaccaggcgaagaaatgttagcgccgcagaaattggtggaggacggtatagtaacctggacaggctatattagcaaggttgccattcagggtgggccgtctatgagtgaacctttcccgggagtggagtgtcctacgagaagatacgactttgaagttttcatgcatgccggcgtgctgcggctattcgcattgggccctgccgaatcaagcagtgattga

SEQ ID NO: 31 - DNA, 345 bp, synthetic construct
  source 1..345: mol_type = other DNA
atggggtctcctaagatcgtgtactccgccctcataaggaacaccaccacgatttctgtgacggtgcttgtcacctattccatgccgagcgaaatgccccaggaaactgtgcaattgctcattccaccaggcgaagaaaaggaagcgccccagaaattggtggaggaagatacagtaacctggacaggctttattagcaaggttgccgttgagggtgggcagtctatgagtgctcctttcctgggagtggaatctcctacgagaagatacggttttgaagtttacatgcaagccggcatgctgcggctattagcattgggccctgtcgaatcaagcagtgattga

SEQ ID NO: 32 - DNA, 678 bp, synthetic construct
  source 1..678: mol_type = other DNA
atgccaagcacacccaccccgcagtgtgtgcggcggctgcaaaaggagctttccgccctatgccgagaggccgagtcgttttttttcacccgtccctcagcaaagagtattctggtttggtatttcgtcatcaagggtcctgcggataccccttatgaaggcggtcgctactttggcaagctgaattttccccccgactatccaatgaaaccgcctgagattatcattttgacgccaaatggacgttttgagaccaacaagagcatttgtctcaccatgagcaattatcatccggagaattggagccctttgtggggggtccgcaccattcttacggggctgctctcattcatggtgggagacgaactcactactgggtgcatgacgagcagcgatgagttgcggaggaagtatgctcgtgagagccgtcgtttcaatgcagagaaaatgtcagtatacaaggaactgtttcctgaggagtatcaaaaggatttggaggaattgaagcgagaggacagtgagaaaaacggtcgtacttctggaagtgctggttgtggtgcgaatacgaaaggaggaggtgtgatggaatcgcaagaaaaagaacaatggcgtgggttattcccggcacttttgggactttttgctgtgttaatgggagcctacttttggccatggtaa

SEQ ID NO: 33 - DNA, 678 bp, synthetic construct
  source 1..678: mol_type = other DNA
atgccaagcacacccaccccgcagtgtgtgcggcggttgcaaaaggagctttccgccctatgccgagaggccgagtcgttttttttcacccgtccctcagcaaagagtattctggtttggtatttcgtcatcaagggtcctgcggataccccttatgaaggcggtcgctactttggcaagctgaatttcccccccgactatccaatgaaaccgcctgagattatcattttgacgccaaatggacgttttgagaccaacaagagcatttgtctcaccatgagcaattatcatccggagaattggagccctttgtggggggtccgcaccattcttacggggttgctctctttcatggtgggagacgaactcactactgggtgcatgacgagcagcgatgagttgcggaggaagtacgctcgtgagagccgtcgtttcaatgcagagaaaatgccagtatacaaggaactgtttccagaggagtatcagaaggacttggaggaattgaagcgagaggacaatgagaaaaacggtcgtatttctggaagtgctggctgtggtgcgaatacgaaaggaggaggtgtgatggaatcgcaagaaaaagagcaatggcgtgggttattcccggcacttttgggactttttgctgtgttaatgggagcctacttttggccatggtaa

SEQ ID NO: 34 - DNA, 339 bp, synthetic construct
  source 1..339: mol_type = other DNA
atgtccatgaagtacctcgccgcatacgctcttgcttcgctgaacaaaccaacgccaggcgccgccgatgtggaggccatctgcaaggcctgcggtatcgaagttgagagcgacgcactctcgtttgtcatggaatccattgccggccggagcgttgccactctcgtggcggagggcgcggcgaagatgagcgctgttgccgtctccgctgctcctgctgccggtggtgcagccgctcctgctgctgctgctggcggtgccgccgcccctgccgctgctgacgccaagaaggaagaagaggaggaggacgatgacatgggctttggtctgtttgactaa

SEQ ID NO: 35 - DNA, 798 bp, synthetic construct
  source 1..798: mol_type = other DNA
atggcgccaccggctgatatgaggggggcgttgagagaggtgttgggagccatgcagaaggcgcaggagtatgctgacgaggctaaccggcactgcgtgcaggcaagaatgagcgctgagagtgcgcgggagcatgaagagggggctaagaatgctttgaggaagctcggctctgaggctacaaggatgagcagggcgctgcagcaagcggacgaggctgtgaaattggccgatgctgccgtggccgaatgcaaggcggcggaggaggctgcacaggcggcggggataatgacgcttgatgccgttggggaggtgctgaagcatctgaaggacgagaagaccaaggttggaagtggaccggagctgttgaagagggcggcggagcagactgtgctttctctggagaaggcaaaggaggcggaggcggaggctgagaaggcggcagcggcggcgcagaaaacccgggaagcagcagagaaggcagcagcggcgcggaccttggcacaagatgttgccgcaacggccagtgcgctgctgcggcagcgggagaaggaggaggagaggcgaagagcgagggaccaggaggtggcggaggccgcgaagaaggctgccgttgctgaggtgatgaaaaaatttgctgcgaaggggaatgacacagcgcctggcaggaattccacatccacccgctttcaaaggacgaggccacgggtggatggcggcggcatcccattgcttttgcgtgcaccgcttctgatgcttgctgccgtggcatccgttttcggcttcttatcgtgctag

SEQ ID NO: 36 - DNA, 1035 bp, synthetic construct
  source 1..1035: mol_type = other DNA
atgacgcgtaataggctttttttccctctgcttcttctactctcctgcagcgtaattgtcggcgcaaatgcaacagaaaagaaagcgtcaacgccaaggaaagcagagggagtgcagccgcaatcggtctcaccgtcttcgtcgtttccaggggatgggacgggtgtgccgctcaaattggaactgggggaactgagggacaaagcattgctggcagcaaaggatgcttttggcaatacgacaggggcggcaatgcaatgcatgcaggccaagacggatgtcgaagagaccaagaaatacgccgaagaggcgaaaaagctttttgataagattggcggggactatgtgtcaaaaagtgctgctctggcggatgcagtgaaagctagcaccgacgccgaagaggcgctgaaaagctgtgtggaggcggaaaaggccgctgttgatgctgataccgcggttttagctgctgtcctggaggtgctgcaacattccaagttttggcgaagggacactgcagtttcgactgaaaaattggcgaatgtcagtaaacattcggcgaacgccacaaatgaggcgcaaaaggcagggattcaagcgtcgaaggcggcagaagcggcgaagagggcagcggagtcgaaaaaaaaagctgcagcagctctggatacggtcaaggaagtcgttgcgatggccgagatgttgaaggaaaagtttttcgagaatgagaggctgcaaaaggaaaaacatgaggctcaattggaagccgaaagacagttcattcaggaagaggtacagaagaaggaggcggaggccgaaaaggcactcaatcgcgctgctgcggctgataaacgtgtcgccgagttggaacttgccagacaaaagcagagcaaagagcaggggaatgaaggaagaggccataggcgagtcagacgcagtgggagtgacagcagcagcaactatgcgcctgcatatgaaccacggctactgttactgcctctgctttctttcacactgttctgttttgttgcatggtgctag

SEQ ID NO: 37 - DNA, 603 bp, synthetic construct
  source 1..603: mol_type = other DNA
atgcctgccaaaaccgccgtttcgaaggctgctgcgcccaaaaaggccgctgcgcccaagaaggccgctgcaccacaaaaggctgctgcgcccaagaaggctgctgcgcccaagaaggctgctgcaccccaaaaggctgctgtcgccaagaaggccgtcagggaggcccccaaaaagggtgtcaagaagaccgccaagaagggcgcgccggccgctatgacgaaggtggtgaaggtcacgaagcgcaaggcgtacacccgcccgcagttccgtcgtccgcacacgtaccggaggccgtcgatccccaagccgagcaacaacatgagtgcgattcccaacaagtgggatgcgtttcgtgtgatccgctacccgctgaccaccgacaaggcgatgaagaagattgaggagaacaatacgctgaccttcattgtggactcgaacgccaacaagacggaaatcaagaaggccatgcgcaagctctaccaggtgaaggccgtgaaggtgaacaccctcatccgaccggacggccttaagaaggcgtacatccgcctctccgcctcgtacgacgccctcgagacagccaacaagatgggtctgctgtag

SEQ ID NO: 38 - DNA, 822 bp, synthetic construct
  source 1..822: mol_type = other DNA
atgaccaagaagcacctgaagcgcctttatgcccccaaggactggatgctgagcaagctcacgggcgtgttcgctccacgtccccgtgctggaccccacaagctgcgtgagtgcatgactcttatgatcatcatccgcaatcgtctgaagtatgcgctgaacgccgccgaggctcagatgattctccgtcagggccttgtgtgcgtggacggtaagccccgcaaggacaccaagtatccggttggcttcatggacgttgtggagatcccacggaccggggatcgtttccgcattctgtacgacgtgaagggccgctttgccctcgtgaaggttggcgaggctgaggggaacatcaagctcctgaaggtggagaacgtctacacaagcactggtcgcattcctgttgccatgacacacgacggtcaccgcattcgttaccccgacccccgcacccaccgtggcgacaccctggtgtacaacctgaaggagaagaaggtggtggacctcatcaagtccagcaacggcaaggtggtgatggtcaccggcggcgcgaaccgcggccgtattggcgagatcatgtcgattgagcgccaccctggtgcgttcgacattgcacgcctgaaggatgcggcgggacacgagtttgctacccgagcgtccaacatttttgtgattggcaaggacatgcagagcgttcctgtgacgctgccgaagcaacagggtctccgcatcaacgtgattcaggagcgtgaggagaagcttatcgctgctgaggcacgcaagaatatgcagactcgcggcgtacgcaaggcccgcaaatag

SEQ ID NO: 39 - DNA, 579 bp, synthetic construct
  source 1..579: mol_type = other DNA
atgatgcgttttacccggttccttgtcgttgcagcaaagcggagtgccaccagcgccaaactcggtaagagtgttggactcaccgcggcgctgagtcccaagcaaaggtcccttccccgcgtctcagtgacgaagttgatgaagcccagcgggagcgggaaacacgttacgtcgtcattcttgttgaaggacaagaagaaggtggccaccgcaaaagttgctgtgccgccgaaaaagaagagggctttaaaggtgaggaagggccgcagcagcggcaaaaaggccgcggctctctatgtgcgcttttatcacgccttgaagaagagcggacttgtgaaggggaagcgacgcatgcagaaaacgggtgagctgtggcgtgccacaaagaaggcgaaggacttcaagaagcgcgttgaggcggcgatgaggcttgcaaagaagggacaaaaaagcagggctcgtaagctgaaggcgcagaagaaggcaaagggcaaaaagtcggcgaagggcgtcaggagggtctaccggagggtcagcaggaagaagactgtcacgagcaccgtgccgcctctcccttaa

SEQ ID NO: 40 - DNA, 657 bp, synthetic construct
  source 1..657: mol_type = other DNA
atgccgaagggaaaaaacgcgatcccccacgtgcaccagaggaagcactggaacccgtgctcttcccagaagggtaatgtgaaggttttcctcaaccagcccgcacagaagctgcgccgtcgccgcctacgtcttttgaaggcgaagaagacgttcccacgcccactcaaggcgctgcgcccgcaggtgaattgccccacggtgcgtcacaacatgaagaagcgcctgggccgtggctttaccgttgaggagctgaaggctgccggcatcaaccctcgttttgccccgacgattggcatccgtgtggatcgtcgccgcaagaacaagagcgaggagggcatgagcatcaacatccagcgcctgaagacgtacatgagcaagctggtgctcttccccatgagctacaagaacgtgcagaagggcgaggccactgaggaggaggtgaagtctgccactcaggaccgcacacgctttggtactgcggctgttggtggttttgtgacgcctgctcccgaggcaccacgcaaggtgacagaggaggagcgcacaaagaacgtgtacaagttcctcaagaagaaccacagcgctgttcgcttctttggcattcgcagggcacgtcaggaacgcagggaggccaaggagaacgagaagaagtaa

SEQ ID NO: 41 - DNA, 1125 bp, synthetic construct
  source 1..1125: mol_type = other DNA
atggatacgacgctttacagtgaggtgaatcgtctcgaacgcggtgactttcttctttttcactgtgtgcagctctcacaacacgagcgtgacgtgcagcggtacttctttggatgctactttccgcgctggcgtgggttctacctggaggaggtgagggatatgccgggccctctaggctacaaggtgcagcgacactttcctgcgtatccctttgacgtgtatctgaaggacaatggtgaacactttctcacggatgacttccaggagggttctatattcactttgggagcctcgcaaaatcagcgtgacggcgactcgaagcgatataaagtagtgcactgcgacgatagtcgtttgcgcacgcgcacgggtacgactcttgcagacattggcaatgacatcacgacgaagttgaatcaaacacaccgtgtccctggcgaggtgatagatctcctgcgtgagattagagatgcgtatgttgtgtatgccggcaatggcattcctgagattggtatcaaggcaatgggacgtcactttcgccacgtcagcgaggatggaaagcggtggatgtcgttggagaacattggaaagcttgttcgtgactctcgtgccttttccaacacattgtcatttgaggacacgcagaggacgaattccacgattagcaataatgcaaggagcattcatgaagcctttccgcagaatgaagaaggctgcattgactatgatttattcatggactacgttcgtggaccgatgagccaaaaaaggaaggatgccgtctgggaaatattccgcaagcttgactttgatggagacggctacctcaacatcttagacattcaggcccgctacaatgcgcagcagcaccctgtggtggcggtggagagactcttctccgcggacaaactgctcaagggcttcctcaccgtttgggatgagaacaaacaatacgggttgatcccatacgccgagtttatcgactactacaacggcgtcagcgcggtaattgcggacgactacatcttttttgatattctccggaatcaatggaaggtcatgcgtgactggggagggacggtggggacgaggggagggaattgtgagttcccgacgatgtaa

SEQ ID NO: 42 - DNA, 1125 bp, synthetic construct
  source 1..1125: mol_type = other DNA
atggatacgacgctttacagtgaggtgaatcgtctcgaacgcggtgactttcttttttttcactgtgtgcagctctcacaacacgagcgtgacgtgcagcggtacttctttggatgctactttccgcgctggcgtgggttctacctggaggaggtgagggatatgccaggccctctaggctacaaggtgcagcgacactttcctgcgtatccctttgacgtgtatctgaaggacaatggtgaacactttctcacggatgacttccaggagggttctatattcactttgggagcctcgcaaaatcagcgtgacggcgagtcgaagcgatataaagtagtgcactgcgacgatagtcgtctgcgcacgcgcacgggcacgactcttgcagacattggcaatgacatcacgacgaggttgaatcaaacacaccgtgtccctggcgaggtgatagatctcctgcgtgagattagagatgcgtatgttgtgtatgccggcaatggcattcctgagattggtatcaaggcaatgggacgtcactttcgccacgtcagcgaggatggaaagcggtggatgtcgttggagaacattggaaagcttgttcgtgactctcgtgccttttccaccacattgtcatttgaggacacgcagaagacgaattccacgattagcaataatgcaaggagcattcatgaagcctttccgcagaatgaagaaggctgcattgactatgatttattcatggactacgttcgtgggccgatgagccaaaaacggaaggatgccgtctgggaaatattccgcaagcttgactttgatggagacggctacctcaacatcttagacattcaggcccgctacaatgcgcagcagcaccctgtggtggcggtggagagactcttctccgcggacaaactgcttaagggcttcctcaccgtttgggatgagaacaaacaatacgggttgatcccatacgccgagtttatcgactactacaacggcgtcagcgcggtaattgcggacgactacatcttttttgatattctccggaatcaatggaaggtcatgcgtgactggggagggacggtggggacgaggagagggaagagtgaggtttcgacgatgtaa

SEQ ID NO: 43 - DNA, 348 bp, synthetic construct
  source 1..348: mol_type = other DNA
atggccgataaggttgaagcgaacgacacgctggcgtgcacctacgccgccctcatgctcagcgacgcgggtctgcccatcacggcggagggcattgaggccgcgtgtgtggctgccggtctgaaggtgcgcaacaccctgcccgttatttttgctcgctttctcgaaaagaagccgctggagactctctttgccgctgccgctgctacggcacctgcagagggcgccgctgctgctcctgccgctggcagtgccgcccctgccgccgcagctgccggtgctgcgccagaaaaggacacaaaggaggaggaggaagacgacgatatgggttttggcttgtttgactag

SEQ ID NO: 44 - DNA, 348 bp, synthetic construct
  source 1..348: mol_type = other DNA
atggccgataaggttgaagcgaacgacacgctggcgtgcacctacgccgccctcatgcttagcgacgcgggtctgcccatcacggcggagggcattgaggccgcgtgtgtggctgccggtctgaaggtgcgcaacaccctgcccgttatttttgctcgctttcttgaaaagaagccgctggagagtctcttcgctgctgccgctgctacggctcctgcagagggcgccgctgctgttcctgccgctggcagtgccgcccctgctgccgcagctgccgctgctgcgccagcaaaggacacaaaggaggaggaggaagacgacgatatgggttttggcttgtttgactag

SEQ ID NO: 45 - protein, 6 aa, synthetic construct
GSGSGS

SEQ ID NO: 46 - protein, 6 aa, synthetic construct
ASGGGS

SEQ ID NO: 47 - protein, 15 aa, synthetic construct
GGGGSGGGGSGGGGS

SEQ ID NO: 48 - protein, 20 aa, synthetic construct
GGGGSGGGGSGGGGSGGGGS

SEQ ID NO: 49 - protein, 8 aa, synthetic construct
DYKDDDDK

SEQ ID NO: 50 - protein, 6 aa, synthetic construct
YPYDVP

SEQ ID NO: 51 - protein, 10 aa, synthetic construct
ILKKATAYIL

SEQ ID NO: 52 - protein, 10 aa, synthetic construct
EQKLISEEDL

SEQ ID NO: 53 - protein, 373 aa, synthetic construct
MYKFGGEAKDLRNIYNFGDMSQRETEPPKDLSLAENKAYLVDVEVHSDNNEEEMGNRESQQPNSRVSPTAHGVPQSSAFFPEFSHSSGPDVPRKPSMESTSEQKNSKEKQKENSKVKIAKEVLGINKKNTSGMSPEEKERVLLEERWKRAMAEENRLNALEEQVTHREQATNSSGLLPNFPPKFLCIKPLVHHDISSVPEVRRQFVRFNFINWIATCVLLLVNMIIVIAVVFASHKEDAKKFHTSQNTVLAILYLMGAPLSFIVWYWQIYSACSTGRHTKHLLALSGLVIALAFDIFMIVGRTNYAACGVSLAIDISKTKSKLAVLPVIVVLFFWVVEAVILCYCIAKQWMYYRLDVNAQEEVRRQMRNVIGI

SEQ ID NO: 54 - protein, 243 aa, synthetic construct
MGKKYAQLETLHNVNGRVVIVGDIHGCLAQLEDILSVTDFARGRDQLITAGDMVNKGPDSFGVVRLLKSLGARGVIGNHDAKLLKLRKKIRKHGTLHGTNSQSSLAPLAMSLPQDVEEYLLQLPHILRIPAHNILVVHAGLHVQHPLERQLVKEVTTMRNLILQDDGLYRASEDTTDGVPWASLWQGPETVVFGHDARRGLQRHPHAIGLDTRCVYGGELTALVCPGEHLVSVPGWTSNRSKV

SEQ ID NO: 55 - protein, 373 aa, synthetic construct
MYKFGGEAKDLRNIYNFGDMSQRETEPQKELSLAENRAYLVDVEVHSDNNEEEMGHRESQQPNSRVSPTAQGVPQSSAFFSEFSHSSGIDFPQKPSMENTSDQKNSNEKPKENSKVKIAKEVLGINKKNTSGMSPEEKERVLLEERWKRAMAEENRLNALEEQVTHREQATNSSGLLPNFPPKFLFIKPLVHHDISSVPEVRRQFVRFNFINWIATCVLLLVNMIIVIAVVFASHKEDAKKFNTSQNTVLAILYLVGAPLSFIVWYWQIYSACSTGRHTKHLLALSGLVIALAFVIFMIVGRTNYAACGVSLAIDISKTKSKFAVLPVIIVLFFWVVEAVILCYCIVKQWIYYRLDVNAQEEVRRQMRNVIGI

SEQ ID NO: 56 - protein, 243 aa, synthetic construct
MGKKYAQLETLHNVNGRVVIVGDIHGCLAQLEDILSVTEFARGRDQLITAGDMVNKGPDSFGVVRLLKSLGARGVIGNHDAKLLKLRKKIRKHGALHGKNSQSSLAPLAMSLPQDVEEYLSQLPHILRIPAHNILVVHAGLHVQHPLERQLVKEVTTMRNLILQDDGLYRASEDTTDGVPWASLWQGPETVVFGHDARRGLQRYPHAIGLDTRCVYGGELTALVCPGEHLVSVPGWTSNRSKV

SEQ ID NO: 57 - protein, 282 aa, synthetic construct
MLAVMVMRPFLCALLFFALCRCFPNSACAASATANNATENASAMAPPADMRGALREVLGAMQKAQEYADEANRHCVQARMSAESAREHEEGAKNALRKLGSEATRMSRALQQADEAVKLADAAVAECKAAEEAAQAAGIMTLDAVGEVLKHLKDEKTKVGSGPELLKRAAEQTVLSLEKAKEAEAEAEKAAAAAQKTRDAAEKAAAARTLAQDVAATASALLRQREKEEERRRARDRVRAYVGNERAENAMRVAWLDWVECCVAVLVNEGAEGGNGVFFPIF

SEQ ID NO: 58 - protein, 310 aa, synthetic construct
MLAVMVMRPFLCALLFFALCCCFPNSVCAADDTAANTTEDVNASAIPTNMKEAFDWAFKAMFKAREEVDEASQHCVQAKLSAAKAAGLEREAEMALKKLGAEAMTLSKALRDARGANSEAKAAVTECEAAEEAAQQAEIATLDAAYEVLNHVKTDRRSKNSKTEGLLDEAAKHTAIAVKKAKEAEAESEKAAAAARKTLEAAEKAAAARTLAQDVAATASALLRQREREEERRRAKDREAAEAAKKAAVAEVMKKFAAKKGNDAASGRNSTTTRIQRTRPRVDGGGIPLLLRAPLLMLAAVASVFGFLSC

SEQ ID NO: 59 - protein, 309 aa, synthetic construct
MLAVMVKRPFLCAPLFFALCCCFPNSVCAASATANNATENVSSMAPPADMRGALREVLGAMQKAQEYADEANRHCVQAGMSAKNAREHEEGAKNALRKLGSEATRMSRALQQAEEAVKLADAAVAECKAAEEAAQAAGIMTLDAVGEVLKHVKDEKTKVGSGPELLKRAAEQTVLSLEKAKEAEAETEKAAAAAQKTREAAEKAAAAQTLAQDVAATAIALLRQREKEEERRRARDREEAEAAKKAAVAEVMNKFAAKKGNDAAPGRNSTATRIQRTRPRVDGGGIPLLLRAPLLMLAAVASVFGFLSC

SEQ ID NO: 60 - DNA, 1122 bp, synthetic construct
  source 1..1122: mol_type = other DNA
atgtataagtttggaggtgaggcgaaggatcttagaaacatttataattttggcgatatgagccaacgagaaacggagccaccgaaggacttatcattagcagaaaataaagcttatttggtggatgtagaggtgcattctgataataatgaagaggaaatggggaatcgtgagagccaacaacccaattccagggtctcaccgacggctcatggagttcctcaatcctccgcgttttttccggaattttcacactcttctggacctgatgttcctcgaaaaccctcaatggaaagtacttcggaacaaaaaaactcaaaggaaaaacaaaaggagaatagtaaagtaaagattgcaaaagaagttttaggaataaacaagaaaaatacctctgggatgtcacctgaagagaaggagcgtgtattacttgaagaaaggtggaaaagagccatggcagaggagaatcgtttgaacgcactcgaagagcaagtaactcatcgtgagcaagcgactaattcttcaggccttcttccaaacttccctcccaagttcttatgtattaagccacttgtacaccatgatatttcgagtgttcccgaggtgagaagacaatttgtcaggtttaattttataaattggattgccacatgtgttttgctccttgtcaatatgattattgttattgctgtggtatttgcatctcataaagaagatgcaaaaaaattccatacttctcaaaacactgttttagccattttgtacctgatgggagcccctttaagctttattgtttggtattggcagatttattctgcttgttccacaggacgtcatactaaacatcttttggctctaagtgggttggttatagctcttgcctttgatatatttatgattgttggtcggacaaactatgctgcatgcggtgtatctcttgcaatagatatatcgaaaacgaaaagtaagcttgccgtattgcccgtgatcgttgttctttttttctgggttgtagaggctgttatattgtgttactgtatcgcaaaacagtggatgtactatcggttggatgtgaacgcgcaagaagaagtgagacgccagatgcggaatgtgattggaatttag

SEQ ID NO: 61 - DNA, 732 bp, synthetic construct
  source 1..732: mol_type = other DNA
atgggaaaaaaatacgcacagttagagactctccacaacgtgaatgggcgggttgtcattgtgggcgacattcatggctgccttgcccaactggaggacattttatcagtcacagactttgcgaggggaagggatcagttaatcaccgctggggacatggtgaacaaagggccagactcgtttggcgttgtgcgtctgctgaagagccttggagcacgcggtgtgattggcaatcatgacgccaagcttctcaaacttcggaaaaagatacgaaaacatgggacgctgcacgggacgaatagccaatcgagtttggccccgcttgccatgtcgctaccgcaggatgttgaagagtatttattacaactgccgcatattctccgcattcctgcacacaacattctggtggtacatgcgggccttcacgttcaacacccactcgagcggcaattggttaaggaggtcactacgatgcgcaacctcattttgcaggatgacgggctgtacagggcatctgaggatacaacggacggtgtgccctgggcatcgctgtggcagggtccggagactgttgtctttggccacgacgccagacgaggcctccaacgccaccctcatgcgatcgggttggacactcggtgtgtgtatggcggggagctcactgctcttgtgtgtcccggtgaacacctcgtttccgtgcctggatggacttccaatagatcgaaggtgtga

SEQ ID NO: 62 - DNA, 1122 bp, synthetic construct
  source 1..1122: mol_type = other DNA
atgtataagtttggaggtgaggcgaaggatcttcgaaacatttataattttggcgatatgagccaacgagaaacggagccacagaaggaattatcattggcagaaaatagagcttatttggtggatgtagaggtgcattctgataataatgaagaggaaatggggcatcgtgagagccaacaacccaactccagagtctcaccgacggctcagggagttcctcagtcctccgcgtttttttcggaattttcacactcttctggaattgattttcctcaaaaaccctcaatggaaaatacttcggaccaaaaaaactcaaacgaaaaaccaaaggagaatagtaaagtaaagatcgcaaaagaagttttaggaataaataagaaaaatacctctgggatgtcacctgaagagaaggagcgtgtattacttgaagaaagatggaaaagagccatggcagaggagaatcgtttgaacgcactcgaagagcaagtaactcatcgtgagcaagcgactaattcttcaggtcttcttcccaacttccctcccaagttcttatttattaagccacttgtacaccatgatatttcgagtgttcccgaggtcagaagacaatttgtcaggtttaattttataaattggatcgccacatgtgttttgctccttgtcaatatgattattgttattgctgtggtatttgcatctcataaagaagatgcaaaaaaattcaatacttctcaaaacactgttttagccattttgtacctggtgggagcccctttaagctttattgtttggtattggcagatttattctgcttgttccacaggacgtcataccaaacatcttttggctctaagtgggttggttatagcacttgcctttgttatatttatgattgttggtcggacaaactatgctgcatgcggtgtatctcttgcaatagatatatcgaaaacgaaaagcaagtttgccgtattgcccgtgatcattgttctttttttctgggttgtagaggctgttatattgtgttactgtatcgtaaaacagtggatctactatcggttggatgtgaacgcgcaagaagaagtgaggcgccagatgcggaatgtgattggaatttag

SEQ ID NO: 63 - DNA, 732 bp, synthetic construct
  source 1..732: mol_type = other DNA
atgggaaaaaaatacgcacagttggagactctccacaacgtgaatgggcgggttgtgattgtaggcgacattcatggctgccttgcccaactggaggacattttatcagtcacagaatttgcgaggggaagggatcagttaatcaccgctggggacatggtgaacaaagggccagactcgtttggcgttgtgcgtctgctgaagagccttggagcacgcggtgtgattggcaatcatgacgccaagcttctcaaacttcggaaaaagatacgaaaacatggggcgctgcacgggaagaatagccaatcgagtttagccccgcttgccatgtcgctaccgcaggatgttgaagagtatttatcacaactgccgcatattctccgcattcccgcacacaacattctggtggtacatgcgggccttcacgttcaacacccgcttgagcggcaattggttaaggaggtcactacgatgcgcaacctcattttgcaggatgacgggctgtacagggcatctgaggatacaacggacggtgtgccgtgggcatcgctgtggcagggtccggagactgttgtctttggccacgacgccagacgaggcctccaacgctaccctcatgcgatcggattggacactcggtgtgtgtatggcggggagctcactgctcttgtgtgtcccggtgaacacctcgtttccgtgcctggatggacttccaatagatcgaaggtgtga

SEQ ID NO: 64 - DNA, 849 bp, synthetic construct
  source 1..849: mol_type = other DNA
atgctggcggtgatggtgatgcgccctttcttgtgtgccctgctattttttgcgctctgccgctgctttcctaattccgcgtgtgcggcttctgccacagccaataatgccactgagaatgccagtgctatggcgccaccggctgatatgaggggggcgttgagagaggtgttgggagccatgcagaaggcgcaggagtatgctgacgaggctaaccggcactgcgtgcaggcaagaatgagcgctgagagtgcgcgggagcatgaagagggggctaagaatgctttgaggaagctcggctctgaggctacaaggatgagcagggcgctgcagcaagcggacgaggctgtgaaattggccgatgctgccgtggccgaatgcaaggcggcggaggaggctgcacaggcggcggggataatgacgcttgatgccgttggggaggtgctgaagcatctgaaggacgagaagaccaaggttggaagtggaccggagctgttgaagagggcggcagagcagactgtgctttctctggagaaggcaaaggaggcggaggcggaggctgagaaggcggcagcggcggcgcagaaaacccgggacgcagcagagaaggcagcagcggcgcggaccttggcacaagatgttgccgcaacggccagtgcgctgctgcggcagcgggagaaggaggaggagaggcgaagagcgagggacagggtgagggcttacgttggaaatgaacgcgccgagaatgccatgagggttgcgtggctggactgggtggagtgttgtgttgcagttcttgtcaatgaaggagctgaaggtggcaatggcgttttttttcctatattttaa

SEQ ID NO: 65 - DNA, 933 bp, synthetic construct
  source 1..933: mol_type = other DNA
atgctggcggtgatggtgatgcgccctttcttgtgtgccctgctgttttttgcgctctgctgctgctttcctaattccgtgtgtgcggcggatgatacagctgctaataccactgaggatgtcaatgcttcggcgataccgactaatatgaaagaagcttttgactgggcattcaaggcgatgtttaaggcgcgggaggaggtggatgaggccagccagcattgtgtgcaagctaaactaagtgcggcgaaggcagctgggcttgagagagaagcagagatggctttaaaaaagcttggcgcggaggctatgacactgagcaaggcattacgagatgcgaggggagccaacagtgaagctaaggctgctgtgaccgaatgcgaggctgcagaggaggctgcacaacaagcagagattgcgacgctcgatgccgcatacgaggtgctgaaccatgttaagaccgataggaggagcaagaattccaaaacagagggtcttttggacgaggcggcaaagcatactgcaattgccgtaaagaaggcaaaggaggcggaggcggagtctgagaaggcggcagcggcggcgcggaaaaccctggaagcagcagagaaggcagcagcggcgcggaccttggcacaagacgttgccgcaacggccagtgcgctgctgcggcagcgggagagggaggaggagagacgaagagcgaaggaccgggaggcggcggaggccgcgaaaaaggctgccgttgctgaggtgatgaagaaatttgctgcgaagaaggggaatgacgcggcgtctggcaggaattccacgaccacccgcattcaaaggacgagaccgcgggtggatggcggcggcattccattgcttttgcgtgcaccgcttctgatgcttgctgccgtggcatccgttttcggcttcttatcgtgctag

SEQ ID NO: 66 - DNA, 930 bp, synthetic construct
  source 1..930: mol_type = other DNA
atgctggcggtgatggtgaagcgccctttcttgtgtgccccgcttttttttgcgctctgctgctgctttcctaattccgtgtgtgcggcttctgccacagccaataatgccactgagaatgtcagttctatggcgccaccggctgatatgaggggggcgttgagagaggtgttgggagccatgcagaaggcgcaggagtatgctgatgaggctaaccggcactgcgtgcaagcaggaatgagcgctaagaatgcgcgggagcatgaagagggggctaagaatgctttgaggaagctcggctctgaggctacaaggatgagcagggcgctgcagcaagcggaggaggctgtgaaattggccgatgctgccgtggccgaatgcaaggcggcagaggaggctgcacaggcggcggggataatgacgcttgatgccgttggggaggtgctgaagcatgtgaaggacgagaagaccaaggttggaagtggaccggagctgttgaagagggcggcggagcagactgtgctttctctggagaaggcaaaggaggcggaggcggagactgagaaggcggcagcggcggcgcagaaaacccgggaagcagcagagaaggcagcagcggcgcagaccttggcacaagatgttgccgcaacggccattgcgctgctgcggcagcgggagaaggaggaggagaggcgaagagcgagggaccgggaggaggcggaggccgcgaagaaggctgccgttgctgaggtgatgaataaatttgctgcgaagaaggggaatgacgcagcgcctggcaggaattccacagccacccgcattcaaaggacgagaccacgggtggatggcggcggcattccattgcttttgcgtgcaccgcttctgatgcttgctgccgtggcatccgttttcggcttcttatcgtgctag

SEQ ID NO: 67 - protein, 226 aa, synthetic construct
MSSKQQLACTYAALILADSGKTDMDSLLKVTKAAGVDVSKGMASAFASILKNVDINDVLSKVSFGGVAPAAGGATAAPAAAAAAAAPAAAAAKKEEEEEDDDMGFGLFDGGGGSMSMKYLAAYALASLNKPTPGAADVEAICKACGIEVESDALSFVMESIAGRSVATLVAEGAAKMSAVAVSAAPAAGGAAAPAAAAGGAAAPAAADAKKEEEEEDDDMGFGLFD

SEQ ID NO: 68 - protein, 606 aa, synthetic construct
MVSLKLQARLAADILRCGRHRVWLDPNEASEISNANSRKSVRKLIKDGLIIRKPVKVHSRSRWRHMKEAKSMGRHEGAGRREGTREARMPSKELWMRRLRILRRLLRKYREEKKIDRHIYRELYVKAKGNVFRNKRNLMEHIHKVKNEKKKERQLAEQLAAKRLKDEQHRHKARKQELRKREKDRERARREDAAAAAAAKQKAAAKKAAAPSGKKSAKASAPAKAATAPAKATAAPAKAAAAPAKATAAPAKATAAPAKAAAAPAKATAAPAKAAAAPAKAAAAPAKAAAAPAKAAAAPAKATAAPAKAAAAPAKVAAAPAKAAAAPVGKKAGGKKGGGGSMAPPADMRGALREVLGAMQKAQEYADEANRHCVQARMSAESAREHEEGAKNALRKLGSEATRMSRALQQADEAVKLADAAVAECKAAEEAAQAAGIMTLDAVGEVLKHLKDEKTKVGSGPELLKRAAEQTVLSLEKAKEAEAEAEKAAAAAQKTREAAEKAAAARTLAQDVAATASALLRQREKEEERRRARDQEVAEAAKKAAVAEVMKKFAAKGNDTAPGRNSTSTRFQRTRPRVDGGGIPLLLRAPLLMLAAVASVFGFLSC

SEQ ID NO: 69 - protein, 589 aa, synthetic construct
MPAKSANKPASKPAAKPAAKPAAKAPAPKAAAPAPKAAAAAPKPAVRDAKQRSDAANHNGLYVKNWGQGSVDDARALFGTAGKVVGVRVRRRRYAIIFFENAAAVKKAIDLFNGKEFMGNVLSVVPAKTTPKPDPHANSSVVFVSPIFRASTTKKQILELFSGMKVLRLRTYRNNYAYVYLDTPAAAQRAVKEKNGAEFRGKQLRVALSTRSLAKDRARAERARLLIAAQKFNKRKNHTKGGGGSMTRNRLFFPLLLLLSCSVIVGANATEKKASTPRKAEGVQPQSVSPSSSFPGDGTGVPLKLELGELRDKALLAAKDAFGNTTGAAMQCMQAKTDVEETKKYAEEAKKLFDKIGGDYVSKSAALADAVKASTDAEEALKSCVEAEKAAVDADTAVLAAVLEVLQHSKFWRRDTAVSTEKLANVSKHSANATNEAQKAGIQASKAAEAAKRAAESKKKAAAALDTVKEVVAMAEMLKEKFFENERLQKEKHEAQLEAERQFIQEEVQKKEAEAEKALNRAAAADKRVAELELARQKQSKEQGNEGRGHRRVRRSGSDSSSNYAPAYEPRLLLLPLLSFTLFCFVAWC

SEQ ID NO: 70 - protein, 391 aa, synthetic construct
MAFCIISESRGMSLWDMLAWHRPKVTGVLLGTVLSVLTFFCLMKYTMVTFLCRILQLVLLAGVLLGFTNRWHLTSDDIHEAVNRLVDCATPRLVTALESMHQLVTWRDYRRSGLVTLVSFVVALLGNLVSDAAFLTFFLLLAFTVPAVYEKKKDLIDKWISAATAQVEKYMGKIKTKVEEATKKKEGGGGSMPAKTAVSKAAAPKKAAAPKKAAAPQKAAAPKKAAAPKKAAAPQKAAVAKKAVREAPKKGVKKTAKKGAPAAMTKVVKVTKRKAYTRPQFRRPHTYRRPSIPKPSNNMSAIPNKWDAFRVIRYPLTTDKAMKKIEENNTLTFIVDSNANKTEIKKAMRKLYQVKAVKVNTLIRPDGLKKAYIRLSASYDALETANKMGLL

SEQ ID NO: 71 - protein, 593 aa, synthetic construct
MPGKEVKKAAKPAAKTAAKPAAKSAAKPAAKPAAKPAAKTAAKPAAKTAAKPAKKPAVKPTVKPAAKAAAPYKKPAAISPFVARPKNFGIGHDVPYARDLSRFMRWPTFVTMQRKKRVLQRRLKVPPALHQFTKVLDRSSRNELLKLVKKYPSETRRARRQRLFDVATEKKKNPEAASKKAPLSVVTGLQEVTRTIEKKTARLVMIANNVDPIELVLWMPTLCRANKVPYAIVKDKARLGDAVGRKTATCVAITDVNAEDEAALKNLIRSVNARFLARSDVIRRQWGGLQLSLRSRAELRKKRARNAGKDAAAVMGGGGSMTKKHLKRLYAPKDWMLSKLTGVFAPRPRAGPHKLRECMTLMIIIRNRLKYALNAAEAQMILRQGLVCVDGKPRKDTKYPVGFMDVVEIPRTGDRFRILYDVKGRFALVKVGEAEGNIKLLKVENVYTSTGRIPVAMTHDGHRIRYPDPRTHRGDTLVYNLKEKKVVDLIKSSNGKVVMVTGGANRGRIGEIMSIERHPGAFDIARLKDAAGHEFATRASNIFVIGKDMQSVPVTLPKQQGLRINVIQEREEKLIAAEARKNMQTRGVRKARK

SEQ ID NO: 72 - protein, 436 aa, synthetic construct
MTTIGTYNEEGVNVDLYIPRKCHATNNLITSYDHSAVQIAIANVDANGVLNGTTTTFCIAGYLRRQAESDHAINHLAISKGIIRIKTGKKPRAKKLKNVKGLGVRGLPRGALQQRGARVLPTQRGVAQRGGAQKGNVRKLQPQPQKQRSQLNQRSQQQHGARPTRKEEGGRTQRGGRDAPQARKQQGRNEPQARRQQGRNEPQARRQQGRNEPQARKQQGRDAPQARKQQGRNAPRSQKAGGGGSMMRFTRFLVVAAKRSATSAKLGKSVGLTAALSPKQRSLPRVSVTKLMKPSGSGKHVTSSFLLKDKKKVATAKVAVPPKKKRALKVRKGRSSGKKAAALYVRFYHALKKSGLVKGKRRMQKTGELWRATKKAKDFKKRVEAAMRLAKKGQKSRARKLKAQKKAKGKKSAKGVRRVYRRVSRKKTVTSTVPPL

SEQ ID NO: 73 - protein, 444 aa, synthetic construct
MGIVRSRLHKRKITGGKTKIHRKRMKAELGRLPAHTKLGARRVSPVRARGGNFKLRGLRLDTGNFAWGTEAIAQRARILDVVYNATSNELVRTKTLVKNCIVVVDAAPFKLWYAKHYGIDLDAAKSKKTAQSTTEKKKSKKTSHAMTEKYDVKKASDELKRKWMLRRENHKIEKAVADQLKEGRLLARITSRPGQTARADGALLEGAELQFYLKKLEKKKRGGGGSMPKGKNAIPHVHQRKHWNPCSSQKGNVKVFLNQPAQKLRRRRLRLLKAKKTFPRPLKALRPQVNCPTVRHNMKKRLGRGFTVEELKAAGINPRFAPTIGIRVDRRRKNKSEEGMSINIQRLKTYMSKLVLFPMSYKNVQKGEATEEEVKSATQDRTRFGTAAVGGFVTPAPEAPRKVTEEERTKNVYKFLKKNHSAVRFFGIRRARQERREAKENEKK

SEQ ID NO: 74 - protein, 621 aa, synthetic construct
MYKFGGEAKDLRNIYNFGDMSQRETEPPKDLSLAENKAYLVDVEVHSDNNEEEMGNRESQQPNSRVSPTAHGVPQSSAFFPEFSHSSGPDVPRKPSMESTSEQKNSKEKQKENSKVKIAKEVLGINKKNTSGMSPEEKERVLLEERWKRAMAEENRLNALEEQVTHREQATNSSGLLPNFPPKFLCIKPLVHHDISSVPEVRRQFVRFNFINWIATCVLLLVNMIIVIAVVFASHKEDAKKFHTSQNTVLAILYLMGAPLSFIVWYWQIYSACSTGRHTKHLLALSGLVIALAFDIFMIVGRTNYAACGVSLAIDISKTKSKLAVLPVIVVLFFWVVEAVILCYCIAKQWMYYRLDVNAQEEVRRQMRNVIGIGGGGSMGKKYAQLETLHNVNGRVVIVGDIHGCLAQLEDILSVTDFARGRDQLITAGDMVNKGPDSFGVVRLLKSLGARGVIGNHDAKLLKLRKKIRKHGTLHGTNSQSSLAPLAMSLPQDVEEYLLQLPHILRIPAHNILVVHAGLHVQHPLERQLVKEVTTMRNLILQDDGLYRASEDTTDGVPWASLWQGPETVVFGHDARRGLQRHPHAIGLDTRCVYGGELTALVCPGEHLVSVPGWTSNRSKV

SEQ ID NO: 75 - protein, 621 aa, synthetic construct
MYKFGGEAKDLRNIYNFGDMSQRETEPQKELSLAENRAYLVDVEVHSDNNEEEMGHRESQQPNSRVSPTAQGVPQSSAFFSEFSHSSGIDFPQKPSMENTSDQKNSNEKPKENSKVKIAKEVLGINKKNTSGMSPEEKERVLLEERWKRAMAEENRLNALEEQVTHREQATNSSGLLPNFPPKFLFIKPLVHHDISSVPEVRRQFVRFNFINWIATCVLLLVNMIIVIAVVFASHKEDAKKFNTSQNTVLAILYLVGAPLSFIVWYWQIYSACSTGRHTKHLLALSGLVIALAFVIFMIVGRTNYAACGVSLAIDISKTKSKFAVLPVIIVLFFWVVEAVILCYCIVKQWIYYRLDVNAQEEVRRQMRNVIGIGGGGSMGKKYAQLETLHNVNGRVVIVGDIHGCLAQLEDILSVTEFARGRDQLITAGDMVNKGPDSFGVVRLLKSLGARGVIGNHDAKLLKLRKKIRKHGALHGKNSQSSLAPLAMSLPQDVEEYLSQLPHILRIPAHNILVVHAGLHVQHPLERQLVKEVTTMRNLILQDDGLYRASEDTTDGVPWASLWQGPETVVFGHDARRGLQRYPHAIGLDTRCVYGGELTALVCPGEHLVSVPGWTSNRSKV

SEQ ID NO: 76 - protein, 493 aa, synthetic construct
MGAPQIVYSALITNTTTIAVTVVVTYTMPNEMPPETLELLIQPGEEMLAPQKLVEDGIVTWTGYISKVAIQGGPSMSEPFPGVECPTRRYDFEVFMHAGVLRLFALGPAESSSDGGGGSMDTTLYSEVNRLERGDFLLFHCVQLSQHERDVQRYFFGCYFPRWRGFYLEEVRDMPGPLGYKVQRHFPAYPFDVYLKDNGEHFLTDDFQEGSIFTLGASQNQRDGDSKRYKVVHCDDSRLRTRTGTTLADIGNDITTKLNQTHRVPGEVIDLLREIRDAYVVYAGNGIPEIGIKAMGRHFRHVSEDGKRWMSLENIGKLVRDSRAFSNTLSFEDTQRTNSTISNNARSIHEAFPQNEEGCIDYDLFMDYVRGPMSQKRKDAVWEIFRKLDFDGDGYLNILDIQARYNAQQHPVVAVERLFSADKLLKGFLTVWDENKQYGLIPYAEFIDYYNGVSAVIADDYIFFDILRNQWKVMRDWGGTVGTRGGNCEFPTM

SEQ ID NO: 77 - protein, 493 aa, synthetic construct
MGSPKIVYSALIRNTTTISVTVLVTYSMPSEMPQETVQLLIPPGEEKEAPQKLVEEDTVTWTGFISKVAVEGGQSMSAPFLGVESPTRRYGFEVYMQAGMLRLLALGPVESSSDGGGGSMDTTLYSEVNRLERGDFLFFHCVQLSQHERDVQRYFFGCYFPRWRGFYLEEVRDMPGPLGYKVQRHFPAYPFDVYLKDNGEHFLTDDFQEGSIFTLGASQNQRDGESKRYKVVHCDDSRLRTRTGTTLADIGNDITTRLNQTHRVPGEVIDLLREIRDAYVVYAGNGIPEIGIKAMGRHFRHVSEDGKRWMSLENIGKLVRDSRAFSTTLSFEDTQKTNSTISNNARSIHEAFPQNEEGCIDYDLFMDYVRGPMSQKRKDAVWEIFRKLDFDGDGYLNILDIQARYNAQQHPVVAVERLFSADKLLKGFLTVWDENKQYGLIPYAEFIDYYNGVSAVIADDYIFFDILRNQWKVMRDWGGTVGTRRGKSEVSTM

SEQ ID NO: 78 - protein, 345 aa, synthetic construct
MPSTPTPQCVRRLQKELSALCREAESFFFTRPSAKSILVWYFVIKGPADTPYEGGRYFGKLNFPPDYPMKPPEIIILTPNGRFETNKSICLTMSNYHPENWSPLWGVRTILTGLLSFMVGDELTTGCMTSSDELRRKYARESRRFNAEKMSVYKELFPEEYQKDLEELKREDSEKNGRTSGSAGCGANTKGGGVMESQEKEQWRGLFPALLGLFAVLMGAYFWPWGGGGSMADKVEANDTLACTYAALMLSDAGLPITAEGIEAACVAAGLKVRNTLPVIFARFLEKKPLETLFAAAAATAPAEGAAAAPAAGSAAPAAAAAGAAPEKDTKEEEEDDDMGFGLFD

SEQ ID NO: 79 - protein, 345 aa, synthetic construct
MPSTPTPQCVRRLQKELSALCREAESFFFTRPSAKSILVWYFVIKGPADTPYEGGRYFGKLNFPPDYPMKPPEIIILTPNGRFETNKSICLTMSNYHPENWSPLWGVRTILTGLLSFMVGDELTTGCMTSSDELRRKYARESRRFNAEKMPVYKELFPEEYQKDLEELKREDNEKNGRISGSAGCGANTKGGGVMESQEKEQWRGLFPALLGLFAVLMGAYFWPWGGGGSMADKVEANDTLACTYAALMLSDAGLPITAEGIEAACVAAGLKVRNTLPVIFARFLEKKPLESLFAAAAATAPAEGAAAVPAAGSAAPAAAAAAAAPAKDTKEEEEDDDMGFGLFD

SEQ ID NO: 80 - protein, 343 aa, synthetic construct
KHLKDEKTKVGSGPELLKRAAEQTVLSLEKAKEAEAEAEKAAAAAQKTRDAAEKAAAARTLAQDVAATASALLRQREKEEERRRARDRVRAYVGNERAENAMRVAWLDWVEGGGGSNHVKTDRRSKNSKTEGLLDEAAKHTAIAVKKAKEAEAESEKAAAAARKTLEAAEKAAAARTLAQDVAATASALLRQREREEERRRAKDREAAEAAKKAAVAEVMKKFAAKKGGGGSKHVKDEKTKVGSGPELLKRAAEQTVLSLEKAKEAEAETEKAAAAAQKTREAAEKAAAAQTLAQDVAATAIALLRQREKEEERRRARDREEAEAAKKAAVAEVMNKFAAKKG

SEQ ID NO: 81 - DNA, 678 bp, synthetic construct
  source 1..678: mol_type = other DNA
atgtcctccaaacagcagcttgcctgcacctacgccgccctgattcttgccgatagcggcaagacggatatggacagcctgttgaaagtgacaaaggccgccggtgttgacgtcagcaaagggatggcctcggcgtttgccagcatcctcaagaacgttgacatcaacgacgtgctctccaaagtgagctttggtggtgttgctcctgctgccggtggtgccaccgctgctcctgctgctgctgctgctgccgccgcccctgccgccgccgccgcaaagaaggaagaggaagaggaagacgacgatatgggctttggtctgtttgacggaggtggtggctctatgtccatgaagtacctcgccgcatacgctcttgcttcgctgaacaaaccaacgccaggcgccgccgatgtggaggccatctgcaaggcctgcggtatcgaagttgagagcgacgcactctcgtttgtcatggaatccattgccggccggagcgttgccactctcgtggcggagggcgcggcgaagatgagcgctgttgccgtctccgctgctcctgctgccggtggtgcagccgctcctgctgctgctgctggcggtgccgccgcccctgccgctgctgacgccaagaaggaagaagaggaggaggacgatgacatgggctttggtctgtttgac

SEQ ID NO: 82 - DNA, 678 bp, synthetic construct
  source 1..678: mol_type = other DNA
atgagcagcaagcagcaactggcgtgcacctatgcggcgctgatcctggcggacagcggtaaaaccgacatggatagcctgctgaaggtgaccaaagcggcgggtgtggatgttagcaagggcatggcgagcgcgttcgcgagcatcctgaagaacgtggacattaacgatgtgctgagcaaagttagctttggtggcgttgcgccggcggcgggtggcgcgaccgcggcgccggctgctgctgcggcggcggcggcgccggcggcggcggcggcgaagaaagaggaagaggaagaggacgatgacatgggtttcggcctgtttgacggtggcggtggcagcatgagcatgaagtacctggcggcgtatgcgctggcgagcctgaacaaaccgaccccgggtgcggcggatgttgaggcgatctgcaaggcgtgcggcattgaggtggaaagcgatgcgctgagcttcgttatggagagcattgcgggtcgtagcgtggcgaccctggttgcggagggtgcggcgaaaatgagcgcggtggcggttagcgcggctcctgcggcgggtggcgcggctgctccggcggcggcggcgggtggcgcggccgctccggcggcggcggacgcgaagaaagaagaggaagaggaagatgacgatatgggctttggcctgtttgat

SEQ ID NO: 83 - DNA, 1818 bp, synthetic construct
  source 1..1818: mol_type = other DNA
atggtgtcgctgaagctgcaggctcgtttggcggcggacattctccgctgcggtcgccaccgtgtgtggctggaccctaatgaggcctctgagatttccaatgcaaactcgcgcaagagcgtgcgcaagttgatcaaggatggtctgattattcgcaagcctgtcaaggtgcactcgcgctcccgctggcgccacatgaaggaggcgaagagcatgggccgccacgagggcgctgggcgccgcgagggtacccgcgaagcccgcatgccgagcaaggagctgtggatgcgccgtctgcgcattctccgccgcctgctgcgcaagtaccgcgaggagaagaagattgaccgccacatttaccgcgagctgtacgtgaaggcgaaggggaacgtgtttcgcaacaagcgtaacctcatggagcacatccacaaggtgaagaacgagaagaagaaggaaaggcagctggctgagcagctcgcggcgaagcgcctgaaggatgagcagcaccgtcacaaggcccgcaagcaggagctgcgtaagcgcgagaaggaccgcgagcgtgcgcgtcgcgaagatgctgctgctgccgccgccgcgaagcagaaggcagctgcgaagaaggccgctgctccctctggcaagaagtccgcgaaggcttctgcacctgccaaggccgctactgcacccgcgaaggccactgccgcacccgcgaaggctgctgctgcacccgcgaaggccactgctgcacccgcgaaggccactgctgcacccgcgaaggctgctgctgcacccgcgaaggccactgctgcacccgcgaaggccgctgctgcacccgcgaaggctgctgctgcacccgcgaaggccgctgctgcacccgcgaaggctgctgctgcacccgcgaaggccactgctgcacccgcgaaggccgctgctgcacctgcgaaggtcgctgctgcacccgcgaaggctgctgccgctcccgttggaaagaaggctggtggcaagaagggaggtggtggctctatggcgccaccggctgatatgaggggggcgttgagagaggtgttgggagccatgcagaaggcgcaggagtatgctgacgaggctaaccggcactgcgtgcaggcaagaatgagcgctgagagtgcgcgggagcatgaagagggggctaagaatgctttgaggaagctcggctctgaggctacaaggatgagcagggcgctgcagcaagcggacgaggctgtgaaattggccgatgctgccgtggccgaatgcaaggcggcggaggaggctgcacaggcggcggggataatgacgcttgatgccgttggggaggtgctgaagcatctgaaggacgagaagaccaaggttggaagtggaccggagctgttgaagagggcggcggagcagactgtgctttctctggagaaggcaaaggaggcggaggcggaggctgagaaggcggcagcggcggcgcagaaaacccgggaagcagcagagaaggcagcagcggcgcggaccttggcacaagatgttgccgcaacggccagtgcgctgctgcggcagcgggagaaggaggaggagaggcgaagagcgagggaccaggaggtggcggaggccgcgaagaaggctgccgttgctgaggtgatgaaaaaatttgctgcgaaggggaatgacacagcgcctggcaggaattccacatccacccgctttcaaaggacgaggccacgggtggatggcggcggcatcccattgcttttgcgtgcaccgcttctgatgcttgctgccgtggcatccgttttcggcttcttatcgtgc

SEQ ID NO: 84 - DNA, 1818 bp, synthetic construct
  source 1..1818: mol_type = other DNA
atggtgagcctgaagctgcaagcgcgtctggcggcggacatcctgcgttgcggccgtcaccgtgtgtggctggatccgaacgaagcgagcgagatcagcaacgcgaacagccgtaaaagcgttcgtaaactgattaaggacggtctgatcattcgtaaaccggtgaaggttcacagccgtagccgttggcgtcacatgaaagaggcgaaaagcatgggtcgtcatgagggtgcgggtcgtcgtgaaggcacccgtgaggcgcgtatgccgagcaaggaactgtggatgcgtcgtctgcgtatcctgcgtcgtctgctgcgcaaataccgtgaggaaaagaaaatcgaccgtcacatttaccgtgaactgtatgtgaaagcgaagggcaacgttttccgtaacaagcgtaacctgatggagcacatccacaaggttaagaacgaaaagaaaaaggagcgtcagctggcggagcaactggcggcgaaacgtctgaaggatgaacagcaccgtcacaaagcgcgtaagcaagagctgcgtaaacgtgaaaaggaccgtgagcgtgcgcgtcgtgaggatgcggctgctgcggcggcggcgaaacagaaagcggcggcgaaaaaggcggcggcgccgagcggtaaaaagagcgcgaaagcgagcgcgccggcgaaggcggcgaccgcgccggcgaaagcgaccgcggcgccggcgaaagcggcggcggcgccggcgaaggctactgcggcgccggcgaaggccactgctgctccggcgaaggctgctgctgctcctgctaaagctactgctgctcctgccaaggctgctgctgctcccgctaaagctgctgctgctcctgccaaagctgctgctgctcccgccaaagctgctgctgccccggcgaaggctacagcggctcctgcgaaggctgctgcggcgccggcgaaagttgcggctgctcctgctaaagccgctgcggcgccggttggcaaaaaggcgggtggcaaaaagggtggcggtggcagcatggctccgccggcggacatgcgtggcgcgctgcgtgaagtgctgggtgcgatgcagaaggcgcaagaatatgcggatgaggcgaaccgtcactgcgttcaggcgcgtatgagcgcggaaagcgcgcgtgagcacgaggaaggcgcgaaaaacgcgctgcgtaagctgggtagcgaggcgacccgtatgagccgtgcgctgcagcaagcggatgaagcggtgaagctggcggatgcggcggttgcggagtgcaaggcggcggaggaagcggcgcaagcggcgggtatcatgaccctggatgcggtgggcgaggttctgaaacacctgaaggatgaaaaaaccaaagtgggcagcggtccggaactgctgaaacgtgcggcggagcagaccgttctgagcctggaaaaagcgaaggaagcggaagcggaagcggaaaaggcggcggcggcggcgcaaaagacccgtgaagcggcggagaaagcggcggcggcgcgtaccctggcgcaggacgtggcggcgaccgcgagcgcgctgctgcgtcaacgtgaaaaggaagaggagcgtcgtcgtgcgcgtgatcaggaagttgcggaagctgcgaaaaaggcggctgtggcggaggttatgaaaaagtttgcggcgaagggtaacgacaccgcgccgggtcgtaacagcaccagcacccgttttcaacgtacccgtccgcgtgtggatggtggcggtattccgctgctgctgcgtgcgccgctgctgatgctggcggcggtggcgagcgttttcggttttctgagctgc

SEQ ID NO: 85 - DNA, 1767 bp, synthetic construct
  source 1..1767: mol_type = other DNA
atgcccgccaagtctgccaacaagcctgcatccaagcctgccgccaagcccgctgcgaagcctgccgccaaggctcccgcacccaaagctgctgcccctgctcccaaggctgctgcggctgcgcccaagccagctgtgagggacgcaaagcagcgctctgatgccgccaatcacaacggcttgtacgtgaagaactggggccagggttctgtggacgacgccagggcgctttttggcactgctgggaaggttgtgggtgtgagagtgcgtcgtcgccgttacgccattatcttctttgagaacgcagcggctgtgaagaaggccattgatcttttcaacgggaaagaatttatgggcaatgttttgtccgttgttcccgccaagacgactccgaagccggatccgcatgcgaactcctctgttgtgtttgtttccccgatattccgcgcgtcgactacaaagaagcagattcttgagcttttttcaggcatgaaggtactgcgcctgcgcacgtaccgcaacaactacgcatacgtctatctggacaccccagctgccgcgcaaagggctgtgaaggagaagaacggtgcagagttccgtggcaagcaactcagagttgccctctcgactcgttctcttgcgaaggacagggctcgtgcggagcgtgcaagacttcttatagccgcccaaaagttcaacaagagaaagaaccacacgaagggaggtggtggctctatgacgcgtaataggctttttttccctctgcttcttctactctcctgcagcgtaattgtcggcgcaaatgcaacagaaaagaaagcgtcaacgccaaggaaagcagagggagtgcagccgcaatcggtctcaccgtcttcgtcgtttccaggggatgggacgggtgtgccgctcaaattggaactgggggaactgagggacaaagcattgctggcagcaaaggatgcttttggcaatacgacaggggcggcaatgcaatgcatgcaggccaagacggatgtcgaagagaccaagaaatacgccgaagaggcgaaaaagctttttgataagattggcggggactatgtgtcaaaaagtgctgctctggcggatgcagtgaaagctagcaccgacgccgaagaggcgctgaaaagctgtgtggaggcggaaaaggccgctgttgatgctgataccgcggttttagctgctgtcctggaggtgctgcaacattccaagttttggcgaagggacactgcagtttcgactgaaaaattggcgaatgtcagtaaacattcggcgaacgccacaaatgaggcgcaaaaggcagggattcaagcgtcgaaggcggcagaagcggcgaagagggcagcggagtcgaaaaaaaaagctgcagcagctctggatacggtcaaggaagtcgttgcgatggccgagatgttgaaggaaaagtttttcgagaatgagaggctgcaaaaggaaaaacatgaggctcaattggaagccgaaagacagttcattcaggaagaggtacagaagaaggaggcggaggccgaaaaggcactcaatcgcgctgctgcggctgataaacgtgtcgccgagttggaacttgccagacaaaagcagagcaaagagcaggggaatgaaggaagaggccataggcgagtcagacgcagtgggagtgacagcagcagcaactatgcgcctgcatatgaaccacggctactgttactgcctctgctttctttcacactgttctgttttgttgcatggtgc

SEQ ID NO: 86 - DNA, 1767 bp, synthetic construct
  source 1..1767: mol_type = other DNA
atgccggcgaaaagcgcgaacaaaccggcgagcaaaccggcggcgaagccggcggcgaaaccggcggcgaaagcgccggcgccgaaagcggcggcgccggcgccgaaggcggcggcggcggcgccgaaaccggcggtgcgtgatgcgaagcagcgtagcgatgcggcgaaccacaacggcctgtatgtgaaaaactggggtcaaggcagcgttgatgatgcgcgtgcgctgtttggcaccgcgggcaaggtggttggtgtgcgtgtgcgtcgtcgtcgttacgcgatcattttctttgaaaacgcggcggcggtgaagaaagcgatcgacctgttcaacggcaaagagtttatgggtaacgttctgagcgtggttccggcgaaaaccaccccgaagccggatccgcacgcgaacagcagcgtggttttcgtgagcccgatctttcgtgcgagcaccaccaagaaacagattctggaactgttcagcggtatgaaggttctgcgtctgcgtacctatcgtaacaactacgcgtatgtgtatctggacaccccggcggcggcgcaacgtgcggttaaagaaaagaacggcgcggagttccgtggtaaacaactgcgtgttgcgctgagcacccgtagcctggcgaaggatcgtgcgcgtgcggagcgtgcgcgtctgctgattgcggcgcaaaaatttaacaagcgtaagaaccacaccaagggtggcggtggcagcatgacccgtaaccgtctgttctttccgctgctgctgctgctgagctgcagcgtgattgttggcgcgaacgcgaccgaaaagaaagcgagcaccccgcgtaaagcggagggtgtgcagccgcaaagcgttagcccgagcagcagctttccgggtgatggcaccggtgtgccgctgaagctggaactgggcgagctgcgtgacaaagcgctgctggcggcgaaggatgcgtttggcaacaccaccggtgcggcgatgcagtgcatgcaagcgaaaaccgatgttgaggaaaccaagaaatatgcggaggaagcgaagaaactgttcgacaaaattggtggcgattatgtgagcaaaagcgcggcgctggcggatgcggttaaggcgagcaccgatgcggaggaagcgctgaaaagctgcgtggaggcggaaaaagcggcggtggatgcggataccgcggttctggcggcggtgctggaagttctgcagcacagcaaattttggcgtcgtgacaccgcggtgagcaccgaaaaactggcgaacgttagcaagcacagcgcgaacgcgaccaacgaggcgcagaaggcgggtatccaagcgagcaaagcggcggaagcggcgaagcgtgcggcggagagcaagaaaaaggcggcggcggcgctggataccgttaaagaggtggttgcgatggcggaaatgctgaaagagaagttctttgagaacgaacgtctgcagaaagaaaagcacgaggcgcaactggaggcggaacgtcagttcattcaagaggaagtgcagaagaaagaggcggaagcggagaaagcgctgaaccgtgcggcggcggcggataagcgtgttgcggaactggagctggcgcgtcagaaacaaagcaaggaacaaggcaacgagggtcgtggtcaccgtcgtgtgcgtcgtagcggtagcgacagcagcagcaactatgcgccggcgtatgaaccgcgtctgctgctgctgccgctgctgagctttaccctgttctgctttgttgcgtggtgc

SEQ ID NO: 87 - DNA, 1173 bp, synthetic construct
  source 1..1173: mol_type = other DNA
atggcgttttgtatcatttctgagagcaggggcatgtctctgtgggatatgctagcgtggcaccgcccaaaagttacgggtgtacttcttggaaccgtactttccgtcctgacgtttttttgccttatgaaatacacaatggtgacgttcctctgccgcatcctgcagttggtcctattggccggcgttctgttgggcttcacgaatcgatggcacctcacctccgacgacatccacgaggccgtcaaccgccttgtggactgcgccacgccccggctggtgacggcccttgagtccatgcaccaactcgtgacgtggcgtgactaccgccgctccgggctcgtcacgctggtgagcttcgtggttgctcttctcggcaacctcgtctccgacgccgcctttctcacgttttttcttttgttggccttcaccgttcctgcggtgtacgagaagaagaaggatttgatcgacaagtggatcagcgctgccacggctcaggtggagaagtacatggggaagatcaaaacaaaggtggaagaggcgaccaagaagaaagagggaggtggtggctctatgcctgccaaaaccgccgtttcgaaggctgctgcgcccaaaaaggccgctgcgcccaagaaggccgctgcaccacaaaaggctgctgcgcccaagaaggctgctgcgcccaagaaggctgctgcaccccaaaaggctgctgtcgccaagaaggccgtcagggaggcccccaaaaagggtgtcaagaagaccgccaagaagggcgcgccggccgctatgacgaaggtggtgaaggtcacgaagcgcaaggcgtacacccgcccgcagttccgtcgtccgcacacgtaccggaggccgtcgatccccaagccgagcaacaacatgagtgcgattcccaacaagtgggatgcgtttcgtgtgatccgctacccgctgaccaccgacaaggcgatgaagaagattgaggagaacaatacgctgaccttcattgtggactcgaacgccaacaagacggaaatcaagaaggccatgcgcaagctctaccaggtgaaggccgtgaaggtgaacaccctcatccgaccggacggccttaagaaggcgtacatccgcctctccgcctcgtacgacgccctcgagacagccaacaagatgggtctgctg

SEQ ID NO: 88 - DNA, 1173 bp, synthetic construct
  source 1..1173: mol_type = other DNA
atggcgttctgcatcattagcgaaagccgtggtatgagcctgtgggacatgctggcgtggcaccgtccgaaagtgaccggtgttctgctgggcaccgtgctgagcgttctgaccttcttttgcctgatgaagtacaccatggtgaccttcctgtgccgtattctgcagctggtgctgctggcgggtgttctgctgggctttaccaaccgttggcacctgaccagcgacgatatccacgaggcggtgaaccgtctggttgactgcgcgaccccgcgtctggtgaccgcgctggaaagcatgcaccaactggttacctggcgtgattatcgtcgtagcggtctggtgaccctggttagctttgtggttgcgctgctgggcaacctggtgagcgatgcggcgttcctgaccttctttctgctgctggcgtttaccgtgccggcggtttacgagaagaaaaaggacctgatcgataaatggattagcgcggcgaccgcgcaggtggaaaagtatatgggcaagattaaaaccaaggttgaggaagcgaccaaaaagaaagagggtggcggtggcagcatgccggcgaaaaccgcggttagcaaagcggcggcgccgaagaaagcggctgctccgaagaaagcggcggcgccgcagaaggctgctgcgccgaagaaagcggccgctccgaagaaagcggctgcgccgcaaaaggcggcggtggcgaagaaagcggttcgtgaggcgccgaagaaaggtgtgaagaaaaccgcgaagaaaggcgcgccggcggcgatgaccaaagtggttaaggttaccaagcgtaaggcgtacacccgtccgcagttccgtcgtccgcacacctatcgtcgtccgagcatcccgaaaccgagcaacaacatgagcgcgattccgaacaagtgggacgcgttccgtgttatccgttacccgctgaccaccgataaagcgatgaagaaaatcgaggaaaacaacaccctgacctttattgtggacagcaacgcgaacaaaaccgaaatcaagaaagcgatgcgtaagctgtatcaagttaaagcggtgaaggttaacaccctgattcgtccggacggtctgaagaaagcgtacatccgtctgagcgcgagctatgatgcgctggaaaccgcgaacaagatgggcctgctg

SEQ ID NO: 89 - DNA, 1779 bp, synthetic construct
  source 1..1779: mol_type = other DNA
atgcccggcaaggaagtgaaaaaggccgccaagcccgctgccaagactgctgcaaagcctgctgccaagtctgctgccaagccagctgccaagccagctgccaagccagccgcgaagaccgctgcgaagccggccgcgaagactgctgccaagcccgctaagaagcccgctgtgaagcccactgtcaagcctgctgccaaggcagccgcgccctacaagaagcctgcggccatctcaccttttgtggcgcggccgaaaaactttggtattggccacgatgttccgtacgcccgtgatctttctcgctttatgcggtggcccacgtttgtgacgatgcagcggaagaagcgtgtactgcagcgccgtctgaaggtgccgcccgcgctccaccaatttacgaaggtgcttgaccgctccagtcgcaacgagctgctgaagctggtgaagaagtatccttccgagacgcgcagggcccgcaggcagcgcctgtttgacgtggcgactgagaaaaagaagaatccagaggcggcgtccaagaaggccccgctcagcgtcgttaccggtctgcaggaggtaacccgcaccattgagaagaagaccgcacgccttgtgatgatcgcgaacaatgtggaccccattgagctggtgctgtggatgccgactttgtgccgtgccaacaaagtcccatacgcgattgtgaaggacaaggcacgtctcggcgacgcggtgggccggaagaccgccacgtgcgttgcaatcaccgatgtgaatgccgaggacgaggccgctttgaagaatctcatccgctctgtgaatgcacgcttcctggcccgtagcgatgttatccgtcgccaatggggaggcctgcagctctcactgcgttctcgagccgagctgcgcaagaagcgtgcccgcaacgccggcaaggatgctgccgccgtaatgggaggtggtggctctatgaccaagaagcacctgaagcgcctttatgcccccaaggactggatgctgagcaagctcacgggcgtgttcgctccacgtccccgtgctggaccccacaagctgcgtgagtgcatgactcttatgatcatcatccgcaatcgtctgaagtatgcgctgaacgccgccgaggctcagatgattctccgtcagggccttgtgtgcgtggacggtaagccccgcaaggacaccaagtatccggttggcttcatggacgttgtggagatcccacggaccggggatcgtttccgcattctgtacgacgtgaagggccgctttgccctcgtgaaggttggcgaggctgaggggaacatcaagctcctgaaggtggagaacgtctacacaagcactggtcgcattcctgttgccatgacacacgacggtcaccgcattcgttaccccgacccccgcacccaccgtggcgacaccctggtgtacaacctgaaggagaagaaggtggtggacctcatcaagtccagcaacggcaaggtggtgatggtcaccggcggcgcgaaccgcggccgtattggcgagatcatgtcgattgagcgccaccctggtgcgttcgacattgcacgcctgaaggatgcggcgggacacgagtttgctacccgagcgtccaacatttttgtgattggcaaggacatgcagagcgttcctgtgacgctgccgaagcaacagggtctccgcatcaacgtgattcaggagcgtgaggagaagcttatcgctgctgaggcacgcaagaatatgcagactcgcggcgtacgcaaggcccgcaaa

SEQ ID NO: 90 - DNA, 1779 bp, synthetic construct
  source 1..1779: mol_type = other DNA
atgccgggtaaagaggtgaagaaggcggcgaagccggcggcgaaaaccgcggcgaaaccggcggcgaaaagcgctgctaagccggctgctaaaccggctgctaaaccggcggcgaagaccgctgctaagcctgctgcgaaaaccgcggcgaagccggcgaagaaaccggcggtgaaaccgaccgttaagccggcggcgaaagcggcggcgccgtacaagaagccggcggcgatcagcccgtttgttgcgcgtccgaagaactttggtattggccacgacgtgccgtatgcgcgtgatctgagccgtttcatgcgttggccgacctttgttaccatgcagcgtaagaaacgtgttctgcaacgtcgtctgaaagtgccgccggcgctgcaccagttcaccaaggttctggaccgtagcagccgtaacgagctgctgaaactggtgaagaaatacccgagcgaaacccgtcgtgcgcgtcgtcagcgtctgtttgatgtggcgaccgagaagaaaaagaacccggaagcggcgagcaaaaaggcgccgctgagcgtggttaccggcctgcaagaggttacccgtaccatcgagaagaagaccgcgcgtctggtgatgatcgcgaacaacgttgacccgattgagctggtgctgtggatgccgaccctgtgccgtgcgaacaaagtgccgtatgcgatcgttaaagataaagcgcgtctgggtgatgcggtgggtcgtaagaccgcgacctgcgtggcgattaccgacgttaacgcggaggatgaagcggcgctgaaaaacctgatccgtagcgttaacgcgcgtttcctggcgcgtagcgatgtgattcgtcgtcagtggggtggcctgcaactgagcctgcgtagccgtgcggaactgcgtaaaaagcgtgcgcgtaacgcgggtaaagatgcggcggcggtgatgggtggcggtggcagcatgaccaaaaagcacctgaagcgtctgtacgcgccgaaagattggatgctgagcaaactgaccggtgtttttgcgccgcgtccgcgtgcgggtccgcacaaactgcgtgagtgcatgaccctgatgatcattatccgtaaccgtctgaagtatgcgctgaacgcggcggaagcgcagatgatcctgcgtcaaggtctggtgtgcgttgacggcaaaccgcgtaaggataccaaatacccggttggttttatggacgtggttgagatcccgcgtaccggcgaccgtttccgtattctgtatgatgtgaaaggtcgttttgcgctggtgaaggttggcgaggcggaaggcaacattaagctgctgaaagtggaaaacgtttacaccagcaccggtcgtattccggttgcgatgacccatgatggtcaccgtattcgttacccggacccgcgtacccaccgtggcgataccctggtttataacctgaaggagaaaaaggtggttgatctgatcaagagcagcaacggtaaagtggttatggtgaccggtggcgcgaaccgtggtcgtattggcgagatcatgagcattgaacgtcacccgggcgcgtttgacattgcgcgtctgaaagatgcggcgggtcatgaatttgcgacccgtgcgagcaacatctttgttattggcaaagatatgcaaagcgtgccggttaccctgccgaagcagcaaggtctgcgtatcaacgtgattcaggagcgtgaggaaaaactgatcgcggcggaagcgcgtaagaacatgcaaacccgtggtgttcgtaaggcgcgtaaa

SEQ ID NO: 91 - DNA, 1311 bp, synthetic construct
  source 1..1311: mol_type = other DNA
atgacgacaatcggtacgtacaacgaggagggtgttaacgtggacctgtacatcccacgcaagtgccacgcgacaaacaaccttatcacgtcatacgaccactccgccgtgcagattgccattgcgaatgtggacgccaacggtgtgctaaacggcacgacgacaaccttctgcattgctggctatcttcgtcgccaggctgagtctgaccacgcaatcaaccacctggcgatttcgaagggcattatccgcatcaagaccggcaagaagcctcgcgcgaagaagcttaagaatgtgaagggccttggcgtacgcggcttaccaaggggtgctctgcaacagaggggagctcgtgtcctcccaacccagaggggtgtcgcgcagcgtggcggcgctcagaagggcaacgtccgcaagctgcagccacagccgcagaagcaaaggtcacagctgaatcaaaggtcacagcagcagcacggcgcccggccgacccggaaggaagagggcggtcgcacgcagcgtggtggcagggatgcgcctcaagctcgcaagcagcaaggcaggaacgagcctcaagctcgcaggcagcaaggcaggaacgagcctcaagctcgcaggcagcaaggcaggaacgagcctcaagctcgcaagcagcaaggcagggatgcgcctcaagctcgtaagcagcaaggcaggaatgcacctcgttcccagaaggcaggaggtggtggctctatgatgcgttttacccggttccttgtcgttgcagcaaagcggagtgccaccagcgccaaactcggtaagagtgttggactcaccgcggcgctgagtcccaagcaaaggtcccttccccgcgtctcagtgacgaagttgatgaagcccagcgggagcgggaaacacgttacgtcgtcattcttgttgaaggacaagaagaaggtggccaccgcaaaagttgctgtgccgccgaaaaagaagagggctttaaaggtgaggaagggccgcagcagcggcaaaaaggccgcggctctctatgtgcgcttttatcacgccttgaagaagagcggacttgtgaaggggaagcgacgcatgcagaaaacgggtgagctgtggcgtgccacaaagaaggcgaaggacttcaagaagcgcgttgaggcggcgatgaggcttgcaaagaagggacaaaaaagcagggctcgtaagctgaaggcgcagaagaaggcaaagggcaaaaagtcggcgaagggcgtcaggagggtctaccggagggtcagcaggaagaagactgtcacgagcaccgtgccgcctctccct

SEQ ID NO: 92 - DNA, 1311 bp, synthetic construct
  source 1..1311: mol_type = other DNA
atgaccaccatcggcacctacaacgaggaaggcgtgaacgttgacctgtatatcccgcgtaagtgccacgcgaccaacaacctgattaccagctacgaccacagcgcggtgcagatcgcgattgcgaacgtggatgcgaacggtgttctgaacggcaccaccaccaccttctgcatcgcgggttatctgcgtcgtcaggcggaaagcgatcacgcgatcaaccacctggcgattagcaagggtatcattcgtattaaaaccggcaagaaaccgcgtgcgaagaaactgaagaacgtgaaaggtctgggtgttcgtggtctgccgcgtggcgcgctgcagcaacgtggtgcgcgtgtgctgccgacccagcgtggcgttgcgcagcgtggtggcgcgcaaaagggcaacgttcgtaaactgcaaccgcagccgcaaaaacagcgtagccaactgaaccagcgtagccagcaacagcatggtgcgcgtccgacccgtaaggaagagggtggtcgtacccaacgtggtggccgtgatgcgccgcaagcgcgtaaacaacagggtcgtaacgagccgcaggcgcgtcgtcaacagggtcgtaacgaaccgcaagcgcgtcgtcagcaaggccgcaatgaaccgcaggcgcgtaaacaacagggccgcgacgcgccgcaagcgcgtaagcaacagggtcgtaacgcgccgcgtagccagaaagcgggtggcggtggcagcatgatgcgtttcacccgttttctggttgttgcggcgaagcgtagcgcgaccagcgcgaagctgggtaaaagcgtgggcctgaccgcggcgctgagcccgaaacagcgtagcctgccgcgtgtgagcgttaccaagctgatgaaaccgagcggtagcggcaagcacgtgaccagcagctttctgctgaaagacaagaaaaaggttgcgaccgcgaaggtggcggttccgccgaaaaagaaacgtgcgctgaaggttcgtaaaggtcgtagcagcggcaagaaagcggcggcgctgtacgtgcgtttctatcacgcgctgaagaaaagcggtctggttaagggcaaacgtcgtatgcagaagaccggtgaactgtggcgtgcgaccaagaaagcgaaagattttaagaaacgtgtggaggcggcgatgcgtctggcgaagaaaggtcagaagagccgtgcgcgtaagctgaaagcgcaaaagaaagcgaagggtaagaaaagcgcgaaaggcgtgcgtcgtgtttaccgtcgtgttagccgtaagaaaaccgtgaccagcaccgttccgccgctgccg

SEQ ID NO: 93 - DNA, 1332 bp, synthetic construct
  source 1..1332: mol_type = other DNA
atgggtatcgttcgcagccgcctgcataagcgcaagatcaccggtggaaagacgaagatccaccggaagcgcatgaaggccgaactcggccgtcttcccgcgcacacgaaacttggcgcccgccgcgtgagtcccgtccgcgcccgcggtgggaacttcaagctccgcggtcttcgcctggacaccggcaattttgcgtggggcacagaagccattgctcagcgggcccgtatcctcgacgtcgtgtacaacgccacttctaacgagctggtgcgcacgaagacgcttgtgaagaactgcattgttgtggtggacgccgcgcccttcaagttatggtacgcgaagcactacggtatcgatcttgacgccgcgaagagcaagaagacggcgcagagcacgacggagaagaagaagtcgaagaagacctcacacgccatgactgagaagtacgacgtcaagaaggcctccgacgagctgaagcgcaagtggatgctccgccgcgagaaccacaagattgagaaggcagtcgctgatcagctcaaggagggccgtctgctcgcccgcatcaccagccgccctggccagacagcccgcgccgatggtgcactgctggagggcgccgaactgcagttctatctgaagaagctcgagaagaagaagcggggaggtggtggctctatgccgaagggaaaaaacgcgatcccccacgtgcaccagaggaagcactggaacccgtgctcttcccagaagggtaatgtgaaggttttcctcaaccagcccgcacagaagctgcgccgtcgccgcctacgtcttttgaaggcgaagaagacgttcccacgcccactcaaggcgctgcgcccgcaggtgaattgccccacggtgcgtcacaacatgaagaagcgcctgggccgtggctttaccgttgaggagctgaaggctgccggcatcaaccctcgttttgccccgacgattggcatccgtgtggatcgtcgccgcaagaacaagagcgaggagggcatgagcatcaacatccagcgcctgaagacgtacatgagcaagctggtgctcttccccatgagctacaagaacgtgcagaagggcgaggccactgaggaggaggtgaagtctgccactcaggaccgcacacgctttggtactgcggctgttggtggttttgtgacgcctgctcccgaggcaccacgcaaggtgacagaggaggagcgcacaaagaacgtgtacaagttcctcaagaagaaccacagcgctgttcgcttctttggcattcgcagggcacgtcaggaacgcagggaggccaaggagaacgagaagaag

SEQ ID NO: 94 - DNA, 1332 bp, synthetic construct
  source 1..1332: mol_type = other DNA
atgggtatcgttcgtagccgtctgcacaagcgtaaaatcaccggtggcaagaccaaaattcaccgtaagcgtatgaaagcggagctgggtcgtctgccggcgcacaccaaactgggtgcgcgtcgtgttagcccggtgcgtgcgcgtggtggcaacttcaagctgcgtggcctgcgtctggacaccggtaactttgcgtggggcaccgaggcgattgcgcagcgtgcgcgtattctggatgtggtttacaacgcgaccagcaacgaactggttcgtaccaagaccctggtgaaaaactgcatcgtggttgtggacgcggcgccgttcaagctgtggtacgcgaaacactatggtattgacctggatgcggcgaaaagcaagaaaaccgcgcagagcaccaccgaaaagaaaaagagcaaaaagaccagccacgcgatgaccgagaagtacgacgttaaaaaggcgagcgatgaactgaagcgtaaatggatgctgcgtcgtgagaaccacaagatcgaaaaagcggtggcggaccaactgaaagagggtcgtctgctggcgcgtattaccagccgtccgggtcagaccgcgcgtgcggatggtgcgctgctggagggcgcggaactgcaattttatctgaaaaagctggagaagaagaagcgtggtggtggtggtagcatgccgaagggtaaaaacgcgatcccgcacgtgcaccagcgtaagcactggaacccgtgcagcagccaaaagggcaacgttaaagtgttcctgaaccagccggcgcaaaagctgcgtcgtcgtcgtctgcgtctgctgaaagcgaagaaaacctttccgcgtccgctgaaggcgctgcgtccgcaggttaactgcccgaccgtgcgtcacaacatgaagaaacgtctgggtcgtggcttcaccgttgaggaactgaaagcggcgggtattaacccgcgttttgcgccgaccatcggcattcgtgtggaccgtcgtcgtaagaacaaaagcgaggaaggtatgagcatcaacattcaacgtctgaagacctacatgagcaaactggttctgttcccgatgagctataagaacgtgcagaaaggcgaggcgaccgaggaagaggttaaaagcgcgacccaagatcgtacccgttttggcaccgcggcggttggtggcttcgtgaccccggcgccggaagcgccgcgtaaggttaccgaagaggaacgtaccaagaacgtgtacaagttcctgaagaaaaaccacagcgcggtgcgtttctttggtattcgtcgtgcgcgtcaagagcgtcgtgaagcgaaggagaacgaaaagaaa

SEQ ID NO: 95 - DNA, 1863 bp, synthetic construct
  source 1..1863: mol_type = other DNA
atgtataagtttggaggtgaggcgaaggatcttagaaacatttataattttggcgatatgagccaacgagaaacggagccaccgaaggacttatcattagcagaaaataaagcttatttggtggatgtagaggtgcattctgataataatgaagaggaaatggggaatcgtgagagccaacaacccaattccagggtctcaccgacggctcatggagttcctcaatcctccgcgttttttccggaattttcacactcttctggacctgatgttcctcgaaaaccctcaatggaaagtacttcggaacaaaaaaactcaaaggaaaaacaaaaggagaatagtaaagtaaagattgcaaaagaagttttaggaataaacaagaaaaatacctctgggatgtcacctgaagagaaggagcgtgtattacttgaagaaaggtggaaaagagccatggcagaggagaatcgtttgaacgcactcgaagagcaagtaactcatcgtgagcaagcgactaattcttcaggccttcttccaaacttccctcccaagttcttatgtattaagccacttgtacaccatgatatttcgagtgttcccgaggtgagaagacaatttgtcaggtttaattttataaattggattgccacatgtgttttgctccttgtcaatatgattattgttattgctgtggtatttgcatctcataaagaagatgcaaaaaaattccatacttctcaaaacactgttttagccattttgtacctgatgggagcccctttaagctttattgtttggtattggcagatttattctgcttgttccacaggacgtcatactaaacatcttttggctctaagtgggttggttatagctcttgcctttgatatatttatgattgttggtcggacaaactatgctgcatgcggtgtatctcttgcaatagatatatcgaaaacgaaaagtaagcttgccgtattgcccgtgatcgttgttctttttttctgggttgtagaggctgttatattgtgttactgtatcgcaaaacagtggatgtactatcggttggatgtgaacgcgcaagaagaagtgagacgccagatgcggaatgtgattggaattggaggtggtggctctatgggaaaaaaatacgcacagttagagactctccacaacgtgaatgggcgggttgtcattgtgggcgacattcatggctgccttgcccaactggaggacattttatcagtcacagactttgcgaggggaagggatcagttaatcaccgctggggacatggtgaacaaagggccagactcgtttggcgttgtgcgtctgctgaagagccttggagcacgcggtgtgattggcaatcatgacgccaagcttctcaaacttcggaaaaagatacgaaaacatgggacgctgcacgggacgaatagccaatcgagtttggccccgcttgccatgtcgctaccgcaggatgttgaagagtatttattacaactgccgcatattctccgcattcctgcacacaacattctggtggtacatgcgggccttcacgttcaacacccactcgagcggcaattggttaaggaggtcactacgatgcgcaacctcattttgcaggatgacgggctgtacagggcatctgaggatacaacggacggtgtgccctgggcatcgctgtggcagggtccggagactgttgtctttggccacgacgccagacgaggcctccaacgccaccctcatgcgatcgggttggacactcggtgtgtgtatggcggggagctcactgctcttgtgtgtcccggtgaacacctcgtttccgtgcctggatggacttccaatagatcgaaggtg

SEQ ID NO: 96 - DNA, 1863 bp, synthetic construct
  source 1..1863: mol_type = other DNA
atgtacaagttcggtggcgaagcgaaagatctgcgtaacatctataactttggcgacatgagccagcgtgaaaccgagccgccaaaggatctgagcctggcggaaaacaaagcgtatctggtggacgttgaggtgcacagcgataacaacgaggaagagatgggtaaccgtgaaagccagcaaccgaacagccgtgttagcccgaccgcgcatggcgtgccgcaaagcagcgcgttctttccggagtttagccacagcagcggtccggatgttccgcgtaagccgagcatggaaagcaccagcgagcagaaaaacagcaaggaaaaacaaaaggagaacagcaaagttaagatcgcgaaggaagtgctgggcattaacaagaaaaacaccagcggtatgagcccggaagagaaggagcgtgttctgctggaagagcgttggaaacgtgcgatggcggaagagaaccgtctgaacgcgctggaagagcaggtgacccaccgtgaacaagcgaccaacagcagcggcctgctgccgaacttcccgccgaagtttctgtgcatcaaaccgctggttcaccacgatattagcagcgttccggaagtgcgtcgtcagttcgtgcgtttcaactttatcaactggattgcgacctgcgttctgctgctggtgaacatgatcattgttatcgcggtggttttcgcgagccacaaggaagacgcgaagaaatttcacaccagccagaacaccgttctggcgattctgtacctgatgggtgcgccgctgagctttatcgtgtggtactggcaaatttatagcgcgtgcagcaccggccgtcacaccaaacacctgctggcgctgagcggtctggtgattgcgctggcgttcgatatctttatgattgttggccgtaccaactatgcggcgtgcggtgtgagcctggcgatcgacattagcaaaaccaagagcaaactggcggttctgccggtgattgtggttctgttcttttgggtggttgaggcggttatcctgtgctattgcattgcgaaacagtggatgtactatcgtctggatgttaacgcgcaggaagaggtgcgtcgtcaaatgcgtaacgtgatcggcattggtggcggtggcagcatgggtaagaaatacgcgcaactggaaaccctgcacaacgttaacggtcgtgtggttatcgtgggcgacattcacggttgcctggcgcagctggaggacatcctgagcgttaccgatttcgcgcgtggccgtgaccaactgattaccgcgggtgatatggtgaacaagggcccggacagctttggtgtggttcgtctgctgaaaagcctgggtgcgcgtggcgtgatcggtaaccacgatgcgaagctgctgaaactgcgtaagaaaattcgtaagcacggcaccctgcatggcaccaacagccagagcagcctggcgccgctggcgatgagcctgccgcaggacgttgaagagtatctgctgcaactgccgcacatcctgcgtattccggcgcacaacatcctggtggttcatgcgggcctgcatgtgcagcacccgctggaacgtcaactggttaaagaggtgaccaccatgcgtaacctgattctgcaggacgatggtctgtatcgtgcgagcgaagacaccaccgatggcgttccgtgggcgagcctgtggcagggtccggaaaccgtggttttcggccacgatgcgcgtcgtggtctgcaacgtcacccgcacgcgatcggtctggacacccgttgcgtttacggtggcgaactgaccgcgctggtgtgcccgggtgaacacctggttagcgtgccgggttggaccagcaaccgtagcaaggtg

SEQ ID NO: 97 - DNA, 1863 bp, synthetic construct
  source 1..1863: mol_type = other DNA
atgtataagtttggaggtgaggcgaaggatcttcgaaacatttataattttggcgatatgagccaacgagaaacggagccacagaaggaattatcattggcagaaaatagagcttatttggtggatgtagaggtgcattctgataataatgaagaggaaatggggcatcgtgagagccaacaacccaactccagagtctcaccgacggctcagggagttcctcagtcctccgcgtttttttcggaattttcacactcttctggaattgattttcctcaaaaaccctcaatggaaaatacttcggaccaaaaaaactcaaacgaaaaaccaaaggagaatagtaaagtaaagatcgcaaaagaagttttaggaataaataagaaaaatacctctgggatgtcacctgaagagaaggagcgtgtattacttgaagaaagatggaaaagagccatggcagaggagaatcgtttgaacgcactcgaagagcaagtaactcatcgtgagcaagcgactaattcttcaggtcttcttcccaacttccctcccaagttcttatttattaagccacttgtacaccatgatatttcgagtgttcccgaggtcagaagacaatttgtcaggtttaattttataaattggatcgccacatgtgttttgctccttgtcaatatgattattgttattgctgtggtatttgcatctcataaagaagatgcaaaaaaattcaatacttctcaaaacactgttttagccattttgtacctggtgggagcccctttaagctttattgtttggtattggcagatttattctgcttgttccacaggacgtcataccaaacatcttttggctctaagtgggttggttatagcacttgcctttgttatatttatgattgttggtcggacaaactatgctgcatgcggtgtatctcttgcaatagatatatcgaaaacgaaaagcaagtttgccgtattgcccgtgatcattgttctttttttctgggttgtagaggctgttatattgtgttactgtatcgtaaaacagtggatctactatcggttggatgtgaacgcgcaagaagaagtgaggcgccagatgcggaatgtgattggaattggaggtggtggctctatgggaaaaaaatacgcacagttggagactctccacaacgtgaatgggcgggttgtgattgtaggcgacattcatggctgccttgcccaactggaggacattttatcagtcacagaatttgcgaggggaagggatcagttaatcaccgctggggacatggtgaacaaagggccagactcgtttggcgttgtgcgtctgctgaagagccttggagcacgcggtgtgattggcaatcatgacgccaagcttctcaaacttcggaaaaagatacgaaaacatggggcgctgcacgggaagaatagccaatcgagtttagccccgcttgccatgtcgctaccgcaggatgttgaagagtatttatcacaactgccgcatattctccgcattcccgcacacaacattctggtggtacatgcgggccttcacgttcaacacccgcttgagcggcaattggttaaggaggtcactacgatgcgcaacctcattttgcaggatgacgggctgtacagggcatctgaggatacaacggacggtgtgccgtgggcatcgctgtggcagggtccggagactgttgtctttggccacgacgccagacgaggcctccaacgctaccctcatgcgatcggattggacactcggtgtgtgtatggcggggagctcactgctcttgtgtgtcccggtgaacacctcgtttccgtgcctggatggacttccaatagatcgaaggtg

SEQ ID NO: 98 - DNA, 1863 bp, synthetic construct
  source 1..1863: mol_type = other DNA
atgtacaaattcggtggcgaagcgaaggatctgcgtaacatttataactttggcgacatgagccagcgtgaaaccgagccgcaaaaagaactgagcctggcggagaaccgtgcgtatctggtggacgttgaagtgcacagcgataacaacgaggaagagatgggtcaccgtgagagccagcaaccgaacagccgtgtgagcccgaccgcgcagggcgttccgcaaagcagcgcgttctttagcgaattcagccacagcagcggtatcgattttccgcagaaaccgagcatggagaacaccagcgaccaaaagaacagcaacgaaaagccgaaagagaacagcaaggtgaaaattgcgaaggaagttctgggcatcaacaagaaaaacaccagcggtatgagcccggaagagaaagagcgtgtgctgctggaagagcgttggaagcgtgcgatggcggaagagaaccgtctgaacgcgctggaagagcaggttacccaccgtgaacaagcgaccaacagcagcggcctgctgccgaacttcccgccgaaattcctgtttattaagccgctggtgcaccacgatatcagcagcgttccggaagtgcgtcgtcagtttgttcgtttcaactttatcaactggattgcgacctgcgtgctgctgctggttaacatgatcattgtgattgcggtggttttcgcgagccacaaagaggacgcgaagaaatttaacaccagccagaacaccgtgctggcgatcctgtacctggttggtgcgccgctgagcttcattgtttggtactggcaaatctatagcgcgtgcagcaccggtcgtcacaccaagcacctgctggcgctgagcggtctggtgattgcgctggcgttcgtgatctttatgattgttggccgtaccaactacgcggcgtgcggtgttagcctggcgatcgatattagcaagaccaaaagcaagtttgcggttctgccggtgatcattgttctgttcttttgggtggttgaagcggtgatcctgtgctattgcattgttaaacagtggatctactatcgtctggacgtgaacgcgcaggaagaggttcgtcgtcaaatgcgtaacgtgatcggcattggtggcggtggcagcatgggtaagaaatacgcgcaactggaaaccctgcacaacgtgaacggtcgtgtggttatcgttggcgatattcacggttgcctggcgcagctggaagacattctgagcgtgaccgagtttgcgcgtggccgtgaccaactgatcaccgcgggtgatatggttaacaaaggcccggacagctttggtgtggttcgtctgctgaagagcctgggtgcgcgtggcgttattggtaaccacgatgcgaaactgctgaagctgcgtaagaaaatccgtaaacacggcgcgctgcacggcaagaacagccagagcagcctggcgccgctggcgatgagcctgccgcaggacgtggaagagtatctgagccaactgccgcacatcctgcgtattccggcgcacaacattctggtggttcatgcgggcctgcatgttcagcacccgctggaacgtcaactggtgaaagaggttaccaccatgcgtaacctgatcctgcaggacgatggtctgtaccgtgcgagcgaagacaccaccgatggcgtgccgtgggcgagcctgtggcagggtccggaaaccgtggttttcggccacgatgcgcgtcgtggtctgcaacgttacccgcacgcgatcggtctggacacccgttgcgtgtatggtggcgaactgaccgcgctggtttgcccgggcgagcacctggttagcgtgccgggttggaccagcaaccgtagcaaggtt

SEQ ID NO: 99 - DNA, 1479 bp, synthetic construct
  source 1..1479: mol_type = other DNA
atgggggctcctcagatcgtgtactccgccctcataacgaacaccaccacaattgctgtgacggtggttgtcacctacaccatgccgaacgaaatgcccccggagactctggaattgctcattcaaccaggcgaagaaatgttagcgccgcagaaattggtggaggacggtatagtaacctggacaggctatattagcaaggttgccattcagggtgggccgtctatgagtgaacctttcccgggagtggagtgtcctacgagaagatacgactttgaagttttcatgcatgccggcgtgctgcggctattcgcattgggccctgccgaatcaagcagtgatggaggtggtggctctatggatacgacgctttacagtgaggtgaatcgtctcgaacgcggtgactttcttctttttcactgtgtgcagctctcacaacacgagcgtgacgtgcagcggtacttctttggatgctactttccgcgctggcgtgggttctacctggaggaggtgagggatatgccgggccctctaggctacaaggtgcagcgacactttcctgcgtatccctttgacgtgtatctgaaggacaatggtgaacactttctcacggatgacttccaggagggttctatattcactttgggagcctcgcaaaatcagcgtgacggcgactcgaagcgatataaagtagtgcactgcgacgatagtcgtttgcgcacgcgcacgggtacgactcttgcagacattggcaatgacatcacgacgaagttgaatcaaacacaccgtgtccctggcgaggtgatagatctcctgcgtgagattagagatgcgtatgttgtgtatgccggcaatggcattcctgagattggtatcaaggcaatgggacgtcactttcgccacgtcagcgaggatggaaagcggtggatgtcgttggagaacattggaaagcttgttcgtgactctcgtgccttttccaacacattgtcatttgaggacacgcagaggacgaattccacgattagcaataatgcaaggagcattcatgaagcctttccgcagaatgaagaaggctgcattgactatgatttattcatggactacgttcgtggaccgatgagccaaaaaaggaaggatgccgtctgggaaatattccgcaagcttgactttgatggagacggctacctcaacatcttagacattcaggcccgctacaatgcgcagcagcaccctgtggtggcggtggagagactcttctccgcggacaaactgctcaagggcttcctcaccgtttgggatgagaacaaacaatacgggttgatcccatacgccgagtttatcgactactacaacggcgtcagcgcggtaattgcggacgactacatcttttttgatattctccggaatcaatggaaggtcatgcgtgactggggagggacggtggggacgaggggagggaattgtgagttcccgacgatg

SEQ ID NO: 100 - DNA, 1479 bp, synthetic construct
  source 1..1479: mol_type = other DNA
atgggtgcgccgcagatcgtgtacagcgcgctgattaccaacaccaccaccatcgcggttaccgtggttgtgacctataccatgccgaacgagatgccgccagaaaccctggaactgctgattcagccgggcgaggaaatgctggcgccgcaaaagctggttgaagatggtatcgtgacctggaccggctacatcagcaaagtggcgattcaaggtggcccgagcatgagcgaaccgttcccgggtgttgaatgcccgacccgtcgttatgacttcgaggtttttatgcacgcgggtgttctgcgtctgtttgcgctgggtccggcggaaagcagcagcgatggtggcggtggcagcatggacaccaccctgtacagcgaggtgaaccgtctggaacgtggtgatttcctgctgtttcactgcgttcagctgagccaacacgagcgtgacgtgcagcgttacttctttggttgctatttcccgcgttggcgtggcttttacctggaggaagttcgtgatatgccgggtccgctgggctataaggtgcagcgtcacttcccggcgtacccgtttgatgtttatctgaaagacaacggcgagcacttcctgaccgacgattttcaagaaggtagcatcttcaccctgggcgcgagccagaaccaacgtgacggcgatagcaagcgttacaaagttgtgcactgcgacgatagccgtctgcgtacccgtaccggcaccaccctggcggatatcggcaacgacattaccaccaagctgaaccagacccaccgtgttccgggcgaagtgattgatctgctgcgtgaaatccgtgacgcgtacgttgtgtatgcgggtaacggcattccggaaatcggtattaaagcgatgggccgtcacttccgtcacgtgagcgaggacggcaagcgttggatgagcctggaaaacatcggcaaactggttcgtgatagccgtgcgttcagcaacaccctgagctttgaggacacccagcgtaccaacagcaccattagcaacaacgcgcgtagcatccacgaagcgttcccgcaaaacgaggaaggttgcattgactacgatctgtttatggattatgtgcgtggcccgatgagccagaagcgtaaagacgcggtttgggagatcttccgtaagctggactttgatggtgacggctacctgaacatcctggatattcaagcgcgttataacgcgcagcaacacccggttgtggcggtggaacgtctgttcagcgcggataagctgctgaaaggttttctgaccgtttgggacgagaacaagcagtacggcctgattccgtatgcggaattcatcgactactataacggtgttagcgcggtgatcgcggacgattacatcttctttgatattctgcgtaaccaatggaaagttatgcgtgactggggtggcaccgtgggcacccgtggtggcaactgcgagtttccgaccatg

SEQ ID NO: 101 - DNA, 1479 bp, synthetic construct
  source 1..1479: mol_type = other DNA
atggggtctcctaagatcgtgtactccgccctcataaggaacaccaccacgatttctgtgacggtgcttgtcacctattccatgccgagcgaaatgccccaggaaactgtgcaattgctcattccaccaggcgaagaaaaggaagcgccccagaaattggtggaggaagatacagtaacctggacaggctttattagcaaggttgccgttgagggtgggcagtctatgagtgctcctttcctgggagtggaatctcctacgagaagatacggttttgaagtttacatgcaagccggcatgctgcggctattagcattgggccctgtcgaatcaagcagtgatggaggtggtggctctatggatacgacgctttacagtgaggtgaatcgtctcgaacgcggtgactttcttttttttcactgtgtgcagctctcacaacacgagcgtgacgtgcagcggtacttctttggatgctactttccgcgctggcgtgggttctacctggaggaggtgagggatatgccaggccctctaggctacaaggtgcagcgacactttcctgcgtatccctttgacgtgtatctgaaggacaatggtgaacactttctcacggatgacttccaggagggttctatattcactttgggagcctcgcaaaatcagcgtgacggcgagtcgaagcgatataaagtagtgcactgcgacgatagtcgtctgcgcacgcgcacgggcacgactcttgcagacattggcaatgacatcacgacgaggttgaatcaaacacaccgtgtccctggcgaggtgatagatctcctgcgtgagattagagatgcgtatgttgtgtatgccggcaatggcattcctgagattggtatcaaggcaatgggacgtcactttcgccacgtcagcgaggatggaaagcggtggatgtcgttggagaacattggaaagcttgttcgtgactctcgtgccttttccaccacattgtcatttgaggacacgcagaagacgaattccacgattagcaataatgcaaggagcattcatgaagcctttccgcagaatgaagaaggctgcattgactatgatttattcatggactacgttcgtgggccgatgagccaaaaacggaaggatgccgtctgggaaatattccgcaagcttgactttgatggagacggctacctcaacatcttagacattcaggcccgctacaatgcgcagcagcaccctgtggtggcggtggagagactcttctccgcggacaaactgcttaagggcttcctcaccgtttgggatgagaacaaacaatacgggttgatcccatacgccgagtttatcgactactacaacggcgtcagcgcggtaattgcggacgactacatcttttttgatattctccggaatcaatggaaggtcatgcgtgactggggagggacggtggggacgaggagagggaagagtgaggtttcgacgatg

SEQ ID NO: 102 - DNA, 1479 bp, synthetic construct
  source 1..1479: mol_type = other DNA
atgggtagcccgaagatcgtgtacagcgcgctgattcgtaacaccaccaccatcagcgtgaccgttctggtgacctatagcatgccgagcgagatgccgcaggaaaccgttcaactgctgattccgccgggtgaagaaaaagaggcgccgcagaaactggttgaggaagataccgtgacctggaccggtttcatcagcaaagttgcggtggaaggtggccaaagcatgagcgcgccgttcctgggtgttgagagcccgacccgtcgttacggttttgaagtgtatatgcaagcgggtatgctgcgtctgctggcgctgggtccggtggagagcagcagcgatggtggcggtggcagcatggacaccaccctgtacagcgaggttaaccgtctggaacgtggtgattttctgttctttcactgcgttcagctgagccaacacgaacgtgacgtgcaacgttacttctttggctgctatttcccgcgttggcgtggtttttacctggaggaagttcgtgatatgccgggtccgctgggctataaggtgcaacgtcacttcccggcgtacccgtttgatgtttatctgaaagacaacggcgagcacttcctgaccgacgattttcaggaaggtagcattttcaccctgggcgcgagccagaaccaacgtgacggcgagagcaagcgttacaaagtggttcactgcgacgatagccgtctgcgtacccgtaccggcaccaccctggcggatatcggcaacgacattaccacccgtctgaaccagacccaccgtgttccgggcgaagtgattgatctgctgcgtgaaatccgtgacgcgtacgtggtttatgcgggtaacggcattccggaaatcggtattaaggcgatgggccgtcactttcgtcacgtgagcgaggacggcaagcgttggatgagcctggaaaacatcggtaaactggttcgtgatagccgtgcgttcagcaccaccctgagctttgaggacacccagaaaaccaacagcaccattagcaacaacgcgcgtagcatccacgaagcgttcccgcaaaacgaggaaggctgcattgactacgatctgtttatggattatgtgcgtggtccgatgagccaaaagcgtaaagacgcggtttgggagatcttccgtaagctggactttgatggtgacggctacctgaacatcctggatattcaggcgcgttataacgcgcagcaacacccggtggttgcggtggaacgtctgttcagcgcggataagctgctgaaaggctttctgaccgtttgggacgagaacaaacaatacggtctgattccgtatgcggaattcatcgactactataacggtgttagcgcggtgatcgcggacgattacatcttctttgatattctgcgtaaccagtggaaggttatgcgtgactggggtggcaccgtgggcacccgtcgtggcaaaagcgaggtgagcaccatg

SEQ ID NO: 103 - DNA, 1035 bp, synthetic construct
  source 1..1035: mol_type = other DNA
atgccaagcacacccaccccgcagtgtgtgcggcggctgcaaaaggagctttccgccctatgccgagaggccgagtcgttttttttcacccgtccctcagcaaagagtattctggtttggtatttcgtcatcaagggtcctgcggataccccttatgaaggcggtcgctactttggcaagctgaattttccccccgactatccaatgaaaccgcctgagattatcattttgacgccaaatggacgttttgagaccaacaagagcatttgtctcaccatgagcaattatcatccggagaattggagccctttgtggggggtccgcaccattcttacggggctgctctcattcatggtgggagacgaactcactactgggtgcatgacgagcagcgatgagttgcggaggaagtatgctcgtgagagccgtcgtttcaatgcagagaaaatgtcagtatacaaggaactgtttcctgaggagtatcaaaaggatttggaggaattgaagcgagaggacagtgagaaaaacggtcgtacttctggaagtgctggttgtggtgcgaatacgaaaggaggaggtgtgatggaatcgcaagaaaaagaacaatggcgtgggttattcccggcacttttgggactttttgctgtgttaatgggagcctacttttggccatggggaggtggtggctctatggccgataaggttgaagcgaacgacacgctggcgtgcacctacgccgccctcatgctcagcgacgcgggtctgcccatcacggcggagggcattgaggccgcgtgtgtggctgccggtctgaaggtgcgcaacaccctgcccgttatttttgctcgctttctcgaaaagaagccgctggagactctctttgccgctgccgctgctacggcacctgcagagggcgccgctgctgctcctgccgctggcagtgccgcccctgccgccgcagctgccggtgctgcgccagaaaaggacacaaaggaggaggaggaagacgacgatatgggttttggcttgtttgac

SEQ ID NO: 104 - DNA, 1035 bp, synthetic construct
  source 1..1035: mol_type = other DNA
atgccgagcaccccgaccccgcagtgcgttcgtcgtctgcaaaaagaactgagcgcgctgtgccgtgaggcggaaagcttctttttcacccgtccgagcgcgaaaagcattctggtgtggtactttgttatcaaaggtccggcggataccccgtatgagggtggccgttattttggcaaactgaacttcccgccggactatccgatgaagccgccagaaatcattatcctgaccccgaacggtcgttttgaaaccaacaaaagcatttgcctgaccatgagcaactaccacccggaaaactggagcccgctgtggggcgttcgtaccatcctgaccggtctgctgagcttcatggtgggcgatgaactgaccaccggttgcatgaccagcagcgacgagctgcgtcgtaagtatgcgcgtgagagccgtcgttttaacgcggaaaagatgagcgtttacaaagagctgttcccggaggaatatcagaaggatctggaggaactgaaacgtgaggacagcgaaaagaacggtcgtaccagcggtagcgcgggttgcggtgcgaacaccaaaggtggcggtgtgatggaaagccaggagaaggaacaatggcgtggcctgtttccggcgctgctgggtctgttcgcggttctgatgggtgcgtacttttggccgtggggcggtggcggtagcatggcggataaagtggaggcgaacgacaccctggcgtgcacctatgcggcgctgatgctgagcgatgcgggtctgccgattaccgcggaaggtattgaagcggcgtgcgtggcggcgggtctgaaggttcgtaacaccctgccggtgatttttgcgcgtttcctggagaagaaaccgctggaaaccctgttcgcggcggcggcggcgaccgcgccggcggagggtgcggcggcggcgccggcggcgggtagcgcggcgccggcggcggcggcggcgggtgcggcgccggagaaggataccaaagaggaagaggaagacgatgacatgggctttggtctgttcgac

SEQ ID NO: 105 - DNA, 1035 bp, synthetic construct
  source 1..1035: mol_type = other DNA
atgccaagcacacccaccccgcagtgtgtgcggcggttgcaaaaggagctttccgccctatgccgagaggccgagtcgttttttttcacccgtccctcagcaaagagtattctggtttggtatttcgtcatcaagggtcctgcggataccccttatgaaggcggtcgctactttggcaagctgaatttcccccccgactatccaatgaaaccgcctgagattatcattttgacgccaaatggacgttttgagaccaacaagagcatttgtctcaccatgagcaattatcatccggagaattggagccctttgtggggggtccgcaccattcttacggggttgctctctttcatggtgggagacgaactcactactgggtgcatgacgagcagcgatgagttgcggaggaagtacgctcgtgagagccgtcgtttcaatgcagagaaaatgccagtatacaaggaactgtttccagaggagtatcagaaggacttggaggaattgaagcgagaggacaatgagaaaaacggtcgtatttctggaagtgctggctgtggtgcgaatacgaaaggaggaggtgtgatggaatcgcaagaaaaagagcaatggcgtgggttattcccggcacttttgggactttttgctgtgttaatgggagcctacttttggccatggggaggtggtggctctatggccgataaggttgaagcgaacgacacgctggcgtgcacctacgccgccctcatgcttagcgacgcgggtctgcccatcacggcggagggcattgaggccgcgtgtgtggctgccggtctgaaggtgcgcaacaccctgcccgttatttttgctcgctttcttgaaaagaagccgctggagagtctcttcgctgctgccgctgctacggctcctgcagagggcgccgctgctgttcctgccgctggcagtgccgcccctgctgccgcagctgccgctgctgcgccagcaaaggacacaaaggaggaggaggaagacgacgatatgggttttggcttgtttgac

SEQ ID NO: 106 - DNA, 1035 bp, synthetic construct
  source 1..1035: mol_type = other DNA
atgccgagcaccccgaccccgcagtgcgttcgtcgtctgcaaaaagaactgagcgcgctgtgccgtgaggcggaaagcttctttttcacccgtccgagcgcgaaaagcattctggtgtggtactttgttatcaaaggtccggcggataccccgtatgagggtggccgttattttggcaaactgaacttcccgccggactatccgatgaagccgccagaaatcattatcctgaccccgaacggtcgttttgaaaccaacaaaagcatttgcctgaccatgagcaactaccacccggaaaactggagcccgctgtggggcgttcgtaccatcctgaccggtctgctgagcttcatggtgggcgatgaactgaccaccggttgcatgaccagcagcgacgagctgcgtcgtaagtatgcgcgtgagagccgtcgttttaacgcggaaaagatgccggtttacaaagagctgttcccggaggaatatcagaaggatctggaggaactgaaacgtgaggacaacgaaaagaacggtcgtattagcggtagcgcgggttgcggtgcgaacaccaaaggtggcggtgtgatggaaagccaggagaaggaacaatggcgtggcctgtttccggcgctgctgggtctgttcgcggttctgatgggtgcgtacttttggccgtggggcggtggcggtagcatggcggataaagtggaggcgaacgacaccctggcgtgcacctatgcggcgctgatgctgagcgatgcgggtctgccgattaccgcggaaggtattgaagcggcgtgcgtggcggcgggtctgaaggttcgtaacaccctgccggtgatctttgcgcgtttcctggagaagaaaccgctggaaagcctgtttgcggcggcggcggcgaccgcgccggcggagggtgcggcggcggtgccggcggcgggcagcgcggcgccggctgctgcggcggcggcggcggcgccggcgaaggataccaaagaggaagaggaagacgatgacatgggctttggtctgttcgac

SEQ ID NO: 107 - DNA, 1029 bp, synthetic construct
  source 1..1029: mol_type = other DNA
aagcatctgaaggacgagaagaccaaggttggaagtggaccggagctgttgaagagggcggcagagcagactgtgctttctctggagaaggcaaaggaggcggaggcggaggctgagaaggcggcagcggcggcgcagaaaacccgggacgcagcagagaaggcagcagcggcgcggaccttggcacaagatgttgccgcaacggccagtgcgctgctgcggcagcgggagaaggaggaggagaggcgaagagcgagggacagggtgagggcttacgttggaaatgaacgcgccgagaatgccatgagggttgcgtggctggactgggtggagggaggtggtggctctaaccatgttaagaccgataggaggagcaagaattccaaaacagagggtcttttggacgaggcggcaaagcatactgcaattgccgtaaagaaggcaaaggaggcggaggcggagtctgagaaggcggcagcggcggcgcggaaaaccctggaagcagcagagaaggcagcagcggcgcggaccttggcacaagacgttgccgcaacggccagtgcgctgctgcggcagcgggagagggaggaggagagacgaagagcgaaggaccgggaggcggcggaggccgcgaaaaaggctgccgttgctgaggtgatgaagaaatttgctgcgaagaagggaggtggtggctctaagcatgtgaaggacgagaagaccaaggttggaagtggaccggagctgttgaagagggcggcggagcagactgtgctttctctggagaaggcaaaggaggcggaggcggagactgagaaggcggcagcggcggcgcagaaaacccgggaagcagcagagaaggcagcagcggcgcagaccttggcacaagatgttgccgcaacggccattgcgctgctgcggcagcgggagaaggaggaggagaggcgaagagcgagggaccgggaggaggcggaggccgcgaagaaggctgccgttgctgaggtgatgaataaatttgctgcgaagaagggg

SEQ ID NO: 108 - DNA, 1029 bp, synthetic construct
  source 1..1029: mol_type = other DNA
aagcacctaaaagatgaaaaaacaaaagtaggctctggtccggaattgctgaaacgtgctgcggagcagaccgtgctgagcctggaaaaggcaaaagaggcggaggcagaggcggagaaggccgcagccgccgcacaaaaaactcgcgacgcagccgagaaggcggcggcggcgcgtaccctggctcaagatgttgctgcgaccgcgagcgcactgttgcgtcagcgtgaaaaagaagaagagcgtcgtcgggcgcgtgaccgtgttcgtgcatacgtgggcaacgaaagagccgagaacgcaatgcgtgtcgcgtggctggattgggttgaaggtggtggtggctctaatcatgtgaagaccgatcgccgtagcaaaaacagcaaaacggaaggcctgttagatgaagcggcgaagcacaccgcgatcgctgtgaaaaaagcgaaagaagctgaggctgagagcgaaaaggccgcggcggcggctcgtaagaccttggaggcggcggagaaagcggcagcggctcgcaccttggctcaagatgtggccgccacggcttcggcactgttgcgtcagcgtgagcgcgaggaagagcgccgtagagctaaggacagagaagcggcggaggcggcaaagaaggccgctgtggcagaggtaatgaagaaattcgcagcgaagaaaggtggcggtggcagcaaacacgttaaggacgaaaaaacaaaagttggttccggtccggaactgctgaagcgcgccgcagaacagactgttctgtccctggagaaagcgaaagaagcggaggcggaaaccgagaaggccgctgcggcggcgcagaaaacgcgtgaggcggcggaaaaggcagcagcagcgcaaacccttgcccaggacgtggcggctaccgcgattgcactgctccgtcaacgtgaaaaggaggaagaacgtcgccgcgctcgcgaccgcgaagaggcagaggcggccaagaaggccgcggtcgcagaggtcatgaataaatttgcagcgaaaaagggc

SEQ ID NO: 109 - protein, 5 aa, synthetic construct
GGGGS

SEQ ID NO: 110 - DNA, 21 bp, synthetic construct
  source 1..21: mol_type = other DNA
gatttacgttgaccagcctgc

SEQ ID NO: 111 - DNA, 18 bp, synthetic construct
  source 1..18: mol_type = other DNA
acgttgaccagcctgcag

SEQ ID NO: 112 - DNA, 18 bp, synthetic construct
  source 1..18: mol_type = other DNA
tgtgtatgggtctgtgag

SEQ ID NO: 113 - DNA, 18 bp, synthetic construct
  source 1..18: mol_type = other DNA
tgtaaaacgacggccagt

SEQ ID NO: 114 - DNA, 18 bp, synthetic construct
  source 1..18: mol_type = other DNA
tgtgtatgggtctgtgag

SEQ ID NO: 115 - DNA, 25 bp, synthetic construct
  source 1..25: mol_type = other DNA
atgctccctgcggcagcagcagtaa

SEQ ID NO: 116 - DNA, 26 bp, synthetic construct
  source 1..26: mol_type = other DNA
ttacacagcctcctctggcaaaggct

SEQ ID NO: 117 - DNA, 20 bp, synthetic construct
  source 1..20: mol_type = other DNA
tgaaacagcagcatgccaac

SEQ ID NO: 118 - DNA, 20 bp, synthetic construct
  source 1..20: mol_type = other DNA
cgcgtctgtctaccatcaga

SEQ ID NO: 119 - DNA, 22 bp, synthetic construct
  source 1..22: mol_type = other DNA
cggctcattctgatggtagaca

SEQ ID NO: 120 - DNA, 20 bp, synthetic construct
  source 1..20: mol_type = other DNA
tgtgcgttgcacactgaatg

SEQ ID NO: 121 - DNA, 20 bp, synthetic construct
  source 1..20: mol_type = other DNA
aagtgcggcatcaactacca

SEQ ID NO: 122 - DNA, 19 bp, synthetic construct
  source 1..19: mol_type = other DNA
accctcctccataccctca

SEQ ID NO: 123 - DNA, 20 bp, synthetic construct
  source 1..20: mol_type = other DNA
tctgattgcggaaagcacaa

SEQ ID NO: 124 - protein, 8 aa, synthetic construct
  SITE 8: note = BV421 (Brilliant Violet 421) fluorescent dye label
ANYKFTLV